(19) United States
(12) Patent Application Publication
(10) Pub. No.: US 2025/0011736 A1
(43) Pub. Date: Jan. 9, 2025

(54) TRANSPOSABLE MOBILE ELEMENTS WITH ENHANCED GENOMIC SITE SELECTION

(71) Applicants: Saliogen Therapeutics, Inc., Lexington, MA (US); University of Hawai'i, Honolulu, HI (US)

(72) Inventors: Joseph J. HIGGINS, Lexington, MA (US); Jesse B. OWENS, Honolulu, HI (US); Nancy CRAIG, Lexington, MA (US)

(21) Appl. No.: 18/706,257

(22) PCT Filed: Nov. 4, 2022
(86) PCT No.: PCT/US22/79294

§ 371 Date: Apr. 30, 2024

Related U.S. Application Data
(60) Provisional application No. 63/408,184, filed on Sep. 20, 2022.
(60) Provisional application No. 63/275,785, filed on Nov. 4, 2021.

(51) Int. Cl. C12N 9/12; C12N 9/22; C12N 15/90

(52) U.S. Cl. CPC C12N 9/1241; C12N 9/22; C12N 15/907; C07K 2319/81; C12N 2310/20

(57) ABSTRACT

Gene therapy compositions and methods related to transposition are provided.

### CROSS-REFERENCE TO RELATED APPLICATIONS

This application claims priority to and the benefit of U.S. Provisional Patent Application No. 63/275,785, filed on Nov. 4, 2021 and U.S. Provisional Application No. 63/408,184, filed on Sep. 20, 2022, the entire content of which are hereby incorporated herein by reference in its entirety.

### FIELD

The present disclosure relates to recombinant transposon systems and uses thereof.

### SEQUENCE LISTING

The contents of the text file submitted electronically herewith are incorporated herein by reference in their entirety: A computer readable format copy of the Sequence Listing (filename: Sequence_Listing_SAL-014PC/126933-5014.xml″; date recorded: Nov. 4, 2022; file size: 1,015,808 bytes).

### BACKGROUND

Transposable elements are genetic sequences that are found, with small exceptions, in all living organisms. Transposable elements, or transposons, have deep evolutionary origins and diversification, and have an astonishing variety of forms and shapes. See Bourque, G., Burns, K. H., Gehring, M., Gorbunova, V., Seluanov, A., Hammell, M., . . . & Feschotte, C. (2018). Ten things you should know about transposable elements. Genome Biology, 19 (1), 1-12.

A transposon movement to a new location in the human genome is performed by the action of a helper transposase enzyme that binds to an “end sequence” and inserts a donor DNA sequence at a specific DNA sequence such as the tetranucleotide, TTAA, by a “cut and paste” mechanism. The donor DNA is flanked by end sequences in living organisms such as insects (e.g., Trichnoplusia ni). Genomic DNA is excised by double strand cleavage at the hosts' donor site and the donor DNA is integrated or inserted into a specific DNA sequence such as TTAA. A dual system that uses bioengineered transposons and transposases includes (1) a source of an active helper enzyme that “excises” a donor DNA flanked by the end recognition sequences and (2) inserts the donor sequence at a specific nucleotide sequence such as TTAA. Mobilization of the DNA sequences permits the intervening nucleic acid, or a transgene, to be inserted at the specific nucleotide sequence (i.e., TTAA) without a DNA footprint.

The piggyBac transposon, from the looper moth, Trichnoplusa ni, is a bioengineered movable genetic element that transposes between vectors and human chromosomes through a “cut-and-paste” mechanism. See Zhao, S., Jiang, E., Chen, S., Gu, Y., Shangguan, A. J., Lv, T., Luo, L., & Yu, Z. (2016). PiggyBac transposon vectors: the tools of the human gene encoding. Translational Lung Cancer Research, 5 (1), 120-125. During transposition, the helper enzyme recognizes sequences located on both ends of the donor DNA, excises precisely, and then integrates the donor DNA into TTAA chromosomal sites. See Elick, T. A., Bauser, C. A., & Fraser, M. J. (1996). Excision of the piggyBac transposable element in vitro is a precise event that is enhanced by the expression of its encoded transposase. Genetica, 98 (1), 33-41; Zhao, S., Jiang, E., Chen, S., Gu, Y., Shangguan, A. J., Lv, T., Luo, L., & Yu, Z. (2016). PiggyBac transposon vectors: the tools of the human gene encoding. Translational Lung Cancer Research, 5 (1), 120-125. Because it can excise precisely, a helper enzyme is especially useful if a transgene is only transiently required. Transient integration and expression of transcription factors are important approaches to generate transgene-free induced pluripotent stem cells (iPSCs) as well as directed differentiation of specific cell types for both research and clinical use. See Woltjen, K., Michael, I. P., Mohseni, P., Desai, R., Mileikovsky, M., Hämäläinen, R., . . . & Nagy, A. (2009). piggyBac transposition reprograms fibroblasts to induced pluripotent stem cells. Nature, 458 (7239), 766-770; Yusa, K., Rad, R., Takeda, J., & Bradley, A. (2009). Generation of transgene-free induced pluripotent mouse stem cells by the piggyBac transposon. Nature Methods, 6 (5), 363-369. Removal of the transgenes is key for potential therapeutic applications of iPSCs. DNA Helper enzymes have been used as vectors for reversible integration; but reintegration can occur in 40-50% of cells. See Wang, W., Lin, C., Lu, D., Ning, Z., Cox, T., Melvin, D., . . . & Liu, P. (2008). Chromosomal transposition of PiggyBac in mouse embryonic stem cells. Proceedings of the National Academy of Sciences, 105 (27), 9290-9295. To generate iPSCs without any genetic change, a helper enzyme mutant that promotes only excision (Exc+) and not integration (Int−), would be a useful tool.

Gene therapy involves replacing a mutated gene (which causes a disease) with a healthy copy of the gene, inactivating or silencing a mutated gene that is functioning improperly (or any other gene), or introducing a new gene into chromosomes. The ability to integrate genes safely and efficiently into a host genome is essential for successful gene therapy in humans.

Currently, the most commonly used vectors for permanent or transient transfer of genes in gene therapy trials are virus-based. However, viral vectors have been shown to have serious disadvantages and safety concerns, including the risk of immunogenicity, insertional mutagenesis or oncogenesis. Viral systems are also limited in cargo size, restricting the size and number of transgenes and their regulatory elements. Accordingly, limitations of viral vectors, such as pathogenicity, immunogenicity, expensive production, and systemic instability, have proven to be major obstacles to the use of viral-based systems. In fact, re-administration of viral-based vectors can promote immune responses that can result in life threatening systemic effects and limit gene-transfer efficacy. See Hernandez, Y. J., Wang, J., Kearns, W. G., Loiler, S., Poirier, A., & Flotte, T. R. (1999). Latent adeno-associated virus infection elicits humoral but not cell-mediated immune responses in a nonhuman primate model. Journal of Virology, 73 (10), 8549-8558; M. A. Kay, D. Liu & P. M. Hoogerbrugge, Gene Therapy, 94 Proceedings of the National Academy of Sciences 12744-12746 (1997).

As compared to viral-based gene transfer systems, bioengineered transposons are less likely to activate a protooncogene than lentivirus or other retroviruses because of TTAA site specificity. Nevertheless, the main concern in transposase-based gene therapy is insertional mutagenesis due to integration, albeit mostly at known sequences (e.g., TTAA sequences), near or within loci that activate oncogenes, interrupt tumor-suppressor genes, or disrupt the transcription of normal genes.

Thus, while non-viral, transposon gene therapy approaches have great promise for treating individuals with genetic disorders, a challenge is to reduce the risk of insertional mutagenesis or oncogenesis. Significant efforts are required to adapt transposable elements for safe and effective use in humans.

There is a need for novel transposable elements that are suitable for use in humans and that efficiently target human genomes with reduced risk of off-target effects.

### SUMMARY

Accordingly, the present disclosure provides, inter alia, excision competent/integration defective (Exc+Int−) helper enzymes (hyperactive transposases) by introducing novel Exc+Int− mutations into a heretofore undescribed hyperactive version of piggyBac helper with 11 mutations and optionally an accompanying fusion protein with, e.g., a Cas9 protein devoid of nucleolytic activity or a TALE DNA binding domain or a zinc finger DNA binding domain. In embodiments, the helper enzymes are capable of specifically targeting to a location in the genome with or without being identified as Exe+Int−. The present disclosure, in embodiments, provides a composition comprising a recombinant transposase enzyme that has bioengineered enhanced gene cleavage [Excision (Exc+)] and/or integration deficient (Int−) and/or integration efficient (Int+) gene activity, and DNA binding domains (e.g., without limitation, a dCas9 or TALE or zinc finger) that guide donor insertion to specific genomic sites.

The present disclosure also provides, inter alia, unique helper enzyme fusion molecules with, e.g., helper enzymes operationally linked to the N- or C-terminus or a DNA binding domain, in which the DNA binding domain, is located within the helper enzyme, e.g., not at the N- or C-termini, that guide donor insertion to specific genomic sites. In embodiments, the helper enzyme is an engineered form of a transposase enzyme reconstructed from Trichnoplusa ni. In embodiments, the transposase enzyme includes but is not limited to an engineered version that is a monomer, dimer, tetramer (or another multimer), hyperactive (Exc+), and/or has a reduced interaction with target DNA (Int−), of a helper enzyme reconstructed from Trichnoplusa ni or a predecessor thereof.

In some embodiments, the helper enzyme, having gene cleavage (Exc) and/or gene integration (Int) activity, has at least about 90% identity to the amino acid sequence of SEQ ID NO: 1, SEQ ID NO: 2, or SEQ ID NO: 3, or a nucleotide sequence encoding the same. In some embodiments, the helper enzyme has at least about 95%, or at least about 96%, at least about 97%, at least about 98%, at least about 99% identity to the amino acid sequence variants or combination thereof shown in TABLE 3 and TABLE 4, or a nucleotide sequence encoding the same.

In embodiments, the enzyme of the present disclosure comprises an amino acid sequence having at least about 80% sequence identity to SEQ ID NO: 11, and wherein the enzyme comprises an insertion at positions V371-1378, Y312-V322, K407-M413, S385-T392, A424-K432, and/or R275-K290 or positions V390, R315, G321, R376, S387, K409, and/or E428 of SEQ ID NO: 11.

In embodiments, the enzyme of the present disclosure comprises an amino acid sequence having at least about 80% sequence identity to SEQ ID NO: 11, and wherein the enzyme comprises an insertion in a loop domain of piggyBac. In embodiments, the enzyme of the present disclosure comprises an amino acid sequence having at least about 80% sequence identity to SEQ ID NO: 11, and wherein the enzyme comprises an insertion in a loop domain of piggyBac selected from one or more the domains of TABLE 12 (with reference to SEQ ID NO: 11).

In aspects, there is provided a composition comprising an enzyme and a targeting element which directs the enzyme to a target site, optionally a genomic safe harbor site (GSHS), wherein the enzyme is a piggyBac transposase which comprises one or more mutations which cause decreased or ablated integration activity relative to an enzyme comprising an amino acid sequence of SEQ ID NO: 11 or functional equivalent thereof.

In embodiments, the piggyBac transposase comprises at least one substitution at positions corresponding to: 315, 372, 312, 324, 347, 374, and/or 375 of SEQ ID NO: 11, and/or wherein the enzyme comprises at least one substitution selected from R315A, R372A, Y312A, L324A, N347A, N374A, and K375A, wherein the positions are corresponding to positions of SEQ ID NO: 11. In embodiments, the piggyBac transposase comprises one of R315A/R372A, R372A/K375A, N347A/R315A, L324A/Y312A, N374A, L324A/R315A, R315A/R372A/K375A, and L324A/N347A substitutions, wherein the positions are corresponding to positions of SEQ ID NO: 11. In embodiments, the piggyBac transposase comprises R315A and R372A substitutions, wherein the positions are corresponding to positions of SEQ ID NO: 11. In embodiments, the piggyBac transposase has an amino acid sequence of at least 90% identity to SEQ ID NO: 11 and R315A and R372A substitutions, wherein the positions are corresponding to positions of SEQ ID NO: 11. In embodiments, the piggyBac transposase has an amino acid sequence of at least 95% identity to SEQ ID NO: 11 and R315A and R372A substitutions, wherein the positions are corresponding to positions of SEQ ID NO: 11. In embodiments, the piggyBac transposase has an amino acid sequence of at least 98% identity to SEQ ID NO: 11 and R315A and R372A substitutions, wherein the positions are corresponding to positions of SEQ ID NO: 11. In embodiments, the piggyBac transposase comprises at least one substitution selected from FIG. 8's mutations, wherein the positions are corresponding to positions of SEQ ID NO: 11

In embodiments, the GSHS is selected from adeno-associated virus site 1 (AAVS1), chemokine (C-C motif) receptor 5 (CCR5) gene, HIV-1 coreceptor, and human Rosa26 locus.

In embodiments, the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11. In embodiments, the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N terminal deletion. In embodiments, the N terminal deletion yields reduced or ablated off-target effects of the enzyme compared to the enzyme without the N-terminal deletion. In embodiments, the enzyme comprises one or more N-terminal deletions of TABLE 11.

In embodiments, the enzyme and the targeting element are fused to one another or linked via a linker or linker domain to one another. In embodiments, the targeting element and/or the linker or linker domain are fused to the N- or C-terminus of the enzyme or inserted into the enzyme at one or more internal loops of the enzyme. In embodiments, the enzyme comprises an insertion in a loop domain of selected from one or more the domains of TABLE 12, with reference to SEQ ID NO: 11. In embodiments, the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11.

In aspects, there is provided a composition comprising an enzyme and a targeting element which directs the enzyme to a target site, optionally a genomic safe harbor site (GSHS) and optionally a linker or linking domain which connects the enzyme and targeting element, wherein the enzyme is a piggyBac transposase and the targeting element and/or linker or linking domain are fused to the N- or C-terminus of the piggyBac transposase or inserted into the piggyBac transposase at one or more internal loops of the enzyme.

In embodiments, the enzyme comprises an insertion in a loop domain of selected from one or more the domains of TABLE 12, with reference to SEQ ID NO: 11. In embodiments, the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11. In embodiments, the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N or C terminal deletion. In embodiments, the N or C terminal deletion yields reduced or ablated off-target effects of the enzyme compared to the enzyme without the N- or C-terminal deletion. In embodiments, the enzyme comprises one or more N- or C-terminal deletions of TABLE 11.

In embodiments, the composition is a nucleic acid, optionally an RNA. In embodiments, the composition further comprises a donor nucleic acid and/or is suitable for inserting a donor nucleic acid into a genome. In embodiments, the donor nucleic acid is or comprises DNA. In embodiments, the composition is in the form of a lipid nanoparticle (LNP). In embodiments, the nucleic acid encoding the enzyme and the donor nucleic acid are in the same LNP. In embodiments, the present disclosure provides a host cell comprising the LNP of the present disclosure.

In aspects, there is provided a method for inserting a gene into the genome of a cell, comprising contacting a cell with the composition described herein. In aspects, there is provided a method for treating a disease or disorder ex vivo, comprising contacting a cell with the composition described herein and administering the cell to a subject in need thereof. In aspects, there is provided a method for treating a disease or disorder in vivo, comprising administering the composition described herein to a subject in need thereof.

In embodiments, the helper enzyme has one or more mutations which confer hyperactivity. In some embodiments, the transposase enzyme has an amino acid sequence having mutations at positions which correspond to at least one of 130V, S103P, G165S, M282V, S509G, N538K, N571S, D450N, 182N, V109A, and Q591R mutations relative to the amino acid sequence of SEQ ID NO: 1 (hyperactive transposase) or a functional equivalent thereof.

In embodiments, the helper enzyme has one or more mutations which confer hyperactivity. In some embodiments, the transposase enzyme has an amino acid sequence having mutations at positions which correspond to at least one of 130V, S103P, G165S, M282V, S509G, N538K, N571S, D450N, 182N, V109A, and Q591R mutations relative to the amino acid sequence of SEQ ID NO: 1 (hyperactive transposase) and fused to the amino acid sequence of SEQ ID NO: 4 (dCas9), or a functional equivalent thereof (e.g., without limitation, by the specific insertion as described herein).

In some embodiments, the transposase enzyme has an amino acid sequence having mutations in at least one of positions 30, 82, 103, 109, 165, 282, 450, 509, 538, 571, and 591, relative to the amino acid sequence of SEQ ID NO: 1 or a functional equivalent thereof.

In some embodiments, the transposase enzyme has an amino acid sequence having mutations in at least one of positions 30, 82, 103, 109, 165, 282, 450, 509, 538, 571, and 591, relative to the amino acid sequence of SEQ ID NO: 2 or a functional equivalent thereof.

In some embodiments, the transposase enzyme has an amino acid sequence having a mutation in positions 30, 82, 103, 109, 165, 282, 450, 509, 538, 571, and 591, relative to the amino acid sequence of SEQ ID NO: 3 or a functional equivalent thereof.

In some embodiments, the transposase enzyme has an amino acid sequence having a mutation in positions 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, 504, relative to the amino acid sequence of SEQ ID NO: 1 or a functional equivalent or combination thereof.

In some embodiments, the transposase enzyme has an amino acid sequence having a mutation in positions 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, 504, relative to the amino acid sequence of SEQ ID NO: 2 or a functional equivalent or combination thereof.

In some embodiments, the transposase enzyme has an amino acid sequence having a mutation in positions 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, 504, relative to the amino acid sequence of SEQ ID NO: 3 or a functional equivalent or combination thereof.

In some embodiments, the transposase enzyme has the nucleotide sequence having about 90% identity to SEQ ID NO: 7, or a codon-optimized form thereof.

In some embodiments, the dCas9 fused to the transposase enzyme has the nucleotide sequence having about 90% identity to SEQ ID NO: 10, or a codon-optimized form thereof.

In some embodiments, the composition comprises a gene transfer construct. The gene transfer donor DNA construct can be or can comprise a vector comprising a transposon comprising one or more end sequences recognized by the transposase enzyme. In some embodiments, the end sequences are left and right end sequences that are recombinant or synthetic sequences. In embodiments, the end sequences are selected from Trichnoplusia ni, or end sequences with similarity to piggyBac-like mobile elements and exhibit duplications of their presumed TTAA target sites. In some embodiments, the end sequences are selected from nucleotide sequences of SEQ ID NO: 5, and SEQ ID NO: 6, or a nucleotide sequence having at least about 90% identity thereto.

In some embodiments, the end sequences include at least one repeat from a nucleotide sequence having at least about 90% identity to the nucleotide sequence of SEQ ID NO: 5, and wherein the at least one repeat from the nucleotide sequence having at least about 90% identity to the nucleotide sequence of SEQ ID NO: 5 is positioned at the 5′ end of the transposon. The end sequences can further include at least one repeat from a nucleotide sequence having at least about 90% identity to the nucleotide sequence of SEQ ID NO: 6, and wherein the at least one repeat from the nucleotide sequence having at least about 90% identity to the nucleotide sequence of SEQ ID NO: 6 is positioned at the 3′ end of the transposon. The end sequences, which can be, e.g., Trichnoplusia ni, are optionally flanked by a TTAA sequence.

In some embodiments, the transposase enzyme is included in the gene transfer construct. In some embodiments, the composition comprises a nucleic acid binding component of a gene-editing system. In some embodiments, the gene-editing system is included in the gene transfer construct.

In some embodiments, the gene-editing system comprises Cas9, or a variant thereof. In some embodiments, the gene-editing system comprises a nuclease-deficient dCas9. In some embodiments, the gene-editing system comprises Cas12, or a variant thereof. For example, the gene-editing system comprises a nuclease-deficient dCas12. In some embodiments, the gene-editing system comprises Cas12j, such as, for example, nuclease-deficient dCas12j.

In some embodiments, the composition has the transposase enzyme and the nucleic acid binding component of the gene-editing system.

In some embodiments, the composition comprises a chimeric transposase construct comprising the transposase enzyme and the nucleic acid binding component of the gene-editing system fused or linked thereto. The transposase enzyme and the nucleic acid binding component of the gene-editing system can be fused or linked to one another via a linker, which can be a flexible linker. The flexible linker can be substantially comprised of glycine and serine residues, optionally wherein the flexible linker comprises (Gly4Ser)ₙ, where n is from about 1 to about 12. In some embodiments, the flexible linker is of or about 50, or about 100, or about 150, or about 200 amino acid residues. In some embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In some embodiments, the flexible linker comprises from about 450 nt to about 500 nt. In some embodiments, the transposase enzyme is capable of inserting a transposon at a TA dinucleotide site or a TTAA tetranucleotide site in a target site or a genomic safe harbor site (GSHS) of a nucleic acid molecule.

In some embodiments, the transposon comprises a gene encoding a complete polypeptide. In some embodiments, the transposon comprises a gene which is defective or substantially absent in a disease state.

In some aspects, a composition is provided comprising (a) a nucleic acid binding component of a gene-editing system, and (b) a recombinant mammalian transposase enzyme, the transposase enzyme having at least about 90% identity to the amino acid sequence of SEQ ID NO: 1, or a nucleotide sequence encoding the same. In some embodiments, the transposase enzyme has at least about 95%, or at least about 96%, at least about 97%, at least about 98%, at least about 99% identity to the amino acid sequence of SEQ ID NO: 1, or a nucleotide sequence encoding the same.

In some embodiments, a transposase construct comprises a transposase (both herein called “transposase”) fused or linked to a DNA binding domain (DBD), or inactive Cas protein (dCas9) programmed by a guide RNA (gRNA) as shown in FIGS. 1A-C. Another Cas protein such as, e.g., inactive dCas12a or dCas12j can be used in the transposase construct shown in FIGS. 1A-C or in a similar transposase construct.

A composition comprising a recombinant mammalian transposase enzyme in accordance with embodiments of the present disclosure can include one or more non-viral vectors. Also, the recombinant mammalian transposase enzyme can be disposed on the same (cis) or different vector (trans) than a transposon with a transgene. Accordingly, in some embodiments, the recombinant mammalian transposase enzyme and the transposon encompassing a transgene are in cis configuration such that they are included in the same vector. In some embodiments, the recombinant mammalian transposase enzyme and the transposon encompassing a transgene are in trans configuration such that they are included in different vectors. The vector is any non-viral vector in accordance with the present disclosure.

In some aspects, a nucleic acid encoding a recombinant mammalian transposase enzyme in accordance with embodiments of the present disclosure is provided. The nucleic acid can be DNA or RNA. In some embodiments, the nucleic acid is DNA. In some embodiments, the nucleic acid is RNA that has a 5′-m7G cap (cap 0, cap1, or cap2) with pseudouridine or N-methyl-pseudouridine substitution, and a poly-A tail of or about 30, or about 50, or about 100, of about 150 nucleotides in length. In some embodiments, the recombinant mammalian transposase enzyme is incorporated into a vector. In some embodiments, the vector is a non-viral vector.

In some aspects, a host cell comprising the nucleic acid in accordance with embodiments of the present disclosure is provided.

In some embodiments, a composition or a nucleic acid in accordance with embodiments of the present disclosure is provided wherein the composition is in the form of a lipid nanoparticle (LNP). The composition can comprise one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly (lactic-co-glycolic acid) (PLGA), and N-Acetylgalactosamine (GalNAc).

In some embodiments, an LNP can be as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In some aspects, a method for inserting a gene into the genome of a cell is provided that comprises contacting a cell with a recombinant mammalian transposase enzyme in accordance with embodiments of the present disclosure. The method can be in vivo or ex vivo method.

In some embodiments, the cell is contacted with a nucleic acid encoding the transposase enzyme. In some embodiments, the nucleic acid further comprises a transposon having a gene. In some embodiments, the cell is contacted with a construct comprising a transposon having a gene.

In some embodiments, the cell is contacted with an RNA encoding the transposase enzyme.

In some embodiments, the cell is contacted with a DNA encoding the transposase enzyme. In some embodiments, the cell is contacted with a DNA encoding the transposon. In some embodiments, the transposon is flanked by one or more end sequences, such as left and right end sequences. In some embodiments, the transposon can be under control of a tissue-specific promoter. In some embodiments, the transposon is an ATP Binding Cassette Subfamily A Member 4 gene (ABC) transporter gene (ABCA4), or functional fragment thereof. As another example, in some embodiments, the transposon is a very low-density lipoprotein receptor gene (VLDLR) or a low-density lipoprotein receptor gene (LDLR), or a functional fragment thereof.

In some embodiments, the transposon is a gene encoding a complete polypeptide. In some embodiments, the transposon is a gene which is defective or substantially absent in a disease state.

In some embodiments, a kit is provided that comprises a recombinant mammalian transposase enzyme and/or or a nucleic acid according to any embodiments, or combination thereof, of the present disclosure, and instructions for introducing DNA into a cell using the recombinant mammalian transposase.

In embodiments, the present method, which makes use of a recombinant mammalian transposase identified in accordance with embodiments of the present disclosure, provides reduced insertional mutagenesis or oncogenesis as compared to a method with a non-chimeric transposase and as compared to non-mammalian transposases.

In embodiments, the method is used to treat an inherited or acquired disease in a patient in need thereof.

For example, in some embodiments, the method is used for treating and/or mitigating a class of Inherited Macular Degeneration (IMDs) (also referred to as Macular dystrophies (MDs), including Stargardt disease (STGD), Best disease, X-linked retinoschisis, pattern dystrophy, Sorsby fundus dystrophy and autosomal dominant drusen. The STGD can be STGD Type 1 (STGD1). In some embodiments, the STGD can be STGD Type 3 (STGD3) or STGD Type 4 (STGD4) disease. The IMD can be characterized by one or more mutations in one or more of ABCA4, ELOVL4, PROM1, BEST1, and PRPH2. The gene therapy can be performed using transposon-based vector systems, with the assistance by chimeric transposases in accordance with the present disclosure, which are provided on the same vector as the gene to be transferred (cis) or on a different vector (trans) or as RNA. The transposon can comprise an ATP binding cassette subfamily A member 4 (ABCA4), or functional fragment thereof, and the transposon-based vector systems can operate under the control of a retina-specific promoter.

In some embodiments, the method is used for treating and/or mitigating familial hypercholesterolemia (FH), such as homozygous FH(HoFH) or heterozygous FH(HeFH) or disorders associated with elevated levels of low-density lipoprotein cholesterol (LDL-C) or triglycerides. The gene therapy can be performed using transposon-based vector systems, with the assistance by chimeric transposases in accordance with the present disclosure, which are provided on the same vector (cis) as the gene to be transferred or on a different vector (trans). The transposon can comprise a very low-density lipoprotein receptor gene (VLDLR) or a low-density lipoprotein receptor gene (LDLR), or a functional fragment thereof. The transposon-based vector systems can operate under control of a liver-specific promoter. In some embodiments, the liver-specific promoter is an LP1 promoter. The LP1 promoter can be a human LP1 promoter, which can be constructed as described, e.g., in Nathwani et al. Blood vol. 107 (7) (2006): 2653-61.

In some embodiments, the promoter is a cytomegalovirus (CMV) or cytomegalovirus (CMV) enhancer fused to the chicken β-actin (CAG) promoter. See Alexopoulou et al., BMC Cell Biol. 2008; 9:2. Published 2008 Jan. 11.

It should be appreciated that any other inherited or acquired diseases can be treated and/or mitigated using the method in accordance with the present disclosure.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIGS. 1A-C depict three illustrative bioengineered helper constructs that are contained in a bacterial replication backbone (e.g., plasmid or miniplasmid) with a chicken beta-actin/cytomegalovirus (CMV) enhancer promoter (CAG), human beta-globin 5′-UTR, nuclear localization signal and a helper enzyme with 11 mutations in the Trichnoplusia ni transposase (hyperactive transposase, SEQ ID NO: 1) followed by a poly-alanine tail. FIG. 1A depicts the hyperactive transposase without a DBD used as negative control. A dead Cas9 (dCas9) (FIG. 1B) binding protein (SEQ ID NO: 4) or TALE protein (FIG. 1C) (TABLE 7, TABLE 8) were joined by a linker to the N-terminus of hyperactive transposase to target the genomic safe harbor sites hROSA 26 (FIG. 6 and FIG. 21) or AAVS1 (FIG. 22). These constructs were used to identify hyperactive transposase excision positive (EXC+) (TABLE 3) and integration deficient (INT−) mutants (TABLE 4).

FIGS. 2A-C depict three illustrative bioengineered helper constructs that are contained in a bacterial replication backbone (e.g., plasmid or miniplasmid) with a CMV promoter, nuclear localization signal and a helper enzyme with 11 mutations in the Trichnoplusia ni transposase (hyperactive transposase, SEQ ID NO: 1) followed by a poly-alanine tail.

FIG. 2A depicts the hyperactive transposase without a DBD used as negative control. FIG. 2B depicts a zinc finger DBD joined by a linker to the N-terminus of hyperactive transposase to target a sequence on a reporter plasmid.

FIG. 2C depicts a zinc finger DBD inserted into an internal flexible loop domain of hyperactive transposase to target a sequence on a reporter plasmid.

FIGS. 3A-E depict five illustrative bioengineered helper constructs that are contained in a bacterial replication backbone (e.g., plasmid or miniplasmid) with a CMV promoter, nuclear localization signal and a helper enzyme with 11 mutations in the Trichnoplusia ni transposase (hyperactive transposase, SEQ ID NO: 1) fused to a linking domain or a DBD fused to a linking domain used to bridge two helper proteins, followed by a poly-alanine tail. A linking domain joined by a linker was fused to a ZF (FIG. 3A), dCas9 (FIG. 3B), or a TALE (FIG. 3C). A second linking domain that bridges the proteins together was fused to the N-terminus, joined by a linker, (FIG. 3D) or into an internal flexible loop domain joined by a linker, (FIG. 3E) to the hyperactive transposase.

FIG. 4 depicts an illustrative core donor construct that is contained in a bacterial replication backbone (e.g., plasmid or miniplasmid) with a CMV promoter driving GFP expression and IRES to co-express a bacterial selection gene and, when used with the hyperactive transposase-Cas9 fusion helper, guide RNAs (TABLE 5 and TABLE 6) are included in the construct. Terminal inverted repeat (TIR) recognition sequences are included at the 5′-(SEQ ID NO: 5) and 3′-ends (SEQ ID NO: 6).

FIGS. 5A-B show excision activity. FIG. 5A depicts a 1% agarose gel showing the results of a PCR-based assay to test for excision activity. FIG. 5B depicts a DNA sequencing chromatogram that confirms that the samples in lanes 2-7 of FIG. 5A no longer contain the donor GFP insertion.

FIG. 6 depicts the human Rosa26 gene safe harbor target sequence. The location of the target sites that guide RNAs were designed to bind are annotated. The intended TTAA hotspot and the sequence of the eleven guide RNAs that were tested for directing insertion to Rosa26 are listed.

FIG. 7 lists genomic insertions recovered at Rosa26 from HEK293 cells following transfection of the donor plasmid and helper plasmid encoding dCas9 fused to the hyperactive transposase (FIG. 1B). Nested genomic PCR with forward primers located at the Rosa26 target sequence and reverse primers oriented out from the transposon (FIG. 4) were sequenced and aligned to the human genome using BLAST. The guide combinations are listed as well as the orientation of the insert in the genome and the flanking sequence and location of the targeted genomic insertions.

FIG. 8 depicts the results of integration and excision assays on hyperactive transposase fused to dCas9 with DNA binding domain mutants by integration activity. For excision assays, a reporter plasmid containing a PB transposon interrupting a zsGreen gene was co-transfected with a helper plasmid encoding the hyperactive transposase. Cells are cultured for 4 days. Upon successful excision of the transposon, the excision reporter plasmid reconstitutes a complete zsGreen gene and expresses zsGreen. For integration assays, the donor plasmid depicted in FIG. 4 was co-transfected with a helper plasmid encoding the hyperactive transposase. Cells are cultured for >2 weeks without antibiotic selection. Upon successful integration of the transposon into the genome the cells express GFP. Bars represent % GFP cells measured by flow cytometry. Mutations were designed to disrupt the binding of the hyperactive transposase to the target DNA. Arrows indicate mutants that have reduced integration but maintain excision. Number denotes the position of the amino acid residue relative to SEQ ID NO: 1.

FIG. 9 depicts sites along the dimerization surface of hyperactive transposase (D191, D198, R189, and D201) that decrease integration activity when mutated. Mutations in the dimerization domain cause the transposase to be integration negative. Upon re-location to the target site by tethering of a DNA binding protein such as a ZF, TALE or dCas9, the dimers become bound and restore activity leading to insertions at the target sequence.

FIG. 10 depicts the results of integration and excision assays on hyperactive transposase fused to dCas9 with dimerization domain mutants by integration activity. Bars represent % GFP cells measured by flow cytometry. Mutations were designed to disrupt the binding of the dimerization domain of the hyperactive transposase. Arrows indicate mutants that have reduced integration but maintain excision. Number denotes the position of the amino acid residue relative to SEQ ID NO: 1.

FIG. 11 depicts the results of excision and integration assays on the hyperactive transposase that contains different deletions at the N- and C-termini. Bars represent % GFP cells measured by flow cytometry. The hyperactive pB designated as “pB N0” known for high excision activity was used as a positive control. Stuffer DNA (pB Neg) that did not show expression served as negative controls. Abbreviations of test conditions are found in TABLE 11.

FIG. 12 depicts the results of excision assays on the hyperactive transposase that contains linker domain insertions at several flexible loop domains. Bars represent % GFP cells measured by flow cytometry. The hyperactive PB was a positive control known for high excision activity. Stuffer DNA did not express PB and serves as a negative control. Five E dimer loop fusions at the indicated amino acid in PB were tested for excision activities demonstrating that insertions at these loop domains are tolerated by the transposase and maintain activity.

FIG. 13 depicts the excision activity of piggyBac containing a ZF DBD insertion into loops. An excision reporter plasmid containing a PB transposon interrupting a zsGreen gene was co-transfected with a helper plasmid encoding PB containing a zinc finger DNA binding domain designed to bind a sequence in AAVS1 and that was fused into a permissive loop domain of the transposase. Upon successful excision of the transposon, the excision reporter plasmid reconstitutes a complete zsGreen gene and expresses zsGreen. Bars represent % GFP cells measured by flow cytometry. Hyperactive PB is a positive control known to successfully excise. Stuffer DNA does not express PB. Six fusions at the indicated amino acid in PB were tested demonstrating that insertions at these loop domains are tolerated by the transposase and maintain activity.

FIG. 14 depicts evidence of directing piggyBac insertions to a specific target sequence using linking domains and DNA binding domains which were fused into the loops of PB. For the plasmid to plasmid targeting assay, an on-target reporter plasmid containing an E2C ZF target sequence expresses GFP following successful insertion of the donor plasmid at the target site. An off-target reporter that was absent for the E2C target sequence reports off-target insertion not driven by E2C binding. Targeting was achieved by using a two-part bridging mechanism in which the E2C ZF was linked to PB via two linking domains that bind with each other to link the ZF to PB. One domain was fused to the ZF and the other to the N-terminus or a loop of PB. Targeting was achieved by direct fusion of the E2C ZF to the N-terminus or to the loop of PB. Description of labels: E2C R315 loop PB, the E2C ZF fused to the loop domain of PB after amino acid R315 (example of direct fusion of a DNA binding domain to a loop); E2C S387 loop R315A/R372A PB, the E2C ZF fused to the loop domain of PB after amino acid S387, PB contains the R315A/R372A excision+/integration− mutations (example of direct fusion of a DNA binding domain to a loop); E2C E428 loop R315A/R372A PB, the E2C ZF fused to the loop domain of PB after amino acid E428, PB contains the R315A/R372A excision+/integration− mutations (example of direct fusion of a DNA binding domain to a loop); E2C Nterm R315A/R372A PB, the E2C ZF fused to the N-terminus of PB, PB contains the R315A/R372A excision+/integration-mutations (example of direct fusion of a DNA binding domain to the N terminus); E2C NbAlfa+Alfa Nterm R315A/R372A PB, the E2C ZF fused to the Alfa nanobody cotransfected with the Alfa tag fused to the N-terminus of PB, PB contains the R315A/R372A excision+/integration− mutations (example of a bridging fusion approach by fusion the N terminus); E2C NbAlfa+Alfa E428 loop R315A/R372A PB, the E2C ZF fused to the Alfa nanobody cotransfected with the Alfa tag fused to the loop domain of PB after amino acid E428, PB contains the R315A/R372A excision+/integration− mutations (example of a bridging fusion approach by fusion to a loop); Puc57 stuffer, negative control DNA that does not express PB; Hyperactive PB, PB without a targeting fusion. Bars indicate percent GFP glowing cells from either the on-target reporter or the off-target reporter. E2C NbAlfa, a camelid VHH against ALFA-tagged proteins, has a nucleotide sequence of SEQ ID NO: 515 and an amino acid sequence of SEQ ID NO: 516; Alfa Nterm R315 R372 has a nucleotide sequence of SEQ ID NO: 517 and an amino acid sequence of SEQ ID NO: 518; and Alfa E428 loop R315 R372 has a nucleotide sequence of SEQ ID NO: 519 and an amino acid sequence of SEQ ID NO: 520.

FIG. 15 depicts evidence of directing piggyBac insertions to a specific target sequence using DNA binding domains which were fused into the N terminal flexible loop region of pB that comprises of amino acids: 24-128. The plasmid to plasmid targeting assay is described in FIG. 14. Description of labels: E2C E71 loop R372A PB, E2C E71 loop R372A PB, and E2C E71 loop R372A PB, each contain the E2C ZF fused to the loop domain of PB after indicated amino acid R315 (example of direct fusion of a DNA binding domain to a loop). Bars indicate percent GFP glowing cells from either the on-target reporter or the off-target reporter.

FIG. 16 depicts evidence of directing piggyBac insertions to a specific target sequence using DNA binding domains fused onto N terminal truncation mutants described in FIG. 11. Both direct fusions as well as fusions using the bridging strategy described above using one linking domain fused to the DBD and a second fused to N terminal truncated PB, resulted in targeted insertion. The plasmid to plasmid targeting assay is described in FIG. 14. Description of labels: R372A PB, transposase without an E2C fusion does not show an increase in targeting; E2C Nterm R372A PB, the E2C ZF fused to the N-terminus of PB; E2C truncation R372A PB, the E2C ZF was fused to the N-terminus of PB at the indicated truncation (24-594, 39-594, 87-594, 116-594). E2C NbAlfa+Alfa truncation R372A PB, co-transfection of two helper plasmids containing the Alfa tag and NbAlfa (nanobody) linking domains used to bridge the proteins. The Alfa tag was fused to the N-terminus of PB at the indicated truncation (87-594, 116-594); Alfa truncation R372A PB, control transfection omitting the DBD helper plasmid from the bridging approach. The Alfa tag was fused to the N-terminus of PB at the indicated truncation (87-594, 116-594) does not show an increase in targeting; Targeting rescue was demonstrated due to the addition of the second bridging helper containing the E2C DBD, compare E2C NbAlfa+Alfa 116-594 truncation R372A PB (targeting) to Alfa 116-594 truncation R372A PB (no targeting). Bars indicate percent GFP glowing cells from either the on-target reporter or the off-target reporter.

FIG. 17 depicts evidence that an excision+/integration− mutant R315A/R372A, highlighted in FIG. 8, fails to insert into a target plasmid. Addition of the E2C DNA binding domain rescues the targeting ability of the protein. Both covalent and non-covalent strategies rescue integration into the target plasmid. The plasmid to plasmid targeting assay is described in FIG. 14. Insertion rescue and sequence targeting was achieved by using a two-part bridging mechanism described above in which the E2C ZF was linked to PB via two linking domains that bind with each other to link the ZF to PB. One domain was fused to the ZF and the other to the N-terminus. Insertion rescue and sequence targeting was achieved by direct fusion of the E2C ZF to the N-terminus. Description of labels: R315A/R372A PB, Hyperactive PB containing the R315A/R372A excision+/integration− mutations without a targeting fusion that fails to target; E2C Nterm R315A/R372A PB, the E2C ZF fused to the N-terminus of PB, PB contains the R315A/R372A excision+/integration-mutations (example of direct fusion of a DNA binding domain to the N terminus); E2C NbAlfa+Alfa Nterm R315A/R372A PB, the E2C ZF fused to the Alfa nanobody cotransfected with the Alfa tag fused to the N-terminus of PB, PB contains the R315A/R372A excision+/integration− mutations (example of a bridging fusion approach by fusion the N terminus); Puc57 stuffer, negative control DNA that does not express PB; Hyperactive PB, PB without a targeting fusion. Bars indicate percent GFP glowing cells from either the on-target reporter or the off-target reporter.

FIGS. 18A-C depict evidence of directing piggyBac insertions to a specific target sequence using DNA binding domains fused into the loops of PB. The E2C ZF fused at the V390 loop was compared to a helper with an E2C fused at the N terminus and compared against a helper without an E2C fusion. The E2C loop insert simultaneously reduced off-target insertion, FIG. 18A (lowered off-target compared to hyperactive pB) and FIG. 18B (decreased PCR products) and increased the specificity of insertion at the target sequence FIG. 18B (single targeted PCR product near 400 bp). The plasmid to plasmid targeting assay is described in FIG. 14. The PCR was performed using cell lysates as template containing the two combined plasmids from the plasmid to plasmid targeting assay. The forward primer bound the reporter plasmid and the reverse primer bound the transposon. Products arose from a transfer of the transposon to the reporter plasmid. A product of approximately 400 bp indicates an insertion near the E2C target sequence. Both E2C Nterm R372A and E2C V390 loop R372A resulted in targeting the E2C sequence. The hyperactive PB without an E2C DBD did not result in a visible product at 400 bp. Reporters without an E2C target sequence did not result in targeting to the same location on the reporter plasmid (FIG. 18B). FIG. 18C shows chromatogram sequence verification that the PCR product from E2C V390 loop R372A results from a targeted insertion 9 bp from the E2C sequence on the reporter plasmid. Description of labels: E2C Nterm R372A PB, the E2C ZF fused to the N-terminus of PB (example of direct fusion of a DNA binding domain to the N terminus); E2C V390 loop R372A PB, the E2C ZF fused to the loop domain of PB after amino acid V390 (example of direct fusion of a DNA binding domain to a loop); Puc57 stuffer, negative control DNA that does not express PB; Hyperactive PB, PB without a targeting fusion. Bars indicate percent GFP glowing cells from either the on-target reporter or the off-target reporter.

FIG. 19 shows a non-limiting AlphaFold image that was used to predict the structure of the E2C DBD fused to the V390 flexible loop domain of PB. The structure suggests that the ZF (purple) contacts the target DNA near the TTAA.

FIGS. 20A-C depict evidence of directing piggyBac insertions to a specific target sequence using direct fusions and linking domain fusions of DNA binding domains at the N terminus of PB. The plasmid to plasmid targeting assay was used as template for PCR as described in FIG. 18. In FIG. 20A, a direct fusion of E2C (Nterm) or fusions using the bridging strategy with linking domains Alfa tag and Monobody resulted in insertions near the E2C target sequence (expected size ˜400 bp) for reporter plasmids containing the E2C site (+) but not for plasmids without the E2C site (−).

FIG. 20B shows sequence chromatogram verification that the PCR products from FIG. 20A resulted from targeted insertion 24 bp from the E2C sequence. FIG. 20C shows amplicon sequencing of all PCR products from FIG. 20A that was used to measure the frequency of insertions at the target TTAA located at bp 953-956 on the reporter plasmid that is 24 bp from the E2C site. Both bridging strategies using Alfa tag and monobody linking domains as well as the direct fusion to the N terminus resulted in high levels of targeted insertions on the reporter plasmid containing the E2C site (pos) but not for the reporter plasmid without the E2C site (neg).

FIG. 21 depicts the TTAA site in hROSA26 (hg38 chr3:9,396, 133-9,396,305) that is targeted by guideRNAs (TABLE 5) or TALES (TABLE 7).

FIG. 22 depicts two TTAA sites in AAVS1 (hg38 chr19:55, 112,851-55,113,324) that are targeted by guideRNAs (TABLE 6) or TALES (TABLE 8).

### DETAILED DESCRIPTION

The present disclosure is based, in part, on the discovery that the off-target insertion can be reduced by introducing mutations to prevent the transposase from integrating without a DNA binding domain. In embodiments, mutations were introduced into the DNA binding domain of the transposase to disrupt its native binding. In embodiments, the present disclosure provides piggyBac variants with mutations in the dimerization domain. In embodiments, the piggyBac variants with mutations in the dimerization domain cannot dimerize on their own independently. In embodiments, the targeting of the piggyBac can be redirect with DNA binding domains. In embodiments, such redirection with DNA binding domains can be carried out with or without a mutation in the piggyBac.

In embodiments, the present disclosure provides a piggyBac with its N-terminus truncated, in whole or in part.

In embodiments, a linking domain is fused to the N-terminal region of the present piggyBac. In embodiments, the linking domains are inserted into the flexible loop domains of the piggyBac. Without wishing to be bound by theory, the linking domains, e.g., fused to the N-terminus or inserted into the flexible loop domains, links the transposase to a separate linking domain fused to a DNA binding domain. In embodiments, the DNA binding domain is inserted into the flexible loop domains. In embodiments, the insertion of the linking domains into the loops of piggyBac is tolerated. In embodiments, excision activity is maintained after inserting the linking domains into the loops of piggyBac.

In embodiments, the insertion of a ZF directed to bind AAVS1 is provided. In embodiments, excision activity is maintained upon inserting a ZF directed to bind AAVS1.

In embodiments, the present disclosure provides a method to measure targeting events. In embodiments, a transposon that is targeted to a target sequence on a plasmid results in GFP expression. In embodiments, the present disclosure provides an assay that tests for successful targeting to a target sequence. In embodiments, the assay can be used to evaluate different targeting strategies.

In embodiments, integration-negative mutant can target when the DNA binding domain is located within loops, when bound to the N-terminal, when separated by a linking domain at the N-terminal, and/or when separated by a linking domain inserted into a loop. In embodiments, exemplary integration-negative mutant is an R315A/R372A mutant, or a mutant corresponding thereto with reference to SEQ ID NO: 11.

In embodiments, the DNA binding domain can be inserted into the N-terminal flexible region. In embodiments, the insertion of the DNA binding domain into the N-terminal flexible region can result in targeting. In embodiments, the N-terminal flexible region is positions at amino acids 24-128, or positions corresponding thereto, of SEQ ID NO: 11.

In embodiments, the fusion of the DNA binding domain to the N-terminal truncation mutants that are integration-negative is suitable for targeting. In embodiments, direct fusions to the N-terminal truncation results in targeting. In embodiments, using a linking domain results in targeting. In embodiments, when the DNA binding domain is omitted, no integration occurs. Without wishing to be bound by theory, the addition of the DNA binding domain rescues targeted integration.

In embodiments, in the absence of the DNA binding domain, the integration negative mutant has minimal or no integration activity, as assessed by the targeting assay. In embodiments, the integration negative mutant is R315A/R372A mutant, or a mutant corresponding thereto with reference to SEQ ID NO: 11. In embodiments, the targeted integration is rescued when the DNA binding domain is fused to the N-terminal. In embodiments, the targeted integration is rescued when it is connected by a linking domain.

In embodiments, the insertion of the E2C (or another linker domain) into a loop domain promotes highly specific targeting. In embodiments, an exemplary loop domain is V390 (or a position corresponding thereto with reference to SEQ ID NO: 11). In embodiments, the off-target is reduced significantly for the V390 loop insert. In embodiments, most of the inserts occur near the E2C target sequence, e.g., as shown by PCR of the plasmid to plasmid. In embodiments, the products were sequenced to verify the location of the inserts.

In aspects, there is provided a composition comprising an enzyme and a targeting element which directs the enzyme to a target site, optionally a genomic safe harbor site (GSHS), wherein the enzyme is a piggyBac transposase which comprises one or more mutations which cause decreased or ablated integration activity relative to an enzyme comprising an amino acid sequence of SEQ ID NO: 11 or functional equivalent thereof.

In embodiments, the piggyBac transposase comprises at least one substitution at positions corresponding to: 315, 372, 312, 324, 347, 374, and/or 375 of SEQ ID NO: 11, and/or wherein the enzyme comprises at least one substitution selected from R315A, R372A, Y312A, L324A, N347A, N374A, and K375A, wherein the positions are corresponding to positions of SEQ ID NO: 11. In embodiments, the piggyBac transposase comprises one of R315A/R372A, R372A/K375A, N347A/R315A, L324A/Y312A, N374A, L324A/R315A, R315A/R372A/K375A, and L324A/N347A substitutions, wherein the positions are corresponding to positions of SEQ ID NO: 11. In embodiments, the piggyBac transposase comprises R315A and R372A substitutions, wherein the positions are corresponding to positions of SEQ ID NO: 11. In embodiments, the piggyBac transposase has an amino acid sequence of at least 90% identity to SEQ ID NO: 11 and R315A and R372A substitutions, wherein the positions are corresponding to positions of SEQ ID NO: 11. In embodiments, the piggyBac transposase has an amino acid sequence of at least 95% identity to SEQ ID NO: 11 and R315A and R372A substitutions, wherein the positions are corresponding to positions of SEQ ID NO: 11. In embodiments, the piggyBac transposase has an amino acid sequence of at least 98% identity to SEQ ID NO: 11 and R315A and R372A substitutions, wherein the positions are corresponding to positions of SEQ ID NO: 11.

In embodiments, the GSHS is selected from adeno-associated virus site 1 (AAVS1), chemokine (C-C motif) receptor 5 (CCR5) gene, HIV-1 coreceptor, and human Rosa26 locus.

In embodiments, the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11. In embodiments, the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N or C terminal deletion. In embodiments, the N or C terminal deletion yields reduced or ablated off-target effects of the enzyme compared to the enzyme without the N- or C-terminal deletion. In embodiments, the enzyme comprises one or more N- or C-terminal deletions of TABLE 11.

In embodiments, the enzyme and the targeting element are fused to one another or linked via a linker or linker domain to one another. In embodiments, the targeting element and/or the linker or linker domain are fused to the N- or C-terminus of the enzyme or inserted into the enzyme at one or more internal loops of the enzyme. In embodiments, the enzyme comprises an insertion in a loop domain of selected from one or more the domains of TABLE 12, with reference to SEQ ID NO: 11. In embodiments, the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11.

In aspects, there is provided a composition comprising an enzyme and a targeting element which directs the enzyme to a target site, optionally a genomic safe harbor site (GSHS) and optionally a linker or linking domain which connects the enzyme and targeting element, wherein the enzyme is a piggyBac transposase and the targeting element and/or linker or linking domain are fused to the N- or C-terminus of the piggyBac transposase or inserted into the piggyBac transposase at one or more internal loops of the enzyme.

In embodiments, the enzyme comprises an insertion in a loop domain of selected from one or more the domains of TABLE 12, with reference to SEQ ID NO: 11. In embodiments, the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11. In embodiments, the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N or C terminal deletion. In embodiments, the N or C terminal deletion yields reduced or ablated off-target effects of the enzyme compared to the enzyme without the N- or C-terminal deletion. In embodiments, the enzyme comprises one or more N- or C-terminal deletions of TABLE 11.

In embodiments, the composition is a nucleic acid, optionally an RNA. In embodiments, the composition further comprises a donor nucleic acid and/or is suitable for inserting a donor nucleic acid into a genome. In embodiments, the donor nucleic acid is or comprises DNA. In embodiments, the composition is in the form of a lipid nanoparticle (LNP). In embodiments, the nucleic acid encoding the enzyme and the donor nucleic acid are in the same LNP. In embodiments, the present disclosure provides a host cell comprising the LNP of the present disclosure.

In aspects, there is provided a method for inserting a gene into the genome of a cell, comprising contacting a cell with the composition described herein. In aspects, there is provided a method for treating a disease or disorder ex vivo, comprising contacting a cell with the composition described herein and administering the cell to a subject in need thereof. In aspects, there is provided a method for treating a disease or disorder in vivo, comprising administering the composition described herein to a subject in need thereof.

In embodiments, the present disclosure is based, in part, on the discovery that a novel combination of 11 mutations on the piggyBac transposase enzyme unexpectedly result in a significant increase in transposition activity by the transposase enzyme. In aspects, there is provided the novel piggyBac transposase enzyme, hereinafter “hyperactive transposase”, that is engineered further to be excision positive. In aspects, there is provided a hyperactive transposase that is integration deficient. In yet other aspects, there is provided a hyperactive transposase that is excision positive and integration deficient. In aspects, there is provided a system and method of making a hyperactive transposase that is both excision positive and integration deficient. In some aspects of the present disclosure, the hyperactive transposase, system, or method serves as a novel tool in gene therapy. The transposon system (e.g., without limitation, the hyperactive transposase) utilizes the specificity of a targeting element (e.g., without limitation, a DNA-binding domain) to particular sites within a host genome, which allows using the targeting element to target any desired location in the genome. Without wishing to be bound by theory, the excision positive and integration deficient characteristics of the transposase are due to mutations that disrupt target DNA binding. Insertion at non-targeted sites is prevented due to the excision positive and integration deficient characteristics of the transposase. Without wishing to be bound by theory, the insertion is rescued at the target sequence due to the re-location of the transposase to the target DNA by the DNA binding domain. In this way, the hyperactive transposase, system, or method in accordance with the present disclosure allows achieving targeted integration of a transgene.

The piggyBac transposon became a useful tool for genetic manipulation of mammalian cells beginning in 2005. Woodard, L. E., & Wilson, M. H. (2015). piggyBac-ing models and new therapeutic strategies. Trends in Biotechnology, 33 (9), 525-533. Like other transposons, piggyBac transposon has two components, a transposon and a transposase. The piggyBac transposase facilitates the integration of the transposon specifically at TTAA sites randomly dispersed in the genome. The predicted frequency of TTAA in the genome is 1 in every 256 base-pairs of DNA sequence, making piggyBac transposon very useful for genetic engineering approaches. The piggyBac transposase unique feature of enabling the excision of the transposon in a completely seamless manner, such that it leaves no sequences or mutations behind, make PiggyBac transposon a very valuable tool. Furthermore, piggyBac offers a large cargo-carrying capacity (over 200 kb has been demonstrated) with no known upper limit. Woodard, L. E., & Wilson, M. H. (2015). piggyBac-ing models and new therapeutic strategies. Trends in Biotechnology, 33 (9), 525-533. The piggyBac technology can be used for numerous applications, including transgenesis, gene-trap screens, and gene editing.

### Transposase Enzyme

The instant disclosure provides, in embodiments, a transposase enzyme or a nucleic acid encoding the enzyme, wherein the enzyme comprises an amino acid sequence having at least about 80% sequence identity to SEQ ID NO: 11, and wherein the enzyme comprises an amino acid substitution at the position corresponding to position 450 of SEQ ID NO: 11.

amino acid sequence of piggyBac transposase
(594 aa)
SEQ ID NO: 11:

| | |
|---|---|
| 1 | MGSSLDDEHI LSALLQSDDE LVGEDSDSEI SDHVSEDDVQ |
| | SDTEEAFIDE |
| 51 | VHEVQPTSSG SEILDEQNVI EQPGSSLASN RILTLPORTI |
| | RGKNKHCWST |
| 101 | SKSTRRSRVS ALNIVRSORG PTRMCRNIYD PLLCFKLFFT |
| | DEIISEIVKW |
| 151 | TNAEISLKRR ESMTGATFRD TNEDEIYAFF GILVMTAVRK |
| | DNHMSTDDLF |
| 201 | DRSLSMVYVS VMSRDRFDFL IRCLRMDDKS IRPTLRENDV |
| | FTPVRKIWDL |
| 251 | FIHQCIQNYT PGAHLTIDEQ LLGFRGRCPF RMYIPNKPSK |
| | YGIKILMMCD |
| 301 | SGTKYMINGM PYLGRGTQTN GVPLGEYYVK ELSKPVHGSC |
| | RNITCDNWFT |
| 351 | SIPLAKNLLQ EPYKLTIVGT VRSNKREIPE VLKNSRSRPV |
| | GTSMFCFDGP |
| 401 | LTLVSYKPKP AKMVYLLSSC DEDASINEST GKPQMVMYYN |
| | QTKGGVDTLD |
| 451 | QMCSVMTCSR KTNRWPMALL YGMINIACIN SFIIYSHNVS |
| | SKGEKVQSRK |
| 501 | KFMRNLYMSL TSSFMRKRLE APTLKRYLRD NISNILPNEV |
| | PGTSDDSTEE |
| 551 | PVMKKRTYCT YCPSKIRRKA NASCKKCKKV ICREHNIDMC |
| | QSCF |

In some embodiments, the enzyme comprises an amino acid sequence of at least about 90% identity to SEQ ID NO: 11. In some embodiments, the enzyme comprises an amino acid sequence of at least about 93% identity to SEQ ID NO: 11. In some embodiments, the enzyme comprises an amino acid sequence of at least about 95% identity to SEQ ID NO: 11. In some embodiments, the enzyme comprises an amino acid sequence of at least about 98% identity to SEQ ID NO: 11. In some embodiments, the enzyme comprises an amino acid sequence of at least about 99% identity to SEQ ID NO: 11. In some embodiments, the substitution at position 450 is with an amino acid other than aspartate (D). In some embodiments, the substitution is with a polar uncharged amino acid. In some embodiments, the polar uncharged amino acid is selected from serine(S) threonine (T), cysteine (C), asparagine (N), glutamine (Q), and proline (P). In some embodiments, the polar uncharged amino acid is asparagine (N) or glutamine (Q). In some embodiments, the polar uncharged amino acid is asparagine (N).

In some embodiments, the enzyme comprises at least one, at least five, at least seven, at least nine, or ten substitutions at positions corresponding to: 30, 82, 103, 109, 165, 282, 509, 538, 571, and/or 591 of SEQ ID NO: 11. In some embodiments, the enzyme comprises one, two, three, four, five, six, seven, eight, nine, or ten substitutions at positions corresponding to: 30, 82, 103, 109, 165, 282, 509, 538, 571, and/or 591 of SEQ ID NO: 11. In some embodiments, the enzyme comprises at least one, at least five, at least seven, at least nine, or ten substitutions selected from 130V, S103P, G165S, M282V, S509G, N538K, N571S, 182N, V109A, and Q591R, wherein the positions are corresponding to positions of SEQ ID NO: 11. In some embodiments, the enzyme comprises one, two, three, four, five, six, seven, eight, nine, or ten substitutions selected from 130V, S103P, G165S, M282V, S509G, N538K, N571S, 182N, V109A, and Q591R, wherein the positions are corresponding to positions of SEQ ID NO: 11. In some embodiments, the enzyme comprises, at the positions corresponding to positions of SEQ ID NO: 11, substitutions of D450N, 130V, S103P, G165S, M282V, S509G, N538K, N571S, 182N, V109A, and Q591R.

In some embodiments, the enzyme comprises an amino acid sequence of SEQ ID NO: 1.

| SEQ ID NO: 1: amino acid sequence of hyperactive transposase (594 aa) [includes | |
|---|---|
| eleven mutants I30V, S103P, G165S, M282V, S509G, N538K, and N571S (italicized); and | |
| D450N, I82N, V109A, and Q591R (bold underlined)] | |
| 1 | MGSSLDDEHI LSALLQSDDE LVGEDSDSEV SDHVSEDDVQ SDTEEAFIDE |
| 51 | VHEVQPTSSG SEILDEQNVI EQPGSSLASN RNLTLPQRTI RGKNKHCWST |
| 101 | SKPTRRSRAS ALNIVRSQRG PTRMCRNIYD PLLCFKLFFT DEIISEIVKW |
| 151 | TNAEISLKRR ESMTSATFRD TNEDEIYAFF GILVMTAVRK DNHMSTDDLF |
| 201 | DRSLSMVYVS VMSRDRFDFL IRCLRMDDKS IRPTLRENDV FTPVRKIWDL |
| 251 | FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT |
| 351 | SIPLAKNLLQ EPYKLTIVGT VRSNKREIPE VLKNSRSRPV GTSMFCFDGP |
| 401 | LTLVSYKPKP AKMVYLLSSC DEDASINEST GKPQMVMYYN QTKGGVDTLN |
| 451 | QMCSVMTCSR KTNRWPMALL YGMINIACIN SFIIYSHNVS SKGEKVQSRK |
| 501 | KFMRNLYMGL TSSFMRKRLE APTLKRYLRD NISNILPKEV PGTSDDSTEE |
| 551 | PVMKKRTYCT YCPSKIRRKA SASCKKCKKV ICREHNIDMC RSCF |

In some embodiments, the nucleic acid that encodes the enzyme has a nucleotide sequence of SEQ ID NO: 7, or a nucleotide sequence having at least about 90%, or at least about 93%, or at least about 95%, or at least about 97%, or at least about 98%, or at least about 99% identity thereto or a codon-optimized form thereof.

| SEQ ID NO: 7: nucleotide sequence of hyperactive transposase (1785 bp) | |
|---|---|
| 1 | ATGGGGTCTT CACTGGACGA TGAGCATATT CTGAGCGCCC TGCTGCAGAG |
| 51 | CGATGACGAG CTGGTGGGAG AGGATTCCGA TTCCGAGGTC AGTGACCACG |
| 101 | TGTCAGAGGA CGATGTGCAG AGCGACACTG AGGAAGCCTT CATCGATGAG |
| 151 | GTCCATGAAG TGCAGCCAAC AAGCTCCGGA AGCGAGATCC TGGATGAACA |
| 201 | GAACGTGATT GAACAGCCTG GCTCTAGTCT GGCTTCCAAT AGGAACCTGA |
| 251 | CACTGCCACA GCGAACTATT CGGGGCAAGA ACAAGCACTG CTGGAGCACC |
| 301 | TCCAAGCCTA CACGGAGAAG CCGCGCGTCC GCCCTGAACA TCGTGAGATC |
| 351 | CCAGAGGGGG CCAACCCGCA TGTGCCGAAA TATCTACGAC CCCCTGCTGT |
| 401 | GCTTTAAGCT GTTCTTTACA GATGAGATCA TTAGTGAAAT CGTGAAGTGG |
| 451 | ACTAACGCAG AGATTTCACT GAAAAGGCGC GAATCTATGA CTAGTGCCAC |
| 501 | CTTCAGAGAC ACAAATGAGG ATGAAATCTA CGCTTTCTTT GGCATTCTGG |
| 551 | TCATGACCGC AGTGAGGAAG GACAACCATA TGTCTACAGA CGATCTGTTT |
| 601 | GATCGCTCTC TGAGTATGGT GTATGTCTCA GTGATGAGCA GAGACAGGTT |
| 651 | CGATTTTTTG ATCCGGTGCC TGAGAATGGA CGATAAGAGC ATTCGACCTA |
| 701 | CACTGCGGGA GAATGACGTG TTCACCCCAG TGAGGAAAAT CTGGGATCTG |
| 751 | TTTATCCACC AGTGTATTCA GAACTACACA CCCGGAGCCC ATCTGACTAT |
| 801 | CGACGAACAG CTGCTGGGCT TCCGCGGGCG ATGCCCTTTT CGCGTATACA |
| 851 | TTCCAAATAA GCCCAGCAAA TATGGCATCA AGATTCTGAT GATGTGCGAT |
| 901 | TCCGGGACCA AATACATGAT CAACGGAATG CCATATCTGG GACGGGGCAC |
| 951 | CCAGACAAAT GGAGTCCCCC TGGGCGAGTA CTATGTGAAG GAACTGTCCA |
| 1001 | AACCTGTCCA CGGGTCTTGC AGAAACATCA CCTGTGACAA TTGGTTCACA |
| 1051 | TCTATTCCCC TGGCCAAGAA CCTGCTGCAG GAGCCTTATA AACTGACTAT |
| 1101 | CGTCGGAACC GTGAGAAGCA ACAAGAGGGA GATTCCCGAA GTGCTGAAGA |
| 1151 | ACAGCCGGAG CAGACCTGTC GGCACTTCTA TGTTCTGCTT TGACGGGCCA |
| 1201 | CTGACCCTGG TGAGTTACAA GCCCAAACCT GCTAAAATGG TGTATCTGCT |
| 1251 | GTCAAGCTGT GACGAGGATG CAAGCATCAA TGAATCCACC GGCAAGCCCC |
| 1301 | AGATGGTCAT GTACTATAAC CAGACTAAAG GCGGGGTGGA TACCCTGAAT |
| 1351 | CAGATGTGCT CTGTCATGAC CTGTAGTAGA AAGACAAACA GGTGGCCTAT |
| 1401 | GGCCCTGCTG TACGGGATGA TCAACATTGC TTGCATTAAT TCATTCATCA |
| 1451 | TCTACAGCCA CAACGTGTCC TCTAAGGGGG AGAAAGTCCA GTCCCGCAAG |
| 1501 | AAATTCATGC GAAATCTGTA CATGGGACTG ACCAGTAGCT TCATGAGGAA |
| 1551 | GCGCCTGGAG GCACCCACAC TGAAAAGGTA TCTGCGCGAC AACATCAGCA |
| 1601 | ATATTCTGCC TAAGGAAGTG CCAGGCACTT CCGACGATTC TACCGAGGAA |
| 1651 | CCAGTGATGA AGAAACGGAC ATACTGCACT TATTGTCCCA GCAAGATCCG |
| 1701 | ACGGAAAGCC TCCGCTTCTT GCAAGAAGTG TAAGAAAGTG ATCTGTAGAG |
| 1751 | AGCATAACAT TGATATGTGC CGGTCCTGTT TTTGA |

In some embodiments, the enzyme has increased excision activity relative to an enzyme comprising an amino acid sequence of SEQ ID NO: 11 or functional equivalent thereof. In some embodiments, the enzyme is excision positive. In some embodiments, the enzyme is integration deficient. In some embodiments, the enzyme has decreased integration activity relative to an enzyme comprising an amino acid sequence of SEQ ID NO: 11 or functional equivalent thereof.

In some embodiments, the enzyme has increased excision activity relative to an enzyme comprising an amino acid sequence of SEQ ID NO: 2 or functional equivalent thereof.

| SEQ ID NO: 2: amino acid sequence of WO_2021_110119 hyperactive piggyBac (594 aa) | |
|---|---|
| [includes ten mutants I30V, S103P, G165S, M282V, S509G, N538K, and N571S (italicized); | |
| and I82N, V109A, and Q591R (bold underlined). D450N (underlined not included)] | |
| 1 | MGSSLDDEHI LSALLQSDDE LVGEDSDSEV SDHVSEDDVQ SDTEEAFIDE |
| 51 | VHEVQPTSSG SEILDEQNVI EQPGSSLASN RNLTLPQRTI RGKNKHCWST |
| 101 | SKPTRRSRAS ALNIVRSQRG PTRMCRNIYD PLLCFKLFFT DEIISEIVKW |
| 151 | TNAEISLKRR ESMTSATFRD TNEDEIYAFF GILVMTAVRK DNHMSTDDLF |
| 201 | DRSLSMVYVS VMSRDRFDFL IRCLRMDDKS IRPTLRENDV FTPVRKIWDL |
| 251 | FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT |
| 351 | SIPLAKNLLQ EPYKLTIVGT VRSNKREIPE VLKNSRSRPV GTSMFCFDGP |
| 401 | LTLVSYKPKP AKMVYLLSSC DEDASINEST GKPQMVMYYN QTKGGVDTLD |
| 451 | QMCSVMTCSR KTNRWPMALL YGMINIACIN SFIIYSHNVS SKGEKVQSRK |
| 501 | KFMRNLYMGL TSSFMRKRLE APTLKRYLRD NISNILPKEV PGTSDDSTEE |
| 551 | PVMKKRTYCT YCPSKIRRKA SASCKKCKKV ICREHNIDMC RSCF |

In some embodiments, the enzyme has increased excision activity relative to an enzyme comprising an amino acid sequence of SEQ ID NO: 3 or functional equivalent thereof.

| SEQ ID NO: 3: amino acid sequence of WO_2020_164702 hyperactive piggyBac (594 aa) | |
|---|---|
| [includes seven mutants I30V, G165S, M282V, and N538K (italicized); I82N, V109A, and | |
| Q591R (bold underlined). S103P, D450N, S509G, and N571S (underlined not included)] | |
| 1 | MGSSLDDEHI LSALLQSDDE LVGEDSDSEV SDHVSEDDVQ SDTEEAFIDE |
| 51 | VHEVQPTSSG SEILDEQNVI EQPGSSLASN RNLTLPQRTI RGKNKHCWST |
| 101 | SKSTRRSRAS ALNIVRSQRG PTRMCRNIYD PLLCFKLFFT DEIISEIVKW |
| 151 | TNAEISLKRR ESMTSATFRD TNEDEIYAFF GILVMTAVRK DNHMSTDDLF |
| 201 | DRSLSMVYVS VMSRDRFDFL IRCLRMDDKS IRPTLRENDV FTPVRKIWDL |
| 251 | FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT |
| 351 | SIPLAKNLLQ EPYKLTIVGT VRSNKREIPE VLKNSRSRPV GTSMFCFDGP |
| 401 | LTLVSYKPKP AKMVYLLSSC DEDASINEST GKPQMVMYYN QTKGGVDTLD |
| 451 | QMCSVMTCSR KTNRWPMALL YGMINIACIN SFIIYSHNVS SKGEKVQSRK |
| 501 | KFMRNLYMSL TSSFMRKRLE APTLKRYLRD NISNILPKEV PGTSDDSTEE |
| 551 | PVMKKRTYCT YCPSKIRRKA NASCKKCKKV ICREHNIDMC RSCF |

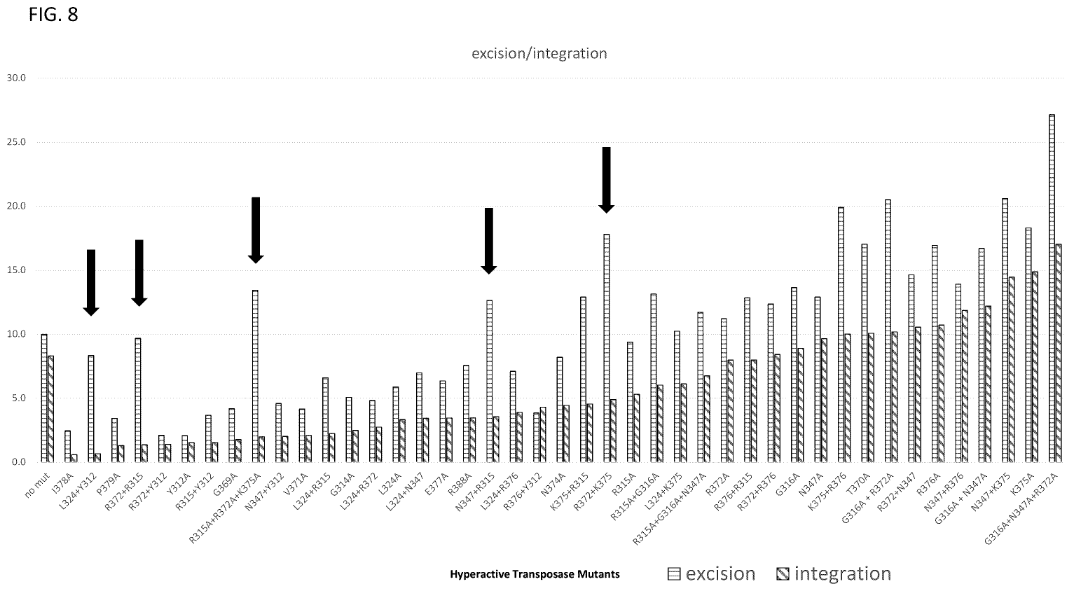

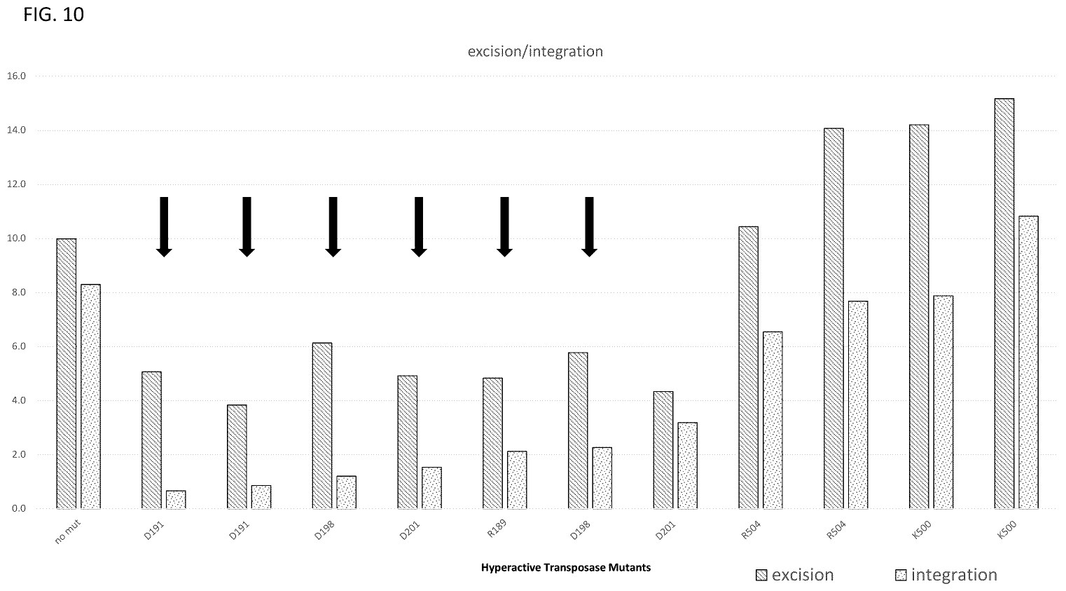

In some embodiments, the enzyme comprises at least one substitution at positions corresponding to: 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, and/or 504 of SEQ ID NO: 11. In some embodiments, the enzyme comprises at least one substitution at positions corresponding to: 312, 315, 324, 347, 372, 374, and/or 375 of SEQ ID NO: 11, and/or wherein the enzyme comprises at least one substitution selected from Y312A, R315A, L324A, N347A, R372A, N374A, and K375A, wherein the positions are corresponding to positions of SEQ ID NO: 11. In embodiments, the enzyme comprises substitution(s) selected from R372A/K375A, R372A/R315A, N347A/R315A, L324A/Y312A, N374A, L324A/R315A, R315A/R372A/K375A, and L324A/N347A, wherein the positions are corresponding to positions of SEQ ID NO: 11. In embodiments, the enzyme comprises substitution(s) selected from those of FIG. 8 or FIG. 10, wherein the positions are corresponding to positions of SEQ ID NO: 11.

In some embodiments, the enzyme comprises a targeting element. In some embodiments, the enzyme is capable of inserting a transposon comprising a transgene in a target site, optionally a genomic safe harbor site (GSHS). In some embodiments, the binding of a GSHS of a nucleic acid molecule in a mammalian cell is with high target specificity, relative to a control.

In some embodiments, the control is a composition comprising an enzyme comprising an amino acid sequence of SEQ ID NO: 11 or a nucleic acid comprising a nucleotide sequence of SEQ ID NO: 12 or a nucleotide sequence having at least about 90%, or at least about 93%, or at least about 95%, or at least about 97%, or at least about 98%, or at least about 99% identity thereto or a codon-optimized form thereof.

| SEQ ID NO: 12: nucleotide sequence encoding the wild-type piggyBac transposase (2472 nt) | |
|---|---|
| 1 | ccctagaaag atagtctgcg taaaattgac gcatgcattc ttgaaatatt gctctctctt |
| 61 | tctaaatagc gcgaatccgt cgctgtgcat ttaggacatc tcagtcgccg cttggagctc |
| 121 | ccgtgaggcg tgcttgtcaa tgcggtaagt gtcactgatt ttgaactata acgaccgcgt |
| 181 | gagtcaaaat gacgcatgat tatcttttac gtgactttta agatttaact catacgataa |
| 241 | ttatattgtt atttcatgtt ctacttacgt gataacttat tatatatata ttttcttgtt |
| 301 | atagatatcg tgactaatat ataataaaat gggtagttct ttagacgatg agcatatcct |
| 361 | ctctgctctt ctgcaaagcg atgacgagct tgttggtgag gattctgaca gtgaaatatc |
| 421 | agatcacgta agtgaagatg acgtccagag cgatacagaa gaagcgttta tagatgaggt |
| 481 | acatgaagtg cagccaacgt caagcggtag tgaaatatta gacgaacaaa atgttattga |
| 541 | acaaccaggt tcttcattgg cttctaacag aatcttgacc ttgccacaga ggactattag |
| 601 | aggtaagaat aaacattgtt ggtcaacttc aaagtccacg aggcgtagcc gagtctctgc |
| 661 | actgaacatt gtcagatctc aaagaggtcc gacgcgtatg tgccgcaata tatatgaccc |
| 721 | acttttatgc ttcaaactat tttttactga tgagataatt tcggaaattg taaaatggac |
| 781 | aaatgctgag atatcattga aacgtcggga atctatgaca ggtgctacat ttcgtgacac |
| 841 | gaatgaagat gaaatctatg ctttctttgg tattctggta atgacagcag tgagaaaaga |
| 901 | taaccacatg tccacagatg acctctttga tcgatctttg tcaatggtgt acgtctctgt |
| 961 | aatgagtcgt gatcgttttg attttttgat acgatgtctt agaatggatg acaaaagtat |
| 1021 | acggcccaca cttcgagaaa acgatgtatt tactcctgtt agaaaaatat gggatctctt |
| 1081 | tatccatcag tgcatacaaa attacactcc aggggctcat ttgaccatag atgaacagtt |
| 1141 | acttggtttt agaggacggt gtccgtttag gatgtatatc ccaaacaagc caagtaagta |
| 1201 | tggaataaaa atcctcatga tgtgtgacag tggtacgaag tatatgataa atggaatgcc |
| 1261 | ttatttggga agaggaacac agaccaacgg agtaccactc ggtgaatact acgtgaagga |
| 1321 | gttatcaaag cctgtgcacg gtagttgtcg taatattacg tgtgacaatt ggttcacctc |
| 1381 | aatccctttg gcaaaaaact tactacaaga accgtataag ttaaccattg tgggaaccgt |
| 1441 | gcgatcaaac aaacgcgaga taccggaagt actgaaaaac agtcgctcca ggccagtggg |
| 1501 | aacatcgatg ttttgttttg acggacccct tactctcgtc tcatataaac cgaagccagc |
| 1561 | taagatggta tacttattat catcttgtga tgaggatgct tctatcaacg aaagtaccgg |
| 1621 | taaaccgcaa atggttatgt attataatca aactaaaggc ggagtggaca cgctagacca |
| 1681 | aatgtgttct gtgatgacct gcagtaggaa gacgaatagg tggcctatgg cattattgta |
| 1741 | cggaatgata aacattgcct gcataaattc ttttattata tacagccata atgtcagtag |
| 1801 | caagggagaa aaggttcaaa gtcgcaaaaa atttatgaga aacctttaca tgagcctgac |
| 1861 | gtcatcgttt atgcgtaagc gtttagaagc tcctactttg aagagatatt tgcgcgataa |
| 1921 | tatctctaat attttgccaa atgaagtgcc tggtacatca gatgacagta ctgaagagcc |
| 1981 | agtaatgaaa aaacgtactt actgtactta ctgcccctct aaaataaggc gaaaggcaaa |
| 2041 | tgcatcgtgc aaaaaatgca aaaaagttat ttgtcgagag cataatattg atatgtgcca |
| 2101 | aagttgtttc tgactgacta ataagtataa tttgtttcta ttatgtataa gttaagctaa |
| 2161 | ttacttattt tataatacaa catgactgtt tttaaagtac aaaataagtt tatttttgta |
| 2221 | aaagagagaa tgtttaaaag ttttgttact ttatagaaga aattttgagt ttttgttttt |
| 2281 | ttttaataaa taaataaaca taaataaatt gtttgttgaa tttattatta gtatgtaagt |
| 2341 | gtaaatataa taaaacttaa tatctattca aattaataaa taaacctcga tatacagacc |
| 2401 | gataaaacac atgcgtcaat tttacgcatg attatcttta acgtacgtca caatatgatt |
| 2461 | atctttctag gg |

In some embodiments, the control is a composition comprising an enzyme comprising an amino acid sequence of SEQ ID NO: 2 or a nucleic acid comprising a nucleotide sequence of SEQ ID NO: 8 or a nucleotide sequence having at least about 90%, or at least about 93%, or at least about 95%, or at least about 97%, or at least about 98%, or at least about 99% identity thereto or a codon-optimized form thereof.

| SEQ ID NO: 8: nucleotide sequence of hyperactive piggyBac from WO_2021_110119 (1785 bp) | |
|---|---|
| 1 | ATGGGCAGCA GCCTGGACGA CGAGCACATC CTGAGCGCCC TGCTGCAGAG |
| 51 | CGACGACGAG CTGGTGGGCG AGGACAGCGA CAGCGAGGTG AGCGACCACG |
| 101 | TGAGCGAGGA CGACGTGCAG AGCGACACCG AGGAGGCCTT CATCGACGAG |
| 151 | GTGCACGAGG TGCAGCCCAC CAGCAGCGGC AGCGAGATCC TGGACGAGCA |
| 201 | GAACGTGATC GAGCAGCCCG GCAGCAGCCT GGCCAGCAAC CGCAACCTGA |
| 251 | CCCTGCCCCA GCGCACCATC CGCGGCAAGA ACAAGCACTG CTGGAGCACC |
| 301 | AGCAAGCCCA CCCGCCGCAG CCGCGCCAGC GCCCTGAACA TCGTGCGCAG |
| 351 | CCAGCGCGGC CCCACCCGCA TGTGCCGCAA CATCTACGAC CCCCTGCTGT |
| 401 | GCTTCAAGCT GTTCTTCACC GACGAGATCA TCAGCGAGAT CGTGAAGTGG |
| 451 | ACCAACGCCG AGATCAGCCT GAAGCGCCGC GAGAGCATGA CCAGCGCCAC |
| 501 | CTTCCGCGAC ACCAACGAGG ACGAGATCTA CGCCTTCTTC GGCATCCTGG |
| 551 | TGATGACCGC CGTGCGCAAG GACAACCACA TGAGCACCGA CGACCTGTTC |
| 601 | GACCGCAGCC TGAGCATGGT GTACGTGAGC GTGATGAGCC GCGACCGCTT |
| 651 | CGACTTCCTG ATCCGCTGCC TGCGCATGGA CGACAAGAGC ATCCGCCCCA |
| 701 | CCCTGCGCGA GAACGACGTG TTCACCCCCG TGCGCAAGAT CTGGGACCTG |
| 751 | TTCATCCACC AGTGCATCCA GAACTACACC CCCGGCGCCC ACCTGACCAT |
| 801 | CGACGAGCAG CTGCTGGGCT TCCGCGGCCG CTGCCCCTTC CGCGTGTACA |
| 851 | TCCCCAACAA GCCCAGCAAA TACGGCATCA AGATCCTGAT GATGTGCGAC |
| 901 | AGCGGCACCA AGTACATGAT CAACGGCATG CCCTACCTGG GCCGCGGCAC |
| 951 | CCAGACCAAC GGCGTGCCCC TGGGCGAGTA CTACGTGAAG GAGCTGAGCA |
| 1001 | AGCCCGTGCA CGGCAGCTGC CGCAACATCA CCTGCGACAA CTGGTTCACC |
| 1051 | AGCATCCCCC TGGCCAAGAA CCTGCTGCAG GAGCCCTACA AGCTGACCAT |
| 1101 | CGTGGGCACC GTGCGCAGCA ACAAGCGCGA GATCCCCGAG GTGCTGAAGA |
| 1151 | ACAGCCGCAG CCGCCCCGTG GGCACCAGCA TGTTCTGCTT CGACGGCCCC |
| 1201 | CTGACCCTGG TGAGCTACAA GCCCAAGCCC GCCAAGATGG TGTACCTGCT |
| 1251 | GAGCAGCTGC GACGAGGACG CCAGCATCAA CGAGAGCACC GGCAAGCCCC |
| 1301 | AGATGGTGAT GTACTACAAC CAGACCAAGG GCGGCGTGGA CACCCTGGAC |
| 1351 | CAGATGTGCA GCGTGATGAC CTGCAGCCGC AAGACCAACC GCTGGCCCAT |
| 1401 | GGCCCTGCTG TACGGCATGA TCAACATCGC CTGCATCAAC AGCTTCATCA |
| 1451 | TCTACAGCCA CAACGTGAGC AGCAAGGGCG AGAAGGTGCA GAGCCGCAAG |
| 1501 | AAGTTCATGC GCAACCTGTA CATGGGCCTG ACCAGCAGCT TCATGCGCAA |
| 1551 | GCGCCTGGAG GCCCCCACCC TGAAGCGCTA CCTGCGCGAC AACATCAGCA |
| 1601 | ACATCCTGCC CAAGGAGGTG CCCGGCACCA GCGACGACAG CACCGAGGAG |
| 1651 | CCCGTGATGA AGAAGCGCAC CTACTGCACC TACTGCCCCA GCAAGATCCG |
| 1701 | CCGCAAGGCC AGCGCCAGCT GCAAGAAGTG CAAGAAGGTG ATCTGCCGCG |
| 1751 | AGCACAACAT CGACATGTGC CGGAGCTGCT TCTAA |

In some embodiments, the control is a composition comprising an enzyme comprising an amino acid sequence of SEQ ID NO: 3 or a nucleic acid comprising a nucleotide sequence of SEQ ID NO: 9 or a nucleotide sequence having at least about 90%, or at least about 93%, or at least about 95%, or at least about 97%, or at least about 98%, or at least about 99% identity thereto or a codon-optimized form thereof.

| SEQ ID NO: 9: nucleotide sequence of hyperactive piggyBac from WO_2020_164702_A1 (1785 bp) | |
|---|---|
| 1 | ATGGGCTCTA GCCTGGACGA CGAGCACATT CTGTCTGCCC TGCTGCAGTC |
| 51 | CGACGATGAA CTCGTGGGCG AAGATTCCGA CTCCGAGATC TCTGACCACG |
| 101 | TGTCCGAGGA CGACGTGCAG TCTGATACCG AGGAAGCCTT CATCGACGAG |
| 151 | GTGCACGAAG TGCAGCCTAC CTCTTCCGGC TCTGAGATCC TGGACGAGCA |
| 201 | GAACGTGATC GAGCAGCCTG GATCCTCTCT GGCCTCCAAC AGAATCCTGA |
| 251 | CACTGCCCCA GAGAACCATC CGGGGCAAGA ACAAGCACTG CTGGTCCACC |
| 301 | TCCAAGTCTA CCCGGCGGTC TAGAGTGTCC GCTCTGAATA TTGTGCGGTC |
| 351 | CCAGAGGGGC CCCACCAGAA TGTGCCGGAA CATCTACGAC CCTCTGCTGT |
| 401 | GTTTCAAGCT GTTCTTCACC GACGAGATCA TCAGCGAGAT CGTGAAGTGG |
| 451 | ACCAACGCCG AGATCAGCCT GAAGCGGCGG GAATCTATGA CCGGCGCCAC |
| 501 | CTTCAGAGAC ACCAACGAGG ATGAGATCTA CGCCTTCTTC GGCATCCTGG |
| 551 | TCATGACAGC CGTGCGGAAG GACAACCACA TGTCCACCGA CGACCTGTTC |
| 601 | GACAGATCCC TGTCCATGGT GTACGTGTCC GTGATGAGCC GGGACAGATT |
| 651 | CGACTTCCTG ATCCGGTGCC TGCGGATGGA CGACAAGTCC ATCAGACCCA |
| 701 | CACTGCGCGA GAACGACGTG TTCACACCTG TGCGGAAGAT CTGGGACCTG |
| 751 | TTCATCCACC AGTGCATCCA GAACTACACC CCTGGCGCTC ACCTGACCAT |
| 801 | CGATGAACAG CTGCTGGGCT TCAGAGGCAG ATGCCCCTTC AGAATGTACA |
| 851 | TCCCCAACAA GCCCTCTAAG TACGGCATCA AGATCCTGAT GATGTGCGAC |
| 901 | TCCGGCACCA AGTACATGAT CAACGGCATG CCCTACCTCG GCAGAGGCAC |
| 951 | CCAAACAAAT GGCGTGCCAC TGGGCGAGTA CTATGTGAAA GAACTGTCCA |
| 1001 | AGCCTGTGCA CGGCTCCTGC AGAAACATCA CCTGTGACAA CTGGTTCACC |
| 1051 | AGCATTCCTC TGGCCAAGAA CCTGCTGCAA GAGCCCTACA AGCTGACAAT |
| 1101 | CGTGGGCACC GTGCGGTCCA ACAAGCGGGA AATTCCTGAG GTGCTGAAGA |
| 1151 | ACTCTCGGTC CAGACCTGTG GGCACCTCCA TGTTCTGTTT CGACGGCCCT |
| 1201 | CTGACACTGG TGTCCTACAA GCCTAAGCCT GCCAAGATGG TGTACCTGCT |
| 1251 | GTCCTCCTGT GACGAGGACG CCAGCATCAA TGAGTCCACC GGCAAGCCCC |
| 1301 | AGATGGTCAT GTACTACAAC CAGACCAAAG GCGGCGTGGA CACCCTGGAC |
| 1351 | CAGATGTGCT CTGTGATGAC CTGCTCCAGA AAGACCAACA GATGGCCCAT |
| 1401 | GGCTCTGCTG TACGGCATGA TCAATATCGC CTGCATCAAC AGCTTCATCA |
| 1451 | TCTACTCCCA CAACGTGTCC TCCAAGGGCG AGAAGGTGCA GTCCCGGAAG |
| 1501 | AAATTCATGC GGAACCTGTA TATGTCCCTG ACCTCCAGCT TCATGAGAAA |
| 1551 | GCGGCTGGAA GCCCCTACTC TGAAGAGATA CCTGCGGGAC AACATCTCCA |
| 1601 | ACATCCTGCC TAACGAGGTG CCCGGCACCA GCGACGATTC TACAGAGGAA |
| 1651 | CCTGTGATGA AGAAGCGGAC CTACTGCACC TACTGTCCCT CCAAGATCCG |
| 1701 | GCGGAAGGCC AACGCCTCTT GCAAAAAGTG CAAGAAAGTG ATCTGCCGCG |
| 1751 | AGCACAACAT CGACATGTGC CAGTCTTGTT TCTGA |

In some embodiments, the targeting element is able to direct a transposition machinery to the GSHS of a nucleic acid molecule in a mammalian cell. In some embodiments, the GSHS is in an open chromatin location in a chromosome. In some embodiments, the GSHS is selected from adeno-associated virus site 1 (AAVS1), chemokine (C-C motif) receptor 5 (CCR5) gene, HIV-1 coreceptor, and human Rosa26 locus. In some embodiments, the GSHS is an adeno-associated virus site 1 (AAVS1). In some embodiments, the GSHS is a human Rosa26 locus. In some embodiments, the GSHS is located on human chromosome 2, 4, 6, 10, 11, 17, or 22.

In some embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4. In some embodiments, the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, CRISPR/Cas enzymes (class I, class II), or their six subtypes (type I-VI), transcriptional activator, a transcriptional repressor, a recombinase, a DNA methyltransferase, a histone methyltransferase, or a paternally expressed gene 10 (PEG10). In some embodiments, the targeting element comprises a TALE DBD. In some embodiments, the TALE DBD comprises one or more repeat sequences. In some embodiments, the TALE DBD comprises about 14, or about 15, or about, 16, or about 17, or about 18, or about 18.5 repeat sequences. In some embodiments, the repeat sequences each independently comprises about 33 or 34 amino acids. In some embodiments, the repeat sequences each independently comprises a repeat variable di-residue (RVD) at residue 12 or 13 of the 33 or 34 amino acids, respectively. In some embodiments, the RVD recognizes one base pair in a target nucleic acid sequence. In some embodiments, the RVD recognizes a C residue in the target nucleic acid sequence and is selected from HD, N (gap), HA, ND, and HI. In some embodiments, the RVD recognizes a G residue in the target nucleic acid sequence and is selected from NN, NH, NK, HN, and NA. In some embodiments, the RVD recognizes an A residue in the target nucleic acid sequence and is selected from NI and NS. In some embodiments, the RVD recognizes a T residue in the target nucleic acid sequence and is selected from NG, HG, H(gap), and IG.

In some embodiments, the targeting element comprises a Cas9 enzyme associated with a gRNA. In some embodiments, the Cas9 enzyme associated with a gRNA comprises a catalytically inactive dCas9 associated with a gRNA. In some embodiments, the catalytically inactive dCas9 comprises at least about 90%, or at least about 95%, or at least about 96%, or at least about 97%, or at least about 98%, or at least about 99% identity to an amino acid sequence of SEQ ID NO: 4 or a nucleic acid comprising a nucleotide sequence of SEQ ID NO: 10 or a nucleotide sequence having at least about 90%, or at least about 93%, or at least about 95%, or at least about 97%, or at least about 98%, or at least about 99% identity thereto or a codon-optimized form thereof. In some embodiments, the targeting element comprises CRISPR/Cas enzymes (class I, class II), or their six subtypes (type I-VI) (e.g., (type I-VI) (e.g., Cas12a, Cas12j, Cas12k) associated with gRNA(s). In some embodiments, the targeting element comprises a catalytically inactive Cas12 associated with a gRNA, optionally wherein the catalytically inactive Cas12 is dCas12j or dCas12a. In some embodiments, the targeting element comprises a nucleic acid binding component of a gene-editing system.

In some embodiments, the enzyme or variant thereof and the targeting element are connected. In some embodiments, the enzyme and the targeting element are fused to one another or linked via a linker to one another. In some embodiments, the linker is a flexible linker. In some embodiments, the flexible linker is substantially comprised of glycine and serine residues, optionally wherein the flexible linker comprises (Gly4Ser)ₙ, where n is an integer from 1-12. In some embodiments, the flexible linker is of about 20, or about 30, or about 40, or about 50, or about 60 amino acid residues. In some embodiments, the enzyme is directly fused to the N-terminus of the dCas9 enzyme.

In some embodiments, the enzyme or variant thereof is able to directly or indirectly cause transposition of a target gene. In some embodiments, the enzyme or variant thereof is able to directly or indirectly interact and/or form a complex with one or more proteins or nucleic acids.

### Binding Domain Insertion

In embodiments, binding domains are fused within the piggyBac (PB) transposase open reading frame. In embodiments, binding domains are fused within the piggyBac (PB) transposase open reading frame covalently. In embodiments, binding domains are fused within the piggyBac (PB) transposase open reading frame at the loop domains. In embodiments, binding domains are covalently fused within the piggyBac (PB) transposase open reading frame at the loop domains. In embodiments, the loop domains tolerate insertions without inactivating the excision activity of piggyBac (PB) transposase.

In embodiments, insertions in the PB loop domains at amino acid positions V371-1378, Y312-V322, K407-M413, S385-T392, A424-K432, and/or R275-K290 relative to SEQ ID NO: 11. In embodiments, specific loop insertion sites are at positions: V390, R315, G321, R376, S387, K409, and/or E428 relative to SEQ ID NO: 11. In embodiments, the hyper active mutant comprises an insertion at one or more of positions V371-1378, Y312-V322, K407-M413, S385-T392, A424-K432, R275-K290, or R315, G321, R376, S387, K409, and E428 relative to SEQ ID NO: 11.

In embodiments, the enzyme of the present disclosure comprises an amino acid sequence having at least about 80% sequence identity to SEQ ID NO: 11, and wherein the enzyme comprises an insertion at one or more of positions V371-1378, Y312-V322, K407-M413, S385-T392, A424-K432, R275-K290, or R315, G321, R376, S387, K409, and E428 relative to SEQ ID NO: 11. In embodiments, the enzyme comprising insertion(s) at said positions may be combined with other embodiments of the present disclosure. In embodiments, the insertion is a DNA binding domain in whole, or a functional fragment that is capable of binding. In embodiments, the insertion is or comprises a DNA binding domain in whole, or a functional fragment that is capable of binding. In embodiments, the DNA binding domain is selected from: zinc finger, TAL effector (TALE), leucine zipper, CRISPR-based DNA targeting nuclease, and/or combinations thereof. In embodiments, the CRISPR-based DNA targeting nuclease is selected from Cas9 and/or dCas9.

In embodiments, the enzyme of the present disclosure comprises an amino acid sequence having at least about 80% sequence identity to SEQ ID NO: 11, and wherein the enzyme comprises an insertion in a loop domain of piggyBac. In embodiments, the enzyme of the present disclosure comprises an amino acid sequence having at least about 80% sequence identity to SEQ ID NO: 11, and wherein the enzyme comprises an insertion in a loop domain of piggyBac selected from one or more the domains of TABLE 12 (with reference to SEQ ID NO: 11):

TABLE 12
PB loop regions

S117-T122
N127-P131
F138-F139
L157-T171
K190-M194
D201-M212
L224-S230
R236-V240
N258-A263
L272-R281
I284-Y290
S301-Y305
Y312-V322
G338-R341
D346-T350
L358-I378
V371-T392
D398-L401
K407-M413
S419-K432
G444-G445
S454-R464
K492-Q497
E520-K525
N534-N571
C574-K579
C582-F594

In embodiments, the enzyme of the present disclosure comprises an amino acid sequence having at least about 80% sequence identity to SEQ ID NO: 11, and wherein the enzyme comprises an insertion at one or more of positions listed in TABLE 12 relative to SEQ ID NO: 11. In embodiments, the enzyme comprising insertion(s) at said positions may be combined with other embodiments of the present disclosure. In embodiments, the insertion is a DNA binding domain in whole, or a functional fragment that is capable of binding. In embodiments, the insertion is or comprises a DNA binding domain in whole, or a functional fragment that is capable of binding. In embodiments, the DNA binding domain is selected from: zinc finger, TAL effector (TALE), leucine zipper, CRISPR-based DNA targeting nuclease, and/or combinations thereof. In embodiments, the CRISPR-based DNA targeting nuclease is selected from Cas9 and/or dCas9.

In embodiments, the fusion molecules include domains capable of binding alternate domains from a second bridging protein. Without wishing to be bound by theory, these fusions are believed to provide a docking site for a second protein to bridge the two proteins that is also fused to a DNA binding domain. In embodiments, the configuration entails the second protein binding a target sequence, either on a plasmid or in the genome, and relocating PB to this sequence through the interaction of the domain on the bridging protein with the domain that is fused to the PB loop. In embodiments, because the PB enzyme is located near the target DNA via this bridging protein, integration occurs within close proximity to this sequence. In embodiments, the bridging protein comprises two molecules. In embodiments, the first molecule is the domain that binds to the domain located within PB. Without limitation, some examples of the domain include a camelid VHH and an antigen. Without limitation, some examples of the domain include a monobody and an antigen. Without limitation, some examples of the domain include VNAR, Fab, and scFv. Without limitation, some examples of the domain include a heterodimer. In embodiments, the heterodimer is a E/K leucine zipper. In embodiments, the second molecule of the bridging protein is a DNA binding domain. Without limitation, some examples of the DNA binding domain include zinc fingers, TALEs, Cas9, dCas9, or other CRISPR-based DNA targeting nucleases.

In embodiments, E2C NbAlfa has a nucleotide sequence of SEQ ID NO: 515 and an amino acid sequence of SEQ ID NO: 516. In embodiments, Alfa Nterm R315 R372 has a nucleotide sequence of SEQ ID NO: 517 and an amino acid sequence of SEQ ID NO: 518. In embodiments, Alfa E428 loop R315 R372 has a nucleotide sequence of SEQ ID NO: 519 and an amino acid sequence of SEQ ID NO: 520. In embodiments, E2C GCN4 has a nucleotide sequence of SEQ ID NO: 521 and an amino acid sequence of SEQ ID NO: 522. In embodiments, E2C GCN4 (GCN4 underlined) has a nucleotide sequence of SEQ ID NO: 523 and an amino acid sequence of SEQ ID NO: 524. In embodiments, Monobody Nterm R315 R372 has a nucleotide sequence of SEQ ID NO: 525 and an amino acid sequence of SEQ ID NO: 526. In embodiments, Monobody Nterm R315 R372 (monobody underlined) has a nucleotide sequence of SEQ ID NO: 527 and an amino acid sequence of SEQ ID NO: 528.

SEQ ID NO: 515: nucleotide sequence of E2C NbAlfa
ATGGCGCCTAAGAAGAAGAGAAAGGTCGGCGGCCTCGAACCAGGCGAGAAGCCTTATGCCTGTCCTGAGTGTGGCAA
ATCCTTCTCAAGAAAAGACTCTCTGGTTAGACACCAGAGAACACATACAGGGGAGAAACCCTATAAATGCCCCGAAT
GCGGAAAGTCCTTTTCCCAGAGCGGCGATCTCCGGAGGCATCAGAGAACTCATACAGGCGAGAAACCATATAAGTGC
CCCGAGTGTGGGAAATCCTTTTCCGATTGTAGAGACCTGGCCAGACATCAAAGGACACATACAGGCAAGAAGACCGC
TAGCGGTGGATCCGGAGGGTCCGGAGGTAGTGGCGGCAGCCTCGAGGAAGTCCAGCTCCAAGAAAGCGGCGGGGGCC
TCGTGCAGCCAGGCGGGTCCCTGAGGCTGAGCTGTACCGCATCTGGAGTCACCATCTCTGCCCTTAACGCAATGGCT
ATGGGGTGGTATCGGCAGGCGCCCGGCGAGAGGAGAGTCATGGTAGCAGCCGTTAGCGAAAGGGGGAATGCGATGTA
CCGAGAGAGTGTTCAGGGACGATTTACTGTCACTCGGGATTTTACGAACAAAATGGTTTCATTGCAAATGGACAATT
TGAAACCAGAGGACACCGCTGTTTACTACTGTCACGTCCTGGAAGATCGAGTAGATAGCTTCCATGACTATTGGGGC
CAAGGAACACAAGTGACTGTCAGCTCC
SEQ ID NO: 516: amino acid sequence of E2C NbAlfa
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEVQLQESGGGLVQPGGSLRLSCTASGVTISALNAMA
MGWYRQAPGERRVMVAAVSERGNAMYRESVQGRFTVTRDFTNKMVSLQMDNLKPEDTAVYYCHVLEDRVDSFHDYWG
QGTQVTVSS
SEQ ID NO: 517: nucleotide sequence of Alfa Nterm R315 R372
ATGGCGCCTAAGAAGAAGAGAAAGGTCGGCGGCTCCAGACTGGAAGAGGAACTGAGAAGAAGGCTCACAGAAGCTAG
CGGTGGATCCGGAGGGTCCGGAGGTAGTGGCGGCAGCCTCGAGGGGTCTTCACTGGACGATGAGCATATTCTGAGCG
CCCTGCTGCAGAGCGATGACGAGCTGGTGGGAGAGGATTCCGATTCCGAGGTCAGTGACCACGTGTCAGAGGACGAT
GTGCAGAGCGACACTGAGGAAGCCTTCATCGATGAGGTCCATGAAGTGCAGCCAACAAGCTCCGGAAGCGAGATCCT
GGATGAACAGAACGTGATTGAACAGCCTGGCTCTAGTCTGGCTTCCAATAGGAACCTGACACTGCCACAGCGAACTA
TTCGGGGCAAGAACAAGCACTGCTGGAGCACCTCCAAGCCTACACGGAGAAGCCGCGCGTCCGCCCTGAACATCGTG
AGATCCCAGAGGGGGCCAACCCGCATGTGCCGAAATATCTACGACCCCCTGCTGTGCTTTAAGCTGTTCTTTACAGA
TGAGATCATTAGTGAAATCGTGAAGTGGACTAACGCAGAGATTTCACTGAAAAGGCGCGAATCTATGACTAGTGCCA
CCTTCAGAGACACAAATGAGGATGAAATCTACGCTTTCTTTGGCATTCTGGTCATGACCGCAGTGAGGAAGGACAAC
CATATGTCTACAGACGATCTGTTTGATCGCTCTCTGAGTATGGTGTATGTCTCAGTGATGAGCAGAGACAGGTTCGA
TTTTTTGATCCGGTGCCTGAGAATGGACGATAAGAGCATTCGACCTACACTGCGGGAGAATGACGTGTTCACCCCAG
TGAGGAAAATCTGGGATCTGTTTATCCACCAGTGTATTCAGAACTACACACCCGGAGCCCATCTGACTATCGACGAA
CAGCTGCTGGGCTTCCGCGGGCGATGCCCTTTTCGCGTATACATTCCAAATAAGCCCAGCAAATATGGCATCAAGAT
TCTGATGATGTGCGATTCCGGGACCAAATACATGATCAACGGAATGCCATATCTGGGAGCCGGCACCCAGACAAATG
GAGTCCCCCTGGGCGAGTACTATGTGAAGGAACTGTCCAAACCTGTCCACGGGTCTTGCAGAAACATCACCTGTGAC
AATTGGTTCACATCTATTCCCCTGGCCAAGAACCTGCTGCAGGAGCCTTATAAACTGACTATCGTCGGAACCGTGGC
CAGCAACAAGAGGGAGATTCCCGAAGTGCTGAAGAACAGCCGGAGCAGACCTGTCGGCACTTCTATGTTCTGCTTTG
ACGGGCCACTGACCCTGGTGAGTTACAAGCCCAAACCTGCTAAAATGGTGTATCTGCTGTCAAGCTGTGACGAGGAT
GCAAGCATCAATGAATCCACCGGCAAGCCCCAGATGGTCATGTACTATAACCAGACTAAAGGCGGGGTGGATACCCT
GAATCAGATGTGCTCTGTCATGACCTGTAGTAGAAAGACAAACAGGTGGCCTATGGCCCTGCTGTACGGGATGATCA
ACATTGCTTGCATTAATTCATTCATCATCTACAGCCACAACGTGTCCTCTAAGGGGGAGAAAGTCCAGTCCCGCAAG
AAATTCATGCGAAATCTGTACATGGGACTGACCAGTAGCTTCATGAGGAAGCGCCTGGAGGCACCCACACTGAAAAG
GTATCTGCGCGACAACATCAGCAATATTCTGCCTAAGGAAGTGCCAGGCACTTCCGACGATTCTACCGAGGAACCAG
TGATGAAGAAACGGACATACTGCACTTATTGTCCCAGCAAGATCCGACGGAAAGCCTCCGCTTCTTGCAAGAAGTGT
AAGAAAGTGATCTGTAGAGAGCATAACATTGATATGTGCCGGTCCTGTTTT
SEQ ID NO: 518: amino acid sequence of Alfa Nterm R315 R372
MAPKKKRKVGGSRLEEELRRRLTEASGGSGGSGGSGGSLEGSSLDDEHILSALLQSDDELVGEDSDSEVSDHVSEDD
VQSDTEEAFIDEVHEVQPTSSGSEILDEQNVIEQPGSSLASNRNLTLPQRTIRGKNKHCWSTSKPTRRSRASALNIV
RSQRGPTRMCRNIYDPLLCFKLFFTDEIISEIVKWTNAEISLKRRESMTSATFRDTNEDEIYAFFGILVMTAVRKDN
HMSTDDLFDRSLSMVYVSVMSRDRFDFLIRCLRMDDKSIRPTLRENDVFTPVRKIWDLFIHQCIQNYTPGAHLTIDE
QLLGFRGRCPFRVYIPNKPSKYGIKILMMCDSGTKYMINGMPYLGAGTQTNGVPLGEYYVKELSKPVHGSCRNITCD
NWFTSIPLAKNLLQEPYKLTIVGTVASNKREIPEVLKNSRSRPVGTSMFCFDGPLTLVSYKPKPAKMVYLLSSCDED
ASINESTGKPQMVMYYNQTKGGVDTLNQMCSVMTCSRKTNRWPMALLYGMINIACINSFIIYSHNVSSKGEKVQSRK
KFMRNLYMGLTSSFMRKRLEAPTLKRYLRDNISNILPKEVPGTSDDSTEEPVMKKRTYCTYCPSKIRRKASASCKKC
KKVICREHNIDMCRSCF
SEQ ID NO: 519: nucleotide sequence of Alfa E428 loop R315 R372
ATGCCGAAAAAAAAACGAAAGGTGTACCCCTACGATGTACCGGACTATGCAGGAAGCGGGTCTTCACTGGACGATGA
GCATATTCTGAGCGCCCTGCTGCAGAGCGATGACGAGCTGGTGGGAGAGGATTCCGATTCCGAGGTCAGTGACCACG
TGTCAGAGGACGATGTGCAGAGCGACACTGAGGAAGCCTTCATCGATGAGGTCCATGAAGTGCAGCCAACAAGCTCC
GGAAGCGAGATCCTGGATGAACAGAACGTGATTGAACAGCCTGGCTCTAGTCTGGCTTCCAATAGGAACCTGACACT
GCCACAGCGAACTATTCGGGGCAAGAACAAGCACTGCTGGAGCACCTCCAAGCCTACACGGAGAAGCCGCGCGTCCG
CCCTGAACATCGTGAGATCCCAGAGGGGGCCAACCCGCATGTGCCGAAATATCTACGACCCCCTGCTGTGCTTTAAG
CTGTTCTTTACAGATGAGATCATTAGTGAAATCGTGAAGTGGACTAACGCAGAGATTTCACTGAAAAGGCGCGAATC
TATGACTAGTGCCACCTTCAGAGACACAAATGAGGATGAAATCTACGCTTTCTTTGGCATTCTGGTCATGACCGCAG
TGAGGAAGGACAACCATATGTCTACAGACGATCTGTTTGATCGCTCTCTGAGTATGGTGTATGTCTCAGTGATGAGC
AGAGACAGGTTCGATTTTTTGATCCGGTGCCTGAGAATGGACGATAAGAGCATTCGACCTACACTGCGGGAGAATGA
CGTGTTCACCCCAGTGAGGAAAATCTGGGATCTGTTTATCCACCAGTGTATTCAGAACTACACACCCGGAGCCCATC
TGACTATCGACGAACAGCTGCTGGGCTTCCGCGGGCGATGCCCTTTTCGCGTATACATTCCAAATAAGCCCAGCAAA
TATGGCATCAAGATTCTGATGATGTGCGATTCCGGGACCAAATACATGATCAACGGAATGCCATATCTGGGAGCCGG
CACCCAGACAAATGGAGTCCCCCTGGGCGAGTACTATGTGAAGGAACTGTCCAAACCTGTCCACGGGTCTTGCAGAA
ACATCACCTGTGACAATTGGTTCACATCTATTCCCCTGGCCAAGAACCTGCTGCAGGAGCCTTATAAACTGACTATC
GTCGGAACCGTGGCCAGCAACAAGAGGGAGATTCCCGAAGTGCTGAAGAACAGCCGGAGCAGACCTGTCGGCACTTC
TATGTTCTGCTTTGACGGGCCACTGACCCTGGTGAGTTACAAGCCCAAATCCAGACTGGAAGAGGAACTGAGAAGAA
GGCTCACAGAACCTGCTAAAATGGTGTATCTGCTGTCAAGCTGTGACGAGGATGCAAGCATCAATGAATCCACCGGC
AAGCCCCAGATGGTCATGTACTATAACCAGACTAAAGGCGGGGTGGATACCCTGAATCAGATGTGCTCTGTCATGAC
CTGTAGTAGAAAGACAAACAGGTGGCCTATGGCCCTGCTGTACGGGATGATCAACATTGCTTGCATTAATTCATTCA
TCATCTACAGCCACAACGTGTCCTCTAAGGGGGAGAAAGTCCAGTCCCGCAAGAAATTCATGCGAAATCTGTACATG
GGACTGACCAGTAGCTTCATGAGGAAGCGCCTGGAGGCACCCACACTGAAAAGGTATCTGCGCGACAACATCAGCAA
TATTCTGCCTAAGGAAGTGCCAGGCACTTCCGACGATTCTACCGAGGAACCAGTGATGAAGAAACGGACATACTGCA
CTTATTGTCCCAGCAAGATCCGACGGAAAGCCTCCGCTTCTTGCAAGAAGTGTAAGAAAGTGATCTGTAGAGAGCAT
AACATTGATATGTGCCGGTCCTGTTTT
SEQ ID NO: 520: amino acid sequence of Alfa E428 loop R315 R372
MPKKKRKVYPYDVPDYAGSGSSLDDEHILSALLQSDDELVGEDSDSEVSDHVSEDDVQSDTEEAFIDEVHEVQPTSS
GSEILDEQNVIEQPGSSLASNRNLTLPQRTIRGKNKHCWSTSKPTRRSRASALNIVRSQRGPTRMCRNIYDPLLCFK
LFFTDEIISEIVKWTNAEISLKRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQLLGFRGRCPFRVYIPNKPSK
YGIKILMMCDSGTKYMINGMPYLGAGTQTNGVPLGEYYVKELSKPVHGSCRNITCDNWFTSIPLAKNLLQEPYKLTI
VGTVASNKREIPEVLKNSRSRPVGTSMFCFDGPLTLVSYKPKSRLEEELRRRLTEPAKMVYLLSSCDEDASINESTG
KPQMVMYYNQTKGGVDTLNQMCSVMTCSRKTNRWPMALLYGMINIACINSFIIYSHNVSSKGEKVQSRKKFMRNLYM
GLTSSFMRKRLEAPTLKRYLRDNISNILPKEVPGTSDDSTEEPVMKKRTYCTYCPSKIRRKASASCKKCKKVICREH
NIDMCRSCF*
SEQ ID NO: 521: nucleotide sequence of E2C GCN4
ATGCCGAAAAAAAAACGAAAGGTGTACCCCTACGATGTACCGGACTATGCAGGAAGCGGGTCTTCACTGGACGATGA
GCATATTCTGAGCGCCCTGCTGCAGAGCGATGACGAGCTGGTGGGAGAGGATTCCGATTCCGAGGTCAGTGACCACG
TGTCAGAGGACGATGTGCAGAGCGACACTGAGGAAGCCTTCATCGATGAGGTCCATGAAGTGCAGCCAACAAGCTCC
GGAAGCGAGATCCTGGATGAACAGAACGTGATTGAACAGCCTGGCTCTAGTCTGGCTTCCAATAGGAACCTGACACT
GCCACAGCGAACTATTCGGGGCAAGAACAAGCACTGCTGGAGCACCTCCAAGCCTACACGGAGAAGCCGCGCGTCCG
CCCTGAACATCGTGAGATCCCAGAGGGGGCCAACCCGCATGTGCCGAAATATCTACGACCCCCTGCTGTGCTTTAAG
CTGTTCTTTACAGATGAGATCATTAGTGAAATCGTGAAGTGGACTAACGCAGAGATTTCACTGAAAAGGCGCGAATC
TATGACTAGTGCCACCTTCAGAGACACAAATGAGGATGAAATCTACGCTTTCTTTGGCATTCTGGTCATGACCGCAG
TGAGGAAGGACAACCATATGTCTACAGACGATCTGTTTGATCGCTCTCTGAGTATGGTGTATGTCTCAGTGATGAGC
AGAGACAGGTTCGATTTTTTGATCCGGTGCCTGAGAATGGACGATAAGAGCATTCGACCTACACTGCGGGAGAATGA
CGTGTTCACCCCAGTGAGGAAAATCTGGGATCTGTTTATCCACCAGTGTATTCAGAACTACACACCCGGAGCCCATC
TGACTATCGACGAACAGCTGCTGGGCTTCCGCGGGCGATGCCCTTTTCGCGTATACATTCCAAATAAGCCCAGCAAA
TATGGCATCAAGATTCTGATGATGTGCGATTCCGGGACCAAATACATGATCAACGGAATGCCATATCTGGGAGCCGG
CACCCAGACAAATGGAGTCCCCCTGGGCGAGTACTATGTGAAGGAACTGTCCAAACCTGTCCACGGGTCTTGCAGAA
ACATCACCTGTGACAATTGGTTCACATCTATTCCCCTGGCCAAGAACCTGCTGCAGGAGCCTTATAAACTGACTATC
GTCGGAACCGTGGCCAGCAACAAGAGGGAGATTCCCGAAGTGCTGAAGAACAGCCGGAGCAGACCTGTCGGCACTTC
TATGTTCTGCTTTGACGGGCCACTGACCCTGGTGAGTTACAAGCCCAAATCCAGACTGGAAGAGGAACTGAGAAGAA
GGCTCACAGAACCTGCTAAAATGGTGTATCTGCTGTCAAGCTGTGACGAGGATGCAAGCATCAATGAATCCACCGGC
AAGCCCCAGATGGTCATGTACTATAACCAGACTAAAGGCGGGGTGGATACCCTGAATCAGATGTGCTCTGTCATGAC
CTGTAGTAGAAAGACAAACAGGTGGCCTATGGCCCTGCTGTACGGGATGATCAACATTGCTTGCATTAATTCATTCA
TCATCTACAGCCACAACGTGTCCTCTAAGGGGGAGAAAGTCCAGTCCCGCAAGAAATTCATGCGAAATCTGTACATG
GGACTGACCAGTAGCTTCATGAGGAAGCGCCTGGAGGCACCCACACTGAAAAGGTATCTGCGCGACAACATCAGCAA
TATTCTGCCTAAGGAAGTGCCAGGCACTTCCGACGATTCTACCGAGGAACCAGTGATGAAGAAACGGACATACTGCA
CTTATTGTCCCAGCAAGATCCGACGGAAAGCCTCCGCTTCTTGCAAGAAGTGTAAGAAAGTGATCTGTAGAGAGCAT
AACATTGATATGTGCCGGTCCTGTTTT
SEQ ID NO: 522: amino acid sequence of E2C GCN4
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEELLSKNYHLENEVARLKK
SEQ ID NO: 523: nucleotide sequence of E2C GCN4 (GCN4 underlined)
ATGGCGCCTAAGAAGAAGAGAAAGGTCGGCGGCCTCGAACCAGGCGAGAAGCCTTATGCCTGTCCTGAGTGTGGCAA
ATCCTTCTCAAGAAAAGACTCTCTGGTTAGACACCAGAGAACACATACAGGGGAGAAACCCTATAAATGCCCCGAAT
GCGGAAAGTCCTTTTCCCAGAGCGGCGATCTCCGGAGGCATCAGAGAACTCATACAGGCGAGAAACCATATAAGTGC
CCCGAGTGTGGGAAATCCTTTTCCGATTGTAGAGACCTGGCCAGACATCAAAGGACACATACAGGCAAGAAGACCGC
TAGCGGTGGATCCGGAGGGTCCGGAGGTAGTGGCGGCAGCCTCGAGGAAGAACTTTTGAGCAAGAATTATCATCTTG
AGAACGAAGTGGCTCGTCTTAAGAAA
SEQ ID NO: 524: amino acid sequence of E2C GCN4 (GCN4 underlined)
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEELLSKNYHLENEVARLKK
SEQ ID NO: 525: nucleotide sequence of Monobody Nterm R315 R372
ATGGCGCCTAAGAAGAAGAGAAAGGTCGGCGGCGGACCTGATATTGTCATGACACAAAGCCCTAGCTCCCTGAGCGC
TAGCGTGGGCGATAGAGTCACAATAACATGCAGATCCAGCACAGGCGCTGTGACTACGTCCAATTACGCTTCTTGGG
TCCAGGAGAAACCTGGAAAGCTGTTCAAAGGGCTCATAGGAGGCACTAATAATCGGGCCCCTGGCGTCCCGTCTAGA
TTTTCCGGCAGCCTCATTGGGGACAAGGCTACCCTGACCATTAGCTCCCTCCAACCAGAGGACTTCGCTACGTATTT
TTGTGCTCTGTGGTATTCCAACCATTGGGTGTTTGGCCAAGGTACTAAGGTCGAACTCAAACGGGGTGGTGGCGGGT
CCGGAGGAGGAGGGTCTGGAGGAGGGGGGTCCTCTGGAGGGGGCAGCGAGGTTAAGCTCCTGGAGTCTGGAGGTGGG
CTTGTCCAACCCGGCGGATCACTGAAACTGAGCTGCGCTGTCTCCGGTTTTTCCCTCACCGACTACGGGGTGAACTG
GGTTCGCCAGGCGCCAGGCCGGGGATTGGAGTGGATTGGTGTAATATGGGGTGATGGGATCACAGACTACAACAGCG
CTCTTAAAGATCGGTTTATCATCTCTAAAGATAATGGCAAGAATACGGTCTATCTGCAAATGTCTAAAGTGAGATCC
GACGACACCGCCCTCTATTACTGTGTCACCGGACTCTTCGACTACTGGGGCCAAGGCACACTGGTCACAGTCAGCAG
CGCTAGCGGTGGATCCGGAGGGTCCGGAGGTAGTGGCGGCAGCCTCGAGGGGTCTTCACTGGACGATGAGCATATTC
TGAGCGCCCTGCTGCAGAGCGATGACGAGCTGGTGGGAGAGGATTCCGATTCCGAGGTCAGTGACCACGTGTCAGAG
GACGATGTGCAGAGCGACACTGAGGAAGCCTTCATCGATGAGGTCCATGAAGTGCAGCCAACAAGCTCCGGAAGCGA
GATCCTGGATGAACAGAACGTGATTGAACAGCCTGGCTCTAGTCTGGCTTCCAATAGGAACCTGACACTGCCACAGC
GAACTATTCGGGGCAAGAACAAGCACTGCTGGAGCACCTCCAAGCCTACACGGAGAAGCCGCGCGTCCGCCCTGAAC
ATCGTGAGATCCCAGAGGGGGCCAACCCGCATGTGCCGAAATATCTACGACCCCCTGCTGTGCTTTAAGCTGTTCTT
TACAGATGAGATCATTAGTGAAATCGTGAAGTGGACTAACGCAGAGATTTCACTGAAAAGGCGCGAATCTATGACTA
GTGCCACCTTCAGAGACACAAATGAGGATGAAATCTACGCTTTCTTTGGCATTCTGGTCATGACCGCAGTGAGGAAG
GACAACCATATGTCTACAGACGATCTGTTTGATCGCTCTCTGAGTATGGTGTATGTCTCAGTGATGAGCAGAGACAG
GTTCGATTTTTTGATCCGGTGCCTGAGAATGGACGATAAGAGCATTCGACCTACACTGCGGGAGAATGACGTGTTCA
CCCCAGTGAGGAAAATCTGGGATCTGTTTATCCACCAGTGTATTCAGAACTACACACCCGGAGCCCATCTGACTATC
GACGAACAGCTGCTGGGCTTCCGCGGGCGATGCCCTTTTCGCGTATACATTCCAAATAAGCCCAGCAAATATGGCAT
CAAGATTCTGATGATGTGCGATTCCGGGACCAAATACATGATCAACGGAATGCCATATCTGGGAGCCGGCACCCAGA
CAAATGGAGTCCCCCTGGGCGAGTACTATGTGAAGGAACTGTCCAAACCTGTCCACGGGTCTTGCAGAAACATCACC
TGTGACAATTGGTTCACATCTATTCCCCTGGCCAAGAACCTGCTGCAGGAGCCTTATAAACTGACTATCGTCGGAAC
CGTGGCCAGCAACAAGAGGGAGATTCCCGAAGTGCTGAAGAACAGCCGGAGCAGACCTGTCGGCACTTCTATGTTCT
GCTTTGACGGGCCACTGACCCTGGTGAGTTACAAGCCCAAACCTGCTAAAATGGTGTATCTGCTGTCAAGCTGTGAC
GAGGATGCAAGCATCAATGAATCCACCGGCAAGCCCCAGATGGTCATGTACTATAACCAGACTAAAGGCGGGGTGGA
TACCCTGAATCAGATGTGCTCTGTCATGACCTGTAGTAGAAAGACAAACAGGTGGCCTATGGCCCTGCTGTACGGGA
TGATCAACATTGCTTGCATTAATTCATTCATCATCTACAGCCACAACGTGTCCTCTAAGGGGGAGAAAGTCCAGTCC
CGCAAGAAATTCATGCGAAATCTGTACATGGGACTGACCAGTAGCTTCATGAGGAAGCGCCTGGAGGCACCCACACT
GAAAAGGTATCTGCGCGACAACATCAGCAATATTCTGCCTAAGGAAGTGCCAGGCACTTCCGACGATTCTACCGAGG
AACCAGTGATGAAGAAACGGACATACTGCACTTATTGTCCCAGCAAGATCCGACGGAAAGCCTCCGCTTCTTGCAAG
AAGTGTAAGAAAGTGATCTGTAGAGAGCATAACATTGATATGTGCCGGTCCTGTTTT
SEQ ID NO: 526: amino acid sequence of Monobody Nterm R315 R372
MAPKKKRKVGGGPDIVMTQSPSSLSASVGDRVTITCRSSTGAVTTSNYASWVQEKPGKLFKGLIGGTNNRAPGVPSR
FSGSLIGDKATLTISSLQPEDFATYFCALWYSNHWVFGQGTKVELKRGGGGSGGGGSGGGGSSGGGSEVKLLESGGG
LVQPGGSLKLSCAVSGFSLTDYGVNWVRQAPGRGLEWIGVIWGDGITDYNSALKDRFIISKDNGKNTVYLQMSKVRS
DDTALYYCVTGLFDYWGQGTLVTVSSASGGSGGSGGSGGSLEGSSLDDEHILSALLQSDDELVGEDSDSEVSDHVSE
DDVQSDTEEAFIDEVHEVQPTSSGSEILDEQNVIEQPGSSLASNRNLTLPQRTIRGKNKHCWSTSKPTRRSRASALN
IVRSQRGPTRMCRNIYDPLLCFKLFFTDEIISEIVKWTNAEISLKRRESMTSATFRDTNEDEIYAFFGILVMTAVRK
DNHMSTDDLFDRSLSMVYVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGAGTQTNGVPLGEYYVKELSKPVHGSCRNIT
CDNWFTSIPLAKNLLQEPYKLTIVGTVASNKREIPEVLKNSRSRPVGTSMFCFDGPLTLVSYKPKPAKMVYLLSSCD
EDASINESTGKPQMVMYYNQTKGGVDTLNQMCSVMTCSRKTNRWPMALLYGMINIACINSFIIYSHNVSSKGEKVQS
RKKFMRNLYMGLTSSFMRKRLEAPTLKRYLRDNISNILPKEVPGTSDDSTEEPVMKKRTYCTYCPSKIRRKASASCK
KCKKVICREHNIDMCRSCF
SEQ ID NO: 527: nucleotide sequence of Monobody Nterm R315 R372 (monobody underlined)
ATGGCGCCTAAGAAGAAGAGAAAGGTCGGCGGCGGACCTGATATTGTCATGACACAAAGCCCTAGCTCCCTGAGCGC
TAGCGTGGGCGATAGAGTCACAATAACATGCAGATCCAGCACAGGCGCTGTGACTACGTCCAATTACGCTTCTTGGG
TCCAGGAGAAACCTGGAAAGCTGTTCAAAGGGCTCATAGGAGGCACTAATAATCGGGCCCCTGGCGTCCCGTCTAGA
TTTTCCGGCAGCCTCATTGGGGACAAGGCTACCCTGACCATTAGCTCCCTCCAACCAGAGGACTTCGCTACGTATTT
TTGTGCTCTGTGGTATTCCAACCATTGGGTGTTTGGCCAAGGTACTAAGGTCGAACTCAAACGGGGTGGTGGCGGGT
CCGGAGGAGGAGGGTCTGGAGGAGGGGGGTCCTCTGGAGGGGGCAGCGAGGTTAAGCTCCTGGAGTCTGGAGGTGGG
CTTGTCCAACCCGGCGGATCACTGAAACTGAGCTGCGCTGTCTCCGGTTTTTCCCTCACCGACTACGGGGTGAACTG
GGTTCGCCAGGCGCCAGGCCGGGGATTGGAGTGGATTGGTGTAATATGGGGTGATGGGATCACAGACTACAACAGCG
CTCTTAAAGATCGGTTTATCATCTCTAAAGATAATGGCAAGAATACGGTCTATCTGCAAATGTCTAAAGTGAGATCC
GACGACACCGCCCTCTATTACTGTGTCACCGGACTCTTCGACTACTGGGGCCAAGGCACACTGGTCACAGTCAGCAG
CGCTAGCGGTGGATCCGGAGGGTCCGGAGGTAGTGGCGGCAGCCTCGAGGGGTCTTCACTGGACGATGAGCATATTC
TGAGCGCCCTGCTGCAGAGCGATGACGAGCTGGTGGGAGAGGATTCCGATTCCGAGGTCAGTGACCACGTGTCAGAG
GACGATGTGCAGAGCGACACTGAGGAAGCCTTCATCGATGAGGTCCATGAAGTGCAGCCAACAAGCTCCGGAAGCGA
GATCCTGGATGAACAGAACGTGATTGAACAGCCTGGCTCTAGTCTGGCTTCCAATAGGAACCTGACACTGCCACAGC
GAACTATTCGGGGCAAGAACAAGCACTGCTGGAGCACCTCCAAGCCTACACGGAGAAGCCGCGCGTCCGCCCTGAAC
ATCGTGAGATCCCAGAGGGGGCCAACCCGCATGTGCCGAAATATCTACGACCCCCTGCTGTGCTTTAAGCTGTTCTT
TACAGATGAGATCATTAGTGAAATCGTGAAGTGGACTAACGCAGAGATTTCACTGAAAAGGCGCGAATCTATGACTA
GTGCCACCTTCAGAGACACAAATGAGGATGAAATCTACGCTTTCTTTGGCATTCTGGTCATGACCGCAGTGAGGAAG
GACAACCATATGTCTACAGACGATCTGTTTGATCGCTCTCTGAGTATGGTGTATGTCTCAGTGATGAGCAGAGACAG
GTTCGATTTTTTGATCCGGTGCCTGAGAATGGACGATAAGAGCATTCGACCTACACTGCGGGAGAATGACGTGTTCA
CCCCAGTGAGGAAAATCTGGGATCTGTTTATCCACCAGTGTATTCAGAACTACACACCCGGAGCCCATCTGACTATC
GACGAACAGCTGCTGGGCTTCCGCGGGCGATGCCCTTTTCGCGTATACATTCCAAATAAGCCCAGCAAATATGGCAT
CAAGATTCTGATGATGTGCGATTCCGGGACCAAATACATGATCAACGGAATGCCATATCTGGGAGCCGGCACCCAGA
CAAATGGAGTCCCCCTGGGCGAGTACTATGTGAAGGAACTGTCCAAACCTGTCCACGGGTCTTGCAGAAACATCACC
TGTGACAATTGGTTCACATCTATTCCCCTGGCCAAGAACCTGCTGCAGGAGCCTTATAAACTGACTATCGTCGGAAC
CGTGGCCAGCAACAAGAGGGAGATTCCCGAAGTGCTGAAGAACAGCCGGAGCAGACCTGTCGGCACTTCTATGTTCT
GCTTTGACGGGCCACTGACCCTGGTGAGTTACAAGCCCAAACCTGCTAAAATGGTGTATCTGCTGTCAAGCTGTGAC
GAGGATGCAAGCATCAATGAATCCACCGGCAAGCCCCAGATGGTCATGTACTATAACCAGACTAAAGGCGGGGTGGA
TACCCTGAATCAGATGTGCTCTGTCATGACCTGTAGTAGAAAGACAAACAGGTGGCCTATGGCCCTGCTGTACGGGA
TGATCAACATTGCTTGCATTAATTCATTCATCATCTACAGCCACAACGTGTCCTCTAAGGGGGAGAAAGTCCAGTCC
CGCAAGAAATTCATGCGAAATCTGTACATGGGACTGACCAGTAGCTTCATGAGGAAGCGCCTGGAGGCACCCACACT
GAAAAGGTATCTGCGCGACAACATCAGCAATATTCTGCCTAAGGAAGTGCCAGGCACTTCCGACGATTCTACCGAGG
AACCAGTGATGAAGAAACGGACATACTGCACTTATTGTCCCAGCAAGATCCGACGGAAAGCCTCCGCTTCTTGCAAG
AAGTGTAAGAAAGTGATCTGTAGAGAGCATAACATTGATATGTGCCGGTCCTGTTTT
SEQ ID NO: 528: amino acid sequence of Monobody Nterm R315 R372 (monobody underlined)
MAPKKKRKVGGGPDIVMTQSPSSLSASVGDRVTITCRSSTGAVTTSNYASWVQEKPGKLFKGLIGGTNNRAPGVPSR
FSGSLIGDKATLTISSLQPEDFATYFCALWYSNHWVFGQGTKVELKRGGGGSGGGGSGGGGSSGGGSEVKLLESGGG
LVQPGGSLKLSCAVSGFSLTDYGVNWVRQAPGRGLEWIGVIWGDGITDYNSALKDRFIISKDNGKNTVYLQMSKVRS
DDTALYYCVTGLFDYWGQGTLVTVSSASGGSGGSGGSGGSLEGSSLDDEHILSALLQSDDELVGEDSDSEVSDHVSE
DDVQSDTEEAFIDEVHEVQPTSSGSEILDEQNVIEQPGSSLASNRNLTLPQRTIRGKNKHCWSTSKPTRRSRASALN
IVRSQRGPTRMCRNIYDPLLCFKLFFTDEIISEIVKWTNAEISLKRRESMTSATFRDTNEDEIYAFFGILVMTAVRK
DNHMSTDDLFDRSLSMVYVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGAGTQTNGVPLGEYYVKELSKPVHGSCRNIT
CDNWFTSIPLAKNLLQEPYKLTIVGTVASNKREIPEVLKNSRSRPVGTSMFCFDGPLTLVSYKPKPAKMVYLLSSCD
EDASINESTGKPQMVMYYNQTKGGVDTLNQMCSVMTCSRKTNRWPMALLYGMINIACINSFIIYSHNVSSKGEKVQS
RKKFMRNLYMGLTSSFMRKRLEAPTLKRYLRDNISNILPKEVPGTSDDSTEEPVMKKRTYCTYCPSKIRRKASASCK
KCKKVICREHNIDMCRSCF

In embodiments, a helper plasmid encodes a PB transposase containing either a loop fusion of a bridging domain or a loop fusion of a zinc finger. In embodiments, a reporter plasmid containing a promotorless-zsGreen fluorescent protein contains the target sequence for the zinc finger. In embodiments, a donor plasmid contains the PB transposon with a CMV promoter oriented pointing outward. In the event of successful targeting, the transposon inserts near the target sequence on the reporter plasmid and aligns the promotor with the zsGreen, which results in zsGreen expression. In embodiments, the expression can be detected by flow cytometry or a comparable fluorescence detection method. In embodiments, upon co-expression of a helper plasmid encoding PB with one of several loop insertions, the donor plasmid containing the transposon and CMV promoter, and the reporter plasmid containing the promotorless-zsGreen, an increase in zsGreen fluorescence is measured which indicates that the loop infusion strategy is successful at targeting PB to the plasmid target sequence.

In embodiments, the reporter plasmid is used to deliver the target sequence into the human genome. In embodiments, the reporter plasmid is used to deliver the target sequence into the human genome by co-transfecting it into HEK293 cells along with a plasmid expressing the Sleeping Beauty transposase. In embodiments, the Sleeping Beauty transposase is used to enzymatically insert the reporter plasmid components, including target sequence and promotorless-zsGreen into random locations within the genome. In embodiments, cell lines are generated containing the reporter components in the genome. Upon co-expression into these cell lines by a helper plasmid encoding PB with one of several loop insertions, the donor plasmid containing the transposon and CMV promoter, an increase in zsGreen fluorescence is measured which indicates that the loop infusion strategy is successful at targeting PB insertion to the genomic target sequence.

In embodiments, DNA binding domains are fused to the N-terminus or loop domains of piggyBac. In embodiments, such fusions result in the PB transposase becoming localized to the target DNA where integration occurs within close proximity to the sequence. In embodiments, the zinc fingers are inserted within the PB transposase open reading frame at loop domains.

In embodiments, successful targeting of PB is achieved by bridging domain insertions into the loops of PB. In embodiments, successful targeting of PB is achieved by direct covalent DNA binding domain insertions into the loops of PB.

### Construct

In some embodiments, the composition (e.g., without limitation, hyperactive transposase of the present disclosure), system, or method further comprising a nucleic acid encoding a transposon comprising a transgene to be integrated. In some embodiments, the transgene comprises a cargo nucleic acid sequence and a first and a second transposon end sequences. In some embodiments, the cargo nucleic acid sequence is flanked by the first and the second transposon end sequences.

In some embodiments, the transposon end sequences are selected from nucleotide sequences of SEQ ID NO: 5 and/or SEQ ID NO: 6, or a nucleotide sequence having at least about 90% identity thereto.

| SEQ ID NO: 5: nucleotide sequence of left hyperactive transposase donor end sequence (310 bp) | |
|---|---|
| 1 | ATCTATAACA AGAAAATATA TATATAATAA GTTATCACGT AAGTAGAACA |
| 51 | TGAAATAACA ATATAATTAT CGTATGAGTT AAATCTTAAA AGTCACGTAA |
| 101 | AAGATAATCA TGCGTCATTT TGACTCACGC GGTCGTTATA GTTCAAAATC |
| 151 | AGTGACACTT ACCGCATTGA CAAGCACGCC TCACGGGAGC TCCAAGCGGC |
| 201 | GACTGAGATG TCCTAAATGC ACAGCGACGG ATTCGCGCTA TTTAGAAAGA |
| 251 | GAGAGCAATA TTTCAAGAAT GCATGCGTCA ATTTTACGCA GACTATCTTT |
| 301 | CTAGGGTTAA |
| SEQ ID NO: 6: nucleotide sequence of right hyperactive transposase donor end sequence (205 bp) | |
| 1 | TTAACCCTAG AAAGATAATC ATATTGTGAC GTACGTTAAA GATAATCATG |
| 51 | CGTAAAATTG ACGCATGTGT TTTATCGGTC TGTATATCGA GGTTTATTTA |
| 101 | TTAATTTGAA TAGATATTAA GTTTTATTAT ATTTACACTT ACATACTAAT |
| 151 | AATAAATTCA ACAAACAATT TATTTATGTT TATTTATTTA TTAAAAAAAA |
| 201 | ACAAA |

In some embodiments, the end sequences include at least one repeat from a nucleotide sequence having at least about 90% identity to the nucleotide sequence of SEQ ID NO: 5. In some embodiments, the at least one repeat from the nucleotide sequence having at least about 90% identity to the nucleotide sequence of SEQ ID NO: 5 is positioned at the 5′ end of the transposon. In some embodiments, the end sequences can further include at least one repeat from a nucleotide sequence having at least about 90% identity to the nucleotide sequence of SEQ ID NO: 6. In some embodiments, the at least one repeat from the nucleotide sequence having at least about 90% identity to the nucleotide sequence of SEQ ID NO: 6 is positioned at the 3′ end of the transposon.

In some embodiments, the enzyme or variant thereof is incorporated into a vector or a vector-like particle. In some embodiments, the vector or a vector-like particle comprises one or more expression cassettes. In some embodiments, the vector or a vector-like particle comprises one expression cassette. In some embodiments, the expression cassette further comprises the enzyme or variant thereof, the transgene, the transposon end sequences, or a combination thereof.

In some embodiments, the enzyme or variant thereof, the transgene, the transposon end sequences, or a combination thereof are incorporated into one or more vectors or vector-like particles. In some embodiments, the enzyme or variant thereof, the transgene, the transposon end sequences, or combination thereof are incorporated into a same vector or vector-like particle. In some embodiments, the enzyme or variant thereof, the transgene, the transposon end sequences, or combination thereof is incorporated into different vectors vector-like particles. In some embodiments, the vector or vector-like particle is nonviral. In some embodiments, the composition comprises DNA, RNA, or both. In some embodiments, the enzyme or variant thereof is in the form of RNA.

### N or C Terminal Deletion Variants

In aspects, the present disclosure further provides a piggyBac with a deletion in either N or C terminus. In embodiments, the piggyBac comprises a deletion in the N-terminus. In embodiments, the piggyBac comprises a deletion in the C-terminus.

In embodiments, the piggyBac comprises a deletion from an N- or C-terminus of the polypeptide having an amino acid sequence of SEQ ID NO: 502, or a sequence having at least about 90% identity thereto.

| SEQ ID NO: 501: Trichnoplusia ni (hyperactive piggyBac) nucleotide sequence (N0) 1782 bp | |
|---|---|
| 1 | ATGGGCTCTT CCTTGGACGA CGAGCACATC CTGAGCGCCC TGCTGCAGTC CGATGACGAG |
| 61 | CTGGTGGGTG AGGACTCCGA CAGTGAGGTG AGCGATCATG TGTCCGAGGA CGATGTGCAG |
| 121 | AGCGACACCG AGGAGGCCTT CATCGATGAG GTGCATGAAG TGCAGCCCAC ATCCTCCGGA |
| 181 | TCCGAGATCC TGGATGAGCA GAACGTGATC GAGCAGCCCG GGAGCTCTCT GGCTTCCAAC |
| 241 | AGAATCCTGA CCCTGCCTCA GAGGACTATC AGAGGGAAAA ATAAGCACTG CTGGTCCACT |
| 301 | TCAAAGCCAA CCAGGCGGTC TAGGGTGTCC GCCCTGAACA TCGTGAGAAG CCAGAGAGGT |
| 361 | CCAACCCGGA TGTGCAGAAA CATCTACGAT CCCCTGCTGT GTTTCAAGCT GTTTTTTACT |
| 421 | GACGAGATTA TCAGCGAGAT CGTGAAGTGG ACCAACGCCG AGATCTCTCT GAAGCGGAGA |
| 481 | GAAAGCATGA CCTCCGCCAC ATTCCGCGAT ACCAACGAAG ACGAGATCTA CGCCTTTTTC |
| 541 | GGCATCTTGG TGATGACTGC CGTGAGGAAG GACAATCACA TGAGCACCGA TGACCTGTTC |
| 601 | GATCGCAGCC TGAGCATGGT GTACGTGTCC GTGATGTCCA GGGACAGGTT CGACTTCCTG |
| 661 | ATCAGGTGCC TGAGAATGGA TGACAAGAGC ATTAGACCTA CCCTGCGCGA GAATGACGTG |
| 721 | TTCACCCCCG TCAGGAAGAT CTGGGACCTG TTTATTCACC AGTGCATTCA GAACTATACC |
| 781 | CCTGGCGCCC ACCTGACCAT TGATGAGCAG CTCCTGGGAT TCAGGGGCCG GTGTCCCTTC |
| 841 | AGAGTGTATA TACCTAACAA GCCCTCTAAG TACGGCATCA AAATCCTGAT GATGTGCGAC |
| 901 | TCCGGAACCA AGTACATGAT TAACGGAATG CCCTATCTGG GACGCGGGAC CCAGACCAAT |
| 961 | GGAGTGCCTC TGGGCGAGTA TTACGTGAAA GAGCTGTCCA AACCCGTGCA CGGGTCCTGT |
| 1021 | CGGAATATCA CCTGCGACAA CTGGTTCACT TCAATCCCCC TGGCCAAAAA CCTGCTGCAG |
| 1081 | GAGCCTTACA AATTGACCAT TGTGGGCACT GTGCGCTCCA ATAAGAGAGA AATTCCTGAG |
| 1141 | GTGCTGAAGA ACTCCAGGTC CAGACCCGTG GGCACTAGCA TGTTCTGCTT CGACGGGCCT |
| 1201 | CTGACCCTGG TGAGCTATAA GCCCAAGCCA GCCAAGATGG TGTACCTGCT GAGCAGCTGC |
| 1261 | GACGAGGATG CCTCAATCAA CGAGAGCACC GGCAAGCCCC AGATGGTGAT GTACTACAAT |
| 1321 | CAGACCAAAG GCGGCGTGGA TACCCTGGAC CAGATGTGTT CCGTGATGAC CTGTAGCAGG |
| 1381 | AAGACCAACC GGTGGCCAAT GGCCCTGCTG TATGGAATGA TCAACATTGC CTGCATCAAC |
| 1441 | AGCTTCATTA TTTACTCCCA CAATGTGTCC TCTAAGGGGG AGAAGGTGCA GTCTAGAAAA |
| 1501 | AAGTTTATGA GGAATCTGTA TATGGGCCTG ACCTCTTCCT TCATGCGGAA GCGCCTGGAG |
| 1561 | GCACCCACAC TGAAGAGGTA CCTTCGCGAC AATATCTCCA ATATTCTGCC GAAAGAGGTG |
| 1621 | CCCGGCACCT CCGACGACAG CACAGAAGAG CCCGTGATGA AAAAGAGGAC CTACTGCACC |
| 1681 | TACTGCCCCT CAAAGATCCG CAGGAAGGCC TCCGCATCCT GTAAGAAGTG CAAGAAGGTC |
| 1741 | ATCTGCAGGG AACACAATAT CGACATGTGC CAGTCATGCT TC |
| SEQ ID NO: 502: Trichnoplusia ni (hyperactive piggyBac) amino acid sequence (N0) 594 aa | |
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPqRTI RGKNKHCWST SKPTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVqSRK KFMRNLYMGL TSSFMRKRLE APTLKRYLRD NISNILPKEV |
| 541 | PGTSDDSTEE PVMKKRTYCT YCPSKIRRKA SASCKKCKKV ICREHNIDMC QSCF |

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an N-terminus of the polypeptide having an amino acid sequence of SEQ ID NO: 502, or a sequence having at least about 90% identity thereto.

In embodiments, the piggyBac with deletion from the N-terminus comprises SEQ ID NO: 504, SEQ ID NO: 506, SEQ ID NO: 508, or SEQ ID NO: 510, or a sequence having at least about 90% identity thereto.

| SEQ ID NO: 503: Trichnoplusia ni (hyperactive piggyBac) nucleotide sequence (N1; | |
|---|---|
| nucleotide 4-72 deletion). 1710 bp | |
| 1 | ATGGACTCCG ACAGTGAGGT GAGCGATCAT GTGTCCGAGG ACGATGTGCA GAGCGACACC |
| 61 | GAGGAGGCCT TCATCGATGA GGTGCATGAA GTGCAGCCCA CATCCTCCGG ATCCGAGATC |
| 121 | CTGGATGAGC AGAACGTGAT CGAGCAGCCC GGGAGCTCTC TGGCTTCCAA CAGAATCCTG |
| 181 | ACCCTGCCTC AGAGGACTAT CAGAGGGAAA AATAAGCACT GCTGGTCCAC TTCAAAGCCA |
| 241 | ACCAGGCGGT CTAGGGTGTC CGCCCTGAAC ATCGTGAGAA GCCAGAGAGG TCCAACCCGG |
| 301 | ATGTGCAGAA ACATCTACGA TCCCCTGCTG TGTTTCAAGC TGTTTTTTAC TGACGAGATT |
| 361 | ATCAGCGAGA TCGTGAAGTG GACCAACGCC GAGATCTCTC TGAAGCGGAG AGAAAGCATG |
| 421 | ACCTCCGCCA CATTCCGCGA TACCAACGAA GACGAGATCT ACGCCTTTTT CGGCATCTTG |
| 481 | GTGATGACTG CCGTGAGGAA GGACAATCAC ATGAGCACCG ATGACCTGTT CGATCGCAGC |
| 541 | CTGAGCATGG TGTACGTGTC CGTGATGTCC AGGGACAGGT TCGACTTCCT GATCAGGTGC |
| 601 | CTGAGAATGG ATGACAAGAG CATTAGACCT ACCCTGCGCG AGAATGACGT GTTCACCCCC |
| 661 | GTCAGGAAGA TCTGGGACCT GTTTATTCAC CAGTGCATTC AGAACTATAC CCCTGGCGCC |
| 721 | CACCTGACCA TTGATGAGCA GCTCCTGGGA TTCAGGGGCC GGTGTCCCTT CAGAGTGTAT |
| 781 | ATACCTAACA AGCCCTCTAA GTACGGCATC AAAATCCTGA TGATGTGCGA CTCCGGAACC |
| 841 | AAGTACATGA TTAACGGAAT GCCCTATCTG GGACGCGGGA CCCAGACCAA TGGAGTGCCT |
| 901 | CTGGGCGAGT ATTACGTGAA AGAGCTGTCC AAACCCGTGC ACGGGTCCTG TCGGAATATC |
| 961 | ACCTGCGACA ACTGGTTCAC TTCAATCCCC CTGGCCAAAA ACCTGCTGCA GGAGCCTTAC |
| 1021 | AAATTGACCA TTGTGGGCAC TGTGCGCTCC AATAAGAGAG AAATTCCTGA GGTGCTGAAG |
| 1081 | AACTCCAGGT CCAGACCCGT GGGCACTAGC ATGTTCTGCT TCGACGGGCC TCTGACCCTG |
| 1141 | GTGAGCTATA AGCCCAAGCC AGCCAAGATG GTGTACCTGC TGAGCAGCTG CGACGAGGAT |
| 1201 | GCCTCAATCA ACGAGAGCAC CGGCAAGCCC CAGATGGTGA TGTACTACAA TCAGACCAAA |
| 1261 | GGCGGCGTGG ATACCCTGGA CCAGATGTGT TCCGTGATGA CCTGTAGCAG GAAGACCAAC |
| 1321 | CGGTGGCCAA TGGCCCTGCT GTATGGAATG ATCAACATTG CCTGCATCAA CAGCTTCATT |
| 1381 | ATTTACTCCC ACAATGTGTC CTCTAAGGGG GAGAAGGTGC AGTCTAGAAA AAAGTTTATG |
| 1441 | AGGAATCTGT ATATGGGCCT GACCTCTTCC TTCATGCGGA AGCGCCTGGA GGCACCCACA |
| 1501 | CTGAAGAGGT ACCTTCGCGA CAATATCTCC AATATTCTGC CGAAAGAGGT GCCCGGCACC |
| 1561 | TCCGACGACA GCACAGAAGA GCCCGTGATG AAAAAGAGGA CCTACTGCAC CTACTGCCCC |
| 1621 | TCAAAGATCC GCAGGAAGGC CTCCGCATCC TGTAAGAAGT GCAAGAAGGT CATCTGCAGG |
| 1681 | GAACACAATA TCGACATGTG CCAGTCATGC TTC |
| SEQ ID NO: 504: Trichnoplusia ni (hyperactive piggyBac) amino acid sequence (N1; amino | |
| acid 2-24 deletion). 571 aa | |
| 1 | MDSDSEVSDH VSEDDVQSDT EEAFIDEVHE VQPTSSGSEI LDEQNVIEQP GSSLASNRIL |
| 61 | TLPQRTIRGK NKHCWSTSKP TRRSRVSALN IVRSQRGPTR MCRNIYDPLL CFKLFFTDEI |
| 121 | ISEIVKWTNA EISLKRRESM TSATFRDTNE DEIYAFFGIL VMTAVRKDNH MSTDDLFDRS |
| 181 | LSMVYVSVMS RDRFDFLIRC LRMDDKSIRP TLRENDVFTP VRKIWDLFIH QCIQNYTPGA |
| 241 | HLTIDEQLLG FRGRCPFRVY IPNKPSKYGI KILMMCDSGT KYMINGMPYL GRGTQTNGVP |
| 301 | LGEYYVKELS KPVHGSCRNI TCDNWFTSIP LAKNLLQEPY KLTIVGTVRS NKREIPEVLK |
| 361 | NSRSRPVGTS MFCFDGPLTL VSYKPKPAKM VYLLSSCDED ASINESTGKP QMVMYYNQTK |
| 421 | GGVDTLDQMC SVMTCSRKTN RWPMALLYGM INIACINSFI IYSHNVSSKG EKVQSRKKFM |
| 481 | RNLYMGLTSS FMRKRLEAPT LKRYLRDNIS NILPKEVPGT SDDSTEEPVM KKRTYCTYCP |
| 541 | SKIRRKASAS CKKCKKVICR EHNIDMCQSC F |
| SEQ ID NO: 505: Trichnoplusia ni (hyperactive piggyBac) nucleotide sequence (N2; | |
| nucleotide 4-117 deletion) 1668 bp | |
| 1 | ATGCAGAGCG ACACCGAGGA GGCCTTCATC GATGAGGTGC ATGAAGTGCA GCCCACATCC |
| 61 | TCCGGATCCG AGATCCTGGA TGAGCAGAAC GTGATCGAGC AGCCCGGGAG CTCTCTGGCT |
| 121 | TCCAACAGAA TCCTGACCCT GCCTCAGAGG ACTATCAGAG GGAAAAATAA GCACTGCTGG |
| 181 | TCCACTTCAA AGCCAACCAG GCGGTCTAGG GTGTCCGCCC TGAACATCGT GAGAAGCCAG |
| 241 | AGAGGTCCAA CCCGGATGTG CAGAAACATC TACGATCCCC TGCTGTGTTT CAAGCTGTTT |
| 301 | TTTACTGACG AGATTATCAG CGAGATCGTG AAGTGGACCA ACGCCGAGAT CTCTCTGAAG |
| 361 | CGGAGAGAAA GCATGACCTC CGCCACATTC CGCGATACCA ACGAAGACGA GATCTACGCC |
| 421 | TTTTTCGGCA TCTTGGTGAT GACTGCCGTG AGGAAGGACA ATCACATGAG CACCGATGAC |
| 481 | CTGTTCGATC GCAGCCTGAG CATGGTGTAC GTGTCCGTGA TGTCCAGGGA CAGGTTCGAC |
| 541 | TTCCTGATCA GGTGCCTGAG AATGGATGAC AAGAGCATTA GACCTACCCT GCGCGAGAAT |
| 601 | GACGTGTTCA CCCCCGTCAG GAAGATCTGG GACCTGTTTA TTCACCAGTG CATTCAGAAC |
| 661 | TATACCCCTG GCGCCCACCT GACCATTGAT GAGCAGCTCC TGGGATTCAG GGGCCGGTGT |
| 721 | CCCTTCAGAG TGTATATACC TAACAAGCCC TCTAAGTACG GCATCAAAAT CCTGATGATG |
| 781 | TGCGACTCCG GAACCAAGTA CATGATTAAC GGAATGCCCT ATCTGGGACG CGGGACCCAG |
| 841 | ACCAATGGAG TGCCTCTGGG CGAGTATTAC GTGAAAGAGC TGTCCAAACC CGTGCACGGG |
| 901 | TCCTGTCGGA ATATCACCTG CGACAACTGG TTCACTTCAA TCCCCCTGGC CAAAAACCTG |
| 961 | CTGCAGGAGC CTTACAAATT GACCATTGTG GGCACTGTGC GCTCCAATAA GAGAGAAATT |
| 1021 | CCTGAGGTGC TGAAGAACTC CAGGTCCAGA CCCGTGGGCA CTAGCATGTT CTGCTTCGAC |
| 1081 | GGGCCTCTGA CCCTGGTGAG CTATAAGCCC AAGCCAGCCA AGATGGTGTA CCTGCTGAGC |
| 1141 | AGCTGCGACG AGGATGCCTC AATCAACGAG AGCACCGGCA AGCCCCAGAT GGTGATGTAC |
| 1201 | TACAATCAGA CCAAAGGCGG CGTGGATACC CTGGACCAGA TGTGTTCCGT GATGACCTGT |
| 1261 | AGCAGGAAGA CCAACCGGTG GCCAATGGCC CTGCTGTATG GAATGATCAA CATTGCCTGC |
| 1321 | ATCAACAGCT TCATTATTTA CTCCCACAAT GTGTCCTCTA AGGGGGAGAA GGTGCAGTCT |
| 1381 | AGAAAAAAGT TTATGAGGAA TCTGTATATG GGCCTGACCT CTTCCTTCAT GCGGAAGCGC |
| 1441 | CTGGAGGCAC CCACACTGAA GAGGTACCTT CGCGACAATA TCTCCAATAT TCTGCCGAAA |
| 1501 | GAGGTGCCCG GCACCTCCGA CGACAGCACA GAAGAGCCCG TGATGAAAAA GAGGACCTAC |
| 1561 | TGCACCTACT GCCCCTCAAA GATCCGCAGG AAGGCCTCCG CATCCTGTAA GAAGTGCAAG |
| 1621 | AAGGTCATCT GCAGGGAACA CAATATCGAC ATGTGCCAGT CATGCTTC |
| SEQ ID NO: 506: Trichnoplusia ni (hyperactive piggyBac) amino acid sequence (N2; amino | |
| acid 2-39 deletion) 556 aa | |
| 1 | MQSDTEEAFI DEVHEVQPTS SGSEILDEQN VIEQPGSSLA SNRILTLPQR TIRGKNKHCW |
| 61 | STSKPTRRSR VSALNIVRSQ RGPTRMCRNI YDPLLCFKLF FTDEIISEIV KWTNAEISLK |
| 121 | RRESMTSATF RDTNEDEIYA FFGILVMTAV RKDNHMSTDD LFDRSLSMVY VSVMSRDRFD |
| 181 | FLIRCLRMDD KSIRPTLREN DVFTPVRKIW DLFIHQCIQN YTPGAHLTID EQLLGFRGRC |
| 241 | PFRVYIPNKP SKYGIKILMM CDSGTKYMIN GMPYLGRGTQ TNGVPLGEYY VKELSKPVHG |
| 301 | SCRNITCDNW FTSIPLAKNL LQEPYKLTIV GTVRSNKREI PEVLKNSRSR PVGTSMFCFD |
| 361 | GPLTLVSYKP KPAKMVYLLS SCDEDASINE STGKPQMVMY YNQTKGGVDT LDQMCSVMTC |
| 421 | SRKTNRWPMA LLYGMINIAC INSFIIYSHN VSSKGEKVQS RKKFMRNLYM GLTSSFMRKR |
| 481 | LEAPTLKRYL RDNISNILPK EVPGTSDDST EEPVMKKRTY CTYCPSKIRR KASASCKKCK |
| 541 | KVICREHNID MCQSCF |
| SEQ ID NO: 507: Trichnoplusia ni (hyperactive piggyBac) nucleotide sequence (N3; | |
| nucleotide 4-258 deletion). 1524 bp | |
| 1 | ATGAGGACTA TCAGAGGGAA AAATAAGCAC TGCTGGTCCA CTTCAAAGCC AACCAGGCGG |
| 61 | TCTAGGGTGT CCGCCCTGAA CATCGTGAGA AGCCAGAGAG GTCCAACCCG GATGTGCAGA |
| 121 | AACATCTACG ATCCCCTGCT GTGTTTCAAG CTGTTTTTTA CTGACGAGAT TATCAGCGAG |
| 181 | ATCGTGAAGT GGACCAACGC CGAGATCTCT CTGAAGCGGA GAGAAAGCAT GACCTCCGCC |
| 241 | ACATTCCGCG ATACCAACGA AGACGAGATC TACGCCTTTT TCGGCATCTT GGTGATGACT |
| 301 | GCCGTGAGGA AGGACAATCA CATGAGCACC GATGACCTGT TCGATCGCAG CCTGAGCATG |
| 361 | GTGTACGTGT CCGTGATGTC CAGGGACAGG TTCGACTTCC TGATCAGGTG CCTGAGAATG |
| 421 | GATGACAAGA GCATTAGACC TACCCTGCGC GAGAATGACG TGTTCACCCC CGTCAGGAAG |
| 481 | ATCTGGGACC TGTTTATTCA CCAGTGCATT CAGAACTATA CCCCTGGCGC CCACCTGACC |
| 541 | ATTGATGAGC AGCTCCTGGG ATTCAGGGGC CGGTGTCCCT TCAGAGTGTA TATACCTAAC |
| 601 | AAGCCCTCTA AGTACGGCAT CAAAATCCTG ATGATGTGCG ACTCCGGAAC CAAGTACATG |
| 661 | ATTAACGGAA TGCCCTATCT GGGACGCGGG ACCCAGACCA ATGGAGTGCC TCTGGGCGAG |
| 721 | TATTACGTGA AAGAGCTGTC CAAACCCGTG CACGGGTCCT GTCGGAATAT CACCTGCGAC |
| 781 | AACTGGTTCA CTTCAATCCC CCTGGCCAAA AACCTGCTGC AGGAGCCTTA CAAATTGACC |
| 841 | ATTGTGGGCA CTGTGCGCTC CAATAAGAGA GAAATTCCTG AGGTGCTGAA GAACTCCAGG |
| 901 | TCCAGACCCG TGGGCACTAG CATGTTCTGC TTCGACGGGC CTCTGACCCT GGTGAGCTAT |
| 961 | AAGCCCAAGC CAGCCAAGAT GGTGTACCTG CTGAGCAGCT GCGACGAGGA TGCCTCAATC |
| 1021 | AACGAGAGCA CCGGCAAGCC CCAGATGGTG ATGTACTACA ATCAGACCAA AGGCGGCGTG |
| 1081 | GATACCCTGG ACCAGATGTG TTCCGTGATG ACCTGTAGCA GGAAGACCAA CCGGTGGCCA |
| 1141 | ATGGCCCTGC TGTATGGAAT GATCAACATT GCCTGCATCA ACAGCTTCAT TATTTACTCC |
| 1201 | CACAATGTGT CCTCTAAGGG GGAGAAGGTG CAGTCTAGAA AAAAGTTTAT GAGGAATCTG |
| 1261 | TATATGGGCC TGACCTCTTC CTTCATGCGG AAGCGCCTGG AGGCACCCAC ACTGAAGAGG |
| 1321 | TACCTTCGCG ACAATATCTC CAATATTCTG CCGAAAGAGG TGCCCGGCAC CTCCGACGAC |
| 1381 | AGCACAGAAG AGCCCGTGAT GAAAAAGAGG ACCTACTGCA CCTACTGCCC CTCAAAGATC |
| 1441 | CGCAGGAAGG CCTCCGCATC CTGTAAGAAG TGCAAGAAGG TCATCTGCAG GGAACACAAT |
| 1501 | ATCGACATGT GCCAGTCATG CTTC |
| SEQ ID NO: 508: Trichnoplusia ni (hyperactive piggyBac) amino acid sequence (N3; amino | |
| acid 2-86 deletion) 508 aa | |
| 1 | MRTIRGKNKH CWSTSKPTRR SRVSALNIVR SQRGPTRMCR NIYDPLLCFK LFFTDEIISE |
| 61 | IVKWTNAEIS LKRRESMTSA TFRDTNEDEI YAFFGILVMT AVRKDNHMST DDLFDRSLSM |
| 121 | VYVSVMSRDR FDFLIRCLRM DDKSIRPTLR ENDVFTPVRK IWDLFIHQCI QNYTPGAHLT |
| 181 | IDEQLLGFRG RCPFRVYIPN KPSKYGIKIL MMCDSGTKYM INGMPYLGRG TQTNGVPLGE |
| 241 | YYVKELSKPV HGSCRNITCD NWFTSIPLAK NLLQEPYKLT IVGTVRSNKR EIPEVLKNSR |
| 301 | SRPVGTSMFC FDGPLTLVSY KPKPAKMVYL LSSCDEDASI NESTGKPQMV MYYNQTKGGV |
| 361 | DTLDQMCSVM TCSRKTNRWP MALLYGMINI ACINSFIIYS HNVSSKGEKV QSRKKFMRNL |
| 421 | YMGLTSSFMR KRLEAPTLKR YLRDNISNIL PKEVPGTSDD STEEPVMKKR TYCTYCPSKI |
| 481 | RRKASASCKK CKKVICREHN IDMCQSCF |
| SEQ ID NO: 509: Trichnoplusia ni (hyperactive piggyBac) nucleotide sequence (N4; | |
| nucleotide 4-348 deletion). 1437 bp | |
| 1 | ATGAGCCAGA GAGGTCCAAC CCGGATGTGC AGAAACATCT ACGATCCCCT GCTGTGTTTC |
| 61 | AAGCTGTTTT TTACTGACGA GATTATCAGC GAGATCGTGA AGTGGACCAA CGCCGAGATC |
| 121 | TCTCTGAAGC GGAGAGAAAG CATGACCTCC GCCACATTCC GCGATACCAA CGAAGACGAG |
| 181 | ATCTACGCCT TTTTCGGCAT CTTGGTGATG ACTGCCGTGA GGAAGGACAA TCACATGAGC |
| 241 | ACCGATGACC TGTTCGATCG CAGCCTGAGC ATGGTGTACG TGTCCGTGAT GTCCAGGGAC |
| 301 | AGGTTCGACT TCCTGATCAG GTGCCTGAGA ATGGATGACA AGAGCATTAG ACCTACCCTG |
| 361 | CGCGAGAATG ACGTGTTCAC CCCCGTCAGG AAGATCTGGG ACCTGTTTAT TCACCAGTGC |
| 421 | ATTCAGAACT ATACCCCTGG CGCCCACCTG ACCATTGATG AGCAGCTCCT GGGATTCAGG |
| 481 | GGCCGGTGTC CCTTCAGAGT GTATATACCT AACAAGCCCT CTAAGTACGG CATCAAAATC |
| 541 | CTGATGATGT GCGACTCCGG AACCAAGTAC ATGATTAACG GAATGCCCTA TCTGGGACGC |
| 601 | GGGACCCAGA CCAATGGAGT GCCTCTGGGC GAGTATTACG TGAAAGAGCT GTCCAAACCC |
| 661 | GTGCACGGGT CCTGTCGGAA TATCACCTGC GACAACTGGT TCACTTCAAT CCCCCTGGCC |
| 721 | AAAAACCTGC TGCAGGAGCC TTACAAATTG ACCATTGTGG GCACTGTGCG CTCCAATAAG |
| 781 | AGAGAAATTC CTGAGGTGCT GAAGAACTCC AGGTCCAGAC CCGTGGGCAC TAGCATGTTC |
| 841 | TGCTTCGACG GGCCTCTGAC CCTGGTGAGC TATAAGCCCA AGCCAGCCAA GATGGTGTAC |
| 901 | CTGCTGAGCA GCTGCGACGA GGATGCCTCA ATCAACGAGA GCACCGGCAA GCCCCAGATG |
| 961 | GTGATGTACT ACAATCAGAC CAAAGGCGGC GTGGATACCC TGGACCAGAT GTGTTCCGTG |
| 1021 | ATGACCTGTA GCAGGAAGAC CAACCGGTGG CCAATGGCCC TGCTGTATGG AATGATCAAC |
| 1081 | ATTGCCTGCA TCAACAGCTT CATTATTTAC TCCCACAATG TGTCCTCTAA GGGGGAGAAG |
| 1141 | GTGCAGTCTA GAAAAAAGTT TATGAGGAAT CTGTATATGG GCCTGACCTC TTCCTTCATG |
| 1201 | CGGAAGCGCC TGGAGGCACC CACACTGAAG AGGTACCTTC GCGACAATAT CTCCAATATT |
| 1261 | CTGCCGAAAG AGGTGCCCGG CACCTCCGAC GACAGCACAG AAGAGCCCGT GATGAAAAAG |
| 1321 | AGGACCTACT GCACCTACTG CCCCTCAAAG ATCCGCAGGA AGGCCTCCGC ATCCTGTAAG |
| 1381 | AAGTGCAAGA AGGTCATCTG CAGGGAACAC AATATCGACA TGTGCCAGTC ATGCTTC |
| SEQ ID NO: 510: Trichnoplusia ni (hyperactive piggyBac) amino acid sequence (N4; amino | |
| acid 2-116 deletion). 479 aa | |
| 1 | MSQRGPTRMC RNIYDPLLCF KLFFTDEIIS EIVKWTNAEI SLKRRESMTS ATFRDTNEDE |
| 61 | IYAFFGILVM TAVRKDNHMS TDDLFDRSLS MVYVSVMSRD RFDFLIRCLR MDDKSIRPTL |
| 121 | RENDVFTPVR KIWDLFIHQC IQNYTPGAHL TIDEQLLGFR GRCPFRVYIP NKPSKYGIKI |
| 181 | LMMCDSGTKY MINGMPYLGR GTQTNGVPLG EYYVKELSKP VHGSCRNITC DNWFTSIPLA |
| 241 | KNLLQEPYKL TIVGTVRSNK REIPEVLKNS RSRPVGTSMF CFDGPLTLVS YKPKPAKMVY |
| 301 | LLSSCDEDAS INESTGKPQM VMYYNQTKGG VDTLDQMCSV MTCSRKTNRW PMALLYGMIN |
| 361 | IACINSFIIY SHNVSSKGEK VQSRKKFMRN LYMGLTSSFM RKRLEAPTLK RYLRDNISNI |
| 421 | LPKEVPGTSD DSTEEPVMKK RTYCTYCPSK IRRKASASCK KCKKVICREH NIDMCQSCF |

In embodiments, the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an C-terminus of the polypeptide having an amino acid sequence of SEQ ID NO: 502.

In embodiments, the piggyBac with deletion from the C-terminus comprises SEQ ID NO: 512 or SEQ ID NO: 514, or a sequence having at least about 90% identity thereto.

| SEQ ID NO: 511: Trichnoplusia ni (hyperactive piggyBac) nucleotide sequence (C1; | |
|---|---|
| nucleotide 1708-1782 deletion). 1707 bp | |
| 1 | ATGGGCTCTT CCTTGGACGA CGAGCACATC CTGAGCGCCC TGCTGCAGTC CGATGACGAG |
| 61 | CTGGTGGGTG AGGACTCCGA CAGTGAGGTG AGCGATCATG TGTCCGAGGA CGATGTGCAG |
| 121 | AGCGACACCG AGGAGGCCTT CATCGATGAG GTGCATGAAG TGCAGCCCAC ATCCTCCGGA |
| 181 | TCCGAGATCC TGGATGAGCA GAACGTGATC GAGCAGCCCG GGAGCTCTCT GGCTTCCAAC |
| 241 | AGAATCCTGA CCCTGCCTCA GAGGACTATC AGAGGGAAAA ATAAGCACTG CTGGTCCACT |
| 301 | TCAAAGCCAA CCAGGCGGTC TAGGGTGTCC GCCCTGAACA TCGTGAGAAG CCAGAGAGGT |
| 361 | CCAACCCGGA TGTGCAGAAA CATCTACGAT CCCCTGCTGT GTTTCAAGCT GTTTTTTACT |
| 421 | GACGAGATTA TCAGCGAGAT CGTGAAGTGG ACCAACGCCG AGATCTCTCT GAAGCGGAGA |
| 481 | GAAAGCATGA CCTCCGCCAC ATTCCGCGAT ACCAACGAAG ACGAGATCTA CGCCTTTTTC |
| 541 | GGCATCTTGG TGATGACTGC CGTGAGGAAG GACAATCACA TGAGCACCGA TGACCTGTTC |
| 601 | GATCGCAGCC TGAGCATGGT GTACGTGTCC GTGATGTCCA GGGACAGGTT CGACTTCCTG |
| 661 | ATCAGGTGCC TGAGAATGGA TGACAAGAGC ATTAGACCTA CCCTGCGCGA GAATGACGTG |
| 721 | TTCACCCCCG TCAGGAAGAT CTGGGACCTG TTTATTCACC AGTGCATTCA GAACTATACC |
| 781 | CCTGGCGCCC ACCTGACCAT TGATGAGCAG CTCCTGGGAT TCAGGGGCCG GTGTCCCTTC |
| 841 | AGAGTGTATA TACCTAACAA GCCCTCTAAG TACGGCATCA AAATCCTGAT GATGTGCGAC |
| 901 | TCCGGAACCA AGTACATGAT TAACGGAATG CCCTATCTGG GACGCGGGAC CCAGACCAAT |
| 961 | GGAGTGCCTC TGGGCGAGTA TTACGTGAAA GAGCTGTCCA AACCCGTGCA CGGGTCCTGT |
| 1021 | CGGAATATCA CCTGCGACAA CTGGTTCACT TCAATCCCCC TGGCCAAAAA CCTGCTGCAG |
| 1081 | GAGCCTTACA AATTGACCAT TGTGGGCACT GTGCGCTCCA ATAAGAGAGA AATTCCTGAG |
| 1141 | GTGCTGAAGA ACTCCAGGTC CAGACCCGTG GGCACTAGCA TGTTCTGCTT CGACGGGCCT |
| 1201 | CTGACCCTGG TGAGCTATAA GCCCAAGCCA GCCAAGATGG TGTACCTGCT GAGCAGCTGC |
| 1261 | GACGAGGATG CCTCAATCAA CGAGAGCACC GGCAAGCCCC AGATGGTGAT GTACTACAAT |
| 1321 | CAGACCAAAG GCGGCGTGGA TACCCTGGAC CAGATGTGTT CCGTGATGAC CTGTAGCAGG |
| 1381 | AAGACCAACC GGTGGCCAAT GGCCCTGCTG TATGGAATGA TCAACATTGC CTGCATCAAC |
| 1441 | AGCTTCATTA TTTACTCCCA CAATGTGTCC TCTAAGGGGG AGAAGGTGCA GTCTAGAAAA |
| 1501 | AAGTTTATGA GGAATCTGTA TATGGGCCTG ACCTCTTCCT TCATGCGGAA GCGCCTGGAG |
| 1561 | GCACCCACAC TGAAGAGGTA CCTTCGCGAC AATATCTCCA ATATTCTGCC GAAAGAGGTG |
| 1621 | CCCGGCACCT CCGACGACAG CACAGAAGAG CCCGTGATGA AAAAGAGGAC CTACTGCACC |
| 1681 | TACTGCCCCT CAAAGATCCG CAGGAAG |
| SEQ ID NO: 512: Trichnoplusia ni (hyperactive piggyBac) amino acid sequence (C1; amino | |
| acid 570-594 deletion). 569 aa | |
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKPTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMGL TSSFMRKRLE APTLKRYLRD NISNILPKEV |
| 541 | PGTSDDSTEE PVMKKRTYCT YCPSKIRRK |
| SEQ ID NO: 513: Trichnoplusia ni (hyperactive piggyBac) nucleotide sequence (C2; | |
| nucleotide 1660-1782 deletion). 1659 bp | |
| 1 | ATGGGCTCTT CCTTGGACGA CGAGCACATC CTGAGCGCCC TGCTGCAGTC CGATGACGAG |
| 61 | CTGGTGGGTG AGGACTCCGA CAGTGAGGTG AGCGATCATG TGTCCGAGGA CGATGTGCAG |
| 121 | AGCGACACCG AGGAGGCCTT CATCGATGAG GTGCATGAAG TGCAGCCCAC ATCCTCCGGA |
| 181 | TCCGAGATCC TGGATGAGCA GAACGTGATC GAGCAGCCCG GGAGCTCTCT GGCTTCCAAC |
| 241 | AGAATCCTGA CCCTGCCTCA GAGGACTATC AGAGGGAAAA ATAAGCACTG CTGGTCCACT |
| 301 | TCAAAGCCAA CCAGGCGGTC TAGGGTGTCC GCCCTGAACA TCGTGAGAAG CCAGAGAGGT |
| 361 | CCAACCCGGA TGTGCAGAAA CATCTACGAT CCCCTGCTGT GTTTCAAGCT GTTTTTTACT |
| 421 | GACGAGATTA TCAGCGAGAT CGTGAAGTGG ACCAACGCCG AGATCTCTCT GAAGCGGAGA |
| 481 | GAAAGCATGA CCTCCGCCAC ATTCCGCGAT ACCAACGAAG ACGAGATCTA CGCCTTTTTC |
| 541 | GGCATCTTGG TGATGACTGC CGTGAGGAAG GACAATCACA TGAGCACCGA TGACCTGTTC |
| 601 | GATCGCAGCC TGAGCATGGT GTACGTGTCC GTGATGTCCA GGGACAGGTT CGACTTCCTG |
| 661 | ATCAGGTGCC TGAGAATGGA TGACAAGAGC ATTAGACCTA CCCTGCGCGA GAATGACGTG |
| 721 | TTCACCCCCG TCAGGAAGAT CTGGGACCTG TTTATTCACC AGTGCATTCA GAACTATACC |
| 781 | CCTGGCGCCC ACCTGACCAT TGATGAGCAG CTCCTGGGAT TCAGGGGCCG GTGTCCCTTC |
| 841 | AGAGTGTATA TACCTAACAA GCCCTCTAAG TACGGCATCA AAATCCTGAT GATGTGCGAC |
| 901 | TCCGGAACCA AGTACATGAT TAACGGAATG CCCTATCTGG GACGCGGGAC CCAGACCAAT |
| 961 | GGAGTGCCTC TGGGCGAGTA TTACGTGAAA GAGCTGTCCA AACCCGTGCA CGGGTCCTGT |
| 1021 | CGGAATATCA CCTGCGACAA CTGGTTCACT TCAATCCCCC TGGCCAAAAA CCTGCTGCAG |
| 1081 | GAGCCTTACA AATTGACCAT TGTGGGCACT GTGCGCTCCA ATAAGAGAGA AATTCCTGAG |
| 1141 | GTGCTGAAGA ACTCCAGGTC CAGACCCGTG GGCACTAGCA TGTTCTGCTT CGACGGGCCT |
| 1201 | CTGACCCTGG TGAGCTATAA GCCCAAGCCA GCCAAGATGG TGTACCTGCT GAGCAGCTGC |
| 1261 | GACGAGGATG CCTCAATCAA CGAGAGCACC GGCAAGCCCC AGATGGTGAT GTACTACAAT |
| 1321 | CAGACCAAAG GGGCGTGGA TACCCTGGAC CAGATGTGTT CCGTGATGAC CTGTAGCAGG |
| 1381 | AAGACCAACC GGTGGCCAAT GGCCCTGCTG TATGGAATGA TCAACATTGC CTGCATCAAC |
| 1441 | AGCTTCATTA TTTACTCCCA CAATGTGTCC TCTAAGGGGG AGAAGGTGCA GTCTAGAAAA |
| 1501 | AAGTTTATGA GGAATCTGTA TATGGGCCTG ACCTCTTCCT TCATGCGGAA GCGCCTGGAG |
| 1561 | GCACCCACAC TGAAGAGGTA CCTTCGCGAC AATATCTCCA ATATTCTGCC GAAAGAGGTG |
| 1621 | CCCGGCACCT CCGACGACAG CACAGAAGAG CCCGTGATG |
| SEQ ID NO: 514: Trichnoplusia ni (hyperactive piggyBac) amino acid sequence (C2; amino | |
| acid 554-594 deletion). 553 aa | |
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKPTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMGL TSSFMRKRLE APTLKRYLRD NISNILPKEV |
| 541 | PGTSDDSTEE PVM |

In embodiments, the piggyBac comprises a deletion at positions about 1-5, or about 1-15, or about 1-25, or about 1-35, or about 1-45, or about 1-55, or about 1-65, or about 1-75, or about 1-85, or about 1-95, or about 1-105, or about 1-115, or about 1-125, or about 1-135, or about 1-145, or about 1-155 or positions corresponding thereto, wherein the positions are relative to SEQ ID NO: 502.

In aspects, the N terminal deletion variant is further fused one or more DNA binding domains. In embodiments, the DNA binding domain comprises, without limitation, dCas9, dCas12j, TALEs, and ZnF. In embodiments, the DNA binding domain guides donor insertion to specific genomic sites. In embodiments, the C terminal deletion variant is further fused one or more DNA binding domains. In embodiments, the N terminal deletion variant is further fused one or more DNA binding domains at the N-terminus. In embodiments, the N terminal deletion variant is further fused one or more DNA binding domains at the C-terminus. In embodiments, the C terminal deletion variant is further fused one or more DNA binding domains at the N-terminus. In embodiments, the C terminal deletion variant is further fused one or more DNA binding domains at the C-terminus.

In embodiments, the piggyBac mutant exhibits improved excision frequencies compared to those without the terminal deletions and/or DNA binding domains. In embodiments, the piggyBac mutant exhibits improved integration frequencies compared to those without the terminal deletions and/or DNA binding domains. In embodiments, the piggyBac mutant exhibits improved excision and integration frequencies compared to those without the terminal deletions and/or DNA binding domains.

In embodiments, the N or C terminal mutant exhibit different Exc+/Int−frequencies compared to those without the terminal deletions and/or DNA binding domains. In embodiments, deletion of either N or C termini can result in piggyBac with increased excision activity compared to those without the terminal deletions and/or DNA binding domains. In embodiments, N-terminal deletion yields a mutant with decreased integration compared to those without the terminal deletions and/or DNA binding domains. In embodiments, C-terminal deletion yields a mutant with reduced excision and no integration compared to those without the terminal deletions and/or DNA binding domains.

### Host Cell

In some aspects, the present disclosure further provides a host cell comprising the composition in accordance with embodiments of the present disclosure.

### Methods

In certain embodiments, the present disclosure provides a method for inserting a gene into the genome of a cell, comprising contacting a cell with the composition of the present disclosure or host cell of the present disclosure. In some embodiments, the method further comprises contacting the cell with a polynucleotide encoding a transposon.

In some embodiments, the transposon comprises a gene encoding a complete polypeptide.

In some embodiments, the transposon comprises a gene which is defective or substantially absent in a disease state.

In certain embodiments, the present disclosure provides a method for treating a disease or disorder ex vivo, comprising contacting a cell with the composition of the present disclosure or host cell of the present disclosure and administering the cell to a subject in need thereof.

In certain embodiments, the present disclosure provides a method for treating a disease or disorder in vivo, comprising administering the composition of the present disclosure or host cell of the present disclosure to a subject in need thereof.

### Transgene

In embodiments, the transgene is an exogenous wild-type gene that, e.g., corrects a defective function of one or more mutations in a recipient. For instance, in embodiments, the recipient may have a mutation that provides a disease phenotype (e.g., a defective or absent gene product). In embodiments, the transposon system or method of the present disclosure provides a correction that restores the gene product and diminishes the disease phenotype.

In embodiments, the transgene is a gene that replaces, inactivates, or provides suicide or helper functions.

In embodiments, the transgene and/or disease to be treated is one or more of:
beta-thalassemia: BCL11a or β-globin or BA-T87Q-globin,
LCA: RPE65,
LHON: ND4,
Achromatopsia: CNGA3 or CNGA3/CNGB3,
Choroideremia: REP1,
PKD: RPK (Red cell PK),
Hemophilia: F8,
ADA-SCID: ADA,
Fabry disease: GLA,
MPS type I: IDUA, and
MPS type II: IDS.

In embodiments, the disease or disorder to be treated comprises all inherited monogenic disorders. In embodiments, the disease or disorder to be treated comprises all inherited polygenic disorders.

In embodiments, the transposon comprises a gene encoding a complete polypeptide. In embodiments, the transposon comprises a gene which is defective or substantially absent in a disease state.

In embodiments, the transfecting of the cell is carried out using electroporation or calcium phosphate precipitation.

In embodiments, the transfecting of the cell is carried out using a lipid vehicle, optionally N-[1-(2,3-dioleoyloxy) propyl]-N,N,N-trimethylammonium chloride (DOTMA), 1,2-bis (oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using a lipid selected from one or more of the following categories: cationic lipids; anionic lipids; neutral lipids; multi-valent charged lipids; and zwitterionic lipids. In embodiments, a cationic lipid may be used to facilitate a charge-charge interaction with nucleic acids. In embodiments, the lipid is a neutral lipid. In embodiments, the neutral lipid is dioleoylphosphatidylethanolamine (DOPE), 1,2-Dioleoyl-sn-glycero-3-phosphocholine (DOPC), or cholesterol. In embodiments, cholesterol is derived from plant sources. In other embodiments, cholesterol is derived from animal, fungal, bacterial, or archaeal sources. In embodiments, the lipid is a cationic lipid. In embodiments, the cationic lipid is N-[1-(2,3-dioleoyloxy) propyl]-N,N, N-trimethylammonium chloride (DOTMA), 1,2-bis (oleoyloxy)-3-3-(trimethylammonia) propane (DOTAP), or 1,2-dioleoyl-3-dimethylammonium-propane (DODAP). In embodiments, one or more of the phospholipids 18:0 PC, 18:1 PC, 18:2 PC, DMPC, DSPE, DOPE, 18:2 PE, DMPE, or a combination thereof are used as lipids. In embodiments, the lipid is DOTMA and DOPE, optionally in a ratio of about 1:1. In embodiments, the lipid is DHDOS and DOPE, optionally in a ratio of about 1:1. In embodiments, the lipid is a commercially available product (e.g., LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation) (Life Technologies).

In embodiments, the transfecting of the cell is carried out using a cationic vehicle, optionally LIPOFECTIN or TRANSFECTAM.

In embodiments, the transfecting of the cell is carried out using a lipid nanoparticle or a liposome.

In embodiments, the method is helper virus-free.

Epigenetic regulatory elements can be used to protect a transgene from unwanted epigenetic effects when placed near the transgene on a vector, including the transgene. See Ley et al., PloS One vol. 8,4 e62784. 30 Apr. 2013, doi: 10.1371/journal.pone.0062784. For example, MARs were shown to increase genomic integration and integration of a transgene while preventing heterochromatin silencing, as exemplified by the human March 1-68. See id.; see also Grandjean et al., Nucleic Acids Res. 2011 August; 39 (15): e104. MARs can also act as insulators and thereby prevent the activation of neighboring cellular genes. Gaussin et al., Gene Ther. 2012 January; 19 (1): 15-24. It has been shown that a piggyBac transposon containing human MARs in CHO cells mediated efficient and sustained expression from a few transgene copies, using cell populations generated without an antibiotic selection procedure. See Ley et al. (2013).

In embodiments, the cell is further transfected with a third nucleic acid having at least one chromatin element, wherein the at least one chromatin element is optionally a Matrix Attachment Region (MAR) element. MARs are expression-enhancing, epigenetic regulator elements which are used to enhance and/or facilitate transgene expression, as described, for example, in PCT/IB2010/002337 (WO2011033375), which is incorporated by reference herein in its entirety. A MAR element can be located in cis or trans to the transgene.

In embodiments, the transgene has a size of 100,000 bases or less, e.g., about 100,000 bases, or about 50,000 bases, or about 30,000 bases, or about 10,000 bases, or about 5,000 bases, or about 10,000 to about 100,000 bases, or about 30,000 to about 100,000 bases, or about 50,000 to about 100,000 bases, or about 10,000 to about 50,000 bases, or about 10,000 to about 30,000 bases, or about 30,000 to about 50,000 bases.

In embodiments, the transgene has a size of about 200,000 bases or less, e.g., about 200,000 bases, or about 10,000 to about 200,000 bases, or about 30,000 to about 200,000 bases, or about 50,000 to about 200,000 bases, or about 100,000 to about 200,000 bases, or about 150,000 to about 200,000 bases.

### Targeting Chimeric Constructs

In aspects, the present disclosure provides for a transposon system, e.g., in embodiments, a transposase enzyme comprises a targeting element.

In embodiments, the transposase enzyme associated with the targeting element, is capable of inserting the transposon comprising a transgene, optionally at a TA dinucleotide site or a TTAA (SEQ ID NO: 440) tetranucleotide site in a target site, optionally a genomic safe harbor site (GSHS).

In embodiments, the transposase enzyme associated with the targeting element has one or more mutations which confer hyperactivity.

In embodiments, the transposase enzyme associated with the targeting element has gene cleavage (Exc) and/or gene integration (Int+) activity.

In embodiments, the transposase enzyme associated with the targeting element has gene cleavage (Exc) and/or a lack of gene integration (Int−) activity.

In embodiments, the targeting element comprises one or more proteins or nucleic acids that are capable of binding to a nucleic acid.

In embodiments, the targeting element comprises one or more of a of a gRNA, optionally associated with a CRISPR/Cas enzyme (class I, class II), or their six subtypes (type I-VI) (e.g., Cas12a, Cas12j, Cas12k), which is optionally catalytically inactive, transcription activator-like effector (TALE), Zinc finger, catalytically inactive transcription factor, nickase, a transcriptional activator, a transcriptional repressor, a recombinase, a DNA methyltransferase, a histone methyltransferase, and paternally expressed gene 10 (PEG10).

In embodiments, the targeting element comprises a transcription activator-like effector (TALE) DNA binding domain (DBD).

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG. In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the GSHS is located on human chromosome 2, 4, 6, 10, 11, or 17. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a CRISPR/Cas enzyme (class I, class II), or their six subtypes (type I-VI) (e.g., Cas12a, Cas12j, Cas12k) guide RNA complex.

In embodiments, a targeting chimeric system or construct, having a DBD fused to the transposase enzyme directs binding of the transposase to a specific sequence (e.g., transcription activator-like effector proteins (TALE) repeat variable di-residues (RVD) or gRNA) near an enzyme recognition site. The enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS.

In embodiments, TALEs described herein can physically sequester the enzyme to GSHS and promote transposition to nearby TTAA (SEQ ID NO: 440) sequences in close proximity to the RVD TALE nucleotide sequences. GSHS in open chromatin sites are specifically targeted based on the predilection for transposases to insert into open chromatin.

In embodiments, the transposase enzyme is capable of targeted genomic integration by transposition is linked to or fused with a TALE DNA binding domain (DBD) or a Cas-based gene-editing system, such as, e.g., Cas9 or a variant thereof.

In embodiments, the targeting element targets the transposase enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the enzyme to a chemokine (C-C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 115)
AAACGGATCTGTTTTCGTGGCTCCC;
(SEQ ID NO: 116)
AAACTGTCCCTGGATCTGTTTTCGC;
(SEQ ID NO: 117)
AAACAGCACCGTGTCCCTGGATCTC;
(SEQ ID NO: 118)
AAACCACTGTCCTAGCACCGTGTCC;
(SEQ ID NO: 119)
AAACGAGGCTGTTGGGTCATTTTCC;
(SEQ ID NO: 120)
AAACAGTGGTCAGGCCGGCCAGGCC;
(SEQ ID NO: 121)
AAACGCCAGGCCTTCAGTGCTCAGC;
(SEQ ID NO: 122)
AAACTCAGTGCTCAGTGGAAACCAC;
(SEQ ID NO: 123)
AAACCGAAAGGACTCCTGGCTATCC;
(SEQ ID NO: 124)
AAACAGTCTGAGCACTGGAAGCGCC;
(SEQ ID NO: 125)
AAACCTTCCCTAGTCTGAGCACTGC;
(SEQ ID NO: 126)
AAACGGCTCTGAAGGAGGAGGGGCC;
(SEQ ID NO: 127)
AAACGGACTCCTGGCTCTGAAGGAC;
(SEQ ID NO: 128)
AAACAGGGTCAAGCTCGGAAACCAC;
(SEQ ID NO: 129)
AAACCCCCAGCAGATACTCTGCAGC;
(SEQ ID NO: 130)
AAACGCAGATACTCTGCAGGAACGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGTGGAGTGGG;
(SEQ ID NO: 328)
GTGGGGGGGGAGTGGGGGG;
(SEQ ID NO: 329)
GGGGGGTGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGTGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO: 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGGGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAACC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGGGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactogacatggagg (SEQ ID NO: 427) and cctgcaggggagtgagcagc (SEQ ID NO: 428).

In embodiments, guide RNAs (gRNAs) for targeting human genomic safe harbor sites are synthesized using any of the oligonucleotide primers elements, e.g., without limitation dCas, in areas of open chromatin are as shown in TABLE 1.

TABLE 1

| GSHS | Identifier | Sequence |
|---|---|---|
| AAVS1 | 14F | ggagccacgaaaacagatcc (SEQ ID NO: 800) |
| AAVS1 | 15F | cgaaaacagatccagggaca (SEQ ID NO: 801) |
| AAVS1 | 16F | agatccagggacacggtgct (SEQ ID NO: 802) |
| AAVS1 | 17F | gacacggtgctaggacagtg (SEQ ID NO: 803) |
| AAVS1 | 18F | gaaaatgacccaacagcctc (SEQ ID NO: 804) |
| AAVS1 | 19F | gcctggccggcctgaccact (SEQ ID NO: 805) |
| AAVS1 | 20F | ctgagcactgaaggcctggc (SEQ ID NO: 806) |
| AAVS1 | 21F | tggtttccactgagcactga (SEQ ID NO: 807) |
| AAVS1 | 22F | gatagccaggagtcctttcg (SEQ ID NO: 808) |
| AAVS1 | 23F | gcgcttccagtgctcagact (SEQ ID NO: 809) |
| AAVS1 | 24F | cagtgctcagactagggaag (SEQ ID NO: 810) |
| AAVS1 | 25F | gcccctcctccttcagagcc (SEQ ID NO: 811) |
| AAVS1 | 26F | tccttcagagccaggagtcc (SEQ ID NO: 812) |
| AAVS1 | 27F | tggtttccgagcttgaccct (SEQ ID NO: 813) |
| AAVS1 | 28F | ctgcagagtatctgctgggg (SEQ ID NO: 814) |
| AAVS1 | 29F | cgttcctgcagagtatctgc (SEQ ID NO: 815) |
| AAVS1 | AAVS1 | tcccctcccagaaagacctg (SEQ ID NO: 131) |
| AAVS1 | gAAVS2 | tgggctccaagcaatcctgg (SEQ ID NO: 132) |
| AAVS1 | gAAVS3 | gtggctcaggaggtacctgg (SEQ ID NO: 133) |
| AAVS1 | gAAVS4 | gagccacgaaaacagatcca (SEQ ID NO: 134) |
| AAVS1 | gAAVS5 | aagtgaacggggaagggagg (SEQ ID NO: 135) |
| AAVS1 | gAAVS6 | gacaaaagccgaagtccagg (SEQ ID NO: 136) |
| AAVS1 | gAAVS7 | gtggttgataaacccacgtg (SEQ ID NO: 137) |
| AAVS1 | gAAVS8 | tgggaacagccacagcaggg (SEQ ID NO: 138) |
| AAVS1 | gAAVS9 | gcaggggaacggggatgcag (SEQ ID NO: 139) |
| AAVS1 | gAAVS10 | gagatggtggacgaggaagg (SEQ ID NO: 140) |
| AAVS1 | gAAVS11 | gagatggctccaggaaatgg (SEQ ID NO: 141) |
| AAVS1 | gAAVS12 | taaggaatctgcctaacagg (SEQ ID NO: 142) |
| AAVS1 | gAAVS13 | tcaggagactaggaaggagg (SEQ ID NO: 143) |
| AAVS1 | gAAVS14 | tataaggtggtcccagctcg (SEQ ID NO: 144) |
| AAVS1 | gAAVS15 | ctggaagatgccatgacagg (SEQ ID NO: 145) |
| AAVS1 | gAAVS16 | gcacagactagagaggtaag (SEQ ID NO: 146) |
| AAVS1 | gAAVS17 | acagactagagaggtaaggg (SEQ ID NO: 147) |
| AAVS1 | gAAVS18 | gagaggtgacccgaatccac (SEQ ID NO: 148) |
| AAVS1 | gAAVS19 | gcacaggccccagaaggaga (SEQ ID NO: 149) |
| AAVS1 | gAAVS20 | ccggagaggacccagacacg (SEQ ID NO: 150) |
| AAVS1 | gAAVS21 | gagaggacccagacacgggg (SEQ ID NO: 151) |
| AAVS1 | gAAVS22 | gcaacacagcagagagcaag (SEQ ID NO: 152) |
| AAVS1 | gAAVS23 | gaagagggagtggaggaaga (SEQ ID NO: 153) |
| AAVS1 | gAAVS24 | aagacggaacctgaaggagg (SEQ ID NO: 154) |
| AAVS1 | gAAVS25 | agaaagcggcacaggcccag (SEQ ID NO: 155) |
| AAVS1 | gAAVS26 | gggaaacagtgggccagagg (SEQ ID NO: 156) |
| AAVS1 | gAAVS27 | gtccggactcaggagagaga (SEQ ID NO: 157) |
| AAVS1 | gAAVS28 | ggcacagcaagggcactcgg (SEQ ID NO: 158) |
| AAVS1 | gAAVS29 | gaagaggggaagtcgaggga (SEQ ID NO: 159) |
| AAVS1 | gAAVS30 | gggaatggtaaggaggcctg (SEQ ID NO: 160) |
| AAVS1 | gAAVS31 | gcagagtggtcagcacagag (SEQ ID NO: 161) |
| AAVS1 | gAAVS32 | gcacagagtggctaagccca (SEQ ID NO: 162) |
| AAVS1 | gAAVS33 | gacggggtgtcagcataggg (SEQ ID NO: 163) |
| AAVS1 | gAAVS34 | gcccagggccaggaacgacg (SEQ ID NO: 164) |
| AAVS1 | gAAVS35 | ggtggagtccagcacggcgc (SEQ ID NO: 165) |
| AAVS1 | gAAVS36 | acaggccgccaggaactcgg (SEQ ID NO: 166) |
| AAVS1 | gAAVS37 | actaggaagtgtgtagcacc (SEQ ID NO: 167) |
| AAVS1 | gAAVS38 | atgaatagcagactgccccg (SEQ ID NO: 168) |
| AAVS1 | gAAVS39 | acacccctaaaagcacagtg (SEQ ID NO: 169) |
| AAVS1 | gAAVS40 | caaggagttccagcaggtgg (SEQ ID NO: 170) |
| AAVS1 | gAAVS41 | aaggagttccagcaggtggg (SEQ ID NO: 171) |
| AAVS1 | gAAVS42 | tggaaagaggagggaagagg (SEQ ID NO: 172) |
| AAVS1 | gAAVS43 | tcgaattcctaactgccccg (SEQ ID NO: 173) |
| AAVS1 | gAAVS44 | gacctgcccagcacaccctg (SEQ ID NO: 174) |
| AAVS1 | gAAVS45 | ggagcagctgcggcagtggg (SEQ ID NO: 175) |
| AAVS1 | gAAVS46 | gggagggagagcttggcagg (SEQ ID NO: 176) |
| AAVS1 | gAAVS47 | gttacgtggccaagaagcag (SEQ ID NO: 177) |
| AAVS1 | gAAVS48 | gctgaacagagaagagctgg (SEQ ID NO: 178) |
| AAVS1 | gAAVS49 | tctgagggtggagggactgg (SEQ ID NO: 179) |
| AAVS1 | gAAVS50 | ggagaggtgagggacttggg (SEQ ID NO: 180) |
| AAVS1 | gAAVS51 | gtgaaccaggcagacaacga (SEQ ID NO: 181) |
| AAVS1 | gAAVS52 | caggtacctcctgagccacg (SEQ ID NO: 182) |
| AAVS1 | gAAVS53 | gggggagtaggggcatgcag (SEQ ID NO: 183) |
| hROSA26 | gHROSA26-1 | gcaaatggccagcaagggtg (SEQ ID NO: 184) |
| hROSA26 | gHROSA26-2 | caaatggccagcaagggtgg (SEQ ID NO: 309) |
| hROSA26 | gHROSA26-3 | gcagaacctgaggatatgga (SEQ ID NO: 310) |
| hROSA26 | gHROSA26-3 | aatacacagaatgaaaatag (SEQ ID NO: 311) |
| hROSA26 | gHROSA26-4 | ctggtgactagaataggcag (SEQ ID NO: 312) |
| hROSA26 | gHROSA26-5 | tggtgactagaataggcagt (SEQ ID NO: 313) |
| hROSA26 | gHROSA26-6 | taaaagaatgtgaaaagatg (SEQ ID NO: 314) |
| hROSA26 | gHROSA26-7 | tcaggagttcaagaccaccc (SEQ ID NO: 315) |
| hROSA26 | gHROSA26-8 | tgtagtcccagttatgcagg (SEQ ID NO: 316) |
| hROSA26 | gHROSA26-9 | gggttcacaccacaaatgca (SEQ ID NO: 317) |
| hROSA26 | gHROSA26-10 | ggcaaatggccagcaagggt (SEQ ID NO: 318) |
| hROSA26 | gHROSA26-11 | agaaaccaatcccaaagcaa (SEQ ID NO: 319) |
| hROSA26 | gHROSA26-12 | gccaaggacaccaaaaccca (SEQ ID NO: 320) |
| hROSA26 | gHROSA26-13 | agtggtgataaggcaacagt (SEQ ID NO: 321) |
| hROSA26 | gHROSA26-14 | cctgagacagaagtattaag (SEQ ID NO: 322) |
| hROSA26 | gHROSA26-15 | aaggtcacacaatgaatagg (SEQ ID NO: 323) |
| hROSA26 | gHROSA26-16 | caccatactagggaagaaga (SEQ ID NO: 324) |
| hROSA26 | gHROSA26-17 | caataccctgcccttagtgg (SEQ ID NO: 327) |
| hROSA26 | gHROSA26-18 | aataccctgcccttagtggg (SEQ ID NO: 325) |
| hROSA26 | gHROSA26-19 | ttagtggggggtggagtggg (SEQ ID NO: 326) |
| hROSA26 | gHROSA26-20 | gtggggggtggagtgggggg (SEQ ID NO: 328) |
| hROSA26 | gHROSA26-21 | ggggggtggagtggggggtg (SEQ ID NO: 329) |
| hROSA26 | gHROSA26-22 | ggggtggagtggggggtggg (SEQ ID NO: 330) |
| hROSA26 | gHROSA26-23 | gggtggagtggggggtgggg (SEQ ID NO: 331) |
| hROSA26 | gHROSA26-24 | gggggtggggaaagacatcg (SEQ ID NO: 332) |
| hROSA26 | gHROSA26-25 | gcaaatggccagcaagggtg (SEQ ID NO: 184) |
| hROSA26 | gHROSA26-26 | caaatggccagcaagggtgg (SEQ ID NO: 309) |
| hROSA26 | gHROSA26-27 | gcagaacctgaggatatgga (SEQ ID NO: 310) |
| hROSA26 | gHROSA26-28 | aatacacagaatgaaaatag (SEQ ID NO: 311) |
| hROSA26 | gHROSA26-29 | ctggtgactagaataggcag (SEQ ID NO: 312) |
| hROSA26 | gHROSA26-30 | tggtgactagaataggcagt (SEQ ID NO: 313) |
| hROSA26 | gHROSA26-31 | taaaagaatgtgaaaagatg (SEQ ID NO: 314) |
| hROSA26 | gHROSA26-32 | tcaggagttcaagaccaccc (SEQ ID NO: 315) |
| hROSA26 | gHROSA26-33 | tgtagtcccagttatgcagg (SEQ ID NO: 316) |
| hROSA26 | gHROSA26-34 | gggttcacaccacaaatgca (SEQ ID NO: 317) |
| hROSA26 | gHROSA26-35 | ggcaaatggccagcaagggt (SEQ ID NO: 318) |
| hROSA26 | gHROSA26-36 | agaaaccaatcccaaagcaa (SEQ ID NO: 319) |
| hROSA26 | gHROSA26-37 | gccaaggacaccaaaaccca (SEQ ID NO: 320) |
| hROSA26 | gHROSA26-38 | agtggtgataaggcaacagt (SEQ ID NO: 321) |
| hROSA26 | gHROSA26-39 | cctgagacagaagtattaag (SEQ ID NO: 322) |
| hROSA26 | gHROSA26-40 | aaggtcacacaatgaatagg (SEQ ID NO: 323) |
| hROSA26 | gHROSA26-41 | caccatactagggaagaaga (SEQ ID NO: 324) |
| hROSA26 | gHROSA26-42 | caataccctgcccttagtgg (SEQ ID NO: 327) |
| hROSA26 | gHROSA26-43 | aataccctgcccttagtggg (SEQ ID NO: 325) |
| hROSA26 | gHROSA26-44 | ttagtggggggtggagtggg (SEQ ID NO: 326) |
| hROSA26 | gHROSA26-45 | gtggggggtggagtgggggg (SEQ ID NO: 328) |
| hROSA26 | gHROSA26-46 | ggggggtggagtggggggtg (SEQ ID NO: 329) |
| hROSA26 | gHROSA26-47 | ggggtggagtggggggtggg (SEQ ID NO: 330) |
| hROSA26 | gHROSA26-48 | gggtggagtggggggtgggg (SEQ ID NO: 331) |
| hROSA26 | gHROSA26-49 | gggggtggggaaagacatcg (SEQ ID NO: 332) |
| hROSA26 | gHROSA26-50 | gcagctgtgaattctgatag (SEQ ID NO: 333) |
| hROSA26 | gHROSA26-51 | gagatcagagaaaccagatg (SEQ ID NO: 334) |
| hROSA26 | gHROSA26-52 | tctatactgattgcagccag (SEQ ID NO: 335) |
| hROSA26 | gHROSA26-1 | gcaaatggccagcaagggtg (SEQ ID NO: 184) |
| hROSA26 | 44F | AATCGAGAAGCGACTCGACA (SEQ ID NO: 185) |
| hROSA26 | 45F | GTCCCTGGGCGTTGCCCTGC (SEQ ID NO: 186) |
| hROSA26 | 46F | CCCTGGGCGTTGCCCTGCAG (SEQ ID NO: 187) |
| hROSA26 | 1nF | ccgtgggaagataaactaat (SEQ ID NO: 188) |
| hROSA26 | 2nF | tcccctgcagggcaacgccc (SEQ ID NO: 189) |
| hROSA26 | 3nF | gtcgagtcgcttctcgatta (SEQ ID NO: 190) |
| hROSA26 | 4nF | ctgctgcctcccgtcttgta (SEQ ID NO: 191) |
| hROSA26 | 5nF | gagtgccgcaatacctttat (SEQ ID NO: 192) |
| hROSA26 | 6nF | ACACTTTGGTGGTGCAGCAA (SEQ ID NO: 193) |
| hROSA26 | 7nF | TCTCAAATGGTATAAAACTC (SEQ ID NO: 194) |
| hROSA26 | 8nF | ccgtgggaagataaactaat (SEQ ID NO: 188) |
| hROSA26 | 9F | aatcccgcccataatcgaga (SEQ ID NO: 195) |
| hROSA26 | 10F | tcccgcccataatcgagaag (SEQ ID NO: 196) |
| hROSA26 | 11F | cccataatcgagaagcgact (SEQ ID NO: 197) |
| hROSA26 | 12F | gagaagcgactcgacatgga (SEQ ID NO: 198) |
| hROSA26 | 13F | gaagcgactcgacatggagg (SEQ ID NO: 199) |
| hROSA26 | 14F | gcgactcgacatggaggcga (SEQ ID NO: 200) |
| hROSA26 | 44F | TGTCGAGTCGCTTCTCGATT (SEQ ID NO: 201) |
| hROSA26 | 45F | GCAGGGCAACGCCCAGGGAC (SEQ ID NO: 202) |
| hROSA26 | 46F | CTGCAGGGCAACGCCCAGGG (SEQ ID NO: 203) |
| CCR5 | 1F | acagggttaatgtgaagtcc (SEQ ID NO: 217) |
| CCR5 | 2F | tccccctctacatttaaagt (SEQ ID NO: 218) |
| CCR5 | 3F | catttaaagttggtttaagt (SEQ ID NO: 219) |
| CCR5 | 4F | ttagaaaatataaagaataa (SEQ ID NO: 220) |
| CCR5 | 5 | TAAATGCTTACTGGTTTGAA (SEQ ID NO: 221) |
| CCR5 | 6F | TCCTGGGTCCAGAAAAAGAT (SEQ ID NO: 222) |
| CCR5 | 7F | TTGGGTGGTGAGCATCTGTG (SEQ ID NO: 223) |
| CCR5 | 8F | CGGGGAGAGTGGAGAAAAAG (SEQ ID NO: 224) |
| CCR5 | 9F | GTTAAAACTCTTTAGACAAC (SEQ ID NO: 225) |
| CCR5 | 10F | GAAAATCCCCACTAAGATCC (SEQ ID NO: 226) |
| CCR5 | gCCR5-1 | agtagcagtaatgaagctgg (SEQ ID NO: 237) |
| CCR5 | gCCR5-2 | atacccagacgagaaagctg (SEQ ID NO: 238) |
| CCR5 | gCCR5-3 | tacccagacgagaaagctga (SEQ ID NO: 239) |
| CCR5 | gCCR5-4 | ggtggtgagcatctgtgtgg (SEQ ID NO: 240) |
| CCR5 | gCCR5-5 | aaatgagaagaagaggcaca (SEQ ID NO: 241) |
| CCR5 | gCCR5-6 | cttgtggcctgggagagctg (SEQ ID NO: 242) |
| CCR5 | gCCR5-7 | gctgtagaaggagacagagc (SEQ ID NO: 243) |
| CCR5 | gCCR5-8 | gagctggttgggaagacatg (SEQ ID NO: 244) |
| CCR5 | gCCR5-9 | ctggttgggaagacatgggg (SEQ ID NO: 245) |
| CCR5 | gCCR5-10 | cgtgaggatgggaaggaggg (SEQ ID NO: 246) |
| CCR5 | gCCR5-11 | atgcagagtcagcagaactg (SEQ ID NO: 247) |
| CCR5 | gCCR5-12 | aagacatcaagcacagaagg (SEQ ID NO: 248) |
| CCR5 | gCCR5-13 | tcaagcacagaaggaggagg (SEQ ID NO: 249) |
| CCR5 | gCCR5-14 | aaccgtcaataggcaaaggg (SEQ ID NO: 250) |
| CCR5 | gCCR5-15 | ccgtatttcagactgaatgg (SEQ ID NO: 251) |
| CCR5 | gCCR5-16 | gagaggacaggtgctacagg (SEQ ID NO: 252) |
| CCR5 | gCCR5-17 | aaccaaggaagggcaggagg (SEQ ID NO: 253) |
| CCR5 | gCCR5-18 | gacctctgggtggagacaga (SEQ ID NO: 254) |
| CCR5 | gCCR5-19 | cagatgaccatgacaagcag (SEQ ID NO: 255) |
| CCR5 | gCCR5-20 | aacaccagtgagtagagcgg (SEQ ID NO: 256) |
| CCR5 | gCCR5-21 | aggaccttgaagcacagaga (SEQ ID NO: 257) |
| CCR5 | gCCR5-22 | tacagaggcagactaaccca (SEQ ID NO: 258) |
| CCR5 | gCCR5-23 | acagaggcagactaacccag (SEQ ID NO: 259) |
| CCR5 | gCCR5-24 | taaatgacgtgctagacctg (SEQ ID NO: 260) |
| CCR5 | gCCR5-25 | agtaaccactcaggacaggg (SEQ ID NO: 261) |
| chr2 | gchr2-1 | accacaaaacagaaacacca (SEQ ID NO: 262) |
| chr2 | gchr2-2 | gtttgaagacaagcctgagg (SEQ ID NO: 263) |
| chr4 | gchr4-1 | gctgaaccccaaaagacagg (SEQ ID NO: 264) |
| chr4 | gchr4-2 | gcagctgagacacacaccag (SEQ ID NO: 265) |
| chr4 | gchr4-3 | aggacaccccaaagaagctg (SEQ ID NO: 266) |
| chr4 | gchr4-4 | ggacaccccaaagaagctga (SEQ ID NO: 267) |
| chr6 | gchr6-1 | ccagtgcaatggacagaaga (SEQ ID NO: 268) |
| chr6 | gchr6-2 | agaagagggagcctgcaagt (SEQ ID NO: 269) |
| chr6 | gchr6-3 | gtgtttgggccctagagcga (SEQ ID NO: 270) |
| chr6 | gchr6-4 | catgtgcctggtgcaatgca (SEQ ID NO: 271) |
| chr6 | gchr6-5 | tacaaagaggaagataagtg (SEQ ID NO: 272) |
| chr6 | gchr6-6 | gtcacagaatacaccactag (SEQ ID NO: 273) |
| chr6 | gchr6-7 | gggttaccctggacatggaa (SEQ ID NO: 274) |
| chr6 | gchr6-8 | catggaagggtattcactcg (SEQ ID NO: 275) |
| chr6 | gchr6-9 | agagtggcctagacaggctg (SEQ ID NO: 276) |
| chr6 | gchr6-10 | catgctggacagctcggcag (SEQ ID NO: 277) |
| chr6 | gchr6-11 | agtgaaagaagagaaaattc (SEQ ID NO: 278) |
| chr6 | gchr6-12 | tggtaagtctaagaaaccta (SEQ ID NO: 279) |
| chr6 | gchr6-13 | cccacagcctaaccacccta (SEQ ID NO: 280) |
| chr6 | gchr6-14 | aatatttcaaagccctaggg (SEQ ID NO: 281) |
| chr6 | gchr6-15 | gcactcggaacagggtctgg (SEQ ID NO: 282) |
| chr6 | gchr6-16 | agataggagctccaacagtg (SEQ ID NO: 283) |
| chr6 | gchr6-17 | aagttagagcagccaggaaa (SEQ ID NO: 284) |
| chr6 | gchr6-18 | tagagcagccaggaaaggga (SEQ ID NO: 285) |
| chr6 | gchr6-19 | tgaatacccttccatgtcca (SEQ ID NO: 286) |
| chr6 | gchr6-20 | cctgcattgcaccaggcaca (SEQ ID NO: 287) |
| chr6 | gchr6-21 | tctagggcccaaacacacct (SEQ ID NO: 288) |
| chr6 | gchr6-22 | tccctccatctatcaaaagg (SEQ ID NO: 289) |
| chr10 | gchr10-1 | agccctgagacagaagcagg (SEQ ID NO: 290) |
| chr10 | gchr10-2 | gccctgagacagaagcaggt (SEQ ID NO: 291) |
| chr10 | gchr10-3 | aggagatgcagtgatacgca (SEQ ID NO: 292) |
| chr10 | gchr10-4 | acaataccaagggtatccgg (SEQ ID NO: 293) |
| chr10 | gchr10-5 | tgataaagaaaacaaagtga (SEQ ID NO: 294) |
| chr10 | gchr10-6 | aaagaaaacaaagtgaggga (SEQ ID NO: 295) |
| chr10 | gchr10-7 | gtggcaagtggagaaattga (SEQ ID NO: 296) |
| chr10 | gchr10-8 | caagtggagaaattgaggga (SEQ ID NO: 297) |
| chr10 | gchr10-9 | gtggtgatgattgcagctgg (SEQ ID NO: 298) |
| chr11 | gchr11-1 | ctatgtgcctgacacacagg (SEQ ID NO: 299) |
| chr11 | gchr11-2 | gggttggaccaggaaagagg (SEQ ID NO: 300) |
| chr17 | gchr17-1 | gatgcctggaaaaggaaaga (SEQ ID NO: 301) |
| chr17 | gchr17-2 | tagtatgcacctgcaagagg (SEQ ID NO: 302) |
| chr17 | gchr17-3 | tatgcacctgcaagaggcgg (SEQ ID NO: 303) |
| chr17 | gchr17-4 | aggggaagaagagaagcaga (SEQ ID NO: 304) |
| chr17 | gchr17-5 | gctgaatcaagagacaagcg (SEQ ID NO: 305) |
| chr17 | gchr17-6 | aagcaaataaatctcctggg (SEQ ID NO: 306) |
| chr17 | gchr17-7 | agatgagtgctagagactgg (SEQ ID NO: 307) |
| chr17 | gchr17-8 | ctgatggttgagcacagcag (SEQ ID NO: 308) |

In embodiments, gRNAs for targeting human genomic safe harbor sites using any of the gRNA-based targeting elements, e.g., without limitation, dCas, in areas of open chromatin are shown in TABLE 5 or TABLE 6.

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises CRISPR/Cas enzymes (class I, class II), or their six subtypes (type I-VI or a variant thereof, e.g., without limitation, Cas12a (e.g., dCas12a), or Cas12j (e.g., dCas12j), or Cas12k (e.g., dCas12k). In embodiments, the targeting element comprises a Cas enzyme guide RNA complex. In embodiments, comprises a nuclease-deficient dCas12 guide RNA complex, optionally dCas12j guide RNA complex or dCas12a guide RNA complex.

In embodiments, the targeting element is selected from a zinc finger (ZF), transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from CRISPR/Cas enzymes (class I, class II), or their six subtypes (type I-VI including but not limited to Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the transposase enzyme of the present disclosure is capable of inserting a transposon at a TA dinucleotide site or a TTAA tetranucleotide site in a target site, optionally a genomic safe harbor site (GSHS) of a nucleic acid molecule. The transposase enzyme of the present disclosure is suitable for causing insertion of the transposon in a GSHS when contacted with a biological cell.

In embodiments, the targeting element is suitable for directing the transposase enzyme of the present disclosure to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9 or Cas12, or variants thereof) DBDs cause the transposase enzyme of the present disclosure to bind specifically to human GSHS. In embodiments, the TALEs or Cas DBDs sequester the transposase to GSHS and promote transposition to nearby TA dinucleotide or a TTAA tetranucleotide sites which can be located in proximity to the repeat variable di-residues (RVD) TALE or gRNA nucleotide sequences. The GSHS regions are located in open chromatin sites that are susceptible to transposase activity. Accordingly, the transposase enzyme of the present disclosure does not only operate based on its ability to recognize TA or TTAA sites, but it also directs a transposon (having a transgene) to specific locations in proximity to a TALE or Cas DBD. The transposase enzyme of the present disclosure in accordance with embodiments of the present disclosure has negligible risk of genotoxicity and exhibits superior features as compared to existing gene therapies.

In embodiments, the transposase enzyme of the present disclosure is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies.

In embodiments, TALE or Cas DBDs are customizable, such as a TALE or Cas DBDs is selected for targeting a specific genomic location. In embodiments, the genomic location is in proximity to a TA dinucleotide site or a TTAA (SEQ ID NO: 440) tetranucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as Fokl nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14 (1): 49-55. doi: 10.1038/nrm3486. The following table, for example, shows such code:

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

Accordingly, in embodiments, the TALE DBD comprises one or more repeat sequences. In embodiments, the TALE DBD comprises about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG.

In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor; and human Rosa26 locus. In embodiments, the GSHS is located on human chromosome 2, 4, 6, 10, 11, or 17.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ NO: 48), TGCAGGGCAACGCCCAGGG (SEQ ID NO: 49), TCTCGATTATGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26) TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32) TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of

NH NH HD HD NH NH HD HD NG NH NI HD HD NI HD NG NH NH,
NH NI NI NH NH HD HD NG NH NH HD HD NH NH HD HD NG NH,
NH NI NH HD NI HD NG NH NI NI NH NH HD HD NG NH NH HD,
HD HD NI HD NG NH NI NH HD NI HD NG NH NI NI NH NH HD,
NH NH NG NG NG HD HD NI HD NG NH NI NH HD NI HD NG NH,
NH NH NH NH NI NI NI NI NG NH NI HD HD HD NI NI HD NI,
NI NH NH NI HD NI NH NG NH NH NH NH NI NI NI NI NG NH,
HD HD NI NH NH NH NI HD NI HD NH NH NG NH HD NG NI NH,
HD NI NH NI NH HD HD NI NH NH NI NH NG HD HD NG NH NH,
HD HD NG NG HD NI NH NI NH HD HD NI NH NH NI NH NG HD,
HD HD NG HD HD NG NG HD NI NH NI NH HD HD NI NH NH NI,
HD HD NI NH HD HD HD HD NG HD HD NG HD HD NG NG HD NI,
HD HD NH NI NH HD NG NG NH NI HD HD HD NG NG NH NH NI,
NH NH NG NG NG HD HD NH NI NH HD NG NG NH NI HD HD HD,
NH NH NH NH NG NH NH NG NG NG HD HD NH NI NH HD NG NG,
HD NG NH HD NG NH NH NH NH NG NH NH NG NG NG HD HD NH,
NH HD NI NH NI NH NG NI NG HD NG NH HD NG NH NH NH NH,
HD HD NI NI NG HD HD HD HD NG HD NI NH NG,
HD NI NH NG NH HD NG HD NI NH NG NH NH NI NI,
NH NI NI NI HD NI NG HD HD NH NH HD NH NI HD NG HD NI,
HD NH HD HD HD HD NG HD NI NI NI NG HD NG NG NI HD NI,
HD NI NI NI NG HD NG NG NI HD NI NH HD NG NH HD NG HD,
HD NG NG NI HD NI NH HD NG NH HD NG HD NI HD NG HD HD,
NI HD NI NH HD NG NH HD NG HD NI HD NG HD HD HD HD NG,
NH HD NG HD NI HD NG HD HD HD HD NG NH HD NI NH NH NH,
HD HD HD HD NG NH HD NI NH NH NH HD NI NI HD NH HD HD,
NH HD NI NH NH NH HD NI NI HD NH HD HD HD NI NH NH NH,
HD NG HD NH NI NG NG NI NG NH NH NH HD NH NH NH NI NG,
HD NH HD NG NG HD NG HD NH NI NG NG NI NG NH NH NH HD,
NH NG HD NH NI NH NG HD NH HD NG NG HD NG HD NH NI NG,
HD HD NI NG NH NG HD NH NI NH NG HD NH HD NG NG HD NG,
HD NH HD HD NG HD HD NI NG NH NG HD NH NI NH NG HD NH,
HD NH NG HD NI NG HD NH HD HD NG HD HD NI NG NH NG HD,
NH NI NG HD NG HD NH NG HD NI NG HD NH HD HD NG HD HD,
NH HD NG NG HD NI NH HD NG NG HD HD NG NI,
HD NG NK NG NH NI NG HD NI NG NH HD HD NI,
NI HD NI NN NG NN NN NG NI HD NI HD NI HD HD NG,
HD HD NI HD HD HD HD HD HD NI HD NG NI NI NN,
HD NI NG NG NN NN HD HD NN NN NN HD NI HD,
NN HD NG NG NN NI NI HD HD HD NI NN NN NI NN NI,
NI HD NI HD HD HD NN NI NG HD HD NI HD NG NN NN NN,
NN HD NG NN HD NI NG HD NI NI HD HD HD HD,
NN NN HD NI HD NN NI NI NI HD NI HD HD HD NG HD HD,
NN NN NG NN NN HD NG HD NI NG NN HD HD NG NN,
NN NI NG NG NG NN HD NI HD NI NN HD NG HD NI NG,
NI NI NH HD NG HD NG NH NI NH NH NI NH HD,
HD HD HD NG NI NK HD NG NH NG HD HD HD HD,
NH HD HD NG NI NH HD NI NG NH HD NG NI NH,
NI NG NH NH NH HD NG NG HD NI HD NH NH NI NG,
NH NI NI NI HD NG NI NG NH HD HD NG NH HD,
NH HD NI HD HD NI NG NG NH HD NG HD HD HD,
NH NI HD NI NG NH HD NI NI HD NG HD NI NH,
NI HD NI HD HD NI HD NG NI NH NH NH NH NG,
NH NG HD NG NH HD NG NI NH NI HD NI NH NH,
NH NH HD HD NG NI NH NI HD NI NH NH HD NG NH,
NH NI NH NH HD NI NG NG HD NG NG NI NG HD NH,
NN HD HD NG NN NN NI NI NI HD NN NG NG HD HD,
NN NG NN HD NG HD NG NN NI HD NI NI NG NI,
NN NG NG NG NG NN HD NI NN HD HD NG HD HD,
NI HD NI NN HD NG NN NG NN NN NI NI HD NN NG,
HD NI NI NN NI HD HD NN NI NN HD NI HD NG NN HD NG NN,
HD NG NI NG HD HD HD NI NI NI NI HD NG HD NG,
NH NI NI NI NI NI HD NG NI NG NH NG NI NG,
NI NH NH HD NI NH NH HD NG NH NH NG NG NH NI,
HD NI NI NG NI HD NI NI HD HD NI HD NN HD,
NI NG NN NI HD NN NN NI HD NG HD NI NI HD NG,
HD NI HD NI NI HD NI NG NG NG NN NG NI NI,
and
NI NG NG NG HD HD NI NN NG NN HD NI HD NI.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
NH NH HD HD NH NH HD HD NG NH NI HD HD NI HD NG NH
NH;
(SEQ ID NO: 24)
TGAAGGCCTGGCCGGCCTG
and
NH NI NI NH NH HD HD NG NH NH HD HD NH NH HD HD NG
NH;
(SEQ ID NO: 25)
TGAGCACTGAAGGCCTGGC
and
NH NI NH HD NI HD NG NH NI NI NH NH HD HD NG NH NH
HD;
(SEQ ID NO: 26)
TCCACTGAGCACTGAAGGC
and
HD HD NI HD NG NH NI NH HD NI HD NG NH NI NI NH NH
HD;
(SEQ ID NO: 27)
TGGTTTCCACTGAGCACTG
and
NH NH NG NG NG HD HD NI HD NG NH NI NH HD NI HD NG
NH;
(SEQ ID NO: 28)
TGGGGAAAATGACCCAACA
and
NH NH NH NH NI NI NI NI NG NH NI HD HD HD NI NI HD
NI;
(SEQ ID NO: 29)
TAGGACAGTGGGGAAAATG
and
NI NH NH NI HD NI NH NG NH NH NH NH NI NI NI NI NG
NH;
(SEQ ID NO: 30)
TCCAGGGACACGGTGCTAG
and
HD HD NI NH NH NH NI HD NI HD NH NH NG NH HD NG NI
NH;
(SEQ ID NO: 31)
TCAGAGCCAGGAGTCCTGG
and
HD NI NH NI NH HD HD NI NH NH NI NH NG HD HD NG NH
NH;
(SEQ ID NO: 32)
TCCTTCAGAGCCAGGAGTC
and
HD HD NG NG HD NI NH NI NH HD HD NI NH NH NI NH NG
HD;
(SEQ ID NO: 33)
TCCTCCTTCAGAGCCAGGA
and
HD HD NG HD HD NG NG HD NI NH NI NH HD HD NI NH NH
NI;
(SEQ ID NO: 34)
TCCAGCCCCTCCTCCTTCA
and
HD HD NI NH HD HD HD HD NG HD HD NG HD HD NG NG HD
NI;
(SEQ ID NO: 35)
TCCGAGCTTGACCCTTGGA
and
HD HD NH NI NH HD NG NG NH NI HD HD HD NG NG NH NH
NI;
(SEQ ID NO: 36)
TGGTTTCCGAGCTTGACCC
and
NH NH NG NG NG HD HD NH NI NH HD NG NG NH NI HD HD
HD;
(SEQ ID NO: 37)
TGGGGTGGTTTCCGAGCTT
and
NH NH NH NH NG NH NH NG NG NG HD HD NH NI NH HD NG
NG;
(SEQ ID NO: 38)
TCTGCTGGGGTGGTTTCCG
and
HD NG NH HD NG NH NH NH NH NG NH NH NG NG NG HD HD
NH;
(SEQ ID NO: 39)
TGCAGAGTATCTGCTGGGG
and
NH HD NI NH NI NH NG NI NG HD NG NH HD NG NH NH NH
NH;
(SEQ ID NO: 40)
CCAATCCCCTCAGT
and
HD HD NI NI NG HD HD HD HD NG HD NI NH NG;
(SEQ ID NO: 41)
CAGTGCTCAGTGGAA
and
HD NI NH NG NH HD NG HD NI NH NG NH NH NI NI;
(SEQ ID NO: 42)
GAAACATCCGGCGACTCA
and
NH NI NI NI HD NI NG HD HD NH NH HD NH NI HD NG HD
NI;
(SEQ ID NO: 43)
TCGCCCCTCAAATCTTACA
and
HD NH HD HD HD HD NG HD NI NI NI NG HD NG NG NI HD
NI;
(SEQ ID NO: 44)
TCAAATCTTACAGCTGCTC
and
HD NI NI NI NG HD NG NG NI HD NI NH HD NG NH HD NG
HD;
(SEQ ID NO: 45)
TCTTACAGCTGCTCACTCC
and
HD NG NG NI HD NI NH HD NG NH HD NG HD NI HD NG HD
HD;
(SEQ ID NO: 46)
TACAGCTGCTCACTCCCCT
and
NI HD NI NH HD NG NH HD NG HD NI HD NG HD HD HD HD
NG;
(SEQ ID NO: 47)
TGCTCACTCCCCTGCAGGG
and
NH HD NG HD NI HD NG HD HD HD HD NG NH HD NI NH NH
NH;
(SEQ ID NO: 48)
TCCCCTGCAGGGCAACGCC
and
HD HD HD HD NG NH HD NI NH NH NH HD NI NI HD NH HD
HD;
(SEQ ID NO: 49)
TGCAGGGCAACGCCCAGGG
and
NH HD NI NH NH NH HD NI NI HD NH HD HD HD NI NH NH
NH;
(SEQ ID NO: 50)
TCTCGATTATGGGGGGGAT
and
HD NG HD NH NI NG NG NI NG NH NH NH HD NH NH NH NI
NG;
(SEQ ID NO: 51)
TCGCTTCTCGATTATGGGC
and
HD NH HD NG NG HD NG HD NH NI NG NG NI NG NH NH NH
HD;
(SEQ ID NO: 52)
TGTCGAGTCGCTTCTCGAT
and
NH NG HD NH NI NH NG HD NH HD NG NG HD NG HD NH NI
NG;
(SEQ ID NO: 53)
TCCATGTCGAGTCGCTTCT
and
HD HD NI NG NH NG HD NH NI NH NG HD NH HD NG NG HD
NG;
(SEQ ID NO: 54)
TCGCCTCCATGTCGAGTCG
and
HD NH HD HD NG HD HD NI NG NH NG HD NH NI NH NG HD
NH;
(SEQ ID NO: 55)
TCGTCATCGCCTCCATGTC
and
HD NH NG HD NI NG HD NH HD HD NG HD HD NI NG NH
NG HD;
(SEQ ID NO: 56)
TGATCTCGTCATCGCCTCC
and
NH NI NG HD NG HD NH NG HD NI NG HD NH HD HD NG
HL HD;
(SEQ ID NO: 57)
GCTTCAGCTTCCTA
and
NH HD NG NG HD NI NH HD NG NG HD HD NG NI;
(SEQ ID NO: 58)
CTGTGATCATGCCA
and
HD NG NK NG NH NI NG HD NI NG NH HD HD NI;
(SEQ ID NO: 59)
ACAGTGGTACACACCT
and
NI HD NI NN NG NN NN NG NI HD NI HD NI HD HD NG;
(SEQ ID NO: 60)
CCACCCCCCACTAAG
and
HD HD NI HD HD HD HD HD HD NI HD NG NI NI NN;
(SEQ ID NO: 61)
CATTGGCCGGGCAC
and
HD NI NG NG NN NN HD HD NN NN NN HD NI HD;
(SEQ ID NO: 62)
GCTTGAACCCAGGAGA
and
NN HD NG NG NN NI NI HD HD HD NI NN NN NI NN NI;
(SEQ ID NO: 63)
ACACCCGATCCACTGGG
and
NI HD NI HD HD HD NN NI NG HD HD NI HD NG NN NN
NN;
(SEQ ID NO: 64)
GCTGCATCAACCCC
and
NN HD NG NN HD NI NG HD NI NI HD HD HD HD;
(SEQ ID NO: 65)
GCCACAAACAGAAATA
and
NN NN HD NI HD NN NI NI NI HD NI HD HD HD NG HD
HD;
(SEQ ID NO: 66)
GGTGGCTCATGCCTG
and
NN NN NG NN NN HD NG HD NI NG NN HD HD NG NN;
(SEQ ID NO: 67)
GATTTGCACAGCTCAT
and
NN NI NG NG NG NN HD NI HD NI NN HD NG HD NI NG;
(SEQ ID NO: 68)
AAGCTCTGAGGAGCA
and
NI NI NH HD NG HD NG NH NI NH NH NI NH HD;
(SEQ ID NO: 69)
CCCTAGCTGTCCC
and
HD HD HD NG NI NK HD NG NH NG HD HD HD HD;
(SEQ ID NO: 70)
GCCTAGCATGCTAG
and
NH HD HD NG NI NH HD NI NG NH HD NG NI NH;
(SEQ ID NO: 71)
ATGGGCTTCACGGAT
and
NI NG NH NH NH HD NG NG HD NI HD NH NH NI NG;
(SEQ ID NO: 72)
GAAACTATGCCTGC
and
NH NI NI NI HD NG NI NG NH HD HD NG NH HD;
(SEQ ID NO: 73)
GCACCATTGCTCCC
and
NH HD NI HD HD NI NG NG NH HD NG HD HD HD;
(SEQ ID NO: 74)
GACATGCAACTCAG
and
NH NI HD NI NG NH HD NI NI HD NG HD NI NH;
(SEQ ID NO: 75)
ACACCACTAGGGGT
and
NI HD NI HD HD NI HD NG NI NH NH NH NH NG;
(SEQ ID NO: 76)
GTCTGCTAGACAGG
and
NH NG HD NG NH HD NG NI NH NI HD NI NH NH;
(SEQ ID NO: 77)
GGCCTAGACAGGCTG
and
NH NH HD HD NG NI NH NI HD NI NH NH HD NG NH;
(SEQ ID NO: 78)
GAGGCATTCTTATCG
and
NH NI NH NH HD NI NG NG HD NG NG NI NG HD NH;
(SEQ ID NO: 79)
GCCTGGAAACGTTCC
and
NN HD HD NG NN NN NI NI NI HD NN NG NG HD HD;
(SEQ ID NO: 80)
GTGCTCTGACAATA
and
NN NG NN HD NG HD NG NN NI HD NI NI NG NI;
(SEQ ID NO: 81)
GTTTTGCAGCCTCC
and
NN NG NG NG NG NN HD NI NN HD HD NG HD HD;
(SEQ ID NO: 82)
ACAGCTGTGGAACGT
and
NI HD NI NN HD NG NN NG NN NN NI NI HD NN NG;
(SEQ ID NO: 83)
GGCTCTCTTCCTCCT
and
HD NI NI NN NI HD HD NN NI NN HD NI HD NG NN HD NG
NN;
(SEQ ID NO: 84)
CTATCCCAAAACTCT
and
HD NG NI NG HD HD HD NI NI NI NI HD NG HD NG;
(SEQ ID NO: 85)
GAAAAACTATGTAT
and
NH NI NI NI NI NI HD NG NI NG NH NG NI NG;
(SEQ ID NO: 86)
AGGCAGGCTGGTTGA
and
NI NH NH HD NI NH NH HD NG NH NH NG NG NH NI;
(SEQ ID NO: 87)
CAATACAACCACGC
and
HD NI NI NG NI HD NI NI HD HD NI HD NN HD;
(SEQ ID NO: 88)
ATGACGGACTCAACT
NI NG NN NI HD NN NN NI HD NG HD NI NI HD NG;
and
(SEQ ID NO: 89)
CACAACATTTGTAA
and
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site or TTAA (SEQ ID NO: 440) tetranucleotide site.

Illustrative DNA binding codes for targeting human genomic safe harbor in areas of open chromatin via TALES, encompassed by various embodiments are provided in TABLE 2.

TABLE 2

| GSHS | ID | Sequence | TALE (DNA binding code) |
|---|---|---|---|
| AAVS1 | 1 | tggccggcctgaccactgg (SEQ ID | NH NH HD HD NH NH HD HD NG NH NI |
| | | NO: 23) | HD HD NI HD NG NH NH |
| AAVS1 | 2 | tgaaggcctggccggcctg (SEQ ID | NH NI NI NH NH HD HD NG NH NH HD |
| | | NO: 24) | HD NH NH HD HD NG NH |
| AAVS1 | 3 | tgagcactgaaggcctggc (SEQ ID | NH NI NH HD NI HD NG NH NI NI NH NH |
| | | NO: 25) | HD HD NG NH NH HD |
| AAVS1 | 4 | tccactgagcactgaaggc (SEQ ID | HD HD NI HD NG NH NI NH HD NI HD |
| | | NO: 26) | NG NH NI NI NH NH HD |
| AAVS1 | 5 | tggtttccactgagcactg (SEQ ID | NH NH NG NG NG HD HD NI HD NG NH |
| | | NO: 27) | NI NH HD NI HD NG NH |
| AAVS1 | 6 | tggggaaaatgacccaaca (SEQ | NH NH NH NH NI NI NI NING NH NI HD |
| | | ID NO: 28) | HD HD NI NI HD NI |
| AAVS1 | 7 | taggacagtggggaaaatg (SEQ | NI NH NH NI HD NI NH NG NH NH NH |
| | | ID NO: 29) | NH NI NI NI NI NG NH |
| AAVS1 | 8 | tccagggacacggtgctag (SEQ ID | HD HD NI NH NH NH NI HO NI HD NH |
| | | NO: 30) | NH NG NH HD NG NI NH |
| AAVS1 | 9 | tcagagccaggagtcctgg (SEQ ID | HD NI NH NI NH HD HD NI NH NH NI NH |
| | | NO: 31) | NG HD HD NG NH NH |
| AAVS1 | 10 | tccttcagagccaggagtc (SEQ ID | HD HD NG NG HD NI NH NI NH HD HD |
| | | NO: 32) | NI NH NH NI NH NG HD |
| AAVS1 | 11 | tcctccttcagagccagga (SEQ ID | HD HD NG HD HD NG NG HD NI NH NI |
| | | NO: 33) | NH HD HD NI NH NH NI |
| AAVS1 | 12 | tccagcccctcctccttca (SEQ ID | HD HD NI NH HD HD HD HD NG HD HD |
| | | NO: 34) | NG HD HD NG NG HD NI |
| AAVS1 | 13 | tccgagcttgacccttgga (SEQ ID | HD HD NH NI NH HD NG NG NH NI HD |
| | | NO: 35) | HD HD NG NG NH NH NI |
| AAVS1 | 14 | tggtttccgagcttgaccc (SEQ ID | NH NH NG NG NG HD HD NH NI NH HD |
| | | NO: 36) | NG NG NH NI HD HD HD |
| AAVS1 | 15 | tggggtggtttccgagctt (SEQ ID | NH NH NH NH NG NH NH NG NG NG |
| | | NO: 37) | HD HD NH NI NH HD NG NG |
| AAVS1 | 16 | tctgctggggtggtttccg (SEQ ID | HD NG NH HD NG NH NH NH NH NG |
| | | NO: 38) | NH NH NG NG NG HD HD NH |
| AAVS1 | 17 | tgcagagtatctgctgggg (SEQ ID | NH HD NI NH NI NH NG NI NG HD NG |
| | | NO: 39) | NH HD NG NH NH NH NH |
| AAVS1 | AVS1 | CCAATCCCCTCAGT (SEQ | HD HD NI NI NG HD HD HD HD NG HD |
| | | ID NO: 40) | NI NH NG |
| AAVS1 | AVS2 | CAGTGCTCAGTGGAA (SEQ | HD NI NH NG NH HD NG HD NI NH NG |
| | | ID NO: 41) | NH NH NI NI |
| AAVS1 | AVS3 | GAAACATCCGGCGACTCA | NH NI NI NI HD NI NG HD HD NH NH HD |
| | | (SEQ ID NO: 42) | NH NI HD NG HD NI |
| hROSA26 | 1F | tcgcccctcaaatcttaca (SEQ ID | HD NH HD HD HD HD NG HD NI NI NI |
| | | NO: 43) | NG HD NG NG NI HD NI |
| hROSA26 | 2F | tcaaatcttacagctgctc (SEQ ID | HD NI NI NI NG HD NG NG NI HD NI NH |
| | | NO: 44) | HD NG NH HD NG HD |
| hROSA26 | 3F | tottacagctgctcactcc (SEQ ID | HD NG NG NI HD NI NH HD NG NH HD |
| | | NO: 45) | NG HD NI HD NG HD HD |
| hROSA26 | 4F | tacagctgctcactcccct (SEQ ID | NI HD NI NH HD NG NH HD NG HD NI |
| | | NO: 46) | HD NG HD HD HD HD NG |
| hROSA26 | 5F | tgctcactcccctgcaggg (SEQ ID | NH HD NG HD NI HD NG HD HD HD HD |
| | | NO: 47) | NG NH HD NI NH NH NH |
| hROSA26 | 6F | tcccctgcagggcaacgcc (SEQ | HD HD HD HD NG NH HD NI NH NH NH |
| | | ID NO: 48) | HD NI NI HD NH HD HD |
| hROSA26 | 7F | tgcagggcaacgcccaggg (SEQ | NH HD NI NH NH NH HD NI NI HD NH |
| | | ID NO: 49) | HD HD HD NI NH NH NH |
| hROSA26 | 8R | tctcgattatgggcgggat (SEQ ID | HD NG HD NH NI NG NG NI NG NH NH |
| | | NO: 50) | NH HD NH NH NH NI NG |
| hROSA26 | 9R | togcttctcgattatgggc (SEQ ID | HD NH HD NG NG HD NG HD NH NI NG |
| | | NO: 51) | NG NI NG NH NH NH HD |
| hROSA26 | 10R | tgtcgagtcgcttctcgat (SEQ ID | NH NG HD NH NI NH NG HD NH HD NG |
| | | NO: 52) | NG HD NG HD NH NI NG |
| hROSA26 | 11R | tccatgtcgagtcgcttct (SEQ ID | HD HD NI NG NH NG HD NH NI NH NG |
| | | NO: 53) | HD NH HD NG NG HD NG |
| hROSA26 | 12R | tcgcctccatgtcgagtcg (SEQ ID | HD NH HD HD NG HD HD NI NG NH NG |
| | | NO: 54) | HD NH NI NH NG HD NH |
| hROSA26 | 13R | tcgtcatcgcctccatgtc (SEQ ID | HD NH NG HD NI NG HD NH HD HD NG |
| | | NO: 55) | HD HD NI NG NH NG HD |
| hROSA26 | 14R | tgatctcgtcatcgcctcc (SEQ ID | NH NI NG HD NG HD NH NG HD NI NG |
| | | NO: 56) | HD NH HD HD NG HD HD |
| hROSA26 | ROSA1 | GCTTCAGCTTCCTA (SEQ | NH HD NG NG HD NI NH HD NG NG HD |
| | | ID NO: 57) | HD NG NI |
| hROSA26 | ROSA2 | CTGTGATCATGCCA (SEQ | HD NG NK NG NH NI NG HD NI NG NH |
| | | ID NO: 58) | HD HD NI |
| hROSA26 | TALER2 | ACAGTGGTACACACCT | NI HD NI NN NG NN NN NG NI HD NI HD |
| | | (SEQ ID NO: 59) | NI HD HD NG |
| hROSA26 | TALER3 | CCACCCCCCACTAAG (SEQ | HD HD NI HD HD HD HD HD HD NI HD |
| | | ID NO: 60) | NG NI NI NN |
| hROSA26 | TALER4 | CATTGGCCGGGCAC (SEQ | HD NI NG NG NN NN HD HD NN NN NN |
| | | ID NO: 61) | HD NI HD |
| hROSA26 | TALER5 | GCTTGAACCCAGGAGA | NN HD NG NG NN NI NI HD HD HD NI |
| | | (SEQ ID NO: 62) | NN NN NI NN NI |
| CCR5 | TALC3 | ACACCCGATCCACTGGG | NI HD NI HD HD HD NN NI NG HD HD |
| | | (SEQ ID NO: 63) | NI HD NG NN NN NN |
| CCR5 | TALC4 | GCTGCATCAACCCC (SEQ | NN HD NG NN HD NI NG HD NI NI HD |
| | | ID NO: 64) | HD HD HD |
| CCR5 | TALC5 | GCCACAAACAGAAATA | NN NN HD NI HD NN NI NI NI HD NI HD |
| | | (SEQ ID NO: 65) | HD HD NG HD HD |
| CCR5 | TALC7 | GGTGGCTCATGCCTG | NN NN NG NN NN HD NG HD NI NG NN |
| | | (SEQ ID NO: 66) | HD HD NG NN |
| CCR5 | TALC8 | GATTTGCACAGCTCAT | NN NI NG NG NG NN HD NI HD NI NN |
| | | (SEQ ID NO: 67) | HD NG HD NI NG |
| Chr 2 | SHCHR2-1 | AAGCTCTGAGGAGCA (SEQ | NI NI NH HD NG HD NG NH NI NH NH |
| | | ID NO: 68) | NI NH HD |
| Chr 2 | SHCHR2-2 | CCCTAGCTGTCCC (SEQ ID | HD HD HD NG NI NK HD NG NH NG HD |
| | | NO: 69) | HD HD HD |
| Chr 2 | SHCHR2-3 | GCCTAGCATGCTAG (SEQ | NH HD HD NG NI NH HD NI NG NH HD |
| | | ID NO: 70) | NG NI NH |
| Chr 2 | SHCHR2-4 | ATGGGCTTCACGGAT (SEQ | NI NG NH NH NH HD NG NG HD NI HD |
| | | ID NO: 71) | NH NH NI NG |
| Chr 4 | SHCHR4-1 | GAAACTATGCCTGC (SEQ | NH NI NI NI HD NG NI NG NH HD HD NG |
| | | ID NO: 72) | NH HD |
| Chr 4 | SHCHR4-2 | GCACCATTGCTCCC (SEQ | NH HD NI HD HD NI NG NG NH HD NG |
| | | ID NO: 73) | HD HD HD |
| Chr 4 | SHCHR4-3 | GACATGCAACTCAG (SEQ | NH NI HD NI NG NH HD NI NI HD NG HD |
| | | ID NO: 74) | NI NH |
| Chr 6 | SHCHR6-1 | ACACCACTAGGGGT (SEQ | NI HD NI HD HD NI HD NG NI NH NH NH |
| | | ID NO: 75) | NH NG |
| Chr 6 | SHCHR6-2 | GTCTGCTAGACAGG (SEQ | NH NG HD NG NH HD NG NI NH NI HD |
| | | ID NO: 76) | NI NH NH |
| Chr 6 | SHCHR6-3 | GGCCTAGACAGGCTG | NH NH HD HD NG NI NH NI HD NI NH |
| | | (SEQ ID NO: 77) | NH HD NG NH |
| Chr 6 | SHCHR6-4 | GAGGCATTCTTATCG (SEQ | NH NI NH NH HD NI NG NG HD NG NG |
| | | ID NO: 78) | NI NG HD NH |
| Chr 10 | SHCHR10-1 | GCCTGGAAACGTTCC (SEQ | NN HD HD NG NN NN NI NI NI HD NN |
| | | ID NO: 79) | NG NG HD HD |
| Chr 10 | SHCHR10-2 | GTGCTCTGACAATA (SEQ | NN NG NN HD NG HD NG NN NI HD NI |
| | | ID NO: 80) | NI NG NI |
| Chr 10 | SHCHR10-3 | GTTTTGCAGCCTCC (SEQ | NN NG NG NG NG NN HD NI NN HD HD |
| | | ID NO: 81) | NG HD HD |
| Chr 10 | SHCHR10-4 | ACAGCTGTGGAACGT (SEQ | NI HD NI NN HD NG NN NG NN NN NI |
| | | ID NO: 82) | NI HD NN NG |
| Chr 10 | SHCHR10-5 | GGCTCTCTTCCTCCT (SEQ | HD NI NI NN NI HD HD NN NI NN HD NI |
| | | ID NO: 83) | HD NG NN HD NG NN |
| Chr 11 | SHCHR11-1 | CTATCCCAAAACTCT (SEQ | HD NG NI NG HD HD HD NI NI NI NI HD |
| | | ID NO: 84) | NG HD NG |
| Chr 11 | SHCHR11-2 | GAAAAACTATGTAT (SEQ ID | NH NI NI NI NI NI HD NG NI NG NH NG |
| | | NO: 85) | NI NG |
| Chr 11 | SHCHR11-3 | AGGCAGGCTGGTTGA | NI NH NH HD NI NH NH HD NG NH NH |
| | | (SEQ ID NO: 86) | NG NG NH NI |
| Chr 17 | SHCHR17-1 | CAATACAACCACGC (SEQ | HD NI NI NG NI HD NI NI HD HD NI HD |
| | | ID NO: 87) | NN HD |
| Chr 17 | SHCHR17-2 | ATGACGGACTCAACT (SEQ | NI NG NN NI HD NN NN NI HD NG HD |
| | | ID NO: 88) | NI NI HD NG |
| Chr 17 | SHCHR17-3 | CACAACATTTGTAA (SEQ ID | HD NI HD NI NI HD NI NG NG NG NN |
| | | NO: 89) | NG NI NI |
| Chr 17 | SHCHR17-4 | ATTTCCAGTGCACA (SEQ | NI NG NG NG HD HD NI NN NG NN HD |
| | | ID NO: 90) | NI HD NI |

Further illustrative DNA binding codes for targeting human genomic safe harbor in areas of open chromatin via TALES, encompassed by embodiments are provided in TABLE 7 and TABLE 8. In embodiments, the transposase enzyme of the present disclosure is capable of inserting a transposon at a TA dinucleotide site. In embodiments, the transposase enzyme of the present disclosure is capable of inserting a transposon at a TTAA (SEQ ID NO: 440) tetranucleotide site.

In embodiments, the present disclosure relates to a system having nucleic acids encoding the enzyme (e.g., without limitation, the transposase enzyme) and the transposon, respectively.

### Linkers

In some embodiments, the targeting element comprises a nucleic acid binding component of a gene-editing system. In some embodiments, the transposase enzyme the targeting element are connected. Without wishing to be bound by a particular theory, the targeting element may refer to a nucleic acid binding component of the gene-editing system. In some embodiments, the transposase enzyme and the targeting element are connected. For example, in embodiments, the transposase enzyme and the targeting element are fused to one another or linked via a linker to one another.

In some embodiments, the linker is a flexible linker. In some embodiments, the flexible linker is substantially comprised of glycine and serine residues, optionally wherein the flexible linker comprises (Gly4Ser)ₙ, where n is an integer from 1 to 12. In some embodiments, the flexible linker is of about 20, or about 30, or about 40, or about 50, or about 60 amino acid residues. In embodiments, the flexible linker is about 50, or about 100, or about 150, or about 200 amino acid residues in length. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

In embodiments, the linker is a nanobody. In embodiments, the nanobody is an Alfa tag and NbAlfa. In embodiments, the linker is a monobody. In embodiments, the linker is an Alfa tag and monobody.

### Inteins

Inteins (INTervening protEINS) are mobile genetic elements that are protein domains, found in nature, with the capability to carry out the process of protein splicing. See Sarmiento & Camarero (2019) Current Protein & Peptide Science, 20 (5), 408-424, which is incorporated by reference herein in its entirety. Protein spicing is a post-translation biochemical modification which results in the cleavage and formation of peptide bonds between precursor polypeptide segments flanking the intein. Id. Inteins apply standard enzymatic strategies to excise themselves post-translationally from a precursor protein via protein splicing. Nanda et al., Microorganisms vol. 8,12 2004. 16 Dec. 2020, doi: 10.3390/microorganisms8122004. An intein can splice its flanking N- and C-terminal domains to become a mature protein and excise itself from a sequence. For example, split inteins have been used to control the delivery of heterologous genes into transgenic organisms. See Wood & Camarero (2014) J. Biol. Chem. 289 (21): 14512-14519. This approach relies on splitting the target protein into two segments, which are then post-translationally reconstituted in vivo by protein trans-splicing (PTS). See Aboye & Camarero (2012) J. Biol. Chem. 287, 27026-27032. More recently, an intein-mediated split-Cas9 system has been developed to incorporate Cas9 into cells and reconstitute nuclease activity efficiently. Truong et al., Nucleic Acids Res. 2015, 43 (13), 6450-6458. The protein splicing excises the internal region of the precursor protein, which is then followed by the ligation of the N-extein and C-extein fragments, resulting in two polypeptides—the excised intein and the new polypeptide produced by joining the C- and N-exteins. Sarmiento & Camarero (2019).

In embodiments, intein-mediated incorporation of DNA binding domains such as, without limitation, dCas9, dCas12j, or TALEs, allows creation of a split-enzyme system such as, without limitation, split transposase system, that permits reconstitution of the full-length enzyme, e.g., transposase, from two smaller fragments. This allows avoiding the need to express DNA binding domains at the N- or C-terminus of an enzyme, e.g., transposase. In this approach, the two portions of an enzyme, e.g., transposase, are fused to the intein and, after co-expression, the intein allows producing a full-length enzyme, e.g., transposase, by post-translation modification. Thus, in embodiments, a nucleic acid encoding the enzyme capable of targeted genomic integration by transposition comprises an intein. In embodiments, the nucleic acid encodes the enzyme in the form of first and second portions with the intein encoded between the first and second portions, such that the first and second portions are fused into a functional enzyme upon post-translational excision of the intein from the enzyme.

In embodiments, an intein is a suitable ligand-dependent intein, for example, an intein selected from those described in U.S. Pat. No. 9,200,045; Mootz et al., J. Am. Chem. Soc. 2002; 124, 9044-9045; Mootz et al., J. Am. Chem. Soc. 2003; 125, 10561-10569; Buskirk et al., Proc. Natl. Acad. Sci. USA. 2004; 101, 10505-10510; Skretas & Wood. Protein Sci. 2005; 14, 523-532; Schwartz, et al., Nat. Chem. Biol. 2007; 3, 50-54; Peck et al., Chem. Biol. 2011; 18 (5), 619-630; the entire contents of each of which are hereby incorporated by reference herein.

In embodiments the intein is NpuN (Intein-N) (SEQ ID NO: 423) and/or NpuC (Intein-C) (SEQ ID NO: 424), or a variant thereof, e.g., a sequence having at least about 90%, or at least about 93%, or at least about 95%, or at least about 97%, or at least about 98%, or at least about 99% identity thereto.

SEQ ID NO: 423:
nucleotide sequence of NpuN (Intein-N)
GGCGGATCTGGCGGTAGTGCTGAGTATTGTCTGAGTTACGAAACG
GAAATACTCACGGTTGAGTATGGGCTTCTTCCAATTGGCAAAATC
GTTGAAAAGCGCATAGAGTGTACGGTGTATTCCGTCGATAACAAC
GGTAATATCTACACCCAGCCGGTAGCTCAGTGGCACGACCGAGGC
GAACAGGAAGTGTTCGAGTATTGCTTGGAAGATGGCTCCCTTATC
CGCGCCACTAAAGACCATAAGTTTATGACGGTTGACGGGCAGATG
CTGCCTATAGACGAAATATTTGAGAGAGAGCTGGACTTGATGAGA
GTCGATAATCTGCCAAAT
SEQ ID NO: 424:
nucleotide sequence of NpuC (Intein-C)
GGCGGATCTGGCGGTAGTGGGGGTTCCGGATCCATAAAGATAGCT
ACTAGGAAATATCTTGGCAAACAAAACGTCTATGACATAGGAGTT
GAGCGAGATCACAATTTTGCTTTGAAGAATGGGTTCATCGCGTCT
AATTGCTTCAACGCTAGCGGCGGGTCAGGAGGCTCTGGTGGAAGC

### Nucleic Acids of the Disclosure

In embodiments, a nucleic acid encoding the enzyme (e.g., without limitation, the transposase enzyme) is RNA. In embodiments, a nucleic acid encoding the transgene is DNA.

In embodiments, the enzyme (e.g., without limitation, the transposase enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine), and optionally a poly-A tail of about 30, or about 50, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a 5′-UTR comprising a sequence that increases RNA stability in vivo, a 3′-UTR comprising a sequence that increases RNA stability in vivo, and/or a 3′ poly(A) tail.

In embodiments, the enzyme (e.g., without limitation, a transposase) is incorporated into a vector or a vector-like particle. In embodiments, the vector is a non-viral vector.

In embodiments, a nucleic acid encoding the enzyme in accordance with embodiments of the present disclosure, is DNA.

In various embodiments, a construct comprising a transposon is any suitable genetic construct, such as a nucleic acid construct, a plasmid, or a vector. In various embodiments, the construct is DNA, which is referred to herein as a donor DNA. In embodiments, sequences of a nucleic acid encoding the transposon is codon optimized to provide improved mRNA stability and protein expression in mammalian systems.

In embodiments, the enzyme and the transposon are included in different vectors. In embodiments, the enzyme and the transposon are included in the same vector.

In various embodiments, a nucleic acid encoding the enzyme capable of targeted genomic integration by transposition (e.g., without limitation, the transposase enzyme) is RNA (e.g., helper RNA), and a nucleic acid encoding a transposon is DNA.

As would be appreciated in the art, a transposon often includes an open reading frame that encodes a transgene at the middle of transposon and terminal repeat sequences at the 5′ and 3′ end of the transposon. The translated transposase (e.g., without limitation, the transposase enzyme) binds to the 5′ and 3′ sequence of the transposon and carries out the transposition function.

In embodiments, a transposon is used interchangeably with transposable elements, which are used to refer to polynucleotides capable of inserting copies of themselves into other polynucleotides. The term transposon is well known to those skilled in the art and includes classes of transposons that can be distinguished on the basis of sequence organization, for example inverted terminal sequences at each end, and/or directly repeated long terminal repeats (LTRs) at the ends. In embodiments, the transposon as described herein may be described as a piggyBac like element, e.g., a transposon element that is characterized by its traceless excision, which recognizes TTAA (SEQ ID NO: 440) sequence and restores the sequence at the insert site back to the original TTAA (SEQ ID NO: 440) sequence after removal of the transposon.

In embodiments, the transposon is flanked by one or more end sequences or terminal ends. In embodiments, the transposon is or comprises a gene encoding a complete polypeptide. In embodiments, the transposon is or comprises a gene which is defective or substantially absent in a disease state.

In embodiments, a transgene is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the transposon (transgene cassette) to reduce transcriptional silencing and position effects imparted by chromosomal sequences. As an additional effect, the insulators can eliminate functional interactions of the transgene enhancer and promoter sequences with neighboring chromosomal sequences. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21 (8): 1536-50, which is incorporated herein by reference in its entirety.

In embodiments, the transgene is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: 1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat Biotechnol 2011; 29:73-8; Bejerano et al. Science 2004; 304:1321-5.

Furthermore, the use of GSHS locations can allow stable transgene expression across multiple cell types. One such site, chemokine C-C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz at al., Viruses 2010; 2:1069-105. A clinical trial has demonstrated safety and efficacy of disrupting CCR5 via targetable nucleases. Tebas at al., HIV. N Engl J Med 2014; 370:901-10.

In embodiments, the transposon is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107 (7): 2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from transposons, from yeast episomes, from insertion elements, from yeast chromosomal elements, and vectors from combinations thereof. The present constructs may contain control regions that regulate as well as engender expression.

In embodiments, the construct comprising the enzyme and/or transgene is codon optimized. Transgene codon optimization is used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the enzyme and/or transgene includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21 (8): 1536-50, which is incorporated herein by reference in its entirety. In embodiments, the gene of the construct comprising the enzyme and/or transgene is capable of transposition in the presence of a transposase. In embodiments, the non-viral vector in accordance with embodiments of the present disclosure comprises a nucleic acid construct encoding a transposase. The transposase (e.g., without limitation, the transposase enzyme of the present disclosure) is an RNA transposase plasmid. In embodiments, the non-viral vector further comprises a nucleic acid construct encoding a DNA transposase plasmid. In embodiments, the transposase is an in vitro-transcribed mRNA transposase. The transposase (e.g., without limitation, the transposase enzyme of the present disclosure) is capable of excising and/or transposing the gene from the construct comprising the enzyme and/or transgene to site- or locus-specific genomic regions.

In embodiments, the enzyme (e.g., without limitation, the transposase enzyme) and the transposon are included in the same vector.

In embodiments, the enzyme is disposed on the same (cis) or different vector (trans) than a transposon with a transgene. Accordingly, in embodiments, the enzyme and the transposon encompassing a transgene are in cis configuration such that they are included in the same vector. In embodiments, the enzyme and the transposon encompassing a transgene are in trans configuration such that they are included in different vectors. The vector is any non-viral vector in accordance with the present disclosure.

In some aspects, a nucleic acid encoding the transposon system of the present disclosure capable of targeted genomic integration by transposition (e.g., a transposase) in accordance with embodiments of the present disclosure is provided. The nucleic acid is or comprises DNA or RNA. In embodiments, the nucleic acid encoding the enzyme is DNA. In embodiments, the nucleic acid encoding the enzyme capable of targeted genomic integration by transposition (e.g., a transposase of the present disclosure) is RNA such as, e.g., helper RNA. In embodiments, the transposase is incorporated into a vector. In embodiments, the vector is a non-viral vector.

In embodiments, a nucleic acid encoding the transgene in accordance with embodiments of the present disclosure is provided. The nucleic acid is or comprises DNA or RNA. In embodiments, the nucleic acid encoding the transgene is DNA. In embodiments, the nucleic acid encoding the transgene is RNA such as, e.g., helper RNA. In embodiments, the transgene is incorporated into a vector. In embodiments, the vector is a non-viral vector.

In embodiments, the present enzyme can be in the form or an RNA or DNA and have one or two N-terminus nuclear localization signal (NLS) to shuttle the protein more efficiently into the nucleus. For example, in embodiments, the present enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K(K/R)X(K/R) (SEQ ID NO: 348). In an embodiment, the NLS comprises the consensus sequence (K/R)(K/R)X₁₀₋₁₂(K/R)_{3/5} (SEQ ID NO: 349), where (K/R)_{3/5} represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions, or deletions (e.g., about 1, or about 2, or about 3, or about 4, or about 5, or about 10 substitutions, additions, or deletions).

In some aspects, a host cell comprising the nucleic acid in accordance with embodiments of the present disclosure is provided.

### Lipids and LNP Delivery

In embodiments, a composition or a nucleic acid in accordance with embodiments of the present disclosure is provided wherein the composition is in the form of a lipid nanoparticle (LNP). In embodiments, the composition is encapsulated in an LNP.

In embodiments, a nucleic acid encoding the enzyme and a nucleic acid encoding the transgene are contained within the same lipid nanoparticle (LNP). In embodiments, the nucleic acid encoding the enzyme and the nucleic acid encoding the transposon are a mixture incorporated into or associated with the same LNP. In embodiments, the polynucleotide encoding the transposase enzyme and the polynucleotide encoding the transposon are in the form of the same LNP, optionally in a co-formulation.

In embodiments, the LNP is selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly (lactic-co-glycolic acid) (PLGA), and N-Acetylgalactosamine (GalNAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In some aspects, the cell in accordance with the present disclosure is prepared via an in vivo genetic modification method. In embodiments, a genetic modification in accordance with the present disclosure is performed via an ex vivo method.

In some aspects, the cell in accordance with the present disclosure is prepared by contacting a cell with an enzyme capable of targeted genomic integration by transposition (e.g., without limitation, the transposase enzyme) in vivo. In embodiments, the cell is contacted with the enzyme ex vivo.

In embodiments, the present method provides high specific targeting as compared to a method that does not use the transposase enzyme with a target selector.

### Therapeutic Applications

In embodiments, the transgene of interest in accordance with embodiments of the present disclosure can encode various genes.

In embodiments, the transposase enzyme and the transposon are included in the same pharmaceutical composition.

In embodiments, the transposase enzyme and the transposon are included in different pharmaceutical compositions.

In embodiments, the transposase enzyme and the transposon are co-transfected.

In embodiments the transposase enzyme and the transposon are transfected separately.

In embodiments, a transfected cell for gene therapy is provided, wherein the transfected cell is generated using the transposase enzyme in accordance with embodiments of the present disclosure.

In embodiments, a method of delivering a cell therapy is provided, comprising administering to a patient in need thereof the transfected cell generated using the transposase enzyme in accordance with embodiments of the present disclosure.

In embodiments, a method of treating a disease or condition using a cell therapy, comprising administering to a patient in need thereof the transfected cell generated using the transposase enzyme in accordance with embodiments of the present disclosure.

In embodiments, the disease or condition may comprise cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs syndrome.

In embodiments, the disease or condition is or comprises an infectious disease. In embodiments, the infectious disease is a coronavirus infection, optionally selected from infection with SAR-COV, MERS-COV, and SARS-COV-2, or variants thereof.

In embodiments, the infectious disease is or comprises a disease comprising a viral infection, a parasitic infection, or a bacterial infection. In embodiments, the viral infection is caused by a virus of family Flaviviridae, a virus of family Picornaviridae, a virus of family Orthomyxoviridae, a virus of family Coronaviridae, a virus of family Retroviridae, a virus of family Paramyxoviridae, a virus of family Bunyaviridae, or a virus of family Reoviridae.

In embodiments, the virus of family Coronaviridae comprises a betacoronavirus or an alphacoronavirus, optionally wherein the betacoronavirus is selected from SARS-COV-2, SARS-COV, MERS—COV, HCoV-HKU1, and HCoV-OC43, or the alphacoronavirus is selected from a HCoV-NL63 and HCOV-229E. In embodiments, the infectious disease comprises a coronavirus infection 2019 (COVID-19).

In embodiments, the method requires a single administration. In embodiments, the method requires a plurality of administrations.

### Isolated Cell

In some aspects of the present disclosure, an isolated cell is provided that comprises the transfected cell in accordance with embodiments of the present disclosure.

In some aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell(s) to the patient. For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient, or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly [1,3-bis (carboxyphenoxy) propane-co-sebacic-acid] (PCPP-SA) matrix, fatty acid dimer-sebacic acid (FAD-SA) copolymer, poly (lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79 (3-4): 141-152.

In embodiments, there is provided a method of transforming a cell using the construct comprising the enzyme and/or transgene described herein in the presence of a transposase (e.g., without limitation, the transposase enzyme) to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a monkey, a panda, a dog, a rabbit, and the like. When the organism is an insect, the organism may include, but is not limited to, a fruit fly, a ladybug, a mosquito, a bollworm, and the like.

### Definitions

The following definitions are used in connection with the disclosure disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### SELECTED SEQUENCES

In embodiments, the present disclosure provides for any of the sequence provided herein, including the below, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

```
SEQ ID NO: 11: amino acid sequence of piggyBac transposase (594 aa)
      1 MGSSLDDEHI LSALLQSDDE LVGEDSDSEI SDHVSEDDVQ SDTEEAFIDE
    51 VHEVQPTSSG SEILDEQNVI EQPGSSLASN RILTLPQRTI RGKNKHCWST
  101 SKSTRRSRVS ALNIVRSQRG PTRMCRNIYD PLLCFKLFFT DEIISEIVKW
  151 TNAEISLKRR ESMTGATFRD TNEDEIYAFF GILVMTAVRK DNHMSTDDLF
  201 DRSLSMVYVS VMSRDRFDFL IRCLRMDDKS IRPTLRENDV FTPVRKIWDL
  251 FIHQCIQNYT PGAHLTIDEQ LLGFRGRCPF RMYIPNKPSK YGIKILMMCD
  301 SGTKYMINGM PYLGRGTQTN GVPLGEYYVK ELSKPVHGSC RNITCDNWFT
  351 SIPLAKNLLQ EPYKLTIVGT VRSNKREIPE VLKNSRSRPV GTSMFCFDGP
  401 LTLVSYKPKP AKMVYLLSSC DEDASINEST GKPQMVMYYN QTKGGVDTLD
  451 QMCSVMTCSR KTNRWPMALL YGMINIACIN SFIIYSHNVS SKGEKVQSRK
  501 KFMRNLYMSL TSSFMRKRLE APTLKRYLRD NISNILPNEV PGTSDDSTEE
  551 PVMKKRTYCT YCPSKIRRKA NASCKKCKKV ICREHNIDMC QSCF
SEQ ID NO: 12: nucleotide sequence encoding the wild-type
piggyBac transposase (2472 nt)
      1 ccctagaaag atagtctgcg taaaattgac gcatgcattc ttgaaatatt gctctctctt
    61 tctaaatagc gcgaatccgt cgctgtgcat ttaggacatc tcagtcgccg cttggagctc
  121 ccgtgaggcg tgcttgtcaa tgcggtaagt gtcactgatt ttgaactata acgaccgcgt
  181 gagtcaaaat gacgcatgat tatcttttac gtgactttta agatttaact catacgataa
  241 ttatattgtt atttcatgtt ctacttacgt gataacttat tatatatata ttttcttgtt
  301 atagatatcg tgactaatat ataataaaat gggtagttct ttagacgatg agcatatcct
  361 ctctgctctt ctgcaaagcg atgacgagct tgttggtgag gattctgaca gtgaaatatc
  421 agatcacgta agtgaagatg acgtccagag cgatacagaa gaagcgttta tagatgaggt
  481 acatgaagtg cagccaacgt caagcggtag tgaaatatta gacgaacaaa atgttattga
  541 acaaccaggt tcttcattgg cttctaacag aatcttgacc ttgccacaga ggactattag
  601 aggtaagaat aaacattgtt ggtcaacttc aaagtccacg aggcgtagcc gagtctctgc
  661 actgaacatt gtcagatctc aaagaggtcc gacgcgtatg tgccgcaata tatatgaccc
  721 acttttatgc ttcaaactat tttttactga tgagataatt tcggaaattg taaaatggac
  781 aaatgctgag atatcattga aacgtcggga atctatgaca ggtgctacat ttcgtgacac
  841 gaatgaagat gaaatctatg ctttctttgg tattctggta atgacagcag tgagaaaaga
  901 taaccacatg tccacagatg acctctttga tcgatctttg tcaatggtgt acgtctctgt
  961 aatgagtcgt gatcgttttg attttttgat acgatgtctt agaatggatg acaaaagtat
1021 acggcccaca cttcgagaaa acgatgtatt tactcctgtt agaaaaatat gggatctctt
1081 tatccatcag tgcatacaaa attacactcc aggggctcat ttgaccatag atgaacagtt
1141 acttggtttt agaggacggt gtccgtttag gatgtatatc ccaaacaagc caagtaagta
1201 tggaataaaa atcctcatga tgtgtgacag tggtacgaag tatatgataa atggaatgcc
1261 ttatttggga agaggaacac agaccaacgg agtaccactc ggtgaatact acgtgaagga
1321 gttatcaaag cctgtgcacg gtagttgtcg taatattacg tgtgacaatt ggttcacctc
1381 aatccctttg gcaaaaaact tactacaaga accgtataag ttaaccattg tgggaaccgt
1441 gcgatcaaac aaacgcgaga taccggaagt actgaaaaac agtcgctcca ggccagtggg
1501 aacatcgatg ttttgttttg acggacccct tactctcgtc tcatataaac cgaagccagc
1561 taagatggta tacttattat catcttgtga tgaggatgct tctatcaacg aaagtaccgg
1621 taaaccgcaa atggttatgt attataatca aactaaaggc ggagtggaca cgctagacca
1681 aatgtgttct gtgatgacct gcagtaggaa gacgaatagg tggcctatgg cattattgta
1741 cggaatgata aacattgcct gcataaattc ttttattata tacagccata atgtcagtag
1801 caagggagaa aaggttcaaa gtcgcaaaaa atttatgaga aacctttaca tgagcctgac
1861 gtcatcgttt atgcgtaagc gtttagaagc tcctactttg aagagatatt tgcgcgataa
1921 tatctctaat attttgccaa atgaagtgcc tggtacatca gatgacagta ctgaagagcc
1981 agtaatgaaa aaacgtactt actgtactta ctgcccctct aaaataaggc gaaaggcaaa
2041 tgcatcgtgc aaaaaatgca aaaaagttat ttgtcgagag cataatattg atatgtgcca
2101 aagttgtttc tgactgacta ataagtataa tttgtttcta ttatgtataa gttaagctaa
2161 ttacttattt tataatacaa catgactgtt tttaaagtac aaaataagtt tatttttgta
2221 aaagagagaa tgtttaaaag ttttgttact ttatagaaga aattttgagt ttttgttttt
2281 ttttaataaa taaataaaca taaataaatt gtttgttgaa tttattatta gtatgtaagt
2341 gtaaatataa taaaacttaa tatctattca aattaataaa taaacctcga tatacagacc
2401 gataaaacac atgcgtcaat tttacgcatg attatcttta acgtacgtca caatatgatt
2461 atctttctag gg
SEQ ID NO: 1: amino acid sequence of hyperactive transposase
(594 aa) [includes eleven mutants 130V,
S103P,G165S, M282V, S509G, N538K, and N571S (italicized);
and D450N, 182N, V109A, and Q591R (bold
underlined)]
      1 MGSSLDDEHI LSALLQSDDE LVGEDSDSEV SDHVSEDDVQ SDTEEAFIDE
    51 VHEVQPTSSG SEILDEQNVI EQPGSSLASN RNLTLPQRTI RGKNKHCWST
  101 SKPTRRSRAS ALNIVRSQRG PTRMCRNIYD PLLCFKLFFT DEIISEIVKW
  151 TNAEISLKRR ESMTSATFRD TNEDEIYAFF GILVMTAVRK DNHMSTDDLF
  201 DRSLSMVYVS VMSRDRFDFL IRCLRMDDKS IRPTLRENDV FTPVRKIWDL
  251 FIHQCIQNYT PGAHLTIDEQ LLGFRGRCPF RVYIPNKPSK YGIKILMMCD
  301 SGTKYMINGM PYLGRGTQTN GVPLGEYYVK ELSKPVHGSC RNITCDNWFT
  351 SIPLAKNLLQ EPYKLTIVGT VRSNKREIPE VLKNSRSRPV GTSMFCFDGP
  401 LTLVSYKPKP AKMVYLLSSC DEDASINEST GKPQMVMYYN QTKGGVDTLN
  451 QMCSVMTCSR KTNRWPMALL YGMINIACIN SFIIYSHNVS SKGEKVQSRK
  501 KFMRNLYMGL TSSFMRKRLE APTLKRYLRD NISNILPKEV PGTSDDSTEE
  551 PVMKKRTYCT YCPSKIRRKA SASCKKCKKV ICREHNIDMC RSCF
SEQ ID NO: 2: amino acid sequence of WO_2021_110119 hyperactive
piggyBac (594 aa) [includes ten mutants 130V, S103P,G165S,
M282V, S509G, N538K, and N571S (italicized); and 182N, V109A,
and Q591R (bold underlined). D450N (underlined not included)]
      1 MGSSLDDEHI LSALLQSDDE LVGEDSDSEV SDHVSEDDVQ SDTEEAFIDE
    51 VHEVQPTSSG SEILDEQNVI EQPGSSLASN RNLTLPQRTI RGKNKHCWST
  101 SKPTRRSRAS ALNIVRSQRG PTRMCRNIYD PLLCFKLFFT DEIISEIVKW
  151 TNAEISLKRR ESMTSATFRD TNEDEIYAFF GILVMTAVRK DNHMSTDDLF
  201 DRSLSMVYVS VMSRDRFDFL IRCLRMDDKS IRPTLRENDV FTPVRKIWDL
  251 FIHQCIQNYT PGAHLTIDEQ LLGFRGRCPF RVYIPNKPSK YGIKILMMCD
  301 SGTKYMINGM PYLGRGTQTN GVPLGEYYVK ELSKPVHGSC RNITCDNWFT
  351 SIPLAKNLLQ EPYKLTIVGT VRSNKREIPE VLKNSRSRPV GTSMFCFDGP
  401 LTLVSYKPKP AKMVYLLSSC DEDASINEST GKPQMVMYYN QTKGGVDTLD
  451 QMCSVMTCSR KTNRWPMALL YGMINIACIN SFIIYSHNVS SKGEKVQSRK
  501 KFMRNLYMGL TSSFMRKRLE APTLKRYLRD NISNILPKEV PGTSDDSTEE
  551 PVMKKRTYCT YCPSKIRRKA SASCKKCKKV ICREHNIDMC RSCF
SEQ ID NO: 3: amino acid sequence of WO_2020_164702 hyperactive
piggyBac (594 aa) [includes seven mutants 130V, G165S, M282V,
and N538K (italicized); 182N, V109A, and Q591R (bold underlined).
S103P, D450N, S509G, and N571S (underlined not included)]
      1 MGSSLDDEHI LSALLQSDDE LVGEDSDSEV SDHVSEDDVQ SDTEEAFIDE
    51 VHEVQPTSSG SEILDEQNVI EQPGSSLASN RNLTLPQRTI RGKNKHCWST
  101 SKSTRRSRAS ALNIVRSQRG PTRMCRNIYD PLLCFKLFFT DEIISEIVKW
  151 TNAEISLKRR ESMTSATFRD TNEDEIYAFF GILVMTAVRK DNHMSTDDLF
  201 DRSLSMVYVS VMSRDRFDFL IRCLRMDDKS IRPTLRENDV FTPVRKIWDL
  251 FIHQCIQNYT PGAHLTIDEQ LLGFRGRCPF RVYIPNKPSK YGIKILMMCD
  301 SGTKYMINGM PYLGRGTQTN GVPLGEYYVK ELSKPVHGSC RNITCDNWFT
  351 SIPLAKNLLQ EPYKLTIVGT VRSNKREIPE VLKNSRSRPV GTSMFCFDGP
  401 LTLVSYKPKP AKMVYLLSSC DEDASINEST GKPQMVMYYN QTKGGVDTLD
  451 QMCSVMTCSR KTNRWPMALL YGMINIACIN SFIIYSHNVS SKGEKVQSRK
  501 KFMRNLYMSL TSSFMRKRLE APTLKRYLRD NISNILPKEV PGTSDDSTEE
  551 PVMKKRTYCT YCPSKIRRKA NASCKKCKKV ICREHNIDMC RSCF
SEQ ID NO: 4: amino acid sequence of dead Cas9 protein
(GENBANK ACC. No. MT882253.1)
      1 MDKKYSIGLA IGTNSVGWAV ITDEYKVPSK KFKVLGNTDR HSIKKNLIGA
    51 LLFDSGETAE ATRLKRTARR RYTRRKNRIC YLQEIFSNEM AKVDDSFFHR
  101 LEESFLVEED KKHERHPIFG NIVDEVAYHE KYPTIYHLRK KLVDSTDKAD
  151 LRLIYLALAH MIKFRGHFLI EGDLNPDNSD VDKLFIQLVQ TYNQLFEENP
  201 INASGVDAKA ILSARLSKSR RLENLIAQLP GEKKNGLFGN LIALSLGLTP
  251 NFKSNFDLAE DAKLQLSKDT YDDDLDNLLA QIGDQYADLF LAAKNLSDAI
  301 LLSDILRVNT EITKAPLSAS MIKRYDEHHQ DLTLLKALVR QQLPEKYKEI
  351 FFDQSKNGYA GYIDGGASQE EFYKFIKPIL EKMDGTEELL VKLNREDLLR
  401 KQRTFDNGSI PHQIHLGELH AILRRQEDFY PFLKDNREKI EKILTFRIPY
  451 YVGPLARGNS RFAWMTRKSE ETITPWNFEE VVDKGASAQS FIERMTNFDK
  501 NLPNEKVLPK HSLLYEYFTV YNELTKVKYV TEGMRKPAFL SGEQKKAIVD
  551 LLFKTNRKVT VKQLKEDYFK KIECFDSVEI SGVEDRFNAS LGTYHDLLKI
  601 IKDKDFLDNE ENEDILEDIV LTLTLFEDRE MIEERLKTYA HLFDDKVMKQ
  651 LKRRRYTGWG RLSRKLINGI RDKQSGKTIL DFLKSDGFAN RNFMQLIHDD
  701 SLTFKEDIQK AQVSGQGDSL HEHIANLAGS PAIKKGILQT VKVVDELVKV
  751 MGRHKPENIV IEMARENQTT QKGQKNSRER MKRIEEGIKE LGSQILKEHP
  801 VENTQLQNEK LYLYYLQNGR DMYVDQELDI NRLSDYDVAA IVPQSFLKDD
  851 SIDNKVLTRS DKARGKSDNV PSEEVVKKMK NYWRQLLNAK LITQRKFDNL
  901 TKAERGGLSE LDKAGFIKRQ LVETRQITKH VAQILDSRMN TKYDENDKLI
  951 REVKVITLKS KLVSDFRKDF QFYKVREINN YHHAHDAYLN AVVGTALIKK
1001 YPKLESEFVY GDYKVYDVRK MIAKSEQEIG KATAKYFFYS NIMNFFKTEI
1051 TLANGEIRKR PLIETNGETG EIVWDKGRDF ATVRKVLSMP QVNIVKKTEV
1101 QTGGFSKESI LPKRNSDKLI ARKKDWDPKK YGGFDSPTVA YSVLVVAKVE
1151 KGKSKKLKSV KELLGITIME RSSFEKNPID FLEAKGYKEV KKDLIIKLPK
1201 YSLFELENGR KRMLASAGEL QKGNELALPS KYVNFLYLAS HYEKLKGSPE
1251 DNEQKQLFVE QHKHYLDEII EQISEFSKRV ILADANLDKV LSAYNKHRDK
1301 PIREQAENII HLFTLINLGA PAAFKYFDTT IDRKRYTSTK EVLDATLIHQ
1351 SITGLYETRI DLSQLGGDSR ADPKKKRKV
SEQ ID NO: 5: nucleotide sequence of left hyperactive
transposase donor end sequence (310 bp)
      1 ATCTATAACA AGAAAATATA TATATAATAA GTTATCACGT AAGTAGAACA
    51 TGAAATAACA ATATAATTAT CGTATGAGTT AAATCTTAAA AGTCACGTAA
  101 AAGATAATCA TGCGTCATTT TGACTCACGC GGTCGTTATA GTTCAAAATC
  151 AGTGACACTT ACCGCATTGA CAAGCACGCC TCACGGGAGC TCCAAGCGGC
  201 GACTGAGATG TCCTAAATGC ACAGCGACGG ATTCGCGCTA TTTAGAAAGA
  251 GAGAGCAATA TTTCAAGAAT GCATGCGTCA ATTTTACGCA GACTATCTTT
  301 CTAGGGTTAA
SEQ ID NO: 6: nucleotide sequence of right hyperactive
transposase donor end sequence (205 bp)
      1 TTAACCCTAG AAAGATAATC ATATTGTGAC GTACGTTAAA GATAATCATG
    51 CGTAAAATTG ACGCATGTGT TTTATCGGTC TGTATATCGA GGTTTATTTA
  101 TTAATTTGAA TAGATATTAA GTTTTATTAT ATTTACACTT ACATACTAAT
  151 AATAAATTCA ACAAACAATT TATTTATGTT TATTTATTTA TTAAAAAAAA
  201 ACAAA
SEQ ID NO: 7:
nucleotide sequence of hyperactive transposase (1785 bp)
      1 ATGGGGTCTT CACTGGACGA TGAGCATATT CTGAGCGCCC TGCTGCAGAG
    51 CGATGACGAG CTGGTGGGAG AGGATTCCGA TTCCGAGGTC AGTGACCACG
  101 TGTCAGAGGA CGATGTGCAG AGCGACACTG AGGAAGCCTT CATCGATGAG
  151 GTCCATGAAG TGCAGCCAAC AAGCTCCGGA AGCGAGATCC TGGATGAACA
  201 GAACGTGATT GAACAGCCTG GCTCTAGTCT GGCTTCCAAT AGGAACCTGA
  251 CACTGCCACA GCGAACTATT CGGGGCAAGA ACAAGCACTG CTGGAGCACC
  301 TCCAAGCCTA CACGGAGAAG CCGCGCGTCC GCCCTGAACA TCGTGAGATC
  351 CCAGAGGGGG CCAACCCGCA TGTGCCGAAA TATCTACGAC CCCCTGCTGT
  401 GCTTTAAGCT GTTCTTTACA GATGAGATCA TTAGTGAAAT CGTGAAGTGG
  451 ACTAACGCAG AGATTTCACT GAAAAGGCGC GAATCTATGA CTAGTGCCAC
  501 CTTCAGAGAC ACAAATGAGG ATGAAATCTA CGCTTTCTTT GGCATTCTGG
  551 TCATGACCGC AGTGAGGAAG GACAACCATA TGTCTACAGA CGATCTGTTT
  601 GATCGCTCTC TGAGTATGGT GTATGTCTCA GTGATGAGCA GAGACAGGTT
  651 CGATTTTTTG ATCCGGTGCC TGAGAATGGA CGATAAGAGC ATTCGACCTA
  701 CACTGCGGGA GAATGACGTG TTCACCCCAG TGAGGAAAAT CTGGGATCTG
  751 TTTATCCACC AGTGTATTCA GAACTACACA CCCGGAGCCC ATCTGACTAT
  801 CGACGAACAG CTGCTGGGCT TCCGCGGGCG ATGCCCTTTT CGCGTATACA
  851 TTCCAAATAA GCCCAGCAAA TATGGCATCA AGATTCTGAT GATGTGCGAT
  901 TCCGGGACCA AATACATGAT CAACGGAATG CCATATCTGG GACGGGGCAC
  951 CCAGACAAAT GGAGTCCCCC TGGGCGAGTA CTATGTGAAG GAACTGTCCA
1001 AACCTGTCCA CGGGTCTTGC AGAAACATCA CCTGTGACAA TTGGTTCACA
1051 TCTATTCCCC TGGCCAAGAA CCTGCTGCAG GAGCCTTATA AACTGACTAT
1101 CGTCGGAACC GTGAGAAGCA ACAAGAGGGA GATTCCCGAA GTGCTGAAGA
1151 ACAGCCGGAG CAGACCTGTC GGCACTTCTA TGTTCTGCTT TGACGGGCCA
1201 CTGACCCTGG TGAGTTACAA GCCCAAACCT GCTAAAATGG TGTATCTGCT
1251 GTCAAGCTGT GACGAGGATG CAAGCATCAA TGAATCCACC GGCAAGCCCC
1301 AGATGGTCAT GTACTATAAC CAGACTAAAG GCGGGGTGGA TACCCTGAAT
1351 CAGATGTGCT CTGTCATGAC CTGTAGTAGA AAGACAAACA GGTGGCCTAT
1401 GGCCCTGCTG TACGGGATGA TCAACATTGC TTGCATTAAT TCATTCATCA
1451 TCTACAGCCA CAACGTGTCC TCTAAGGGGG AGAAAGTCCA GTCCCGCAAG
1501 AAATTCATGC GAAATCTGTA CATGGGACTG ACCAGTAGCT TCATGAGGAA
1551 GCGCCTGGAG GCACCCACAC TGAAAAGGTA TCTGCGCGAC AACATCAGCA
1601 ATATTCTGCC TAAGGAAGTG CCAGGCACTT CCGACGATTC TACCGAGGAA
1651 CCAGTGATGA AGAAACGGAC ATACTGCACT TATTGTCCCA GCAAGATCCG
1701 ACGGAAAGCC TCCGCTTCTT GCAAGAAGTG TAAGAAAGTG ATCTGTAGAG
1751 AGCATAACAT TGATATGTGC CGGTCCTGTT TTTGA
SEQ ID NO: 8: nucleotide sequence of hyperactive piggyBac
from WO_2021_110119 (1785 bp)
      1 ATGGGCAGCA GCCTGGACGA CGAGCACATC CTGAGCGCCC TGCTGCAGAG
    51 CGACGACGAG CTGGTGGGCG AGGACAGCGA CAGCGAGGTG AGCGACCACG
  101 TGAGCGAGGA CGACGTGCAG AGCGACACCG AGGAGGCCTT CATCGACGAG
  151 GTGCACGAGG TGCAGCCCAC CAGCAGCGGC AGCGAGATCC TGGACGAGCA
  201 GAACGTGATC GAGCAGCCCG GCAGCAGCCT GGCCAGCAAC CGCAACCTGA
  251 CCCTGCCCCA GCGCACCATC CGCGGCAAGA ACAAGCACTG CTGGAGCACC
  301 AGCAAGCCCA CCCGCCGCAG CCGCGCCAGC GCCCTGAACA TCGTGCGCAG
  351 CCAGCGCGGC CCCACCCGCA TGTGCCGCAA CATCTACGAC CCCCTGCTGT
  401 GCTTCAAGCT GTTCTTCACC GACGAGATCA TCAGCGAGAT CGTGAAGTGG
  451 ACCAACGCCG AGATCAGCCT GAAGCGCCGC GAGAGCATGA CCAGCGCCAC
  501 CTTCCGCGAC ACCAACGAGG ACGAGATCTA CGCCTTCTTC GGCATCCTGG
  551 TGATGACCGC CGTGCGCAAG GACAACCACA TGAGCACCGA CGACCTGTTC
  601 GACCGCAGCC TGAGCATGGT GTACGTGAGC GTGATGAGCC GCGACCGCTT
  651 CGACTTCCTG ATCCGCTGCC TGCGCATGGA CGACAAGAGC ATCCGCCCCA
  701 CCCTGCGCGA GAACGACGTG TTCACCCCCG TGCGCAAGAT CTGGGACCTG
  751 TTCATCCACC AGTGCATCCA GAACTACACC CCCGGCGCCC ACCTGACCAT
  801 CGACGAGCAG CTGCTGGGCT TCCGCGGCCG CTGCCCCTTC CGCGTGTACA
  851 TCCCCAACAA GCCCAGCAAA TACGGCATCA AGATCCTGAT GATGTGCGAC
  901 AGCGGCACCA AGTACATGAT CAACGGCATG CCCTACCTGG GCCGCGGCAC
  951 CCAGACCAAC GGCGTGCCCC TGGGCGAGTA CTACGTGAAG GAGCTGAGCA
1001 AGCCCGTGCA CGGCAGCTGC CGCAACATCA CCTGCGACAA CTGGTTCACC
1051 AGCATCCCCC TGGCCAAGAA CCTGCTGCAG GAGCCCTACA AGCTGACCAT
1101 CGTGGGCACC GTGCGCAGCA ACAAGCGCGA GATCCCCGAG GTGCTGAAGA
1151 ACAGCCGCAG CCGCCCCGTG GGCACCAGCA TGTTCTGCTT CGACGGCCCC
1201 CTGACCCTGG TGAGCTACAA GCCCAAGCCC GCCAAGATGG TGTACCTGCT
1251 GAGCAGCTGC GACGAGGACG CCAGCATCAA CGAGAGCACC GGCAAGCCCC
1301 AGATGGTGAT GTACTACAAC CAGACCAAGG GCGGCGTGGA CACCCTGGAC
1351 CAGATGTGCA GCGTGATGAC CTGCAGCCGC AAGACCAACC GCTGGCCCAT
1401 GGCCCTGCTG TACGGCATGA TCAACATCGC CTGCATCAAC AGCTTCATCA
1451 TCTACAGCCA CAACGTGAGC AGCAAGGGCG AGAAGGTGCA GAGCCGCAAG
1501 AAGTTCATGC GCAACCTGTA CATGGGCCTG ACCAGCAGCT TCATGCGCAA
1551 GCGCCTGGAG GCCCCCACCC TGAAGCGCTA CCTGCGCGAC AACATCAGCA
1601 ACATCCTGCC CAAGGAGGTG CCCGGCACCA GCGACGACAG CACCGAGGAG
1651 CCCGTGATGA AGAAGCGCAC CTACTGCACC TACTGCCCCA GCAAGATCCG
1701 CCGCAAGGCC AGCGCCAGCT GCAAGAAGTG CAAGAAGGTG ATCTGCCGCG
1751 AGCACAACAT CGACATGTGC CGGAGCTGCT TCTAA
SEQ ID NO: 9: nucleotide sequence of hyperactive piggyBac
from WO_2020_164702_A1 (1785 bp)
      1 ATGGGCTCTA GCCTGGACGA CGAGCACATT CTGTCTGCCC TGCTGCAGTC
    51 CGACGATGAA CTCGTGGGCG AAGATTCCGA CTCCGAGATC TCTGACCACG
  101 TGTCCGAGGA CGACGTGCAG TCTGATACCG AGGAAGCCTT CATCGACGAG
  151 GTGCACGAAG TGCAGCCTAC CTCTTCCGGC TCTGAGATCC TGGACGAGCA
  201 GAACGTGATC GAGCAGCCTG GATCCTCTCT GGCCTCCAAC AGAATCCTGA
  251 CACTGCCCCA GAGAACCATC CGGGGCAAGA ACAAGCACTG CTGGTCCACC
  301 TCCAAGTCTA CCCGGCGGTC TAGAGTGTCC GCTCTGAATA TTGTGCGGTC
  351 CCAGAGGGGC CCCACCAGAA TGTGCCGGAA CATCTACGAC CCTCTGCTGT
  401 GTTTCAAGCT GTTCTTCACC GACGAGATCA TCAGCGAGAT CGTGAAGTGG
  451 ACCAACGCCG AGATCAGCCT GAAGCGGCGG GAATCTATGA CCGGCGCCAC
  501 CTTCAGAGAC ACCAACGAGG ATGAGATCTA CGCCTTCTTC GGCATCCTGG
  551 TCATGACAGC CGTGCGGAAG GACAACCACA TGTCCACCGA CGACCTGTTC
  601 GACAGATCCC TGTCCATGGT GTACGTGTCC GTGATGAGCC GGGACAGATT
  651 CGACTTCCTG ATCCGGTGCC TGCGGATGGA CGACAAGTCC ATCAGACCCA
  701 CACTGCGCGA GAACGACGTG TTCACACCTG TGCGGAAGAT CTGGGACCTG
  751 TTCATCCACC AGTGCATCCA GAACTACACC CCTGGCGCTC ACCTGACCAT
  801 CGATGAACAG CTGCTGGGCT TCAGAGGCAG ATGCCCCTTC AGAATGTACA
  851 TCCCCAACAA GCCCTCTAAG TACGGCATCA AGATCCTGAT GATGTGCGAC
  901 TCCGGCACCA AGTACATGAT CAACGGCATG CCCTACCTCG GCAGAGGCAC
  951 CCAAACAAAT GGCGTGCCAC TGGGCGAGTA CTATGTGAAA GAACTGTCCA
1001 AGCCTGTGCA CGGCTCCTGC AGAAACATCA CCTGTGACAA CTGGTTCACC
1051 AGCATTCCTC TGGCCAAGAA CCTGCTGCAA GAGCCCTACA AGCTGACAAT
1101 CGTGGGCACC GTGCGGTCCA ACAAGCGGGA AATTCCTGAG GTGCTGAAGA
1151 ACTCTCGGTC CAGACCTGTG GGCACCTCCA TGTTCTGTTT CGACGGCCCT
1201 CTGACACTGG TGTCCTACAA GCCTAAGCCT GCCAAGATGG TGTACCTGCT
1251 GTCCTCCTGT GACGAGGACG CCAGCATCAA TGAGTCCACC GGCAAGCCCC
1301 AGATGGTCAT GTACTACAAC CAGACCAAAG GCGGCGTGGA CACCCTGGAC
1351 CAGATGTGCT CTGTGATGAC CTGCTCCAGA AAGACCAACA GATGGCCCAT
1401 GGCTCTGCTG TACGGCATGA TCAATATCGC CTGCATCAAC AGCTTCATCA
1451 TCTACTCCCA CAACGTGTCC TCCAAGGGCG AGAAGGTGCA GTCCCGGAAG
1501 AAATTCATGC GGAACCTGTA TATGTCCCTG ACCTCCAGCT TCATGAGAAA
1551 GCGGCTGGAA GCCCCTACTC TGAAGAGATA CCTGCGGGAC AACATCTCCA
1601 ACATCCTGCC TAACGAGGTG CCCGGCACCA GCGACGATTC TACAGAGGAA
1651 CCTGTGATGA AGAAGCGGAC CTACTGCACC TACTGTCCCT CCAAGATCCG
1701 GCGGAAGGCC AACGCCTCTT GCAAAAAGTG CAAGAAAGTG ATCTGCCGCG
1751 AGCACAACAT CGACATGTGC CAGTCTTGTT TCTGA
SEQ ID NO: 10: nucleotide sequence of dead Cas9 protein
(GENBANK ACC. NO. MT882253.1)
      1 ATGGACAAGA AGTACTCCAT TGGGCTCGCT ATCGGCACAA ACAGCGTCGG CTGGGCCGTC
    61 ATTACGGACG AGTACAAGGT GCCGAGCAAA AAATTCAAAG TTCTGGGCAA TACCGATCGC
  121 CACAGCATAA AGAAGAACCT CATTGGCGCC CTCCTGTTCG ACTCCGGGGA GACGGCCGAA
  181 GCCACGCGGC TCAAAAGAAC AGCACGGCGC AGATATACCC GCAGAAAGAA TCGGATCTGC
  241 TACCTGCAGG AGATCTTTAG TAATGAGATG GCTAAGGTGG ATGACTCTTT CTTCCATAGG
  301 CTGGAGGAGT CCTTTTTGGT GGAGGAGGAT AAAAAGCACG AGCGCCACCC AATCTTTGGC
  361 AATATCGTGG ACGAGGTGGC GTACCATGAA AAGTACCCAA CCATATATCA TCTGAGGAAG
  421 AAGCTTGTAG ACAGTACTGA TAAGGCTGAC TTGCGGTTGA TCTATCTCGC GCTGGCGCAT
  481 ATGATCAAAT TTCGGGGACA CTTCCTCATC GAGGGGGACC TGAACCCAGA CAACAGCGAT
  541 GTCGACAAAC TCTTTATCCA ACTGGTTCAG ACTTACAATC AGCTTTTCGA AGAGAACCCG
  601 ATCAACGCAT CCGGAGTTGA CGCCAAAGCA ATCCTGAGCG CTAGGCTGTC CAAATCCCGG
  661 CGGCTCGAAA ACCTCATCGC ACAGCTCCCT GGGGAGAAGA AGAACGGCCT GTTTGGTAAT
  721 CTTATCGCCC TGTCACTCGG GCTGACCCCC AACTTTAAAT CTAACTTCGA CCTGGCCGAA
  781 GATGCCAAGC TTCAACTGAG CAAAGACACC TACGATGATG ATCTCGACAA TCTGCTGGCC
  841 CAGATCGGCG ACCAGTACGC AGACCTTTTT TTGGCGGCAA AGAACCTGTC AGACGCCATT
  901 CTGCTGAGTG ATATTCTGCG AGTGAACACG GAGATCACCA AAGCTCCGCT GAGCGCTAGT
  961 ATGATCAAGC GCTATGATGA GCACCACCAA GACTTGACTT TGCTGAAGGC CCTTGTCAGA
1021 CAGCAACTGC CTGAGAAGTA CAAGGAAATT TTCTTCGATC AGTCTAAAAA TGGCTACGCC
1081 GGATACATTG ACGGCGGAGC AAGCCAGGAG GAATTTTACA AATTTATTAA GCCCATCTTG
1141 GAAAAAATGG ACGGCACCGA GGAGCTGCTG GTAAAGCTTA ACAGAGAAGA TCTGTTGCGC
1201 AAACAGCGCA CTTTCGACAA TGGAAGCATC CCCCACCAGA TTCACCTGGG CGAACTGCAC
1261 GCTATCCTCA GGCGGCAAGA GGATTTCTAC CCCTTTTTGA AAGATAACAG GGAAAAGATT
1321 GAGAAAATCC TCACATTTCG GATACCCTAC TATGTAGGCC CCCTCGCCCG GGGAAATTCC
1381 AGATTCGCGT GGATGACTCG CAAATCAGAA GAGACCATCA CTCCCTGGAA CTTCGAGGAA
1441 GTCGTGGATA AGGGGGCCTC TGCCCAGTCC TTCATCGAAA GGATGACTAA CTTTGATAAA
1501 AATCTGCCTA ACGAAAAGGT GCTTCCTAAA CACTCTCTGC TGTACGAGTA CTTCACAGTT
1561 TATAACGAGC TCACCAAGGT CAAATACGTC ACAGAAGGGA TGAGAAAGCC AGCATTCCTG
1621 TCTGGAGAGC AGAAGAAAGC TATCGTGGAC CTCCTCTTCA AGACGAACCG GAAAGTTACC
1681 GTGAAACAGC TCAAAGAAGA CTATTTCAAA AAGATTGAAT GTTTCGACTC TGTTGAAATC
1741 AGCGGAGTGG AGGATCGCTT CAACGCATCC CTGGGAACGT ATCACGATCT CCTGAAAATC
1801 ATTAAAGACA AGGACTTCCT GGACAATGAG GAGAACGAGG ACATTCTTGA GGACATTGTC
1861 CTCACCCTTA CGTTGTTTGA AGATAGGGAG ATGATTGAAG AACGCTTGAA AACTTACGCT
1921 CATCTCTTCG ACGACAAAGT CATGAAACAG CTCAAGAGGC GCCGATATAC AGGATGGGGG
1981 CGGCTGTCAA GAAAACTGAT CAATGGGATC CGAGACAAGC AGAGTGGAAA GACAATCCTG
2041 GATTTTCTTA AGTCCGATGG ATTTGCCAAC CGGAACTTCA TGCAGTTGAT CCATGATGAC
2101 TCTCTCACCT TTAAGGAGGA CATCCAGAAA GCACAAGTTT CTGGCCAGGG GGACAGTCTT
2161 CACGAGCACA TCGCTAATCT TGCAGGTAGC CCAGCTATCA AAAAGGGAAT ACTGCAGACC
2221 GTTAAGGTCG TGGATGAACT CGTCAAAGTA ATGGGAAGGC ATAAGCCCGA GAATATCGTT
2281 ATCGAGATGG CCCGAGAGAA CCAAACTACC CAGAAGGGAC AGAAGAACAG TAGGGAAAGG
2341 ATGAAGAGGA TTGAAGAGGG TATAAAAGAA CTGGGGTCCC AAATCCTTAA GGAACACCCA
2401 GTTGAAAACA CCCAGCTTCA GAATGAGAAG CTCTACCTGT ACTACCTGCA GAACGGCAGG
2461 GACATGTACG TGGATCAGGA ACTGGACATC AATCGGCTCT CCGACTACGA CGTGGCTGCT
2521 ATCGTGCCCC AGTCTTTTCT CAAAGATGAT TCTATTGATA ATAAAGTGTT GACAAGATCC
2581 GATAAAGCTA GAGGGAAGAG TGATAACGTC CCCTCAGAAG AAGTTGTCAA GAAAATGAAA
2641 AATTATTGGC GGCAGCTGCT GAACGCCAAA CTGATCACAC AACGGAAGTT CGATAATCTG
2701 ACTAAGGCTG AACGAGGTGG CCTGTCTGAG TTGGATAAAG CCGGCTTCAT CAAAAGGCAG
2761 CTTGTTGAGA CACGCCAGAT CACCAAGCAC GTGGCCCAAA TTCTCGATTC ACGCATGAAC
2821 ACCAAGTACG ATGAAAATGA CAAACTGATT CGAGAGGTGA AAGTTATTAC TCTGAAGTCT
2881 AAGCTGGTCT CAGATTTCAG AAAGGACTTT CAGTTTTATA AGGTGAGAGA GATCAACAAT
2941 TACCACCATG CGCATGATGC CTACCTGAAT GCAGTGGTAG GCACTGCACT TATCAAAAAA
3001 TATCCCAAGC TTGAATCTGA ATTTGTTTAC GGAGACTATA AAGTGTACGA TGTTAGGAAA
3061 ATGATCGCAA AGTCTGAGCA GGAAATAGGC AAGGCCACCG CTAAGTACTT CTTTTACAGC
3121 AATATTATGA ATTTTTTCAA GACCGAGATT ACACTGGCCA ATGGAGAGAT TCGGAAGCGA
3181 CCACTTATCG AAACAAACGG AGAAACAGGA GAAATCGTGT GGGACAAGGG TAGGGATTTC
3241 GCGACAGTCC GGAAGGTCCT GTCCATGCCG CAGGTGAACA TCGTTAAAAA GACCGAAGTA
3301 CAGACCGGAG GCTTCTCCAA GGAAAGTATC CTCCCGAAAA GGAACAGCGA CAAGCTGATC
3361 GCACGCAAAA AAGATTGGGA CCCCAAGAAA TACGGCGGAT TCGATTCTCC TACAGTCGCT
3421 TACAGTGTAC TGGTTGTGGC CAAAGTGGAG AAAGGGAAGT CTAAAAAACT CAAAAGCGTC
3481 AAGGAACTGC TGGGCATCAC AATCATGGAG CGATCAAGCT TCGAAAAAAA CCCCATCGAC
3541 TTTCTGGAGG CGAAAGGATA TAAAGAGGTC AAAAAAGACC TCATCATTAA GCTTCCCAAG
3601 TACTCTCTCT TTGAGCTTGA AAACGGCCGG AAACGAATGC TCGCTAGTGC GGGCGAGCTG
3661 CAGAAAGGTA ACGAGCTGGC ACTGCCCTCT AAATACGTTA ATTTCTTGTA TCTGGCCAGC
3721 CACTATGAAA AGCTCAAAGG GTCTCCCGAA GATAATGAGC AGAAGCAGCT GTTCGTGGAA
3781 CAACACAAAC ACTACCTTGA TGAGATCATC GAGCAAATAA GCGAATTCTC CAAAAGAGTG
3841 ATCCTCGCCG ACGCTAACCT CGATAAGGTG CTTTCTGCTT ACAATAAGCA CAGGGATAAG
3901 CCCATCAGGG AGCAGGCAGA AAACATTATC CACTTGTTTA CTCTGACCAA CTTGGGCGCG
3961 CCTGCAGCCT TCAAGTACTT CGACACCACC ATAGACAGAA AGCGGTACAC CTCTACAAAG
4021 GAGGTCCTGG ACGCCACACT GATTCATCAG TCAATTACGG GGCTCTATGA AACAAGAATC
4081 GACCTCTCTC AGCTCGGTGG AGACAGCAGG GCTGACCCCA AGAAGAAGAG GAAGGTG
SEQ ID NO: 501: Trichnoplusia ni (hyperactive piggyBac) nucleotide
sequence(N0) 1782 bp
      1 ATGGGCTCTT CCTTGGACGA CGAGCACATC CTGAGCGCCC TGCTGCAGTC CGATGACGAG
    61 CTGGTGGGTG AGGACTCCGA CAGTGAGGTG AGCGATCATG TGTCCGAGGA CGATGTGCAG
  121 AGCGACACCG AGGAGGCCTT CATCGATGAG GTGCATGAAG TGCAGCCCAC ATCCTCCGGA
  181 TCCGAGATCC TGGATGAGCA GAACGTGATC GAGCAGCCCG GGAGCTCTCT GGCTTCCAAC
  241 AGAATCCTGA CCCTGCCTCA GAGGACTATC AGAGGGAAAA ATAAGCACTG CTGGTCCACT
  301 TCAAAGCCAA CCAGGCGGTC TAGGGTGTCC GCCCTGAACA TCGTGAGAAG CCAGAGAGGT
  361 CCAACCCGGA TGTGCAGAAA CATCTACGAT CCCCTGCTGT GTTTCAAGCT GTTTTTTACT
  421 GACGAGATTA TCAGCGAGAT CGTGAAGTGG ACCAACGCCG AGATCTCTCT GAAGCGGAGA
  481 GAAAGCATGA CCTCCGCCAC ATTCCGCGAT ACCAACGAAG ACGAGATCTA CGCCTTTTTC
  541 GGCATCTTGG TGATGACTGC CGTGAGGAAG GACAATCACA TGAGCACCGA TGACCTGTTC
  601 GATCGCAGCC TGAGCATGGT GTACGTGTCC GTGATGTCCA GGGACAGGTT CGACTTCCTG
  661 ATCAGGTGCC TGAGAATGGA TGACAAGAGC ATTAGACCTA CCCTGCGCGA GAATGACGTG
  721 TTCACCCCCG TCAGGAAGAT CTGGGACCTG TTTATTCACC AGTGCATTCA GAACTATACC
  781 CCTGGCGCCC ACCTGACCAT TGATGAGCAG CTCCTGGGAT TCAGGGGCCG GTGTCCCTTC
  841 AGAGTGTATA TACCTAACAA GCCCTCTAAG TACGGCATCA AAATCCTGAT GATGTGCGAC
  901 TCCGGAACCA AGTACATGAT TAACGGAATG CCCTATCTGG GACGCGGGAC CCAGACCAAT
  961 GGAGTGCCTC TGGGCGAGTA TTACGTGAAA GAGCTGTCCA AACCCGTGCA CGGGTCCTGT
1021 CGGAATATCA CCTGCGACAA CTGGTTCACT TCAATCCCCC TGGCCAAAAA CCTGCTGCAG
1081 GAGCCTTACA AATTGACCAT TGTGGGCACT GTGCGCTCCA ATAAGAGAGA AATTCCTGAG
1141 GTGCTGAAGA ACTCCAGGTC CAGACCCGTG GGCACTAGCA TGTTCTGCTT CGACGGGCCT
1201 CTGACCCTGG TGAGCTATAA GCCCAAGCCA GCCAAGATGG TGTACCTGCT GAGCAGCTGC
1261 GACGAGGATG CCTCAATCAA CGAGAGCACC GGCAAGCCCC AGATGGTGAT GTACTACAAT
1321 CAGACCAAAG GCGGCGTGGA TACCCTGGAC CAGATGTGTT CCGTGATGAC CTGTAGCAGG
1381 AAGACCAACC GGTGGCCAAT GGCCCTGCTG TATGGAATGA TCAACATTGC CTGCATCAAC
1441 AGCTTCATTA TTTACTCCCA CAATGTGTCC TCTAAGGGGG AGAAGGTGCA GTCTAGAAAA
1501 AAGTTTATGA GGAATCTGTA TATGGGCCTG ACCTCTTCCT TCATGCGGAA GCGCCTGGAG
1561 GCACCCACAC TGAAGAGGTA CCTTCGCGAC AATATCTCCA ATATTCTGCC GAAAGAGGTG
1621 CCCGGCACCT CCGACGACAG CACAGAAGAG CCCGTGATGA AAAAGAGGAC CTACTGCACC
1681 TACTGCCCCT CAAAGATCCG CAGGAAGGCC TCCGCATCCT GTAAGAAGTG CAAGAAGGTC
1741 ATCTGCAGGG AACACAATAT CGACATGTGC CAGTCATGCT TC
SEQ ID NO: 502: Trichnoplusia ni (hyperactive piggyBac) amino acid
sequence(N0) 594 aa
      1 MGSSLDDEHI LSALLQSDDE LVGEDSDSEV SDHVSEDDVQ SDTEEAFIDE VHEVQPTSSG
    61 SEILDEQNVI EQPGSSLASN RILTLPQRTI RGKNKHCWST SKPTRRSRVS ALNIVRSQRG
  121 PTRMCRNIYD PLLCFKLFFT DEIISEIVKW TNAEISLKRR ESMTSATFRD TNEDEIYAFF
  181 GILVMTAVRK DNHMSTDDLF DRSLSMVYVS VMSRDRFDFL IRCLRMDDKS IRPTLRENDV
  241 FTPVRKIWDL FIHQCIQNYT PGAHLTIDEQ LLGFRGRCPF RVYIPNKPSK YGIKILMMCD
  301 SGTKYMINGM PYLGRGTOTN GVPLGEYYVK ELSKPVHGSC RNITCDNWFT SIPLAKNLLQ
  361 EPYKLTIVGT VRSNKREIPE VLKNSRSRPV GTSMFCFDGP LTLVSYKPKP AKMVYLLSSC
  421 DEDASINEST GKPQMVMYYN QTKGGVDTLD QMCSVMTCSR KTNRWPMALL YGMINIACIN
  481 SFIIYSHNVS SKGEKVQSRK KFMRNLYMGL TSSFMRKRLE APTLKRYLRD NISNILPKEV
  541 PGTSDDSTEE PVMKKRTYCT YCPSKIRRKA SASCKKCKKV ICREHNIDMC QSCF
SEQ ID NO: 503: Trichnoplusia ni (hyperactive piggyBac) nucleotide
sequence(N1; nucleotide 4-72 deletion). 1710 bp
      1 ATGGACTCCG ACAGTGAGGT GAGCGATCAT GTGTCCGAGG ACGATGTGCA GAGCGACACC
    61 GAGGAGGCCT TCATCGATGA GGTGCATGAA GTGCAGCCCA CATCCTCCGG ATCCGAGATC
  121 CTGGATGAGC AGAACGTGAT CGAGCAGCCC GGGAGCTCTC TGGCTTCCAA CAGAATCCTG
  181 ACCCTGCCTC AGAGGACTAT CAGAGGGAAA AATAAGCACT GCTGGTCCAC TTCAAAGCCA
  241 ACCAGGCGGT CTAGGGTGTC CGCCCTGAAC ATCGTGAGAA GCCAGAGAGG TCCAACCCGG
  301 ATGTGCAGAA ACATCTACGA TCCCCTGCTG TGTTTCAAGC TGTTTTTTAC TGACGAGATT
  361 ATCAGCGAGA TCGTGAAGTG GACCAACGCC GAGATCTCTC TGAAGCGGAG AGAAAGCATG
  421 ACCTCCGCCA CATTCCGCGA TACCAACGAA GACGAGATCT ACGCCTTTTT CGGCATCTTG
  481 GTGATGACTG CCGTGAGGAA GGACAATCAC ATGAGCACCG ATGACCTGTT CGATCGCAGC
  541 CTGAGCATGG TGTACGTGTC CGTGATGTCC AGGGACAGGT TCGACTTCCT GATCAGGTGC
  601 CTGAGAATGG ATGACAAGAG CATTAGACCT ACCCTGCGCG AGAATGACGT GTTCACCCCC
  661 GTCAGGAAGA TCTGGGACCT GTTTATTCAC CAGTGCATTC AGAACTATAC CCCTGGCGCC
  721 CACCTGACCA TTGATGAGCA GCTCCTGGGA TTCAGGGGCC GGTGTCCCTT CAGAGTGTAT
  781 ATACCTAACA AGCCCTCTAA GTACGGCATC AAAATCCTGA TGATGTGCGA CTCCGGAACC
  841 AAGTACATGA TTAACGGAAT GCCCTATCTG GGACGCGGGA CCCAGACCAA TGGAGTGCCT
  901 CTGGGCGAGT ATTACGTGAA AGAGCTGTCC AAACCCGTGC ACGGGTCCTG TCGGAATATC
  961 ACCTGCGACA ACTGGTTCAC TTCAATCCCC CTGGCCAAAA ACCTGCTGCA GGAGCCTTAC
1021 AAATTGACCA TTGTGGGCAC TGTGCGCTCC AATAAGAGAG AAATTCCTGA GGTGCTGAAG
1081 AACTCCAGGT CCAGACCCGT GGGCACTAGC ATGTTCTGCT TCGACGGGCC TCTGACCCTG
1141 GTGAGCTATA AGCCCAAGCC AGCCAAGATG GTGTACCTGC TGAGCAGCTG CGACGAGGAT
1201 GCCTCAATCA ACGAGAGCAC CGGCAAGCCC CAGATGGTGA TGTACTACAA TCAGACCAAA
1261 GGCGGCGTGG ATACCCTGGA CCAGATGTGT TCCGTGATGA CCTGTAGCAG GAAGACCAAC
1321 CGGTGGCCAA TGGCCCTGCT GTATGGAATG ATCAACATTG CCTGCATCAA CAGCTTCATT
1381 ATTTACTCCC ACAATGTGTC CTCTAAGGGG GAGAAGGTGC AGTCTAGAAA AAAGTTTATG
1441 AGGAATCTGT ATATGGGCCT GACCTCTTCC TTCATGCGGA AGCGCCTGGA GGCACCCACA
1501 CTGAAGAGGT ACCTTCGCGA CAATATCTCC AATATTCTGC CGAAAGAGGT GCCCGGCACC
1561 TCCGACGACA GCACAGAAGA GCCCGTGATG AAAAAGAGGA CCTACTGCAC CTACTGCCCC
1621 TCAAAGATCC GCAGGAAGGC CTCCGCATCC TGTAAGAAGT GCAAGAAGGT CATCTGCAGG
1681 GAACACAATA TCGACATGTG CCAGTCATGC TTC
SEQ ID NO: 504: Trichnoplusia ni (hyperactive piggyBac) amino acid
sequence(N1; amino acid 2-24 deletion). 571 aa
      1 MDSDSEVSDH VSEDDVOSDT EEAFIDEVHE VQPTSSGSEI LDEQNVIEQP GSSLASNRIL
    61 TLPQRTIRGK NKHCWSTSKP TRRSRVSALN IVRSQRGPTR MCRNIYDPLL CFKLFFTDEI
  121 ISEIVKWTNA EISLKRRESM TSATFRDTNE DEIYAFFGIL VMTAVRKDNH MSTDDLFDRS
  181 LSMVYVSVMS RDRFDFLIRC LRMDDKSIRP TLRENDVFTP VRKIWDLFIH QCIQNYTPGA
  241 HLTIDEQLLG FRGRCPFRVY IPNKPSKYGI KILMMCDSGT KYMINGMPYL GRGTQTNGVP
  301 LGEYYVKELS KPVHGSCRNI TCDNWFTSIP LAKNLLQEPY KLTIVGTVRS NKREIPEVLK
  361 NSRSRPVGTS MFCFDGPLTL VSYKPKPAKM VYLLSSCDED ASINESTGKP QMVMYYNQTK
  421 GGVDTLDQMC SVMTCSRKTN RWPMALLYGM INIACINSFI IYSHNVSSKG EKVQSRKKFM
  481 RNLYMGLTSS FMRKRLEAPT LKRYLRDNIS NILPKEVPGT SDDSTEEPVM KKRTYCTYCP
  541 SKIRRKASAS CKKCKKVICR EHNIDMCQSC F
SEQ ID NO: 505: Trichnoplusia ni (hyperactive piggyBac) nucleotide
sequence(N2; nucleotide 4-117 deletion) 1668 bp
      1 ATGCAGAGCG ACACCGAGGA GGCCTTCATC GATGAGGTGC ATGAAGTGCA GCCCACATCC
    61 TCCGGATCCG AGATCCTGGA TGAGCAGAAC GTGATCGAGC AGCCCGGGAG CTCTCTGGCT
  121 TCCAACAGAA TCCTGACCCT GCCTCAGAGG ACTATCAGAG GGAAAAATAA GCACTGCTGG
  181 TCCACTTCAA AGCCAACCAG GCGGTCTAGG GTGTCCGCCC TGAACATCGT GAGAAGCCAG
  241 AGAGGTCCAA CCCGGATGTG CAGAAACATC TACGATCCCC TGCTGTGTTT CAAGCTGTTT
  301 TTTACTGACG AGATTATCAG CGAGATCGTG AAGTGGACCA ACGCCGAGAT CTCTCTGAAG
  361 CGGAGAGAAA GCATGACCTC CGCCACATTC CGCGATACCA ACGAAGACGA GATCTACGCC
  421 TTTTTCGGCA TCTTGGTGAT GACTGCCGTG AGGAAGGACA ATCACATGAG CACCGATGAC
  481 CTGTTCGATC GCAGCCTGAG CATGGTGTAC GTGTCCGTGA TGTCCAGGGA CAGGTTCGAC
  541 TTCCTGATCA GGTGCCTGAG AATGGATGAC AAGAGCATTA GACCTACCCT GCGCGAGAAT
  601 GACGTGTTCA CCCCCGTCAG GAAGATCTGG GACCTGTTTA TTCACCAGTG CATTCAGAAC
  661 TATACCCCTG GCGCCCACCT GACCATTGAT GAGCAGCTCC TGGGATTCAG GGGCCGGTGT
  721 CCCTTCAGAG TGTATATACC TAACAAGCCC TCTAAGTACG GCATCAAAAT CCTGATGATG
  781 TGCGACTCCG GAACCAAGTA CATGATTAAC GGAATGCCCT ATCTGGGACG CGGGACCCAG
  841 ACCAATGGAG TGCCTCTGGG CGAGTATTAC GTGAAAGAGC TGTCCAAACC CGTGCACGGG
  901 TCCTGTCGGA ATATCACCTG CGACAACTGG TTCACTTCAA TCCCCCTGGC CAAAAACCTG
  961 CTGCAGGAGC CTTACAAATT GACCATTGTG GGCACTGTGC GCTCCAATAA GAGAGAAATT
1021 CCTGAGGTGC TGAAGAACTC CAGGTCCAGA CCCGTGGGCA CTAGCATGTT CTGCTTCGAC
1081 GGGCCTCTGA CCCTGGTGAG CTATAAGCCC AAGCCAGCCA AGATGGTGTA CCTGCTGAGC
1141 AGCTGCGACG AGGATGCCTC AATCAACGAG AGCACCGGCA AGCCCCAGAT GGTGATGTAC
1201 TACAATCAGA CCAAAGGCGG CGTGGATACC CTGGACCAGA TGTGTTCCGT GATGACCTGT
1261 AGCAGGAAGA CCAACCGGTG GCCAATGGCC CTGCTGTATG GAATGATCAA CATTGCCTGC
1321 ATCAACAGCT TCATTATTTA CTCCCACAAT GTGTCCTCTA AGGGGGAGAA GGTGCAGTCT
1381 AGAAAAAAGT TTATGAGGAA TCTGTATATG GGCCTGACCT CTTCCTTCAT GCGGAAGCGC
1441 CTGGAGGCAC CCACACTGAA GAGGTACCTT CGCGACAATA TCTCCAATAT TCTGCCGAAA
1501 GAGGTGCCCG GCACCTCCGA CGACAGCACA GAAGAGCCCG TGATGAAAAA GAGGACCTAC
1561 TGCACCTACT GCCCCTCAAA GATCCGCAGG AAGGCCTCCG CATCCTGTAA GAAGTGCAAG
1621 AAGGTCATCT GCAGGGAACA CAATATCGAC ATGTGCCAGT CATGCTTC
SEQ ID NO: 506: Trichnoplusia ni (hyperactive piggyBac) amino acid
sequence(N2; amino acid 2-39 deletion) 556 aa
      1 MQSDTEEAFI DEVHEVQPTS SGSEILDEQN VIEQPGSSLA SNRILTLPQR TIRGKNKHCW
    61 STSKPTRRSR VSALNIVRSQ RGPTRMCRNI YDPLLCFKLF FTDEIISEIV KWTNAEISLK
  121 RRESMTSATF RDTNEDEIYA FFGILVMTAV RKDNHMSTDD LFDRSLSMVY VSVMSRDRFD
  181 FLIRCLRMDD KSIRPTLREN DVFTPVRKIW DLFIHQCIQN YTPGAHLTID EQLLGFRGRC
  241 PFRVYIPNKP SKYGIKILMM CDSGTKYMIN GMPYLGRGTQ TNGVPLGEYY VKELSKPVHG
  301 SCRNITCDNW FTSIPLAKNL LQEPYKLTIV GTVRSNKREI PEVLKNSRSR PVGTSMFCFD
  361 GPLTLVSYKP KPAKMVYLLS SCDEDASINE STGKPQMVMY YNQTKGGVDT LDQMCSVMTC
  421 SRKTNRWPMA LLYGMINIAC INSFIIYSHN VSSKGEKVQS RKKFMRNLYM GLTSSFMRKR
  481 LEAPTLKRYL RDNISNILPK EVPGTSDDST EEPVMKKRTY CTYCPSKIRR KASASCKKCK
  541 KVICREHNID MCQSCF
SEQ ID NO: 507: Trichnoplusia ni (hyperactive piggyBac) nucleotide
sequence(N3; nucleotide 4-258 deletion). 1524 bp
      1 ATGAGGACTA TCAGAGGGAA AAATAAGCAC TGCTGGTCCA CTTCAAAGCC AACCAGGCGG
    61 TCTAGGGTGT CCGCCCTGAA CATCGTGAGA AGCCAGAGAG GTCCAACCCG GATGTGCAGA
  121 AACATCTACG ATCCCCTGCT GTGTTTCAAG CTGTTTTTTA CTGACGAGAT TATCAGCGAG
  181 ATCGTGAAGT GGACCAACGC CGAGATCTCT CTGAAGCGGA GAGAAAGCAT GACCTCCGCC
  241 ACATTCCGCG ATACCAACGA AGACGAGATC TACGCCTTTT TCGGCATCTT GGTGATGACT
  301 GCCGTGAGGA AGGACAATCA CATGAGCACC GATGACCTGT TCGATCGCAG CCTGAGCATG
  361 GTGTACGTGT CCGTGATGTC CAGGGACAGG TTCGACTTCC TGATCAGGTG CCTGAGAATG
  421 GATGACAAGA GCATTAGACC TACCCTGCGC GAGAATGACG TGTTCACCCC CGTCAGGAAG
  481 ATCTGGGACC TGTTTATTCA CCAGTGCATT CAGAACTATA CCCCTGGCGC CCACCTGACC
  541 ATTGATGAGC AGCTCCTGGG ATTCAGGGGC CGGTGTCCCT TCAGAGTGTA TATACCTAAC
  601 AAGCCCTCTA AGTACGGCAT CAAAATCCTG ATGATGTGCG ACTCCGGAAC CAAGTACATG
  661 ATTAACGGAA TGCCCTATCT GGGACGCGGG ACCCAGACCA ATGGAGTGCC TCTGGGCGAG
  721 TATTACGTGA AAGAGCTGTC CAAACCCGTG CACGGGTCCT GTCGGAATAT CACCTGCGAC
  781 AACTGGTTCA CTTCAATCCC CCTGGCCAAA AACCTGCTGC AGGAGCCTTA CAAATTGACC
  841 ATTGTGGGCA CTGTGCGCTC CAATAAGAGA GAAATTCCTG AGGTGCTGAA GAACTCCAGG
  901 TCCAGACCCG TGGGCACTAG CATGTTCTGC TTCGACGGGC CTCTGACCCT GGTGAGCTAT
  961 AAGCCCAAGC CAGCCAAGAT GGTGTACCTG CTGAGCAGCT GCGACGAGGA TGCCTCAATC
1021 AACGAGAGCA CCGGCAAGCC CCAGATGGTG ATGTACTACA ATCAGACCAA AGGCGGCGTG
1081 GATACCCTGG ACCAGATGTG TTCCGTGATG ACCTGTAGCA GGAAGACCAA CCGGTGGCCA
1141 ATGGCCCTGC TGTATGGAAT GATCAACATT GCCTGCATCA ACAGCTTCAT TATTTACTCC
1201 CACAATGTGT CCTCTAAGGG GGAGAAGGTG CAGTCTAGAA AAAAGTTTAT GAGGAATCTG
1261 TATATGGGCC TGACCTCTTC CTTCATGCGG AAGCGCCTGG AGGCACCCAC ACTGAAGAGG
1321 TACCTTCGCG ACAATATCTC CAATATTCTG CCGAAAGAGG TGCCCGGCAC CTCCGACGAC
1381 AGCACAGAAG AGCCCGTGAT GAAAAAGAGG ACCTACTGCA CCTACTGCCC CTCAAAGATC
1441 CGCAGGAAGG CCTCCGCATC CTGTAAGAAG TGCAAGAAGG TCATCTGCAG GGAACACAAT
1501 ATCGACATGT GCCAGTCATG CTTC
SEQ ID NO: 508: Trichnoplusia ni (hyperactive piggyBac)
amino acid sequence(N3; amino acid 2-86 deletion) 508 aa
      1 MRTIRGKNKH CWSTSKPTRR SRVSALNIVR SQRGPTRMCR NIYDPLLCFK LFFTDEIISE
    61 IVKWTNAEIS LKRRESMTSA TFRDTNEDEI YAFFGILVMT AVRKDNHMST DDLFDRSLSM
  121 VYVSVMSRDR FDFLIRCLRM DDKSIRPTLR ENDVFTPVRK IWDLFIHQCI QNYTPGAHLT
  181 IDEQLLGFRG RCPFRVYIPN KPSKYGIKIL MMCDSGTKYM INGMPYLGRG TQTNGVPLGE
  241 YYVKELSKPV HGSCRNITCD NWFTSIPLAK NLLQEPYKLT IVGTVRSNKR EIPEVLKNSR
  301 SRPVGTSMFC FDGPLTLVSY KPKPAKMVYL LSSCDEDASI NESTGKPQMV MYYNQTKGGV
  361 DTLDQMCSVM TCSRKTNRWP MALLYGMINI ACINSFIIYS HNVSSKGEKV QSRKKFMRNL
  421 YMGLTSSFMR KRLEAPTLKR YLRDNISNIL PKEVPGTSDD STEEPVMKKR TYCTYCPSKI
  481 RRKASASCKK CKKVICREHN IDMCQSCF
SEQ ID NO: 509: Trichnoplusia ni (hyperactive piggyBac)
nucleotide sequence(N4; nucleotide 4-348 deletion). 1437 bp
      1 ATGAGCCAGA GAGGTCCAAC CCGGATGTGC AGAAACATCT ACGATCCCCT GCTGTGTTTC
    61 AAGCTGTTTT TTACTGACGA GATTATCAGC GAGATCGTGA AGTGGACCAA CGCCGAGATC
  121 TCTCTGAAGC GGAGAGAAAG CATGACCTCC GCCACATTCC GCGATACCAA CGAAGACGAG
  181 ATCTACGCCT TTTTCGGCAT CTTGGTGATG ACTGCCGTGA GGAAGGACAA TCACATGAGC
  241 ACCGATGACC TGTTCGATCG CAGCCTGAGC ATGGTGTACG TGTCCGTGAT GTCCAGGGAC
  301 AGGTTCGACT TCCTGATCAG GTGCCTGAGA ATGGATGACA AGAGCATTAG ACCTACCCTG
  361 CGCGAGAATG ACGTGTTCAC CCCCGTCAGG AAGATCTGGG ACCTGTTTAT TCACCAGTGC
  421 ATTCAGAACT ATACCCCTGG CGCCCACCTG ACCATTGATG AGCAGCTCCT GGGATTCAGG
  481 GGCCGGTGTC CCTTCAGAGT GTATATACCT AACAAGCCCT CTAAGTACGG CATCAAAATC
  541 CTGATGATGT GCGACTCCGG AACCAAGTAC ATGATTAACG GAATGCCCTA TCTGGGACGC
  601 GGGACCCAGA CCAATGGAGT GCCTCTGGGC GAGTATTACG TGAAAGAGCT GTCCAAACCC
  661 GTGCACGGGT CCTGTCGGAA TATCACCTGC GACAACTGGT TCACTTCAAT CCCCCTGGCC
  721 AAAAACCTGC TGCAGGAGCC TTACAAATTG ACCATTGTGG GCACTGTGCG CTCCAATAAG
  781 AGAGAAATTC CTGAGGTGCT GAAGAACTCC AGGTCCAGAC CCGTGGGCAC TAGCATGTTC
  841 TGCTTCGACG GGCCTCTGAC CCTGGTGAGC TATAAGCCCA AGCCAGCCAA GATGGTGTAC
  901 CTGCTGAGCA GCTGCGACGA GGATGCCTCA ATCAACGAGA GCACCGGCAA GCCCCAGATG
  961 GTGATGTACT ACAATCAGAC CAAAGGCGGC GTGGATACCC TGGACCAGAT GTGTTCCGTG
1021 ATGACCTGTA GCAGGAAGAC CAACCGGTGG CCAATGGCCC TGCTGTATGG AATGATCAAC
1081 ATTGCCTGCA TCAACAGCTT CATTATTTAC TCCCACAATG TGTCCTCTAA GGGGGAGAAG
1141 GTGCAGTCTA GAAAAAAGTT TATGAGGAAT CTGTATATGG GCCTGACCTC TTCCTTCATG
1201 CGGAAGCGCC TGGAGGCACC CACACTGAAG AGGTACCTTC GCGACAATAT CTCCAATATT
1261 CTGCCGAAAG AGGTGCCCGG CACCTCCGAC GACAGCACAG AAGAGCCCGT GATGAAAAAG
1321 AGGACCTACT GCACCTACTG CCCCTCAAAG ATCCGCAGGA AGGCCTCCGC ATCCTGTAAG
1381 AAGTGCAAGA AGGTCATCTG CAGGGAACAC AATATCGACA TGTGCCAGTC ATGCTTC
SEQ ID NO: 510: Trichnoplusia ni (hyperactive piggyBac) amino
acid sequence(N4; amino acid 2-116 deletion). 479 aa
      1 MSQRGPTRMC RNIYDPLLCF KLFFTDEIIS EIVKWTNAEI SLKRRESMTS ATFRDTNEDE
    61 IYAFFGILVM TAVRKDNHMS TDDLFDRSLS MVYVSVMSRD RFDFLIRCLR MDDKSIRPTL
  121 RENDVFTPVR KIWDLFIHQC IQNYTPGAHL TIDEQLLGFR GRCPFRVYIP NKPSKYGIKI
  181 LMMCDSGTKY MINGMPYLGR GTQTNGVPLG EYYVKELSKP VHGSCRNITC DNWFTSIPLA
  241 KNLLQEPYKL TIVGTVRSNK REIPEVLKNS RSRPVGTSMF CFDGPLTLVS YKPKPAKMVY
  301 LLSSCDEDAS INESTGKPQM VMYYNQTKGG VDTLDQMCSV MTCSRKTNRW PMALLYGMIN
  361 IACINSFIIY SHNVSSKGEK VQSRKKFMRN LYMGLTSSFM RKRLEAPTLK RYLRDNISNI
  421 LPKEVPGTSD DSTEEPVMKK RTYCTYCPSK IRRKASASCK KCKKVICREH NIDMCQSCF
SEQ ID NO: 511: Trichnoplusia ni (hyperactive piggyBac) nucleotide
sequence(C1; nucleotide 1708-1782 deletion). 1707 bp
      1 ATGGGCTCTT CCTTGGACGA CGAGCACATC CTGAGCGCCC TGCTGCAGTC CGATGACGAG
    61 CTGGTGGGTG AGGACTCCGA CAGTGAGGTG AGCGATCATG TGTCCGAGGA CGATGTGCAG
  121 AGCGACACCG AGGAGGCCTT CATCGATGAG GTGCATGAAG TGCAGCCCAC ATCCTCCGGA
  181 TCCGAGATCC TGGATGAGCA GAACGTGATC GAGCAGCCCG GGAGCTCTCT GGCTTCCAAC
  241 AGAATCCTGA CCCTGCCTCA GAGGACTATC AGAGGGAAAA ATAAGCACTG CTGGTCCACT
  301 TCAAAGCCAA CCAGGCGGTC TAGGGTGTCC GCCCTGAACA TCGTGAGAAG CCAGAGAGGT
  361 CCAACCCGGA TGTGCAGAAA CATCTACGAT CCCCTGCTGT GTTTCAAGCT GTTTTTTACT
  421 GACGAGATTA TCAGCGAGAT CGTGAAGTGG ACCAACGCCG AGATCTCTCT GAAGCGGAGA
  481 GAAAGCATGA CCTCCGCCAC ATTCCGCGAT ACCAACGAAG ACGAGATCTA CGCCTTTTTC
  541 GGCATCTTGG TGATGACTGC CGTGAGGAAG GACAATCACA TGAGCACCGA TGACCTGTTC
  601 GATCGCAGCC TGAGCATGGT GTACGTGTCC GTGATGTCCA GGGACAGGTT CGACTTCCTG
  661 ATCAGGTGCC TGAGAATGGA TGACAAGAGC ATTAGACCTA CCCTGCGCGA GAATGACGTG
  721 TTCACCCCCG TCAGGAAGAT CTGGGACCTG TTTATTCACC AGTGCATTCA GAACTATACC
  781 CCTGGCGCCC ACCTGACCAT TGATGAGCAG CTCCTGGGAT TCAGGGGCCG GTGTCCCTTC
  841 AGAGTGTATA TACCTAACAA GCCCTCTAAG TACGGCATCA AAATCCTGAT GATGTGCGAC
  901 TCCGGAACCA AGTACATGAT TAACGGAATG CCCTATCTGG GACGCGGGAC CCAGACCAAT
  961 GGAGTGCCTC TGGGCGAGTA TTACGTGAAA GAGCTGTCCA AACCCGTGCA CGGGTCCTGT
1021 CGGAATATCA CCTGCGACAA CTGGTTCACT TCAATCCCCC TGGCCAAAAA CCTGCTGCAG
1081 GAGCCTTACA AATTGACCAT TGTGGGCACT GTGCGCTCCA ATAAGAGAGA AATTCCTGAG
1141 GTGCTGAAGA ACTCCAGGTC CAGACCCGTG GGCACTAGCA TGTTCTGCTT CGACGGGCCT
1201 CTGACCCTGG TGAGCTATAA GCCCAAGCCA GCCAAGATGG TGTACCTGCT GAGCAGCTGC
1261 GACGAGGATG CCTCAATCAA CGAGAGCACC GGCAAGCCCC AGATGGTGAT GTACTACAAT
1321 CAGACCAAAG GCGGCGTGGA TACCCTGGAC CAGATGTGTT CCGTGATGAC CTGTAGCAGG
1381 AAGACCAACC GGTGGCCAAT GGCCCTGCTG TATGGAATGA TCAACATTGC CTGCATCAAC
1441 AGCTTCATTA TTTACTCCCA CAATGTGTCC TCTAAGGGGG AGAAGGTGCA GTCTAGAAAA
1501 AAGTTTATGA GGAATCTGTA TATGGGCCTG ACCTCTTCCT TCATGCGGAA GCGCCTGGAG
1561 GCACCCACAC TGAAGAGGTA CCTTCGCGAC AATATCTCCA ATATTCTGCC GAAAGAGGTG
1621 CCCGGCACCT CCGACGACAG CACAGAAGAG CCCGTGATGA AAAAGAGGAC CTACTGCACC
1681 TACTGCCCCT CAAAGATCCG CAGGAAG
SEQ ID NO: 512: Trichnoplusia ni (hyperactive piggyBac) amino
acid sequence(C1; amino acid 570-594 deletion). 569 aa
      1 MGSSLDDEHI LSALLQSDDE LVGEDSDSEV SDHVSEDDVQ SDTEEAFIDE VHEVQPTSSG
    61 SEILDEQNVI EQPGSSLASN RILTLPQRTI RGKNKHCWST SKPTRRSRVS ALNIVRSQRG
  121 PTRMCRNIYD PLLCFKLFFT DEIISEIVKW TNAEISLKRR ESMTSATFRD TNEDEIYAFF
  181 GILVMTAVRK DNHMSTDDLF DRSLSMVYVS VMSRDRFDFL IRCLRMDDKS IRPTLRENDV
  241 FTPVRKIWDL FIHQCIQNYT PGAHLTIDEQ LLGFRGRCPF RVYIPNKPSK YGIKILMMCD
  301 SGTKYMINGM PYLGRGTQTN GVPLGEYYVK ELSKPVHGSC RNITCDNWFT SIPLAKNLLQ
  361 EPYKLTIVGT VRSNKREIPE VLKNSRSRPV GTSMFCFDGP LTLVSYKPKP AKMVYLLSSC
  421 DEDASINEST GKPQMVMYYN QTKGGVDTLD QMCSVMTCSR KTNRWPMALL YGMINIACIN
  481 SFIIYSHNVS SKGEKVQSRK KFMRNLYMGL TSSFMRKRLE APTLKRYLRD NISNILPKEV
  541 PGTSDDSTEE PVMKKRTYCT YCPSKIRRK
SEQ ID NO: 513: Trichnoplusia ni (hyperactive piggyBac)
nucleotide sequence(C2; nucleotide 1660-1782 deletion).
1659 bp
      1 ATGGGCTCTT CCTTGGACGA CGAGCACATC CTGAGCGCCC TGCTGCAGTC CGATGACGAG
    61 CTGGTGGGTG AGGACTCCGA CAGTGAGGTG AGCGATCATG TGTCCGAGGA CGATGTGCAG
  121 AGCGACACCG AGGAGGCCTT CATCGATGAG GTGCATGAAG TGCAGCCCAC ATCCTCCGGA
  181 TCCGAGATCC TGGATGAGCA GAACGTGATC GAGCAGCCCG GGAGCTCTCT GGCTTCCAAC
  241 AGAATCCTGA CCCTGCCTCA GAGGACTATC AGAGGGAAAA ATAAGCACTG CTGGTCCACT
  301 TCAAAGCCAA CCAGGCGGTC TAGGGTGTCC GCCCTGAACA TCGTGAGAAG CCAGAGAGGT
  361 CCAACCCGGA TGTGCAGAAA CATCTACGAT CCCCTGCTGT GTTTCAAGCT GTTTTTTACT
  421 GACGAGATTA TCAGCGAGAT CGTGAAGTGG ACCAACGCCG AGATCTCTCT GAAGCGGAGA
  481 GAAAGCATGA CCTCCGCCAC ATTCCGCGAT ACCAACGAAG ACGAGATCTA CGCCTTTTTC
  541 GGCATCTTGG TGATGACTGC CGTGAGGAAG GACAATCACA TGAGCACCGA TGACCTGTTC
  601 GATCGCAGCC TGAGCATGGT GTACGTGTCC GTGATGTCCA GGGACAGGTT CGACTTCCTG
  661 ATCAGGTGCC TGAGAATGGA TGACAAGAGC ATTAGACCTA CCCTGCGCGA GAATGACGTG
  721 TTCACCCCCG TCAGGAAGAT CTGGGACCTG TTTATTCACC AGTGCATTCA GAACTATACC
  781 CCTGGCGCCC ACCTGACCAT TGATGAGCAG CTCCTGGGAT TCAGGGGCCG GTGTCCCTTC
  841 AGAGTGTATA TACCTAACAA GCCCTCTAAG TACGGCATCA AAATCCTGAT GATGTGCGAC
  901 TCCGGAACCA AGTACATGAT TAACGGAATG CCCTATCTGG GACGCGGGAC CCAGACCAAT
  961 GGAGTGCCTC TGGGCGAGTA TTACGTGAAA GAGCTGTCCA AACCCGTGCA CGGGTCCTGT
1021 CGGAATATCA CCTGCGACAA CTGGTTCACT TCAATCCCCC TGGCCAAAAA CCTGCTGCAG
1081 GAGCCTTACA AATTGACCAT TGTGGGCACT GTGCGCTCCA ATAAGAGAGA AATTCCTGAG
1141 GTGCTGAAGA ACTCCAGGTC CAGACCCGTG GGCACTAGCA TGTTCTGCTT CGACGGGCCT
1201 CTGACCCTGG TGAGCTATAA GCCCAAGCCA GCCAAGATGG TGTACCTGCT GAGCAGCTGC
1261 GACGAGGATG CCTCAATCAA CGAGAGCACC GGCAAGCCCC AGATGGTGAT GTACTACAAT
1321 CAGACCAAAG GCGGCGTGGA TACCCTGGAC CAGATGTGTT CCGTGATGAC CTGTAGCAGG
1381 AAGACCAACC GGTGGCCAAT GGCCCTGCTG TATGGAATGA TCAACATTGC CTGCATCAAC
1441 AGCTTCATTA TTTACTCCCA CAATGTGTCC TCTAAGGGGG AGAAGGTGCA GTCTAGAAAA
1501 AAGTTTATGA GGAATCTGTA TATGGGCCTG ACCTCTTCCT TCATGCGGAA GCGCCTGGAG
1561 GCACCCACAC TGAAGAGGTA CCTTCGCGAC AATATCTCCA ATATTCTGCC GAAAGAGGTG
1621 CCCGGCACCT CCGACGACAG CACAGAAGAG CCCGTGATG
SEQ ID NO: 514: Trichnoplusia ni (hyperactive piggyBac)
amino acid sequence(C2; amino acid 554-594 deletion).
553 aa
      1 MGSSLDDEHI LSALLQSDDE LVGEDSDSEV SDHVSEDDVQ SDTEEAFIDE VHEVQPTSSG
    61 SEILDEQNVI EQPGSSLASN RILTLPQRTI RGKNKHCWST SKPTRRSRVS ALNIVRSQRG
  121 PTRMCRNIYD PLLCFKLFFT DEIISEIVKW TNAEISLKRR ESMTSATFRD TNEDEIYAFF
  181 GILVMTAVRK DNHMSTDDLF DRSLSMVYVS VMSRDRFDFL IRCLRMDDKS IRPTLRENDV
  241 FTPVRKIWDL FIHQCIQNYT PGAHLTIDEQ LLGFRGRCPF RVYIPNKPSK YGIKILMMCD
  301 SGTKYMINGM PYLGRGTQTN GVPLGEYYVK ELSKPVHGSC RNITCDNWFT SIPLAKNLLQ
  361 EPYKLTIVGT VRSNKREIPE VLKNSRSRPV GTSMFCFDGP LTLVSYKPKP AKMVYLLSSC
  421 DEDASINEST GKPQMVMYYN QTKGGVDTLD QMCSVMTCSR KTNRWPMALL YGMINIACIN
  481 SFIIYSHNVS SKGEKVQSRK KFMRNLYMGL TSSFMRKRLE APTLKRYLRD NISNILPKEV
  541 PGTSDDSTEE PVM
SEQ ID NO: 515: nucleotide sequence of E2C NbAlfa
ATGGCGCCTAAGAAGAAGAGAAAGGTCGGCGGCCTCGAACCAGGCGAGAAGCCTTATGCCTGTCCTGAGTGTGGCAA
ATCCTTCTCAAGAAAAGACTCTCTGGTTAGACACCAGAGAACACATACAGGGGAGAAACCCTATAAATGCCCCGAAT
GCGGAAAGTCCTTTTCCCAGAGCGGCGATCTCCGGAGGCATCAGAGAACTCATACAGGCGAGAAACCATATAAGTGC
CCCGAGTGTGGGAAATCCTTTTCCGATTGTAGAGACCTGGCCAGACATCAAAGGACACATACAGGCAAGAAGACCGC
TAGCGGTGGATCCGGAGGGTCCGGAGGTAGTGGCGGCAGCCTCGAGGAAGTCCAGCTCCAAGAAAGCGGCGGGGGCC
TCGTGCAGCCAGGCGGGTCCCTGAGGCTGAGCTGTACCGCATCTGGAGTCACCATCTCTGCCCTTAACGCAATGGCT
ATGGGGTGGTATCGGCAGGCGCCCGGCGAGAGGAGAGTCATGGTAGCAGCCGTTAGCGAAAGGGGGAATGCGATGTA
CCGAGAGAGTGTTCAGGGACGATTTACTGTCACTCGGGATTTTACGAACAAAATGGTTTCATTGCAAATGGACAATT
TGAAACCAGAGGACACCGCTGTTTACTACTGTCACGTCCTGGAAGATCGAGTAGATAGCTTCCATGACTATTGGGGC
CAAGGAACACAAGTGACTGTCAGCTCC
SEQ ID NO: 516: amino acid sequence of E2C NbAlfa
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEVQLQESGGGLVQPGGSLRLSCTASGVTISALNAMA
MGWYRQAPGERRVMVAAVSERGNAMYRESVQGRFTVTRDFTNKMVSLQMDNLKPEDTAVYYCHVLEDRVDSFHDYWG
QGTQVTVSS
SEQ ID NO: 517: nucleotide sequence of Alfa Nterm R315 R372
ATGGCGCCTAAGAAGAAGAGAAAGGTCGGCGGCTCCAGACTGGAAGAGGAACTGAGAAGAAGGCTCACAGAAGCTAG
CGGTGGATCCGGAGGGTCCGGAGGTAGTGGCGGCAGCCTCGAGGGGTCTTCACTGGACGATGAGCATATTCTGAGCG
CCCTGCTGCAGAGCGATGACGAGCTGGTGGGAGAGGATTCCGATTCCGAGGTCAGTGACCACGTGTCAGAGGACGAT
GTGCAGAGCGACACTGAGGAAGCCTTCATCGATGAGGTCCATGAAGTGCAGCCAACAAGCTCCGGAAGCGAGATCCT
GGATGAACAGAACGTGATTGAACAGCCTGGCTCTAGTCTGGCTTCCAATAGGAACCTGACACTGCCACAGCGAACTA
TTCGGGGCAAGAACAAGCACTGCTGGAGCACCTCCAAGCCTACACGGAGAAGCCGCGCGTCCGCCCTGAACATCGTG
AGATCCCAGAGGGGGCCAACCCGCATGTGCCGAAATATCTACGACCCCCTGCTGTGCTTTAAGCTGTTCTTTACAGA
TGAGATCATTAGTGAAATCGTGAAGTGGACTAACGCAGAGATTTCACTGAAAAGGCGCGAATCTATGACTAGTGCCA
CCTTCAGAGACACAAATGAGGATGAAATCTACGCTTTCTTTGGCATTCTGGTCATGACCGCAGTGAGGAAGGACAAC
CATATGTCTACAGACGATCTGTTTGATCGCTCTCTGAGTATGGTGTATGTCTCAGTGATGAGCAGAGACAGGTTCGA
TTTTTTGATCCGGTGCCTGAGAATGGACGATAAGAGCATTCGACCTACACTGCGGGAGAATGACGTGTTCACCCCAG
TGAGGAAAATCTGGGATCTGTTTATCCACCAGTGTATTCAGAACTACACACCCGGAGCCCATCTGACTATCGACGAA
CAGCTGCTGGGCTTCCGCGGGCGATGCCCTTTTCGCGTATACATTCCAAATAAGCCCAGCAAATATGGCATCAAGAT
TCTGATGATGTGCGATTCCGGGACCAAATACATGATCAACGGAATGCCATATCTGGGAGCCGGCACCCAGACAAATG
GAGTCCCCCTGGGCGAGTACTATGTGAAGGAACTGTCCAAACCTGTCCACGGGTCTTGCAGAAACATCACCTGTGAC
AATTGGTTCACATCTATTCCCCTGGCCAAGAACCTGCTGCAGGAGCCTTATAAACTGACTATCGTCGGAACCGTGGC
CAGCAACAAGAGGGAGATTCCCGAAGTGCTGAAGAACAGCCGGAGCAGACCTGTCGGCACTTCTATGTTCTGCTTTG
ACGGGCCACTGACCCTGGTGAGTTACAAGCCCAAACCTGCTAAAATGGTGTATCTGCTGTCAAGCTGTGACGAGGAT
GCAAGCATCAATGAATCCACCGGCAAGCCCCAGATGGTCATGTACTATAACCAGACTAAAGGCGGGGTGGATACCCT
GAATCAGATGTGCTCTGTCATGACCTGTAGTAGAAAGACAAACAGGTGGCCTATGGCCCTGCTGTACGGGATGATCA
ACATTGCTTGCATTAATTCATTCATCATCTACAGCCACAACGTGTCCTCTAAGGGGGAGAAAGTCCAGTCCCGCAAG
AAATTCATGCGAAATCTGTACATGGGACTGACCAGTAGCTTCATGAGGAAGCGCCTGGAGGCACCCACACTGAAAAG
GTATCTGCGCGACAACATCAGCAATATTCTGCCTAAGGAAGTGCCAGGCACTTCCGACGATTCTACCGAGGAACCAG
TGATGAAGAAACGGACATACTGCACTTATTGTCCCAGCAAGATCCGACGGAAAGCCTCCGCTTCTTGCAAGAAGTGT
AAGAAAGTGATCTGTAGAGAGCATAACATTGATATGTGCCGGTCCTGTTTT
SEQ ID NO: 518: amino acid sequence of Alfa Nterm R315 R372
MAPKKKRKVGGSRLEEELRRRLTEASGGSGGSGGSGGSLEGSSLDDEHILSALLQSDDELVGEDSDSEVSDHVSEDD
VQSDTEEAFIDEVHEVQPTSSGSEILDEQNVIEQPGSSLASNRNLTLPQRTIRGKNKHCWSTSKPTRRSRASALNIV
RSQRGPTRMCRNIYDPLLCFKLFFTDEIISEIVKWTNAEISLKRRESMTSATFRDTNEDEIYAFFGILVMTAVRKDN
HMSTDDLFDRSLSMVYVSVMSRDRFDFLIRCLRMDDKSIRPTLRENDVFTPVRKIWDLFIHQCIQNYTPGAHLTIDE
QLLGFRGRCPFRVYIPNKPSKYGIKILMMCDSGTKYMINGMPYLGAGTQTNGVPLGEYYVKELSKPVHGSCRNITCD
NWFTSIPLAKNLLQEPYKLTIVGTVASNKREIPEVLKNSRSRPVGTSMFCFDGPLTLVSYKPKPAKMVYLLSSCDED
ASINESTGKPQMVMYYNQTKGGVDTLNQMCSVMTCSRKTNRWPMALLYGMINIACINSFIIYSHNVSSKGEKVQSRK
KFMRNLYMGLTSSFMRKRLEAPTLKRYLRDNISNILPKEVPGTSDDSTEEPVMKKRTYCTYCPSKIRRKASASCKKC
KKVICREHNIDMCRSCF
SEQ ID NO: 519: nucleotide sequence of Alfa E428 loop R315 R372
ATGCCGAAAAAAAAACGAAAGGTGTACCCCTACGATGTACCGGACTATGCAGGAAGCGGGTCTTCACTGGACGATGA
GCATATTCTGAGCGCCCTGCTGCAGAGCGATGACGAGCTGGTGGGAGAGGATTCCGATTCCGAGGTCAGTGACCACG
TGTCAGAGGACGATGTGCAGAGCGACACTGAGGAAGCCTTCATCGATGAGGTCCATGAAGTGCAGCCAACAAGCTCC
GGAAGCGAGATCCTGGATGAACAGAACGTGATTGAACAGCCTGGCTCTAGTCTGGCTTCCAATAGGAACCTGACACT
GCCACAGCGAACTATTCGGGGCAAGAACAAGCACTGCTGGAGCACCTCCAAGCCTACACGGAGAAGCCGCGCGTCCG
CCCTGAACATCGTGAGATCCCAGAGGGGGCCAACCCGCATGTGCCGAAATATCTACGACCCCCTGCTGTGCTTTAAG
CTGTTCTTTACAGATGAGATCATTAGTGAAATCGTGAAGTGGACTAACGCAGAGATTTCACTGAAAAGGCGCGAATC
TATGACTAGTGCCACCTTCAGAGACACAAATGAGGATGAAATCTACGCTTTCTTTGGCATTCTGGTCATGACCGCAG
TGAGGAAGGACAACCATATGTCTACAGACGATCTGTTTGATCGCTCTCTGAGTATGGTGTATGTCTCAGTGATGAGC
AGAGACAGGTTCGATTTTTTGATCCGGTGCCTGAGAATGGACGATAAGAGCATTCGACCTACACTGCGGGAGAATGA
CGTGTTCACCCCAGTGAGGAAAATCTGGGATCTGTTTATCCACCAGTGTATTCAGAACTACACACCCGGAGCCCATC
TGACTATCGACGAACAGCTGCTGGGCTTCCGCGGGCGATGCCCTTTTCGCGTATACATTCCAAATAAGCCCAGCAAA
TATGGCATCAAGATTCTGATGATGTGCGATTCCGGGACCAAATACATGATCAACGGAATGCCATATCTGGGAGCCGG
CACCCAGACAAATGGAGTCCCCCTGGGCGAGTACTATGTGAAGGAACTGTCCAAACCTGTCCACGGGTCTTGCAGAA
ACATCACCTGTGACAATTGGTTCACATCTATTCCCCTGGCCAAGAACCTGCTGCAGGAGCCTTATAAACTGACTATC
GTCGGAACCGTGGCCAGCAACAAGAGGGAGATTCCCGAAGTGCTGAAGAACAGCCGGAGCAGACCTGTCGGCACTTC
TATGTTCTGCTTTGACGGGCCACTGACCCTGGTGAGTTACAAGCCCAAATCCAGACTGGAAGAGGAACTGAGAAGAA
GGCTCACAGAACCTGCTAAAATGGTGTATCTGCTGTCAAGCTGTGACGAGGATGCAAGCATCAATGAATCCACCGGC
AAGCCCCAGATGGTCATGTACTATAACCAGACTAAAGGCGGGGTGGATACCCTGAATCAGATGTGCTCTGTCATGAC
CTGTAGTAGAAAGACAAACAGGTGGCCTATGGCCCTGCTGTACGGGATGATCAACATTGCTTGCATTAATTCATTCA
TCATCTACAGCCACAACGTGTCCTCTAAGGGGGAGAAAGTCCAGTCCCGCAAGAAATTCATGCGAAATCTGTACATG
GGACTGACCAGTAGCTTCATGAGGAAGCGCCTGGAGGCACCCACACTGAAAAGGTATCTGCGCGACAACATCAGCAA
TATTCTGCCTAAGGAAGTGCCAGGCACTTCCGACGATTCTACCGAGGAACCAGTGATGAAGAAACGGACATACTGCA
CTTATTGTCCCAGCAAGATCCGACGGAAAGCCTCCGCTTCTTGCAAGAAGTGTAAGAAAGTGATCTGTAGAGAGCAT
AACATTGATATGTGCCGGTCCTGTTTT
SEQ ID NO: 520: amino acid sequence of Alfa E428 loop R315 R372
MPKKKRKVYPYDVPDYAGSGSSLDDEHILSALLQSDDELVGEDSDSEVSDHVSEDDVQSDTEEAFIDEVHEVQPTSS
GSEILDEQNVIEQPGSSLASNRNLTLPQRTIRGKNKHCWSTSKPTRRSRASALNIVRSQRGPTRMCRNIYDPLLCFK
LFFTDEIISEIVKWTNAEISLKRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQLLGFRGRCPFRVYIPNKPSK
YGIKILMMCDSGTKYMINGMPYLGAGTQTNGVPLGEYYVKELSKPVHGSCRNITCDNWFTSIPLAKNLLQEPYKLTI
VGTVASNKREIPEVLKNSRSRPVGTSMFCFDGPLTLVSYKPKSRLEEELRRRLTEPAKMVYLLSSCDEDASINESTG
KPQMVMYYNQTKGGVDTLNQMCSVMTCSRKTNRWPMALLYGMINIACINSFIIYSHNVSSKGEKVQSRKKFMRNLYM
GLTSSFMRKRLEAPTLKRYLRDNISNILPKEVPGTSDDSTEEPVMKKRTYCTYCPSKIRRKASASCKKCKKVICREH
NIDMCRSCF*
SEQ ID NO: 521: nucleotide sequence of E2C GCN4
ATGCCGAAAAAAAAACGAAAGGTGTACCCCTACGATGTACCGGACTATGCAGGAAGCGGGTCTTCACTGGACGATGA
GCATATTCTGAGCGCCCTGCTGCAGAGCGATGACGAGCTGGTGGGAGAGGATTCCGATTCCGAGGTCAGTGACCACG
TGTCAGAGGACGATGTGCAGAGCGACACTGAGGAAGCCTTCATCGATGAGGTCCATGAAGTGCAGCCAACAAGCTCC
GGAAGCGAGATCCTGGATGAACAGAACGTGATTGAACAGCCTGGCTCTAGTCTGGCTTCCAATAGGAACCTGACACT
GCCACAGCGAACTATTCGGGGCAAGAACAAGCACTGCTGGAGCACCTCCAAGCCTACACGGAGAAGCCGCGCGTCCG
CCCTGAACATCGTGAGATCCCAGAGGGGGCCAACCCGCATGTGCCGAAATATCTACGACCCCCTGCTGTGCTTTAAG
CTGTTCTTTACAGATGAGATCATTAGTGAAATCGTGAAGTGGACTAACGCAGAGATTTCACTGAAAAGGCGCGAATC
TATGACTAGTGCCACCTTCAGAGACACAAATGAGGATGAAATCTACGCTTTCTTTGGCATTCTGGTCATGACCGCAG
TGAGGAAGGACAACCATATGTCTACAGACGATCTGTTTGATCGCTCTCTGAGTATGGTGTATGTCTCAGTGATGAGC
AGAGACAGGTTCGATTTTTTGATCCGGTGCCTGAGAATGGACGATAAGAGCATTCGACCTACACTGCGGGAGAATGA
CGTGTTCACCCCAGTGAGGAAAATCTGGGATCTGTTTATCCACCAGTGTATTCAGAACTACACACCCGGAGCCCATC
TGACTATCGACGAACAGCTGCTGGGCTTCCGCGGGCGATGCCCTTTTCGCGTATACATTCCAAATAAGCCCAGCAAA
TATGGCATCAAGATTCTGATGATGTGCGATTCCGGGACCAAATACATGATCAACGGAATGCCATATCTGGGAGCCGG
CACCCAGACAAATGGAGTCCCCCTGGGCGAGTACTATGTGAAGGAACTGTCCAAACCTGTCCACGGGTCTTGCAGAA
ACATCACCTGTGACAATTGGTTCACATCTATTCCCCTGGCCAAGAACCTGCTGCAGGAGCCTTATAAACTGACTATC
GTCGGAACCGTGGCCAGCAACAAGAGGGAGATTCCCGAAGTGCTGAAGAACAGCCGGAGCAGACCTGTCGGCACTTC
TATGTTCTGCTTTGACGGGCCACTGACCCTGGTGAGTTACAAGCCCAAATCCAGACTGGAAGAGGAACTGAGAAGAA
GGCTCACAGAACCTGCTAAAATGGTGTATCTGCTGTCAAGCTGTGACGAGGATGCAAGCATCAATGAATCCACCGGC
AAGCCCCAGATGGTCATGTACTATAACCAGACTAAAGGCGGGGTGGATACCCTGAATCAGATGTGCTCTGTCATGAC
CTGTAGTAGAAAGACAAACAGGTGGCCTATGGCCCTGCTGTACGGGATGATCAACATTGCTTGCATTAATTCATTCA
TCATCTACAGCCACAACGTGTCCTCTAAGGGGGAGAAAGTCCAGTCCCGCAAGAAATTCATGCGAAATCTGTACATG
GGACTGACCAGTAGCTTCATGAGGAAGCGCCTGGAGGCACCCACACTGAAAAGGTATCTGCGCGACAACATCAGCAA
TATTCTGCCTAAGGAAGTGCCAGGCACTTCCGACGATTCTACCGAGGAACCAGTGATGAAGAAACGGACATACTGCA
CTTATTGTCCCAGCAAGATCCGACGGAAAGCCTCCGCTTCTTGCAAGAAGTGTAAGAAAGTGATCTGTAGAGAGCAT
AACATTGATATGTGCCGGTCCTGTTTT
SEQ ID NO: 522: amino acid sequence of E2C GCN4
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEELLSKNYHLENEVARLKK
SEQ ID NO: 523: nucleotide sequence of E2C GCN4 (GCN4 underlined)
ATGGCGCCTAAGAAGAAGAGAAAGGTCGGCGGCCTCGAACCAGGCGAGAAGCCTTATGCCTGTCCTGAGTGTGGCAA
ATCCTTCTCAAGAAAAGACTCTCTGGTTAGACACCAGAGAACACATACAGGGGAGAAACCCTATAAATGCCCCGAAT
GCGGAAAGTCCTTTTCCCAGAGCGGCGATCTCCGGAGGCATCAGAGAACTCATACAGGCGAGAAACCATATAAGTGC
CCCGAGTGTGGGAAATCCTTTTCCGATTGTAGAGACCTGGCCAGACATCAAAGGACACATACAGGCAAGAAGACCGC
TAGCGGTGGATCCGGAGGGTCCGGAGGTAGTGGCGGCAGCCTCGAGGAAGAACTTTTGAGCAAGAATTATCATCTTG
AGAACGAAGTGGCTCGTCTTAAGAAA
SEQ ID NO: 524: amino acid sequence of E2C GCN4 (GCN4 underlined)
MAPKKKRKVGGLEPGEKPYACPECGKSFSRKDSLVRHQRTHTGEKPYKCPECGKSFSQSGDLRRHQRTHTGEKPYKC
PECGKSFSDCRDLARHQRTHTGKKTASGGSGGSGGSGGSLEEELLSKNYHLENEVARLKK
SEQ ID NO: 525: nucleotide sequence of Monobody Nterm R315 R372
ATGGCGCCTAAGAAGAAGAGAAAGGTCGGCGGCGGACCTGATATTGTCATGACACAAAGCCCTAGCTCCCTGAGCGC
TAGCGTGGGCGATAGAGTCACAATAACATGCAGATCCAGCACAGGCGCTGTGACTACGTCCAATTACGCTTCTTGGG
TCCAGGAGAAACCTGGAAAGCTGTTCAAAGGGCTCATAGGAGGCACTAATAATCGGGCCCCTGGCGTCCCGTCTAGA
TTTTCCGGCAGCCTCATTGGGGACAAGGCTACCCTGACCATTAGCTCCCTCCAACCAGAGGACTTCGCTACGTATTT
TTGTGCTCTGTGGTATTCCAACCATTGGGTGTTTGGCCAAGGTACTAAGGTCGAACTCAAACGGGGTGGTGGCGGGT
CCGGAGGAGGAGGGTCTGGAGGAGGGGGGTCCTCTGGAGGGGGCAGCGAGGTTAAGCTCCTGGAGTCTGGAGGTGGG
CTTGTCCAACCCGGCGGATCACTGAAACTGAGCTGCGCTGTCTCCGGTTTTTCCCTCACCGACTACGGGGTGAACTG
GGTTCGCCAGGCGCCAGGCCGGGGATTGGAGTGGATTGGTGTAATATGGGGTGATGGGATCACAGACTACAACAGCG
CTCTTAAAGATCGGTTTATCATCTCTAAAGATAATGGCAAGAATACGGTCTATCTGCAAATGTCTAAAGTGAGATCC
GACGACACCGCCCTCTATTACTGTGTCACCGGACTCTTCGACTACTGGGGCCAAGGCACACTGGTCACAGTCAGCAG
CGCTAGCGGTGGATCCGGAGGGTCCGGAGGTAGTGGCGGCAGCCTCGAGGGGTCTTCACTGGACGATGAGCATATTC
TGAGCGCCCTGCTGCAGAGCGATGACGAGCTGGTGGGAGAGGATTCCGATTCCGAGGTCAGTGACCACGTGTCAGAG
GACGATGTGCAGAGCGACACTGAGGAAGCCTTCATCGATGAGGTCCATGAAGTGCAGCCAACAAGCTCCGGAAGCGA
GATCCTGGATGAACAGAACGTGATTGAACAGCCTGGCTCTAGTCTGGCTTCCAATAGGAACCTGACACTGCCACAGC
GAACTATTCGGGGCAAGAACAAGCACTGCTGGAGCACCTCCAAGCCTACACGGAGAAGCCGCGCGTCCGCCCTGAAC
ATCGTGAGATCCCAGAGGGGGCCAACCCGCATGTGCCGAAATATCTACGACCCCCTGCTGTGCTTTAAGCTGTTCTT
TACAGATGAGATCATTAGTGAAATCGTGAAGTGGACTAACGCAGAGATTTCACTGAAAAGGCGCGAATCTATGACTA
GTGCCACCTTCAGAGACACAAATGAGGATGAAATCTACGCTTTCTTTGGCATTCTGGTCATGACCGCAGTGAGGAAG
GACAACCATATGTCTACAGACGATCTGTTTGATCGCTCTCTGAGTATGGTGTATGTCTCAGTGATGAGCAGAGACAG
GTTCGATTTTTTGATCCGGTGCCTGAGAATGGACGATAAGAGCATTCGACCTACACTGCGGGAGAATGACGTGTTCA
CCCCAGTGAGGAAAATCTGGGATCTGTTTATCCACCAGTGTATTCAGAACTACACACCCGGAGCCCATCTGACTATC
GACGAACAGCTGCTGGGCTTCCGCGGGCGATGCCCTTTTCGCGTATACATTCCAAATAAGCCCAGCAAATATGGCAT
CAAGATTCTGATGATGTGCGATTCCGGGACCAAATACATGATCAACGGAATGCCATATCTGGGAGCCGGCACCCAGA
CAAATGGAGTCCCCCTGGGCGAGTACTATGTGAAGGAACTGTCCAAACCTGTCCACGGGTCTTGCAGAAACATCACC
TGTGACAATTGGTTCACATCTATTCCCCTGGCCAAGAACCTGCTGCAGGAGCCTTATAAACTGACTATCGTCGGAAC
CGTGGCCAGCAACAAGAGGGAGATTCCCGAAGTGCTGAAGAACAGCCGGAGCAGACCTGTCGGCACTTCTATGTTCT
GCTTTGACGGGCCACTGACCCTGGTGAGTTACAAGCCCAAACCTGCTAAAATGGTGTATCTGCTGTCAAGCTGTGAC
GAGGATGCAAGCATCAATGAATCCACCGGCAAGCCCCAGATGGTCATGTACTATAACCAGACTAAAGGCGGGGTGGA
TACCCTGAATCAGATGTGCTCTGTCATGACCTGTAGTAGAAAGACAAACAGGTGGCCTATGGCCCTGCTGTACGGGA
TGATCAACATTGCTTGCATTAATTCATTCATCATCTACAGCCACAACGTGTCCTCTAAGGGGGAGAAAGTCCAGTCC
CGCAAGAAATTCATGCGAAATCTGTACATGGGACTGACCAGTAGCTTCATGAGGAAGCGCCTGGAGGCACCCACACT
GAAAAGGTATCTGCGCGACAACATCAGCAATATTCTGCCTAAGGAAGTGCCAGGCACTTCCGACGATTCTACCGAGG
AACCAGTGATGAAGAAACGGACATACTGCACTTATTGTCCCAGCAAGATCCGACGGAAAGCCTCCGCTTCTTGCAAG
AAGTGTAAGAAAGTGATCTGTAGAGAGCATAACATTGATATGTGCCGGTCCTGTTTT
SEQ ID NO: 526: amino acid sequence of Monobody Nterm R315 R372
MAPKKKRKVGGGPDIVMTQSPSSLSASVGDRVTITCRSSTGAVTTSNYASWVQEKPGKLFKGLIGGTNNRAPGVPSR
FSGSLIGDKATLTISSLQPEDFATYFCALWYSNHWVFGQGTKVELKRGGGGSGGGGSGGGGSSGGGSEVKLLESGGG
LVQPGGSLKLSCAVSGFSLTDYGVNWVRQAPGRGLEWIGVIWGDGITDYNSALKDRFIISKDNGKNTVYLQMSKVRS
DDTALYYCVTGLFDYWGQGTLVTVSSASGGSGGSGGSGGSLEGSSLDDEHILSALLQSDDELVGEDSDSEVSDHVSE
DDVQSDTEEAFIDEVHEVQPTSSGSEILDEQNVIEQPGSSLASNRNLTLPQRTIRGKNKHCWSTSKPTRRSRASALN
IVRSQRGPTRMCRNIYDPLLCFKLFFTDEIISEIVKWTNAEISLKRRESMTSATFRDTNEDEIYAFFGILVMTAVRK
DNHMSTDDLFDRSLSMVYVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGAGTQTNGVPLGEYYVKELSKPVHGSCRNIT
CDNWFTSIPLAKNLLQEPYKLTIVGTVASNKREIPEVLKNSRSRPVGTSMFCFDGPLTLVSYKPKPAKMVYLLSSCD
EDASINESTGKPQMVMYYNQTKGGVDTLNQMCSVMTCSRKTNRWPMALLYGMINIACINSFIIYSHNVSSKGEKVQS
RKKFMRNLYMGLTSSFMRKRLEAPTLKRYLRDNISNILPKEVPGTSDDSTEEPVMKKRTYCTYCPSKIRRKASASCK
KCKKVICREHNIDMCRSCF
SEQ ID NO: 527: nucleotide sequence of Monobody Nterm R315 R372
(monobody underlined)
ATGGCGCCTAAGAAGAAGAGAAAGGTCGGCGGCGGACCTGATATTGTCATGACACAAAGCCCTAGCTCCCTGAGCGC
TAGCGTGGGCGATAGAGTCACAATAACATGCAGATCCAGCACAGGCGCTGTGACTACGTCCAATTACGCTTCTTGGG
TCCAGGAGAAACCTGGAAAGCTGTTCAAAGGGCTCATAGGAGGCACTAATAATCGGGCCCCTGGCGTCCCGTCTAGA
TTTTCCGGCAGCCTCATTGGGGACAAGGCTACCCTGACCATTAGCTCCCTCCAACCAGAGGACTTCGCTACGTATTT
TTGTGCTCTGTGGTATTCCAACCATTGGGTGTTTGGCCAAGGTACTAAGGTCGAACTCAAACGGGGTGGTGGCGGGT
CCGGAGGAGGAGGGTCTGGAGGAGGGGGGTCCTCTGGAGGGGGCAGCGAGGTTAAGCTCCTGGAGTCTGGAGGTGGG
CTTGTCCAACCCGGCGGATCACTGAAACTGAGCTGCGCTGTCTCCGGTTTTTCCCTCACCGACTACGGGGTGAACTG
GGTTCGCCAGGCGCCAGGCCGGGGATTGGAGTGGATTGGTGTAATATGGGGTGATGGGATCACAGACTACAACAGCG
CTCTTAAAGATCGGTTTATCATCTCTAAAGATAATGGCAAGAATACGGTCTATCTGCAAATGTCTAAAGTGAGATCC
GACGACACCGCCCTCTATTACTGTGTCACCGGACTCTTCGACTACTGGGGCCAAGGCACACTGGTCACAGTCAGCAG
CGCTAGCGGTGGATCCGGAGGGTCCGGAGGTAGTGGCGGCAGCCTCGAGGGGTCTTCACTGGACGATGAGCATATTC
TGAGCGCCCTGCTGCAGAGCGATGACGAGCTGGTGGGAGAGGATTCCGATTCCGAGGTCAGTGACCACGTGTCAGAG
GACGATGTGCAGAGCGACACTGAGGAAGCCTTCATCGATGAGGTCCATGAAGTGCAGCCAACAAGCTCCGGAAGCGA
GATCCTGGATGAACAGAACGTGATTGAACAGCCTGGCTCTAGTCTGGCTTCCAATAGGAACCTGACACTGCCACAGC
GAACTATTCGGGGCAAGAACAAGCACTGCTGGAGCACCTCCAAGCCTACACGGAGAAGCCGCGCGTCCGCCCTGAAC
ATCGTGAGATCCCAGAGGGGGCCAACCCGCATGTGCCGAAATATCTACGACCCCCTGCTGTGCTTTAAGCTGTTCTT
TACAGATGAGATCATTAGTGAAATCGTGAAGTGGACTAACGCAGAGATTTCACTGAAAAGGCGCGAATCTATGACTA
GTGCCACCTTCAGAGACACAAATGAGGATGAAATCTACGCTTTCTTTGGCATTCTGGTCATGACCGCAGTGAGGAAG
GACAACCATATGTCTACAGACGATCTGTTTGATCGCTCTCTGAGTATGGTGTATGTCTCAGTGATGAGCAGAGACAG
GTTCGATTTTTTGATCCGGTGCCTGAGAATGGACGATAAGAGCATTCGACCTACACTGCGGGAGAATGACGTGTTCA
CCCCAGTGAGGAAAATCTGGGATCTGTTTATCCACCAGTGTATTCAGAACTACACACCCGGAGCCCATCTGACTATC
GACGAACAGCTGCTGGGCTTCCGCGGGCGATGCCCTTTTCGCGTATACATTCCAAATAAGCCCAGCAAATATGGCAT
CAAGATTCTGATGATGTGCGATTCCGGGACCAAATACATGATCAACGGAATGCCATATCTGGGAGCCGGCACCCAGA
CAAATGGAGTCCCCCTGGGCGAGTACTATGTGAAGGAACTGTCCAAACCTGTCCACGGGTCTTGCAGAAACATCACC
TGTGACAATTGGTTCACATCTATTCCCCTGGCCAAGAACCTGCTGCAGGAGCCTTATAAACTGACTATCGTCGGAAC
CGTGGCCAGCAACAAGAGGGAGATTCCCGAAGTGCTGAAGAACAGCCGGAGCAGACCTGTCGGCACTTCTATGTTCT
GCTTTGACGGGCCACTGACCCTGGTGAGTTACAAGCCCAAACCTGCTAAAATGGTGTATCTGCTGTCAAGCTGTGAC
GAGGATGCAAGCATCAATGAATCCACCGGCAAGCCCCAGATGGTCATGTACTATAACCAGACTAAAGGCGGGGTGGA
TACCCTGAATCAGATGTGCTCTGTCATGACCTGTAGTAGAAAGACAAACAGGTGGCCTATGGCCCTGCTGTACGGGA
TGATCAACATTGCTTGCATTAATTCATTCATCATCTACAGCCACAACGTGTCCTCTAAGGGGGAGAAAGTCCAGTCC
CGCAAGAAATTCATGCGAAATCTGTACATGGGACTGACCAGTAGCTTCATGAGGAAGCGCCTGGAGGCACCCACACT
GAAAAGGTATCTGCGCGACAACATCAGCAATATTCTGCCTAAGGAAGTGCCAGGCACTTCCGACGATTCTACCGAGG
AACCAGTGATGAAGAAACGGACATACTGCACTTATTGTCCCAGCAAGATCCGACGGAAAGCCTCCGCTTCTTGCAAG
AAGTGTAAGAAAGTGATCTGTAGAGAGCATAACATTGATATGTGCCGGTCCTGTTTT
SEQ ID NO: 528: amino acid sequence of Monobody Nterm R315 R372
(monobody underlined)
MAPKKKRKVGGGPDIVMTQSPSSLSASVGDRVTITCRSSTGAVTTSNYASWVQEKPGKLFKGLIGGTNNRAPGVPSR
FSGSLIGDKATLTISSLQPEDFATYFCALWYSNHWVFGQGTKVELKRGGGGSGGGGSGGGGSSGGGSEVKLLESGGG
LVQPGGSLKLSCAVSGFSLTDYGVNWVRQAPGRGLEWIGVIWGDGITDYNSALKDRFIISKDNGKNTVYLQMSKVRS
DDTALYYCVTGLFDYWGQGTLVTVSSASGGSGGSGGSGGSLEGSSLDDEHILSALLQSDDELVGEDSDSEVSDHVSE
DDVQSDTEEAFIDEVHEVQPTSSGSEILDEQNVIEQPGSSLASNRNLTLPQRTIRGKNKHCWSTSKPTRRSRASALN
IVRSQRGPTRMCRNIYDPLLCFKLFFTDEIISEIVKWTNAEISLKRRESMTSATFRDTNEDEIYAFFGILVMTAVRK
DNHMSTDDLFDRSLSMVYVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGAGTQTNGVPLGEYYVKELSKPVHGSCRNIT
CDNWFTSIPLAKNLLQEPYKLTIVGTVASNKREIPEVLKNSRSRPVGTSMFCFDGPLTLVSYKPKPAKMVYLLSSCD
EDASINESTGKPQMVMYYNQTKGGVDTLNQMCSVMTCSRKTNRWPMALLYGMINIACINSFIIYSHNVSSKGEKVQS
RKKFMRNLYMGLTSSFMRKRLEAPTLKRYLRDNISNILPKEVPGTSDDSTEEPVMKKRTYCTYCPSKIRRKASASCK
KCKKVICREHNIDMCRSCF
```

### Numbered Embodiments

1. A composition comprising (a) a transposase enzyme or a nucleic acid encoding the enzyme, wherein the enzyme comprises an amino acid sequence having at least about 80% sequence identity to SEQ ID NO: 11, and wherein the enzyme comprises an amino acid substitution at the position corresponding to position 450 of SEQ ID NO: 11 or (b) a transposase enzyme or a nucleic acid encoding the enzyme and a targeting element which directs the enzyme to target site, optionally a genomic safe harbor site (GSHS), wherein the enzyme is a piggyBac transposase which comprises one or more mutations which cause decreased or ablated integration activity relative to an enzyme comprising an amino acid sequence of SEQ ID NO: 11 or functional equivalent thereof or (c) a transposase enzyme or a nucleic acid encoding the enzyme and a targeting element which directs the enzyme to a target site, optionally a genomic safe harbor site (GSHS) and optionally a linker or linking domain which connects the enzyme and targeting element, wherein the enzyme is a piggyBac transposase and the targeting element and/or linker or linking domain are fused to the N- or C-terminus of the piggyBac transposase or inserted into the piggyBac transposase at one or more internal loops of the enzyme.

2. The composition of Embodiment 1, wherein the enzyme comprises an amino acid sequence of at least about 90% identity to SEQ ID NO: 11.

3. The composition of Embodiment 1 or Embodiment 2, wherein the enzyme comprises an amino acid sequence of at least about 93% identity to SEQ ID NO: 11.

4. The composition of any one of Embodiments 1-3, wherein the enzyme comprises an amino acid sequence of at least about 95% identity to SEQ ID NO: 11.

5. The composition of any one of Embodiments 1-4, wherein the enzyme comprises an amino acid sequence of at least about 98% identity to SEQ ID NO: 11.

6. The composition of any one of Embodiments 1-5, wherein the enzyme comprises an amino acid sequence of at least about 99% identity to SEQ ID NO: 11.

7. The composition of any one of Embodiments 1-6, wherein the substitution at position 450 is with an amino acid other than aspartate (D).

8. The composition of any one of Embodiments 1-7, wherein the substitution is with a polar uncharged amino acid.

9. The composition of Embodiment 8, wherein the polar uncharged amino acid is selected from serine(S) threonine (T), cysteine (C), asparagine (N), glutamine (Q), and proline (P).

10. The composition of Embodiment 9, wherein the polar uncharged amino acid is asparagine (N) or glutamine (Q).

11. The composition of Embodiment 10, wherein the polar uncharged amino acid is asparagine (N).

12. The composition of any one of Embodiments 1-11, wherein the enzyme comprises at least one, at least five, at least seven, at least nine, or ten substitutions at positions corresponding to: 30, 82, 103, 109, 165, 282, 509, 538, 571, and/or 591 of SEQ ID NO: 11.

13. The composition of any one of Embodiments 1-12, wherein the enzyme comprises one, two, three, four, five, six, seven, eight, nine, or ten substitutions at positions corresponding to: 30, 82, 103, 109, 165, 282, 509, 538, 571, and/or 591 of SEQ ID NO: 11.

14. The composition of any one of Embodiments 1-13, wherein the enzyme comprises at least one, at least five, at least seven, at least nine, or ten substitutions selected from 130V, S103P, G165S, M282V, S509G, N538K, N571S, 182N, V109A, and Q591R, wherein the positions are corresponding to positions of SEQ ID NO: 11.

15. The composition of any one of Embodiments 1-14, wherein the enzyme comprises one, two, three, four, five, six, seven, eight, nine, or ten substitutions selected from 130V, S103P, G165S, M282V, S509G, N538K, N571S, 182N, V109A, and Q591R, wherein the positions are corresponding to positions of SEQ ID NO: 11.

16. The composition of any one of Embodiments 1-15, wherein the enzyme comprises, at the positions corresponding to positions of SEQ ID NO: 11, substitutions of D450N, 130V, S103P, G165S, M282V, S509G, N538K, N571S, 182N, V109A, and Q591R.

17. The composition of any one of Embodiments 1-16, wherein the enzyme comprises an amino acid sequence of SEQ ID NO: 1.

18. The composition of any one of Embodiments 1-17, wherein the nucleic acid that encodes the enzyme has a nucleotide sequence of SEQ ID NO: 7 or a codon-optimized form thereof.

19. The composition of any one of Embodiments 1-18, wherein the enzyme has increased excision activity relative to an enzyme comprising an amino acid sequence of SEQ ID NO: 11 or functional equivalent thereof.

20. The composition of any one of Embodiments 1-19, wherein the enzyme is excision positive and/or wherein the enzyme is integration deficient.

21. The composition of any one of Embodiments 1-20, wherein the enzyme comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 502, wherein the deletion comprises an N terminal deletion, wherein the N terminal deletion yields reduced or ablated off-target effects of the enzyme compared to the enzyme without the N terminal deletion, wherein the enzyme comprising the N terminal deletion is listed on TABLE 11.

22. The composition of any one of Embodiments 1-21, wherein the enzyme has decreased integration activity relative to an enzyme comprising an amino acid sequence of SEQ ID NO: 11 or functional equivalent thereof.

23. The composition of any one of Embodiments 1-22, wherein the enzyme has increased excision activity relative to an enzyme comprising an amino acid sequence of SEQ ID NO: 2 or functional equivalent thereof.

24. The composition of any one of Embodiments 1-23, wherein the enzyme has increased excision activity relative to an enzyme comprising an amino acid sequence of SEQ ID NO: 3 or functional equivalent thereof.

25. The composition of any one of Embodiments 1-24, wherein the enzyme comprises at least one substitution at positions corresponding to: 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, and/or 504 of SEQ ID NO: 11.

26. The composition of any one of Embodiments 1-25, wherein the enzyme comprises at least one substitution at positions corresponding to: 312, 315, 324, 347, 372, 374, and/or 375 of SEQ ID NO: 11, and/or wherein the enzyme comprises at least one substitution selected from Y312A, R315A, L324A, N347A, R372A, N374A, and K375A, wherein the positions are corresponding to positions of SEQ ID NO: 11.

27. The composition of any one of Embodiments 1-26, wherein the enzyme comprises substitution(s) selected from R372A/K375A, R372A/R315A, N347A/R315A, L324A/Y312A, N374A, L324A/R315A, R315A/R372A/K375A, and L324A/N347A, wherein the positions are corresponding to positions of SEQ ID NO: 11.

28. The composition of any one of Embodiments 1-27, wherein the enzyme comprises a targeting element.

29. The composition of any one of Embodiments 1-28, wherein the enzyme is capable of inserting a transposon comprising a transgene in a target site, optionally a genomic safe harbor site (GSHS).

30. The composition of Embodiment 29, wherein the binding of a GSHS of a nucleic acid molecule in a mammalian cell is with high target specificity, relative to a control.

31. The composition of Embodiment 30, wherein the control is a composition comprising an enzyme comprising an amino acid sequence of SEQ ID NO: 11 or a nucleic acid comprising a nucleotide sequence of SEQ ID NO: 12 or a codon-optimized form thereof, wherein the control is a composition comprising an enzyme comprising an amino acid sequence of SEQ ID NO: 2 or a nucleic acid comprising a nucleotide sequence of SEQ ID NO: 8 or a codon-optimized form thereof, and/or wherein the control is a composition comprising an enzyme comprising an amino acid sequence of SEQ ID NO: 3 or a nucleic acid comprising a nucleotide sequence of SEQ ID NO: 9 or a codon-optimized form thereof.

32. The composition of any one of Embodiments 28-31, wherein the targeting element is able to direct a transposition machinery to the GSHS of a nucleic acid molecule in a mammalian cell.

33. The composition of any one of Embodiments 28-32, wherein the GSHS is in an open chromatin location in a chromosome.

34. The composition of any one of Embodiments 28-33, wherein the GSHS is selected from adeno-associated virus site 1 (AAVS1), chemokine (C-C motif) receptor 5 (CCR5) gene, HIV-1 coreceptor, and human Rosa26 locus.

35. The composition of any one of Embodiments 28-34, wherein the GSHS is an adeno-associated virus site 1 (AAVS1).

36. The composition of any one of Embodiments 28-34, wherein the GSHS is a human Rosa26 locus.

37. The composition of any one of Embodiments 28-36, wherein the GSHS is located on human chromosome 2, 4, 6, 10, 11, 17, or 22.

38. The composition of any one of Embodiments 28-37, wherein the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

39. The composition of any one of Embodiments 28-38, wherein the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

40. The composition of Embodiment 39, wherein the targeting element comprises a TALE DBD.

41. The composition of Embodiment 40, wherein the TALE DBD comprises one or more repeat sequences.

42. The composition of Embodiment 41, wherein the TALE DBD comprises about 14, or about 15, or about, 16, or about 17, or about 18, or about 18.5 repeat sequences.

43. The composition of Embodiment 41 or Embodiment 42, wherein the repeat sequences each independently comprises about 33 or 34 amino acids.

44. The composition of Embodiment 43, wherein the repeat sequences each independently comprises a repeat variable di-residue (RVD) at residue 12 or 13 of the 33 or 34 amino acids, respectively.

45. The composition of Embodiment 44, wherein the RVD recognizes one base pair in a target nucleic acid sequence.

46. The composition of Embodiment 44 or Embodiment 45, wherein the RVD recognizes a C residue in the target nucleic acid sequence and is selected from HD, N (gap), HA, ND, and HI.

47. The composition of Embodiment 44 or Embodiment 45, wherein the RVD recognizes a G residue in the target nucleic acid sequence and is selected from NN, NH, NK, HN, and NA.

48. The composition of Embodiment 44 or Embodiment 45, wherein the RVD recognizes an A residue in the target nucleic acid sequence and is selected from NI and NS.

49. The composition of Embodiment 44 or Embodiment 45, wherein the RVD recognizes a T residue in the target nucleic acid sequence and is selected from NG, HG, H(gap), and IG.

50. The composition of Embodiment 39, wherein the targeting element comprises a Cas9 enzyme associated with a gRNA.

51. The composition of Embodiment 50, wherein the Cas9 enzyme associated with a gRNA comprises a catalytically inactive dCas9 associated with a gRNA.

52. The composition of Embodiment 51, wherein catalytically inactive dCas9 comprises at least about 90%, or at least about 95%, or at least about 96%, or at least about 97%, or at least about 98%, or at least about 99% identity to an amino acid sequence of SEQ ID NO: 4 or a nucleic acid comprising a nucleotide sequence of SEQ ID NO: 10 or a codon-optimized form thereof.

53. The composition of any one of Embodiments 28-52, wherein the targeting element comprises a Cas12 enzyme associated with a gRNA.

54. The composition of Embodiment 53, wherein the targeting element comprises a catalytically inactive Cas12 associated with a gRNA, optionally wherein the catalytically inactive Cas12 is dCas12j or dCas12a.

55. The composition of any one of Embodiments 28-54, wherein the targeting element comprises a nucleic acid binding component of a gene-editing system.

56. The composition of any one of Embodiments 28-55, wherein the enzyme or variant thereof and the targeting element are connected.

57. The composition of Embodiment 56, wherein the enzyme and the targeting element are fused to one another or linked via a linker to one another.

58. The composition of Embodiment 57, wherein the linker is a flexible linker.

59. The composition of Embodiment 58, wherein the flexible linker is substantially comprised of glycine and serine residues, optionally wherein the flexible linker comprises (Gly4Ser)ₙ, where n is an integer from 1-12.

60. The composition of Embodiment 59, wherein the flexible linker is of about 20, or about 30, or about 40, or about 50, or about 60 amino acid residues.

61. The composition of Embodiment 57, wherein the enzyme is directly fused to the N-terminus of the dCas9 enzyme.

62. The composition of any one of Embodiments 1-61, wherein the enzyme or variant thereof is able to directly or indirectly cause transposition of a target gene.

63. The composition of any one of Embodiments 1-62, wherein the enzyme or variant thereof is able to directly or indirectly interact and/or form a complex with one or more proteins or nucleic acids.

64. The composition of any one of Embodiments 1-63, further comprising a nucleic acid encoding a transposon comprising a transgene to be integrated.

65. The composition of Embodiment 64, wherein the transgene comprises a cargo nucleic acid sequence and a first and a second transposon end sequences.

66. The composition of Embodiment 65, wherein the cargo nucleic acid sequence is flanked by the first and the second transposon end sequences.

67. The composition of any one of Embodiments 65-66, wherein the transposon end sequences are selected from nucleotide sequences of SEQ ID NO: 5 and/or SEQ ID NO: 6, or a nucleotide sequence having at least about 90% identity thereto.

68. The composition of any one of Embodiments 65-67, wherein the end sequences include at least one repeat from a nucleotide sequence having at least about 90% identity to the nucleotide sequence of SEQ ID NO: 5 69. The composition of Embodiment 68, wherein the at least one repeat from the nucleotide sequence having at least about 90% identity to the nucleotide sequence of SEQ ID NO: 5 is positioned at the 5′ end of the transposon.

70. The composition of any one of Embodiments 65-69, wherein the end sequences can further include at least one repeat from a nucleotide sequence having at least about 90% identity to the nucleotide sequence of SEQ ID NO: 6.

71. The composition of any one of Embodiments 68-70, wherein the at least one repeat from the nucleotide sequence having at least about 90% identity to the nucleotide sequence of SEQ ID NO: 6 is positioned at the 3′ end of the transposon.

72. The composition of any one of Embodiments 1-71, wherein the enzyme or a nucleic acid encoding the enzyme or variant thereof is incorporated into a vector or a vector-like particle.

73. The composition of any one of Embodiments 1-72, wherein the vector or a vector-like particle comprises one or more expression cassettes.

74. The composition of Embodiment 73, wherein the vector or a vector-like particle comprises one expression cassette.

75. The composition of Embodiment 74, wherein the expression cassette further comprises the enzyme or variant thereof, the transgene, the transposon end sequences, or a combination thereof.

76. The composition of Embodiment 75, wherein the enzyme or variant thereof, the transgene, the transposon end sequences, or a combination thereof are incorporated into one or more vectors or vector-like particles.

77. The composition of Embodiment 75, wherein the enzyme or variant thereof, the transgene, the transposon end sequences, or combination thereof are incorporated into a same vector or vector-like particle.

78. The composition of Embodiment 75, wherein the enzyme or variant thereof, the transgene, the transposon end sequences, or combination thereof is incorporated into different vectors vector-like particles.

79. The composition of any one of Embodiments 72-78, wherein the vector or vector-like particle is nonviral.

80. The composition of any one of Embodiments 1-79, wherein the composition comprises DNA, RNA, or both.

81. The composition of any one of Embodiments 1-80, wherein the enzyme or variant thereof is in the form of RNA. 82. A host cell comprising the composition any one of Embodiments 1-81.

83. The composition of any one of Embodiments 1-81, wherein the composition is encapsulated in a lipid nanoparticle (LNP).

84. The composition of any one of Embodiments 1-81, wherein the polynucleotide encoding the enzyme or variant thereof and the polynucleotide encoding the transposon are in the form of the same LNP, optionally in a co-formulation.

85. The composition of Embodiment 83 or Embodiment 84, wherein the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly (lactic-co-glycolic acid) (PLGA), and N-Acetylgalactosamine (GalNAc).

86. A method for inserting a gene into the genome of a cell, comprising contacting a cell with the composition of any one of Embodiments 1-81 or 83-85 or host cell of Embodiment 82.

87. The method of Embodiment 86, further comprising contacting the cell with a polynucleotide encoding a transposon.

88. The method of Embodiment 86 or Embodiment 87, wherein the transposon comprises a gene encoding a complete polypeptide.

89. The method of any one of Embodiments 86-88, wherein the transposon comprises a gene which is defective or substantially absent in a disease state.

90. A method for treating a disease or disorder ex vivo, comprising contacting a cell with the composition of any one of Embodiments 1-81 or 83-85 or host cell of Embodiment 82 and administering the cell to a subject in need thereof.

91. A method for treating a disease or disorder in vivo, comprising administering the composition of any one of Embodiments 1-81 or 83-85 or host cell of Embodiment 82 to a subject in need thereof.

92. A composition comprising a transposase enzyme or a nucleic acid encoding the enzyme, wherein the enzyme comprises an amino acid sequence having at least about 80% sequence identity to SEQ ID NO: 11, and wherein the enzyme comprises an insertion at one or more of positions V371-1378, Y312-V322, K407-M413, S385-T392, A424-K432, R275-K290, or R315, G321, R376, S387, K409, and E428 relative to SEQ ID NO: 11.

93. The composition of Embodiment 92 wherein the insertion is or comprises a DNA binding domain in whole, or a functional fragment that is capable of binding.

94. The composition of Embodiment 93 wherein the DNA binding domain is selected from: zinc finger, TAL effector (TALE), leucine zipper, CRISPR-based DNA targeting nuclease, and/or combinations thereof.

95. The composition of Embodiment 93 wherein the CRISPR-based DNA targeting nuclease is selected from Cas9 and/or dCas9.

96. The composition of any one of Embodiments 92-95, wherein the enzyme comprises a substitution at position 450 with an amino acid other than aspartate (D).

97. The composition of Embodiment 96, wherein the substitution is with a polar uncharged amino acid.

98. The composition of Embodiment 97, wherein the polar uncharged amino acid is selected from serine(S) threonine (T), cysteine (C), asparagine (N), glutamine (Q), and proline (P).

99. The composition of Embodiment 98, wherein the polar uncharged amino acid is asparagine (N) or glutamine (Q).

100. The composition of Embodiment 99, wherein the polar uncharged amino acid is asparagine (N).

101. The composition of any one of Embodiments 92-100, wherein the enzyme comprises at least one, at least five, at least seven, at least nine, or ten substitutions at positions corresponding to: 30, 82, 103, 109, 165, 282, 509, 538, 571, and/or 591 of SEQ ID NO: 11.

102. The composition of any one of Embodiments 92-101, wherein the enzyme comprises one, two, three, four, five, six, seven, eight, nine, or ten substitutions at positions corresponding to: 30, 82, 103, 109, 165, 282, 509, 538, 571, and/or 591 of SEQ ID NO: 11.

103. The composition of any one of Embodiments 92-102, wherein the enzyme comprises at least one, at least five, at least seven, at least nine, or ten substitutions selected from 130V, S103P, G165S, M282V, S509G, N538K, N571S, 182N, V109A, and Q591R, wherein the positions are corresponding to positions of SEQ ID NO: 11.

104. The composition of any one of Embodiments 92-103, wherein the enzyme comprises one, two, three, four, five, six, seven, eight, nine, or ten substitutions selected from 130V, S103P, G165S, M282V, S509G, N538K, N571S, 182N, V109A, and Q591R, wherein the positions are corresponding to positions of SEQ ID NO: 11.

105. The composition of any one of Embodiments 92-104, wherein the enzyme comprises, at the positions corresponding to positions of SEQ ID NO: 11, substitutions of D450N, 130V, S103P, G165S, M282V, S509G, N538K, N571S, 182N, V109A, and Q591R.

106. The composition of any one of Embodiments 92-105, wherein the nucleic acid that encodes the enzyme has a nucleotide sequence of SEQ ID NO: 7 or a codon-optimized form thereof.

107. The composition of any one of Embodiments 92-106, wherein the enzyme has increased excision activity relative to an enzyme comprising an amino acid sequence of SEQ ID NO: 11 or functional equivalent thereof.

108. The composition of any one of Embodiments 92-107, wherein the enzyme is excision positive.

109. The composition of any one of Embodiments 92-108, wherein the enzyme is integration deficient.

110. The composition of any one of Embodiments 92-109, wherein the enzyme has decreased integration activity relative to an enzyme comprising an amino acid sequence of SEQ ID NO: 11 or functional equivalent thereof.

111. The composition of any one of Embodiments 92-110, wherein the enzyme has increased excision activity relative to an enzyme comprising an amino acid sequence of SEQ ID NO: 2 or functional equivalent thereof.

112. The composition of any one of Embodiments 92-111, wherein the enzyme has increased excision activity relative to an enzyme comprising an amino acid sequence of SEQ ID NO: 3 or functional equivalent thereof.

113. The composition of any one of Embodiments 92-112, wherein the enzyme comprises at least one substitution at positions corresponding to: 189, 191, 198, 201, 312, 314, 315, 316, 321, 324, 347, 362, 369, 370, 371, 372, 374, 375, 376, 377, 378, 379, 380, 387, 388, 390, 400, 425, 428, 500, and/or 504 of SEQ ID NO: 11.

114. The composition of any one of Embodiments 92-113, wherein the enzyme comprises at least one substitution at positions corresponding to: 312, 315, 324, 347, 372, 374, and/or 375 of SEQ ID NO: 11, and/or wherein the enzyme comprises at least one substitution selected from Y312A, R315A, L324A, N347A, R372A, N374A, and K375A, wherein the positions are corresponding to positions of SEQ ID NO: 11.

115. The composition of any one of Embodiments 92-114, wherein the enzyme comprises substitution(s) selected from R372A/K375A, R372A/R315A, N347A/R315A, L324A/Y312A, N374A, L324A/R315A, R315A/R372A/K375A, and L324A/N347A, wherein the positions are corresponding to positions of SEQ ID NO: 11.

116. The composition of any one of Embodiments 92-115, wherein the insert is or comprises a targeting element.

117. The composition of any one of Embodiments 92-116, wherein the insert is capable of inserting a transposon comprising a transgene in a target site, optionally a genomic safe harbor site (GSHS).

118. The composition of Embodiment 117, wherein the binding of a GSHS of a nucleic acid molecule in a mammalian cell is with high target specificity, relative to a control.

119. The composition of Embodiment 118, wherein the control is a composition comprising an enzyme comprising an amino acid sequence of SEQ ID NO: 11 or a nucleic acid comprising a nucleotide sequence of SEQ ID NO: 12 or a codon-optimized form thereof, wherein the control is a composition comprising an enzyme comprising an amino acid sequence of SEQ ID NO: 2 or a nucleic acid comprising a nucleotide sequence of SEQ ID NO: 8 or a codon-optimized form thereof, and/or wherein the control is a composition comprising an enzyme comprising an amino acid sequence of SEQ ID NO: 3 or a nucleic acid comprising a nucleotide sequence of SEQ ID NO: 9 or a codon-optimized form thereof.

120. The composition of any one of Embodiments 116-119, wherein the targeting element is able to direct a transposition machinery to the GSHS of a nucleic acid molecule in a mammalian cell.

121. The composition of any one of Embodiments 116-120, wherein the GSHS is in an open chromatin location in a chromosome.

122. The composition of any one of Embodiments 116-121, wherein the GSHS is selected from adeno-associated virus site 1 (AAVS1), chemokine (C-C motif) receptor 5 (CCR5) gene, HIV-1 coreceptor, and human Rosa26 locus.

123. The composition of any one of Embodiments 116-122, wherein the GSHS is an adeno-associated virus site 1 (AAVS1).

124. The composition of any one of Embodiments 116-123, wherein the GSHS is a human Rosa26 locus.

125. The composition of any one of Embodiments 116-124, wherein the GSHS is located on human chromosome 2, 4, 6, 10, 11, 17, or 22.

126. The composition of any one of Embodiments 116-125, wherein the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

127. The composition of any one of Embodiments 116-126, wherein the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

128. The composition of Embodiment 127, wherein the targeting element comprises a TALE DBD.

129. The composition of Embodiment 128, wherein the TALE DBD comprises one or more repeat sequences.

130. The composition of Embodiment 129, wherein the TALE DBD comprises about 14, or about 15, or about, 16, or about 17, or about 18, or about 18.5 repeat sequences.

131. The composition of Embodiment 129 or Embodiment 130, wherein the repeat sequences each independently comprises about 33 or 34 amino acids.

132. The composition of Embodiment 131, wherein the repeat sequences each independently comprises a repeat variable di-residue (RVD) at residue 12 or 13 of the 33 or 34 amino acids, respectively.

133. The composition of Embodiment 132, wherein the RVD recognizes one base pair in a target nucleic acid sequence.

134. The composition of Embodiment 132 or Embodiment 133, wherein the RVD recognizes a C residue in the target nucleic acid sequence and is selected from HD, N (gap), HA, ND, and HI.

135. The composition of Embodiment 132 or Embodiment 133, wherein the RVD recognizes a G residue in the target nucleic acid sequence and is selected from NN, NH, NK, HN, and NA.

136. The composition of Embodiment 132 or Embodiment 133, wherein the RVD recognizes an A residue in the target nucleic acid sequence and is selected from NI and NS.

137. The composition of Embodiment 132 or Embodiment 133, wherein the RVD recognizes a T residue in the target nucleic acid sequence and is selected from NG, HG, H(gap), and IG.

138. The composition of Embodiment 127, wherein the targeting element comprises a Cas9 enzyme associated with a gRNA.

139. The composition of Embodiment 138, wherein the Cas9 enzyme associated with a gRNA comprises a catalytically inactive dCas9 associated with a gRNA.

140. The composition of Embodiment 139, wherein catalytically inactive dCas9 comprises at least about 90%, or at least about 95%, or at least about 96%, or at least about 97%, or at least about 98%, or at least about 99% identity to an amino acid sequence of SEQ ID NO: 4 or a nucleic acid comprising a nucleotide sequence of SEQ ID NO: 10 or a codon-optimized form thereof.

141. The composition of any one of Embodiments 116-140, wherein the targeting element comprises a Cas12 enzyme associated with a gRNA.

142. The composition of Embodiment 141, wherein the targeting element comprises a catalytically inactive Cas12 associated with a gRNA, optionally wherein the catalytically inactive Cas12 is dCas12j or dCas12a.

143. The composition of any one of Embodiments 116-142, wherein the targeting element comprises a nucleic acid binding component of a gene-editing system.

144. The composition of any one of Embodiments 116-143, wherein the enzyme or variant thereof and the targeting element are connected.

145. The composition of Embodiment 144, wherein the enzyme and the targeting element are fused to one another or linked via a linker to one another.

146. The composition of Embodiment 145, wherein the linker is a flexible linker.

147. The composition of Embodiment 146, wherein the flexible linker is substantially comprised of glycine and serine residues, optionally wherein the flexible linker comprises (Gly4Ser)ₙ, where n is an integer from 1-12.

148. The composition of Embodiment 147, wherein the flexible linker is of about 20, or about 30, or about 40, or about 50, or about 60 amino acid residues.

149. The composition of Embodiment 148, wherein the enzyme is directly fused to the N-terminus of the dCas9 enzyme.

150. The composition of any one of Embodiments 92-149, wherein the enzyme or variant thereof is able to directly or indirectly cause transposition of a target gene.

151. The composition of any one of Embodiments 92-150, wherein the enzyme or variant thereof is able to directly or indirectly interact and/or form a complex with one or more proteins or nucleic acids.

152. The composition of any one of Embodiments 92-151, further comprising a nucleic acid encoding a transposon comprising a transgene to be integrated.

153. The composition of Embodiment 152, wherein the transgene comprises a cargo nucleic acid sequence and a first and a second transposon end sequences.

154. The composition of Embodiment 153, wherein the cargo nucleic acid sequence is flanked by the first and the second transposon end sequences.

155. The composition of any one of Embodiments 153-154, wherein the transposon end sequences are selected from nucleotide sequences of SEQ ID NO: 5 and/or SEQ ID NO: 6, or a nucleotide sequence having at least about 90% identity thereto.

156. The composition of any one of Embodiments 153-155, wherein the end sequences include at least one repeat from a nucleotide sequence having at least about 90% identity to the nucleotide sequence of SEQ ID NO: 5.

157. The composition of Embodiment 156, wherein the at least one repeat from the nucleotide sequence having at least about 90% identity to the nucleotide sequence of SEQ ID NO: 5 is positioned at the 5′ end of the transposon.

158. The composition of any one of Embodiments 153-157, wherein the end sequences can further include at least one repeat from a nucleotide sequence having at least about 90% identity to the nucleotide sequence of SEQ ID NO: 6.

159. The composition of any one of Embodiments 156-158, wherein the at least one repeat from the nucleotide sequence having at least about 90% identity to the nucleotide sequence of SEQ ID NO: 6 is positioned at the 3′ end of the transposon.

160. The composition of any one of Embodiments 92-159, wherein the enzyme or variant thereof is incorporated into a vector or a vector-like particle.

161. The composition of any one of Embodiments 92-160, wherein the vector or a vector-like particle comprises one or more expression cassettes.

162. The composition of Embodiment 161, wherein the vector or a vector-like particle comprises one expression cassette.

163. The composition of Embodiment 162, wherein the expression cassette further comprises the enzyme or variant thereof, the transgene, the transposon end sequences, or a combination thereof.

164. The composition of Embodiment 163, wherein the enzyme or variant thereof, the transgene, the transposon end sequences, or a combination thereof are incorporated into one or more vectors or vector-like particles.

165. The composition of Embodiment 164, wherein the enzyme or variant thereof, the transgene, the transposon end sequences, or combination thereof are incorporated into a same vector or vector-like particle.

166. The composition of Embodiment 165, wherein the enzyme or variant thereof, the transgene, the transposon end sequences, or combination thereof is incorporated into different vectors vector-like particles.

167. The composition of any one of Embodiments 160-166, wherein the vector or vector-like particle is nonviral.

168. The composition of any one of Embodiments 92-167, wherein the composition comprises DNA, RNA, or both. 169. The composition of any one of Embodiments 92-168, wherein the enzyme or variant thereof is in the form of RNA. 170.A host cell comprising the composition any one of Embodiments 92-169.

171. The composition of any one of Embodiments 92-169, wherein the composition is encapsulated in a lipid nanoparticle (LNP).

172. The composition of any one of Embodiments 92-169, wherein the polynucleotide encoding the enzyme or variant thereof and the polynucleotide encoding the transposon are in the form of the same LNP, optionally in a co-formulation.

173. The composition of Embodiment 171 or Embodiment 172, wherein the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly (lactic-co-glycolic acid) (PLGA), and N-Acetylgalactosamine (GalNAc).

174.A method for inserting a gene into the genome of a cell, comprising contacting a cell with the composition of any one of Embodiments 92-169 or 171-173 or host cell of Embodiment 170.

175. The method of Embodiment 174, further comprising contacting the cell with a polynucleotide encoding a transposon.

176. The method of Embodiment 174 or Embodiment 175, wherein the transposon comprises a gene encoding a complete polypeptide.

177. The method of any one of Embodiments 174-176, wherein the transposon comprises a gene which is defective or substantially absent in a disease state.

178.A method for treating a disease or disorder ex vivo, comprising contacting a cell with the composition of any one of Embodiments 92-169 or 171-173 or host cell of Embodiment 170 and administering the cell to a subject in need thereof.

179.A method for treating a disease or disorder in vivo, comprising administering the composition of any one of Embodiments 92-169 or 171-173 or host cell of Embodiment 170 to a subject in need thereof.

180. The composition of Embodiment 39, wherein the targeting element comprises a Zinc finger.

This invention is further illustrated by the following non-limiting examples.

### Examples

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1-Design of Hyperactive piggyBac Transposon System

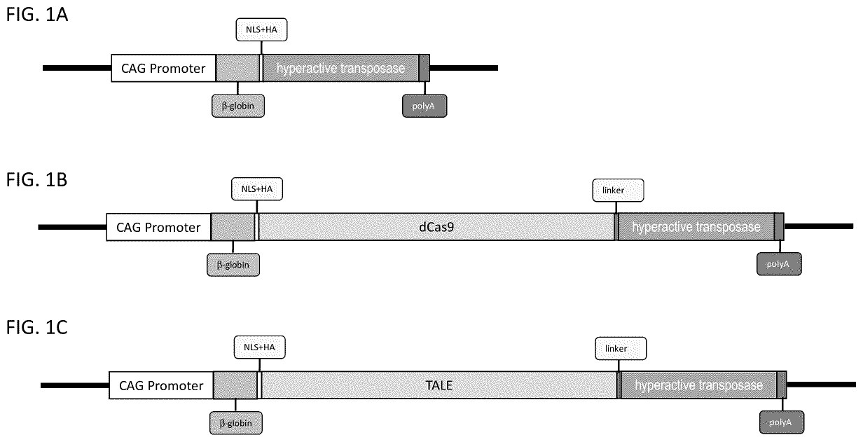

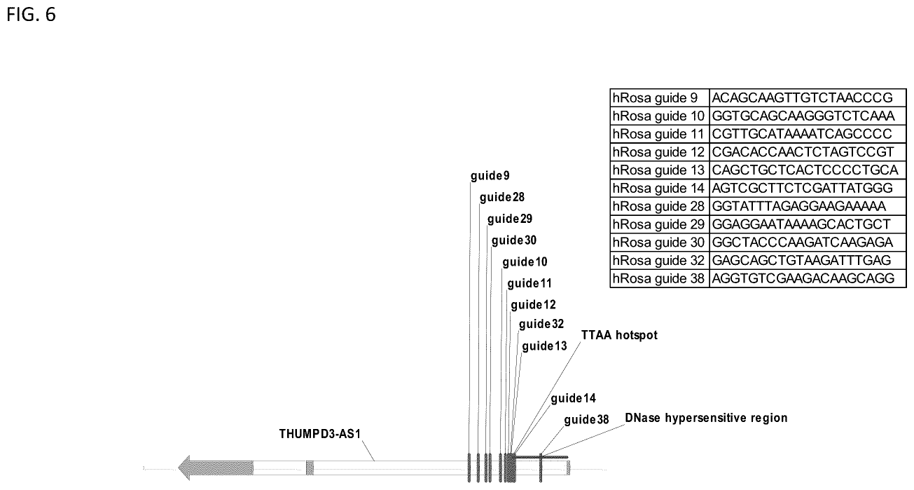

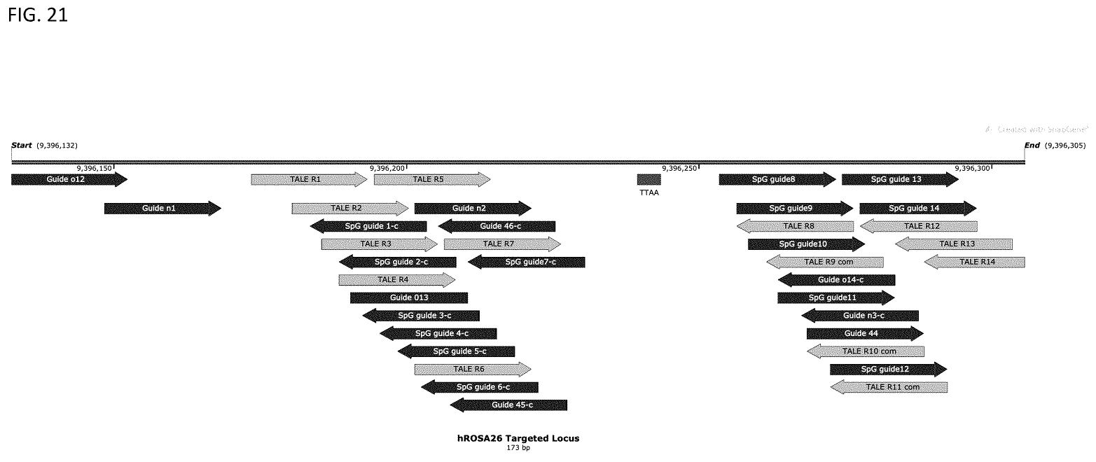

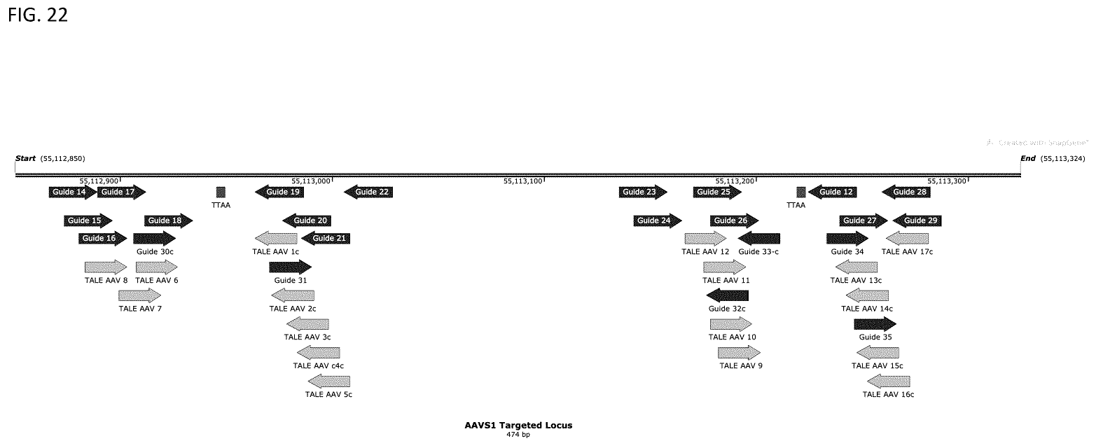

FIGS. 1A-C depict three bioengineered helper constructs that are contained in a bacterial replication backbone (e.g., plasmid or miniplasmid) with a chicken beta-actin/cytomegalovirus (CMV) enhancer promoter (CAG), human beta-globin 5′-UTR, nuclear localization signal and a helper enzyme with 11 mutations in the Trichnoplusia ni transposase (hyperactive transposase, SEQ ID NO: 1) followed by a poly-alanine tail. FIG. 1A depicts the hyperactive transposase without a DBD used as negative control. A dead Cas9 (dCas9) (FIG. 1B) binding protein (SEQ ID NO: 4) or TALE protein (FIG. 1C) (TABLE 7, TABLE 8) were joined by a linker to the N-terminus of hyperactive transposase to target the genomic safe harbor sites hROSA 26 (FIG. 6 and FIG. 21) or AAVS1 (FIG. 22). These constructs were used to identify hyperactive transposase excision positive (EXC+) (TABLE 3) and integration deficient (INT−) mutants (TABLE 4).

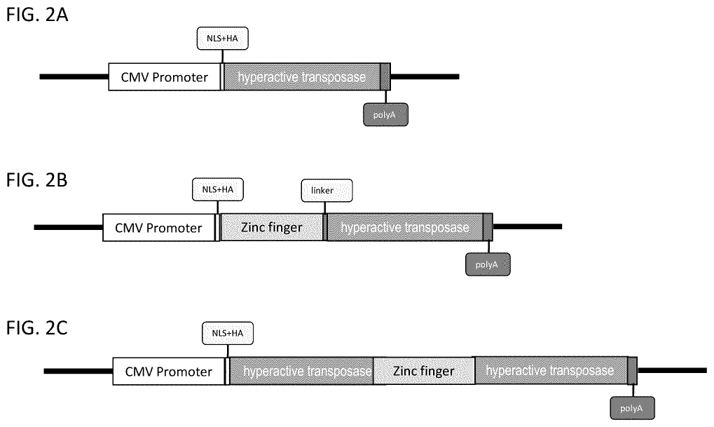

FIGS. 2A-C depict three illustrative bioengineered helper constructs that are contained in a bacterial replication backbone (e.g., plasmid or miniplasmid) with a CMV promoter, nuclear localization signal and a helper enzyme with 11 mutations in the Trichnoplusia ni transposase (hyperactive transposase, SEQ ID NO: 1) followed by a poly-alanine tail. FIG. 2A depicts the hyperactive transposase without a DBD used as negative control. FIG. 2B depicts a zinc finger DBD joined by a linker to the N-terminus of hyperactive transposase to target a sequence on a reporter plasmid. FIG. 2C depicts a zinc finger DBD inserted into an internal flexible loop domain of hyperactive transposase to target a sequence on a reporter plasmid.

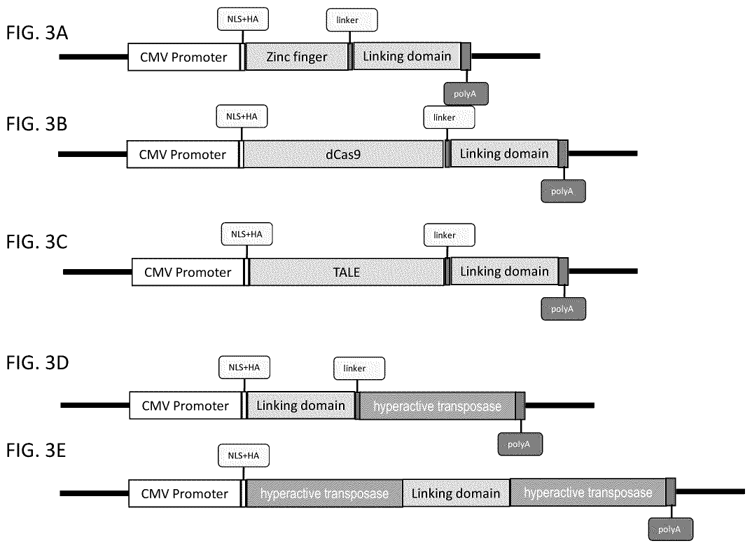

FIGS. 3A-E depict five illustrative bioengineered helper constructs that are contained in a bacterial replication backbone (e.g., plasmid or miniplasmid) with a CMV promoter, nuclear localization signal and a helper enzyme with 11 mutations in the Trichnoplusia ni transposase (hyperactive transposase, SEQ ID NO: 1) fused to a linking domain or a DBD fused to a linking domain used to bridge two helper proteins, followed by a poly-alanine tail. A linking domain joined by a linker was fused to a ZF (FIG. 3A), dCas9 (FIG. 3B), or a TALE (FIG. 3C). A second linking domain that bridges the proteins together was fused to the N-terminus, joined by a linker, (FIG. 3D) or into an internal flexible loop domain joined by a linker, (FIG. 3E) to the hyperactive transposase.

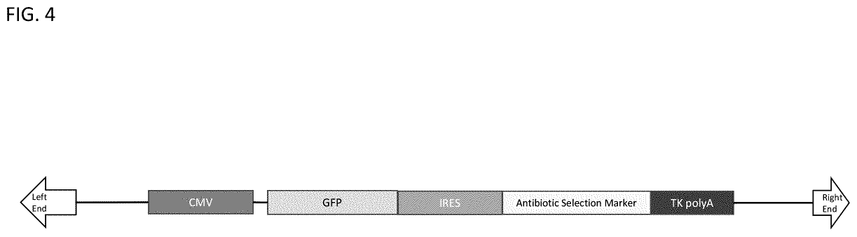

FIG. 4 depicts the core donor construct that is contained in a bacterial replication backbone (e.g., plasmid or miniplasmid) with a CMV promoter driving GFP expression and IRES to co-express a bacterial selection gene and, when used with the hyperactive transposase-Cas9 fusion helper, guide RNAs (TABLE 5 and TABLE 6) are included in the construct. Terminal inverted repeat (TIR) recognition sequences are included at the 5′-(SEQ ID NO: 5) and 3′-ends (SEQ ID NO: 6).

TABLE 3
Hyperactive transposase mutants with excision activity

| | | | | % | |
|---|---|---|---|---|---|
| | | | % | ACTIVITY | % |
| MUTANT 1 | MUTANT 2 | MUTANT 3 | ACTIVITY ≥2.0 | 0.3-1.9 | ACTIVITY <0.29 |
| G321A | | | X | | |
| L324A | K375A | | X | | |
| L324A | N347A | | X | | |
| L324A | R315A | | X | | |
| L324A | R376A | | X | | |
| L324A | Y312A | | X | | |
| N347A | R376A | | X | | |
| N347A | K375A | | X | | |
| N347A | R315A | | X | | |
| R372A | K375A | | X | | |
| R372A | R315A | | X | | |
| R372A | K375A | | X | | |
| R372A | N347A | | X | | |
| R372A | R315A | | X | | |
| R372A | R376A | | X | | |
| N374A | | | X | | |
| K375A | K376A | | X | | |
| K375A | R315A | | X | | |
| R376A | | | X | | |
| R376A | R315A | | X | | |
| S387A | | | X | | |
| S425A | | | X | | |
| E428A | | | X | | |
| D198S | | | | X | |
| Y312A | | | | X | |
| R315A | Y312A | | | X | |
| R315A | | | | X | |
| R315A | R372A | K375A | | X | |
| R315A | Y312A | | | X | |
| G316A | | | | X | |
| G316A | N347A | R372A | | X | |
| G316A | R372A | | | X | |
| G316A | N347A | | | X | |
| L324A | N347A | | | X | |
| L324A | R315A | | | X | |
| L324A | R372A | | | X | |
| L324A | | | | X | |
| L324A | | | | X | |
| L324A | R372A | | | X | |
| N347A | | | | X | |
| N347A | Y312A | | | X | |
| N347A | Y312A | | | X | |
| T370A | | | | X | |
| R372A | | | | X | |
| R372A | Y312A | | | X | |
| R372A | Y312A | | | X | |
| N374A | | | | X | |
| K375A | | | | X | |
| R376A | | | | X | |
| R376A | Y312A | | | X | |
| V390A | | | | X | |
| P400A | | | | X | |
| R189A | | | | | X |
| R189S | | | | | X |
| D191A | | | | | X |
| D191S | | | | | X |
| D198A | | | | | X |
| D201A | | | | | X |
| D201S | | | | | X |
| Y312A | | | | | X |
| G314A | | | | | X |
| G314A | | | | | X |
| P362A | | | | | X |
| G369A | | | | | X |
| G369A | | | | | X |
| V371A | | | | | X |
| V371A | | | | | X |
| N374A | | | | | X |
| R376A | Y312A | | | | X |
| E377A | | | | | X |
| E377A | | | | | X |
| I378A | | | | | X |
| I378A | | | | | X |
| P379A | | | | | X |
| P379A | | | | | X |
| E380A | | | | | X |
| R388A | | | | | X |

TABLE 4
Hyperactive transposase integration deficient mutants

| MUTANT 1 | MUTANT 2 | MUTANT 3 | MUTANT 4 | MUTANT 5 | MUTANT 6 |
|---|---|---|---|---|---|
| R189A | | | | | |
| R189A | D191A | | | | |
| R189A | D191A | D201A | R504A | | |
| R189A | D191A | D201A | R504A | D198S | K500S |
| R189A | D198A | K500A | | | |
| R189S | | | | | |
| R189S | D191S | | | | |
| R189S | D191S | D198S | K500S | | |
| R189S | D191S | D201S | R504S | | |
| R189S | D191S | D201S | R504S | D198A | K500A |
| D191A | | | | | |
| D191S | | | | | |
| D198A | | | | | |
| D198S | | | | | |
| D198S | K500S | | | | |
| D201A | | | | | |
| D201A | R504A | | | | |
| D201A | R504A | D198A | K500A | | |
| D201S | | | | | |
| D201S | R504S | | | | |
| D201S | R504S | D198S | K500S | | |
| Y312A | | | | | |
| G314A | | | | | |
| R315A | R372A | K375A | | | |
| R315A | Y312A | | | | |
| G316A | N347A | R372A | | | |
| L324A | | | | | |
| L324A | R315A | | | | |
| N347A | Y312A | | | | |
| P362A | | | | | |
| G369A | | | | | |
| V371A | | | | | |
| R372A | R315A | | | | |
| R372A | Y312A | | | | |
| N374A | | | | | |
| R376A | Y312A | | | | |
| E377A | | | | | |
| I378A | | | | | |
| P379A | | | | | |
| E380A | | | | | |
| R388A | | | | | |
| V390A | | | | | |
| P400A | | | | | |

TABLE 5
Guide RNA Sequences Targeting the Genomic
Safe Harbor Site, hROSA26.

| HROSA26 | | SEQ ID |
|---|---|---|
| GUIDE NO. | DNA SEQUENCE | NO: |
| GUIDE 44 | AATCGAGAAGCGACTCGACA | 425 |
| GUIDE 45-C | GTCCCTGGGCGTTGCCCTGC | 442 |
| GUIDE 46-C | CCCTGGGCGTTGCCCTGCAG | 443 |
| SPG GUIDE1-C | GAGTGAGCAGCTGTAAGATT | 444 |
| SPG GUIDE2-C | CAGGGGAGTGAGCAGCTGTA | 445 |
| SPG GUIDE3-C | CCTGCAGGGGAGTGAGCAGC | 428 |
| SPG GUIDE4-C | TGCCCTGCAGGGGAGTGAGC | 426 |
| SPG GUIDE5-C | CGTTGCCCTGCAGGGGAGTG | 446 |
| SPG GUIDE6-C | TGGGCGTTGCCCTGCAGGGG | 447 |
| SPG GUIDE7-C | TTGGTCCCTGGGCGTTGCCC | 448 |
| SPG GUIDE8 | AAGAATCCCGCCCATAATCG | 449 |
| SPG GUIDE9 | AATCCCGCCCATAATCGAGA | 450 |
| SPG GUIDE10 | TCCCGCCCATAATCGAGAAG | 451 |
| SPG GUIDE11 | CCCATAATCGAGAAGCGACT | 452 |
| SPG GUIDE12 | GAGAAGCGACTCGACATGGA | 453 |
| SPG GUIDE13 | GAAGCGACTCGACATGGAGG | 427 |
| SPG GUIDE14 | GCGACTCGACATGGAGGCGA | 454 |
| GUIDE N1 | CCGTGGGAAGATAAACTAAT | 455 |
| GUIDE N2 | TCCCCTGCAGGGCAACGCCC | 456 |
| GUIDE N3-C | GTCGAGTCGCTTCTCGATTA | 457 |
| GUIDE 012 | CGACACCAACTCTAGTCCGT | 458 |
| GUIDE 013 | CAGCTGCTCACTCCCCTGCA | 459 |
| GUIDE 014-C | AGTCGCTTCTCGATTATGGG | 460 |

TABLE 6
Guide RNA Sequences Targeting the Genomic
Safe Harbor Site, AAVS1.

| AAVS1 GUIDE | | SEQ ID |
|---|---|---|
| NO. | DNA SEQUENCE | NO: |
| AAV GUIDE 12 | ACCCTTGGAAGGACCTGGCTGGG | 461 |
| AAV GUIDE 13c | TCCGAGCTTGACCCTTGGAA | 462 |
| AAV GUIDE 14 | GGAGCCACGAAAACAGATCCAGG | 463 |
| AAV GUIDE 14c | TGGTTTCCGAGCTTGACCCT | 112 |
| AAV GUIDE 15 | GGAGCCACGAAAACAGATCCAGG | 463 |
| AAV GUIDE 16 | AGATCCAGGGACACGGTGCTAGG | 464 |
| AAV GUIDE 17 | GACACGGTGCTAGGACAGTGGGG | 465 |
| AAV GUIDE 18 | GAAAATGACCCAACAGCCTCTGG | 466 |
| AAV GUIDE 19 | GCCTGGCCGGCCTGACCACTGGG | 467 |
| AAV GUIDE 20 | CTGAGCACTGAAGGCCTGGCCGG | 468 |
| AAV GUIDE 21 | TGGTTTCCACTGAGCACTGAAGG | 469 |
| AAV GUIDE 22 | GGTGCTTTCCTGAGGACCGATAG | 470 |
| AAV GUIDE 23 | GCGCTTCCAGTGCTCAGACTAGG | 471 |
| AAV GUIDE 24 | CAGTGCTCAGACTAGGGAAGAGG | 472 |
| AAV GUIDE 25 | GCCCCTCCTCCTTCAGAGCCAGG | 473 |
| AAV GUIDE 26 | TCCTTCAGAGCCAGGAGTCCTGG | 474 |
| AAV GUIDE 27 | CCAAGGGTCAAGCTCGGAAACCA | 475 |
| AAV GUIDE 28 | CTGCAGAGTATCTGCTGGGGTGG | 476 |
| AAV GUIDE 29 | CGTTCCTGCAGAGTATCTGCTGG | 477 |
| AAV GUIDE 30c | GTGGGGAAAATGACCCAACA | 478 |
| AAV GUIDE 31 | GAAGGCCTGGCCGGCCTGAC | 479 |
| AAV GUIDE 32c | ACTCCTGGCTCTGAAGGAGG | 480 |
| AAV GUIDE 33c | GGGCTGGGGGCCAGGACTCC | 481 |
| AAV GUIDE 34 | GTCCTTCCAAGGGTCAAGCT | 482 |
| AAV GUIDE 35 | TCAAGCTCGGAAACCACCCC | 483 |

TABLE 7
TALE Sequences Targeting the Genomic Safe Harbor Site, hROSA26.

| NAME | DNA SEQUENCE (SEQ ID NO:) | RVD CODE |
|---|---|---|
| R1 | TCGCCCCTCAAATCTTACAG (584) | HD NH HD HD HD HD NG HD NI NI NI NG HD NG NG NI HD |
| | | NI NH |
| R2 | TCAAATCTTACAGCTGCTCA (585) | HD NI NI NI NG HD NG NG NI HD NI NH HD NG NH HD NG |
| | | HD NI |
| R3 | TCTTACAGCTGCTCACTCCC (586) | HD NG NG NI HD NI NH HD NG NH HD NG HD NI HD NG HD |
| | | HD HD |
| R4 | TACAGCTGCTCACTCCCCTG (587) | NI HD NI NH HD NG NH HD NG HD NI HD NG HD HD HD HD |
| | | NG NH |
| R5 | TGCTCACTCCCCTGCAGGGC (588) | NH HD NG HD NI HD NG HD HD HD HD NG NH HD NI NH NH |
| | | NH HD |
| R6 | TCCCCTGCAGGGCAACGCCC (456) | HD HD HD HD NG NH HD NI NH NH NH HD NI NI HD NH HD |
| | | HD HD |
| R7 | TGCAGGGCAACGCCCAGGGA (589) | NH HD NI NH NH NH HD NI NI HD NH HD HD HD NI NH NH |
| | | NH NI |
| R8 | TCTCGATTATGGGCGGGATT (590) | HD NG HD NH NI NG NG NI NG NH NH NH HD NH NH NH NI |
| | | NG NG |
| R9 | TCGCTTCTCGATTATGGGCG (591) | HD NH HD NG NG HD NG HD NH NI NG NG NI NG NH NH NH |
| | | HD NH |
| R10 | TGTCGAGTCGCTTCTCGATT (592) | NH NG HD NH NI NH NG HD NH HD NG NG HD NG HD NH NI |
| | | NG NG |
| R11 | TCCATGTCGAGTCGCTTCTC (593) | HD HD NI NG NH NG HD NH NI NH NG HD NH HD NG NG HD |
| | | NG HD |
| R12 | TCGCCTCCATGTCGAGTCGC (594) | HD NH HD HD NG HD HD NI NG NH NG HD NH NI NH NG HD |
| | | NH HD |
| R13 | TCGTCATCGCCTCCATGTCG (595) | HD NH NG HD NI NG HD NH HD HD NG HD HD NI NG NH NG |
| | | HD NH |
| R14 | TGATCTCGTCATCGCCTCCA (596) | NH NI NG HD NG HD NH NG HD NI NG HD NH HD HD NG HD |
| | | HD NI |

TABLE 8
TALE Sequences Targeting the Genomic Safe
Site, AAVS1.

| NAME | DNA SEQUENCE (SEQ ID NO:) | RVD CODE |
|---|---|---|
| AAV1c | TGGCCGGCCTGACCACTGGG (597) | NH NH HD HD NH NH HD HD NG NH NI HD HD |
| | | NI HD NG NH NH NH |
| AAV2c | TGAAGGCCTGGCCGGCCTGA (598) | NH NI NI NH NH HD HD NG NH NH HD HD NH |
| | | NH HD HD NG NH NI |
| AAV3c | TGAGCACTGAAGGCCTGGCC (599) | NH NI NH HD NI HD NG NH NI NI NH NH HD |
| | | HD NG NH NH HD HD |
| AAV4c | TCCACTGAGCACTGAAGGCC (600) | HD HD NI HD NG NH NI NH HD NI HD NG NH |
| | | NI NI NH NH HD HD |
| AAV5c | TGGTTTCCACTGAGCACTGA (601) | NH NH NG NG NG HD HD NI HD NG NH NI NH |
| | | HD NI HD NG NH NI |
| AAV6 | TGGGGAAAATGACCCAACAG (602) | NH NH NH NH NI NI NI NI NG NH NI HD HD |
| | | HD NI NI HD NI NH |
| AAV7 | TAGGACAGTGGGGAAAATGA (603) | NI NH NH NI HD NI NH NG NH NH NH NH NI |
| | | NI NI NI NG NH NI |
| AAV8 | TCCAGGGACACGGTGCTAGG (604) | HD HD NI NH NH NH NI HD NI HD NH NH NG |
| | | NH HD NG NI NH NH |
| AAV9 | TCAGAGCCAGGAGTCCTGGC (605) | HD NI NH NI NH HD HD NI NH NH NI NH NG |
| | | HD HD NG NH NH HD |
| AAV10 | TCCTTCAGAGCCAGGAGTCC (606) | HD HD NG NG HD NI NH NI NH HD HD NI NH |
| | | NH NI NH NG HD |
| AAV11 | TCCTCCTTCAGAGCCAGGAG (607) | HD HD NG HD HD NG NG HD NI NH NI NH HD |
| | | HD NI NH NH NI NH |
| AAV12 | TCCAGCCCCTCCTCCTTCAG (608) | HD HD NI NH HD HD HD HD NG HD HD NG HD |
| | | HD NG NG HD NI NH |
| AAV13c | TCCGAGCTTGACCCTTGGAA (462) | HD HD NH NI NH HD NG NG NH NI HD HD HD |
| | | NG NG NH NH NI NI |
| AAV14c | TGGTTTCCGAGCTTGACCCT (112) | NH NH NG NG NG HD HD NH NI NH HD NG NG |
| | | NH NI HD HD HD NG |
| AAV15c | TGGGGTGGTTTCCGAGCTTG (609) | NH NH NH NH NG NH NH NG NG NG HD HD NH |
| | | NI NH HD NG NG NH |
| AAV16c | TCTGCTGGGGTGGTTTCCGA (610) | HD NG NH HD NG NH NH NH NH NG NH NH NG |
| | | NG NG HD HD NH NI |
| AAV17c | TGCAGAGTATCTGCTGGGGT (611) | NH HD NI NH NI NH NG NI NG HD NG NH HD |
| | | NG NH NH NH NH NG |

TABLE 9
Guide RNA seqences targeting
chromosome 22 TTAA hotspot.
[hg38 chr22: 35,373,912-35,373,916 (861);
chr22: 35,377,843-35,377,847 (1153)].

| | | SEQ |
|---|---|---|
| | | ID |
| CHR22 GUIDE NO. | DNA SEQUENCE | NO: |
| Guide C22-1 | ATAACACGTGAGCCGTCCTAAGG | 524 |
| Guide C22-2 | GGAAGACTTTTCTCTATACGAGG | 525 |
| Guide C22-3 | GCATTCCTTTCATCCATGGCAGG | 526 |
| Guide C22-4 | GACATATGGTTATAAAAATCAGG | 527 |
| Guide C22-5 | GGAGTGCAGTCCCTGACATATGG | 528 |
| Guide C22-6 | GTGGGTTAGGGTGGTTAACTGGG | 529 |
| Guide C22-7 | AGGTGCAAAAAGGTTGCTGTGGG | 530 |
| Guide C22-8 | CGTGACAAGGCAAAGTGGCGTGG | 531 |
| Guide C22-9 | GAAGGACTGCCCCTGACGTCAGG | 532 |
| Guide C22-10 | CTGCCCCTGACGTCAGGAGTTGG | 533 |
| Guide C22-11 | TGTGGGTTAGGGTGGTTAACTGG | 534 |
| Guide C22-12 | ACCCTTTTAGAGTTTTCTGCTGG | 535 |
| Guide C22-13 | AACTTCCTGCCATGGATGAAAGG | 536 |
| Guide C22-14 | GCAAAAAGGTTGCTGTGGGTTGG | 537 |
| Guide C22-15 | AATTTGGGGGTAGATAGGCATGG | 538 |
| Guide C22-16 | AGAAAACTCTAAAAGGGTATAGG | 539 |
| Guide C22-17 | ATTAGCATTCCTTTCATCCATGG | 540 |
| Guide C22-18 | CCCAGCAGAAAACTCTAAAAGGG | 541 |
| Guide C22-19 | CAGGTGCAAAAAGGTTGCTGTGG | 542 |
| Guide C22-20 | GCAAGAGATGAAATTCCATATGG | 543 |
| Guide C22A1 | GGGCTGTTCTAACGAAGTCTGGG | 544 |
| Guide C22A2 | TGTCCATTCAGCGACCCTAGAGG | 545 |
| Guide C22A3 | GGCTGTTCTAACGAAGTCTGGGG | 546 |
| Guide C22A4 | GTCCATTCAGCGACCCTAGAGGG | 547 |
| Guide C22A5 | GGGGCTGTTCTAACGAAGTCTGG | 548 |
| Guide C22A6 | GGCTGAATCAGCATGCGAAAGGG | 549 |
| Guide C22A7 | TTCCAATGGGGGGCATAGCCTGG | 550 |
| Guide C22A8 | TACCCTCTAGGGTCGCTGAATGG | 551 |
| Guide C22A9 | ATCCTCTTGGGCCTTATAAGAGG | 552 |
| Guide C22A10 | GGCCAGGCTATGCCCCCCATTGG | 553 |
| Guide C22A11 | CTAGAGGACCAGAACAACTCTGG | 554 |
| Guide C22A12 | TCCCTCTTATAAGGCCCAAGAGG | 555 |
| Guide C22A13 | AGGCTGAATCAGCATGCGAAAGG | 556 |
| Guide C22A14 | GGACCAGAACAACTCTGGCCTGG | 557 |
| Guide C22A15 | GGGCTTTTATTTGGCCCAGCAGG | 558 |
| Guide C22A16 | GTCGCTGAATGGACAGACTCTGG | 559 |
| Guide C22A17 | CTCATGAGTTTTACCCTCTAGGG | 560 |
| Guide C22A18 | TCCTCTTGGGCCTTATAAGAGGG | 561 |
| Guide C22A19 | TCTTGGGCCTTATAAGAGGGAGG | 562 |
| Guide C22A20 | TAGAACAGCCCCCCACACAGTGG | 563 |

TABLE 10
TALE sequences targeting the chromosome 22 hotspot.
[hg38 chr22:35,373,912-35,373,916 (861);
chr22:35,377,843-35,377,847 (1153)].

| NAME | DNA SEQUENCE (SEQ ID NO:) | RVD AMINO ACID CODE |
|---|---|---|
| TALE22F- | TCTTCCTAGTCTCTTCTCTACCCAGT (632) | HD NG NG HD HD NG NI NH NG HD NG HD NG NG HD |
| R001 | | NG HD NG NI HD HD HD NI NH NG |
| TALE22- | TACACTCCAGCCTGGGAAACAGAGT (633) | NI HD NI HD NG HD HD NI NH HD HD NG NH NH NH |
| F002 | | NI NI NI HD NI NH NI NH NG |
| TALE22- | TCTTTTCCTTAGGACGGCT (634) | HD NG NG NG NG HD HD NG NG NI NH NH NI HD NH |
| F003 | | NH HD NG |
| TALE22- | TCGCTCAGGCCTGTCAT (635) | HD NH HD NG HD NI NH NH HD HD NG NH NG HD NI |
| F004 | | NG |
| TALE22- | TCCATATGGAAGACTT (636) | HD HD NI NG NI NG NH NH NI NI NH NI HD NG NG |
| F005 | | |
| TALE22- | TACCCAGTTAACCACCCT (637) | NI HD HD HD NI NH NG NG NI NI HD HD NI HD HD |
| F006 | | HD NG |
| TALE22- | TGGCGCATGCCTGTAATCCCAGCTACT (638) | NH NH HD NH HD NI NG NH HD HD NG NH NG NI NI |
| F007 | | NG HD HD HD NH HD NG NI HD NG |
| | | NI |
| TALE22- | TATACGAGGAGAAAATTAGCATTCCT (639) | NI NG NI HD NH NI NH NH NI NH NI NI NI NI NG |
| F008 | | NG NG |
| | | NG NI NH HD NI HD HD NG |
| TALE22- | TCTGCCTCCCAGGTTCACGCAAT (640) | HD NG NH HD HD NG HD HD HD NI NH NH NG NG HD |
| R009 | | NI HD NH HD NI NG |
| | | NI |
| TALE22- | TGCCTTGTCACGTTTTCACAGT (641) | NH HD HD NG NG NH NG HD NI HD NH NG NG NG NG |
| F010 | | HD NI HD NI NH NG |
| TALE22- | TGTCACCTTCTGTATGTGCAACCAT (642) | NH NG HD NI HD HD NG NG HD NG NH NG NI NG NH |
| F001A | | NG NH HD NI NI HD HD NI NG |
| TALE22- | TCTGTATGTGCAACCAT (643) | HD NG NH NG NI NG NH NG NH HD NI NI HD HD NI |
| F002A | | NG |
| TALE22- | TAGTCAAGCAACAGGAT (644) | NI NH NG HD NI NI NH HD NI NI HD NI NH NH NI |
| R03A | | NG |
| TALE22- | TCCAAGATAATTCCCCAT (645) | HD HD NI NI NH NI NG NI NI NG NG HD HD HD HD |
| F004A | | NI NG |
| TALE22- | TCTGCAAGATCCTTTT (646) | HD NG NH HD NI NI NH NI NG HD HD NG NG NG NG |
| F005A | | |
| TALE22- | TGCTATGTAAGGTAGCAAAAAGGTAACCT | NH HD NG NI NG NH NG NI NI NH NH NG NI NH HD |
| F006A | (647) | NI NI NI NI NI NH NH NG NI NI HD HD NG |
| TALE22- | TCTCTCTCCTCCTGCT (648) | HD NG HD NG HD NG HD HD NG HD HD NG NH HD NG |
| R007A | | |
| TALE22- | TCCAAATGCTATTCTCTCT (649) | HD HD NI NI NI NG NH HD NG NI NG NG HD NG HD |
| R008A | | NG HD NG |
| TALE22- | TGCTGATTCAGCCTCCT (650) | NH HD NG NH NI NG NG HD NI NH HD HD NG HD HD |
| R009A | | NG |
| TALE22- | TAGAACAGCCCCCCACACAGT (651) | NI NH NI NI HD NI NH HD HD HD HD HD HD NI HD |
| F010A | | NI HD NI NH NG |

### Example 2-Identification of Excision Positive and Integration Negative Mutants

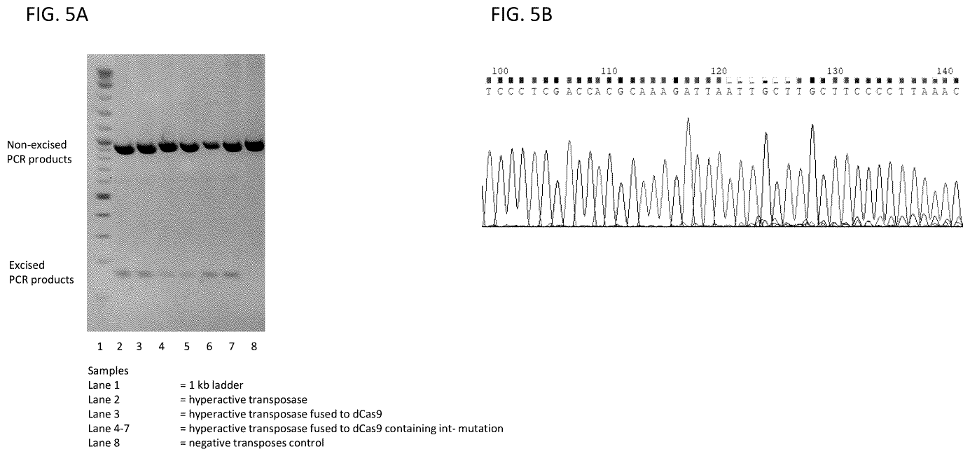

FIG. 5A depicts a 1% agarose gel showing the results of a PCR-based assay to test for excision activity. A HEK293 cell line that expresses GFP at a known genomic site is transfected with helper plasmid alone to excise the donor GFP DNA at the genomic locus by recognizing the end sequences. Lane 1 shows a 1 kb ladder to size the amplicon fragments. PCR primers flanking the end sequences are used to amplify the TTAA donor site. The amplicon is 2.5 kb when the GFP donor insert is present in HEK293 cells and 200 bp when the GFP donor is excised. Lane 2 shows hyperactive transposase, lane 3 shows hyperactive transposase fused to dCas9, lanes 4-7 show hyperactive transposase fused to dCas9 containing int-mutation, and lane 8 shows a negative control absent for transposase.

FIG. 5B depicts a DNA sequencing chromatogram that confirms that the samples in lanes 2-7 of FIG. 5A no longer contain the donor GFP insertion.

Several guide RNAs for targeting to the Rosa26 safe harbor genomic sequence were constructed for this experiment. One example is shown on FIG. 6. FIG. 6 depicts the human Rosa26 gene safe harbor target sequence. The location of the target sites that guide RNAs were designed to bind are annotated. The intended TTAA hotspot and the sequence of the eleven guide RNAs that were tested for directing insertion to Rosa26 are listed.

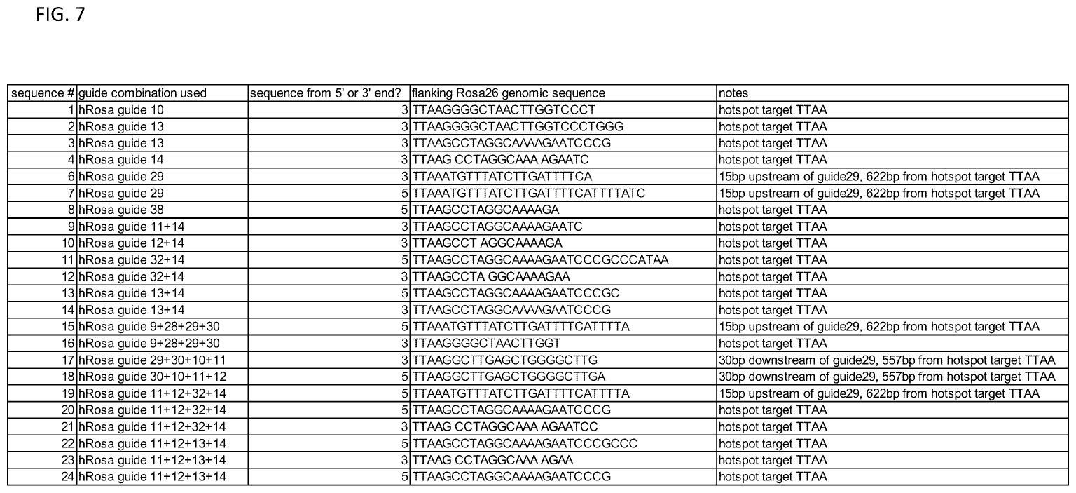

dCas9 was fused to piggyBac. Nested PCR was used to recover genomic insertions at the target site. The results suggest that most insertions occurred at a single hotspot target TTAA. The results suggest that the system of the present disclosure can be used to target the genome. Recovered targeted insertions to the human Rosa26 genome using dCas9-PB are shown in FIG. 7.

FIG. 7 lists genomic insertions recovered at Rosa26 from HEK293 cells following transfection of the donor plasmid and helper plasmid encoding dCas9 fused to the hyperactive transposase (FIG. 1B). Nested genomic PCR with forward primers located at the Rosa26 target sequence and reverse primers oriented out from the transposon (FIG. 4) were sequenced and aligned to the human genome using BLAST. The guide combinations are listed as well as the orientation of the insert in the genome and the flanking sequence and location of the targeted genomic insertions.

Mutations were introduced into the DNA binding domain of the transposase to disrupt its native binding. The results of hyperactive transposase with DNA binding domain mutants by integration activity are shown in FIG. 8.

FIG. 8 depicts the results of integration and excision assays on hyperactive transposase fused to dCas9 with DNA binding domain mutants by integration activity. For excision assays, a reporter plasmid containing a PB transposon interrupting a zsGreen gene was co-transfected with a helper plasmid encoding the hyperactive transposase. Cells are cultured for 4 days. Upon successful excision of the transposon, the excision reporter plasmid reconstitutes a complete zsGreen gene and expresses zsGreen. For integration assays, the donor plasmid depicted in FIG. 4 was co-transfected with a helper plasmid encoding the hyperactive transposase. Cells are cultured for >2 weeks without antibiotic selection. Upon successful integration of the transposon into the genome the cells express GFP. Bars represent % GFP cells measured by flow cytometry. Mutations were designed to disrupt the binding of the hyperactive transposase to the target DNA. Arrows indicate mutants that have reduced integration but maintain excision. Number denotes the position of the amino acid residue relative to SEQ ID NO: 1.

For the integration assay, HEK293 cells are plated in 12-well size plates the day before transfection. The day of the transfection the media is exchanged 1 hour and 30 min before the transfection is performed. A 3:1 ratio of X-tremeGENE™9 DNA Transfection Reagent protocol reagent is used to co-transfect a donor plasmid containing GFP and a helper plasmid in duplicate using 600 ng of DNA each. Forty-eight (48) hrs after the transfection the cells are analyzed by flow cytometry to count the percentage of GFP expressing cells to measure transient transfection efficiency. The cells are gated to distinguish them from debris and 20,000 cells are counted. The cultures are grown for 15-20 days without antibiotic. Cells are passaged 2/3 times per week. Flow cytometry is used to count the percentage of GFP expressing cells to measure integration efficiency at 2 weeks. The final integration efficiency is calculated by dividing the 2-week percentage of GFP cells by the percentage of GFP cell at 48 hr. The excision assay is performed by measuring the percentage of GFP cells in a cell line with a known GFP donor integration. The cells are grown to 80% confluency and analyzed by flow cytometry to count the percentage of GFP expressing cells as a baseline measurement. This percentage is used as the standard (i.e., 100%). X-tremeGENE™ 9 DNA Transfection Reagent protocol reagent is used to transfect helper plasmid in duplicate using 600 ng of DNA. The cells are gated to distinguish them from debris and 20,000 cells are counted. Forty-eight (48) hrs after the transfection the cells are analyzed by flow cytometry to count the percentage of GFP expressing cells. The cells are gated to distinguish them from debris and 20,000 cells are counted. The final integration efficiency is calculated by the baseline percentage of GFP cells by the percentage of GFP cells at 48 hr. The excision results are confirmed by the PCR-excision assay (FIG. 5A and FIG. 5B).

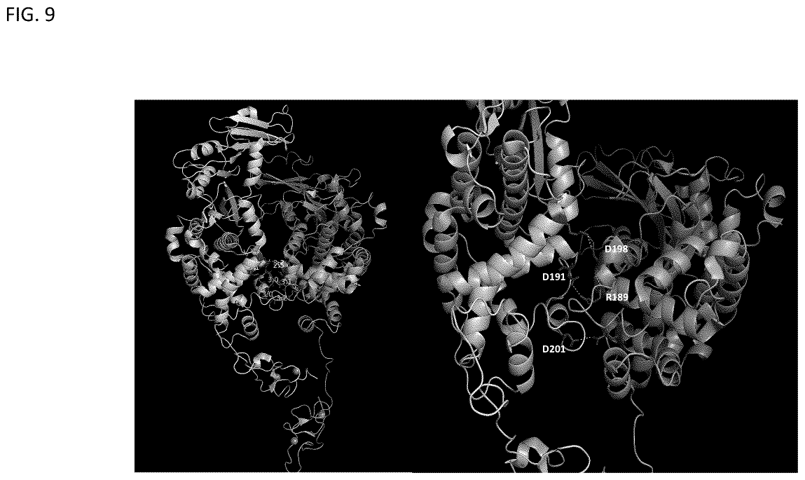

FIG. 9 depicts sites along the dimerization surface of hyperactive transposase (D191, D198, R189, and D201) that decrease integration activity when mutated. Mutations in the dimerization domain cause the transposase to be integration negative. Upon re-location to the target site by tethering of a DNA binding protein such as a TALE or dCas9, the dimers become bound and restore activity leading to insertions at the target sequence.

Based in part on FIG. 9, mutations were introduced into the dimerization domain to disrupt its ability to dimerize on its own. FIG. 10 shows the excision and integration assay of these mutants.

FIG. 10 depicts the results of integration and excision assays on hyperactive transposase fused to dCas9 with dimerization domain mutants by integration activity. Mutations were designed to disrupt the binding of the dimerization domain of the hyperactive transposase. Arrows indicate mutants that have reduced integration but maintain excision. Number denotes the position of the amino acid residue relative to SEQ ID NO: 1.

### Example 3-TTAA Sites in hROSA26 and AAVS1 Targeted by guideRNAs or TALES

FIG. 21 depicts the TTAA site in hROSA26 (hg38 chr3:9,396, 133-9,396,305) that is targeted by guideRNAs (TABLE 5) or TALES (TABLE 7).

FIG. 22 depicts two TTAA sites in AAVS1 (hg38 chr19:55,112,851-55,113,324) that are targeted by guideRNAs (TABLE 6) or TALES (TABLE 8).

### Example 4-Truncation of the N-Terminal or C-Terminal Residues Reduces Off-Target Insertion

piggyBac was further tested for improved excision and integration frequencies by deleting either N or C termini at various positions and various lengths. Illustrative structural rationale, without wishing to be bound by theory, for deleting the N- and C-termini amino acid residues in the piggyBac are shown in TABLE 11.

TABLE 11
Non-limiting structural rationale for deleting
the N- and C-termini amino acid residues.

| Deletion | Amino Acid | |
|---|---|---|
| Name | Deleted* | Non-Limiting/Non-Binding Rationale |
| N1 | 1-Glu24 | Not conserved |
| N2 | 1-Val39 | Not conserved |
| N3 | 1-Gln87 | Removes beta sheet |
| N4 | 1-Arg116 | Beginning of solved structure |
| N5 | 1-Ile128 | Removes small helix at loop |
| N6 | 1-Phe138 | Removes helix at loop (one molecule breaks |
| | | large helix) |
| N7 | 1-Arg159 | Removes large helix |
| N8 | 1-Thr171 | Removes small helix |
| N9 | 1-Lys190 | Remove large helix involved in dimerization |
| C1 | Ala570-594 | Removes conserved Cysteines |
| C2 | Lys554-594 | Removes NLS |

*Numbering of amino acids is relative to SEQ ID NO: 502

FIG. 23 depicts the results of excision and integration assays on piggyBac (pB) transposase that contains different deletions at the N- and C-termini. Bars represent % GFP cells measured by flow cytometry. The hyperactive pB designated as “pB N0” known for high excision activity was used as a positive control. Stuffer DNA (pB Neg) that did not show expression served as negative controls. Abbreviations of test conditions are found in TABLE 11. For each sample, the left histogram is excision, and the right is integration.

The excision assay was performed by measuring the percentage of GFP cells in a cell line with a known GFP donor integration. The cells were grown to 80% confluency and analyzed by flow cytometry to count the percentage of GFP expressing cells as a baseline measurement. This percentage was used as the standard (i.e., 100%). X-tremeGENE™ 9 DNA Transfection Reagent protocol reagent was used to transfect helper plasmid in duplicate using 600 ng of DNA. The cells were gated to distinguish them from debris and 20,000 cells were counted. Forty-eight (48) hrs after the transfection the cells were analyzed by flow cytometry to count the percentage of GFP expressing cells. The cells were gated to distinguish them from debris and 20,000 cells were counted. The final integration efficiency was calculated by the baseline percentage of GFP cells by the percentage of GFP cells at 48 hr. For the integration assay, HEK293 cells were plated in 12-well size plates the day before transfection. The day of the transfection the media was exchanged 1 hour and 30 min before the transfection was performed. A 3:1 ratio of X-tremeGENE™ 9 DNA Transfection Reagent protocol reagent was used to co-transfect a donor plasmid containing GFP and a helper plasmid in duplicate using 600 ng of DNA each. Forty-eight (48) hrs after the transfection the cells were analyzed by flow cytometry to count the percentage of GFP expressing cells to measure transient transfection efficiency. The cells were gated to distinguish them from debris and 20,000 cells were counted. The cultures were grown for 15-20 days without antibiotic. Cells were passaged 2/3 times per week. Flow cytometry was used to count the percentage of GFP expressing cells to measure integration efficiency at 2 weeks. The final integration efficiency was calculated by dividing the 2-week percentage of GFP cells by the percentage of GFP cell at 48 hr.

The present example shows, inter alia, that truncation of the N-terminus, which without wishing to be bound by theory, may be involved in target DNA binding, will reduce off-target insertion.

### Example 5-Flexible Loop Domains of piggyBac

In order to test whether linking domains could be fused to the N-terminal or inserted into the flexible loop domains of piggyBac, the piggyBac loop domains are generated as shown in TABLE 12.

TABLE 12
Flexible loop domains of piggyBac.
PB loop regions

S117-T122
N127-P131
F138-F139
L157-T171
K190-M194
D201-M212
L224-S230
R236-V240
N258-A263
L272-R281
I284-Y290
S301-Y305
Y312-V322
G338-R341
D346-T350
L358-I378
V371-T392
D398-L401
K407-M413
S419-K432
G444-G445
S454-R464
K492-Q497
E520-K525
N534-N571
C574-K579
C582-F594

### Example 6-Fusion piggyBac Constructs with DNA Binding Domains: DNA Binding Domain Insertions into piggyBac Transposase

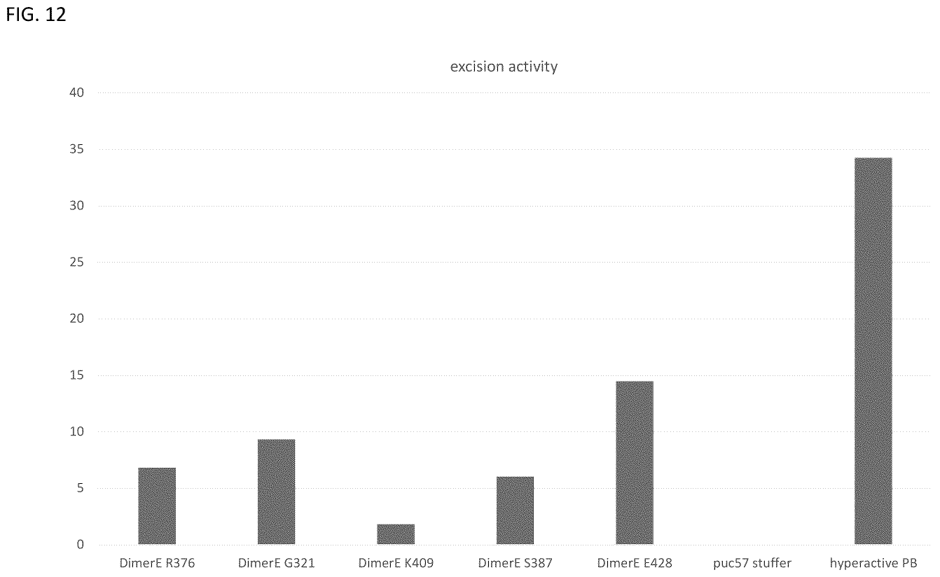

Several binding domains were covalently fused within the piggyBac (PB) transposase open reading frame at the loop domains. These locations are shown to tolerate insertions without inactivating the excision activity of PB (FIG. 12). The amino acids for the loop domains of PB that insertions are made include positions: V371-1378, Y312-V322, K407-M413, S385-T392, A424-K432, and R275-K290 relative to SEQ ID NO: 11. Some examples of specific loop insertion sites are at positions: R315, G321, R376, S387, K409, and E428 relative to SEQ ID NO: 11.

Several zinc fingers, that were designed to target a specific DNA sequence, were inserted within the PB transposase open reading frame at the loop domains (described above). These locations were shown to tolerate insertions without inactivating the excision activity of PB (FIG. 12). This configuration resulted in changing the targeting region of the PB enzyme to a different region of DNA near the binding sequence of the zinc finger molecule, due to the covalent fusion between the two proteins. The insertion of the zinc finger into the loop domain provides steric spacing for the targeting of PB to the desired sequences.

To show that inserting the linking domains into the loops of piggyBac is tolerated and excision activity is maintained, excision activities were measured, and the results are shown in FIG. 12.

FIG. 12 depicts the results of excision assays on the hyperactive transposase that contains linker domain insertions at several flexible loop domains. An excision reporter plasmid containing a PB transposon interrupting a zsGreen gene were co-transfected with a helper plasmid encoding PB containing the E leucine zipper heterodimer fused into a permissive loop domain of the transposase. Upon successful excision of the transposon, the excision reporter plasmid reconstituted a complete zsGreen gene and expressed zsGreen. Bars represent % GFP cells measured by flow cytometry. The hyperactive PB was a positive control known to successfully excise. Stuffer DNA did not express PB. Five E dimer loop fusions at the indicated amino acid in PB were tested for excision activities demonstrating that insertions at these loop domains are tolerated by the transposase and maintain activity. This data demonstrates, inter alia, that functional domains can be inserted into the PB loop locations while maintaining activity of the transposase.

### Example 7-Excision Activity of piggyBac Containing a DNA Binding Domain Insertion into Loops

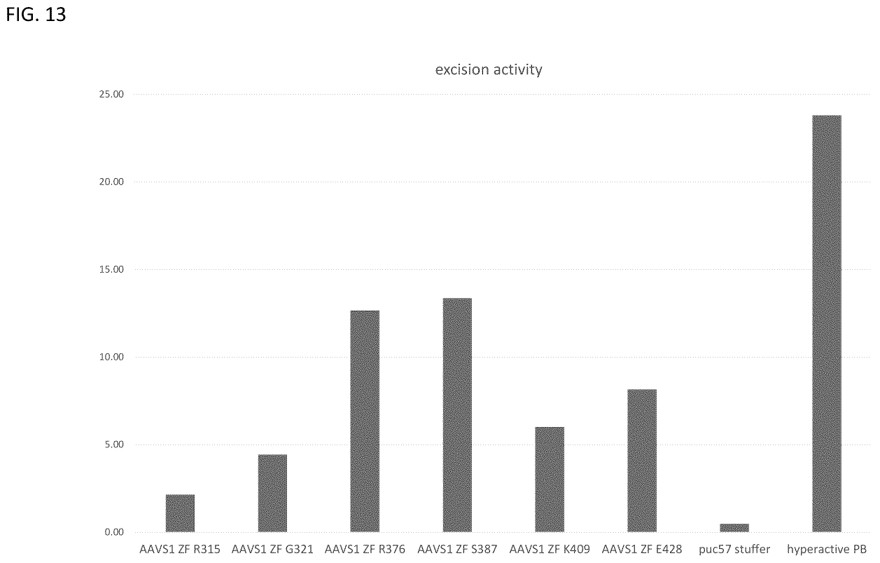

A second approach for redirecting the targeting of PB involves a direct fusion of a DNA binding domain to the N-terminus or loop domains of piggyBac (PB). These fusions result in the PB transposase becoming localized to the target DNA where integration occurs within close proximity to this sequence. Several zinc fingers, that were designed to target a specific DNA sequence, were inserted within the PB transposase open reading frame at loop domains. These locations were shown to tolerate insertions without inactivating the excision activity of PB (FIG. 13). This configuration results in the PB enzyme becoming relocated to the binding sequence of the zinc finger due to the covalent fusion between the two proteins. The insertion of the zinc finger into the loop domain provides an effective steric spacing for the targeting of PB to desired sequences.

To show that inserting a ZF that is made to bind AAVS1 is tolerated and excision activity is maintained, excision activities were measured and results are shown in FIG. 13.

FIG. 13 depicts the excision activity of piggyBac containing a ZF DBD insertion into loops. An excision reporter plasmid containing a PB transposon interrupting a zsGreen gene was co-transfected with a helper plasmid encoding PB containing a zinc finger DNA binding domain designed to bind a sequence in AAVS1 and that was fused into a permissive loop domain of the transposase. Upon successful excision of the transposon, the excision reporter plasmid reconstitutes a complete zsGreen gene and expresses zsGreen. Bars represent % GFP cells measured by flow cytometry. Hyperactive PB is a positive control known to successfully excise. Stuffer DNA does not express PB. Six fusions at the indicated amino acid in PB were tested demonstrating that insertions at these loop domains are tolerated by the transposase and maintain activity. This data demonstrates, inter alia, that DNA binding domains designed to target specific sequences can be inserted into the PB loop locations while maintaining activity of the transposase.

### Example 8-Evidence of Directing piggyBac Insertions to a Target Sequence Using Bridging Domains and DNA Binding domains fused into the loops of PB

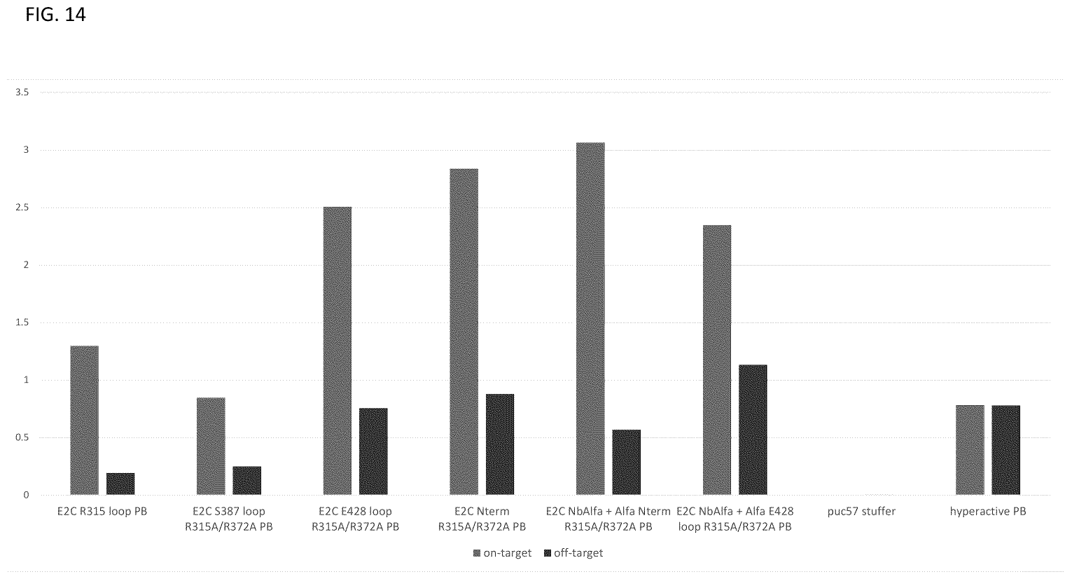

A plasmid to plasmid assay was used to demonstrate successful targeting of PB using both the bridging domain insertions into the loops of PB as well as the direct covalent DNA binding domain insertions into the loops of PB. A reporter assay consisted of a helper plasmid that encodes a PB transposes containing either a loop fusion of a bridging domain or a loop fusion of a zinc finger. An on-target reporter plasmid containing a splice acceptor and a split GFP gene encoding the GFP fluorescent protein contains the target sequence for the zinc finger DNA binding domain. An off-target reporter plasmid containing a splice acceptor and a split GFP gene encoding the GFP fluorescent protein is absent for the target sequence. A donor plasmid contains the PB transposon with a CMV promoter oriented pointing toward the beginning of a split GFP gene followed by a splice donor. In the event of successful targeting, the transposon inserts near the target sequence on the on-target reporter plasmid and aligns the promotor and first part of the split GFP with the second part of the split GFP with an intron between the two parts. This results in more GFP expression from the on-target reporter compared to the off-target reporter that does not contain a target sequence. This expression can be detected with flow cytometry. Upon co-expression of a helper plasmid encoding PB with one of several loop insertions of bridging domains or direct fusion of DNA binding domains, the donor plasmid containing the transposon and CMV promoter, and the reporter plasmid containing the promotorless-GFP, an increase in GFP fluorescence for the on-target reporter was measured, indicating that the loop fusion strategy described above is successful at targeting PB to the plasmid target sequence (FIG. 14).

To demonstrate that an integration-negative mutant, such as R315A/R372A, can be targeted when the DNA binding domain is located within loops, when bound to the N-terminal, when separated by a linking domain at the N-terminal, and/or when separated by a linking domain inserted into a loop, an experiment was carried out and the results are shown in FIG. 14.

FIG. 14 depicts evidence of directing piggyBac insertions to a specific target sequence using linking domains and DNA binding domains which were fused into the loops of PB. For the plasmid to plasmid targeting assay, an on-target reporter plasmid containing an E2C ZF target sequence expresses GFP following successful insertion of the donor plasmid at the target site. An off-target reporter that was absent for the E2C target sequence reports off-target insertion not driven by E2C binding. Targeting was achieved by using a two-part bridging mechanism in which the E2C ZF was linked to PB via two linking domains that bind with each other to link the ZF to PB. One domain was fused to the ZF and the other to the N-terminus or a loop of PB. Targeting was achieved by direct fusion of the E2C ZF to the N-terminus or to the loop of PB. The R315A/R372A mutations shown to reduce integration while maintaining excision activity (FIG. 8) were incorporated into the hyperactive transposase as indicated. These mutations reduce insertion at off-target sequences but insertion is rescued at the intended on-target sequence.

### Description of Labels:

E2C R315 loop PB, the E2C ZF fused to the loop domain of PB after amino acid R315 (example of direct fusion of a DNA binding domain to a loop).

E2C S387 loop R315A/R372A PB, the E2C ZF fused to the loop domain of PB after amino acid S387, PB contains the R315A/R372A excision+/integration− mutations (example of direct fusion of a DNA binding domain to a loop).

E2C E428 loop R315A/R372A PB, the E2C ZF fused to the loop domain of PB after amino acid E428, PB contains the R315A/R372A excision+/integration− mutations (example of direct fusion of a DNA binding domain to a loop).

E2C Nterm R315A/R372A PB, the E2C ZF fused to the N-terminus of PB, PB contains the R315A/R372A excision+/integration− mutations (example of direct fusion of a DNA binding domain to the N terminus).

E2C NbAlfa+Alfa Nterm R315A/R372A PB, the E2C ZF fused to the Alfa nanobody cotransfected with the Alfa tag fused to the N-terminus of PB, PB contains the R315A/R372A excision+/integration− mutations (example of a bridging fusion approach by fusion the N terminus).

E2C NbAlfa+Alfa E428 loop R315A/R372A PB, the E2C ZF fused to the Alfa nanobody cotransfected with the Alfa tag fused to the loop domain of PB after amino acid E428, PB contains the R315A/R372A excision+/integration-mutations (example of a bridging fusion approach by fusion to a loop).

Puc57 stuffer, negative control DNA that does not express PB.

Hyperactive PB, PB without a targeting fusion.

Bars indicate percent GFP glowing cells from either the on-target reporter or the off-target reporter.

E2C NbAlfa, a camelid VHH against ALFA-tagged proteins, has a nucleotide sequence of SEQ ID NO: 515 and an amino acid sequence of SEQ ID NO: 516; Alfa Nterm R315 R372 has a nucleotide sequence of SEQ ID NO: 517 and an amino acid sequence of SEQ ID NO: 518; and Alfa E428 loop R315 R372 has a nucleotide sequence of SEQ ID NO: 519 and an amino acid sequence of SEQ ID NO: 520.

This data demonstrates, inter alia, that the PB transposase can successfully be directed to specifically insert at intended target sequences in human cells. The modifications include mutations such as R315A/R372A that reduce off-target insertion but maintain excision activity (FIG. 8). Without wishing to be bound by theory, these mutations disable target DNA binding. Upon DNA targeting by a DNA binding domain such as the E2C zinc finger, without wishing to be bound by theory, insertion is rescued, and the transposase inserts the transposon near the target sequence on the reporter plasmid. Several successful strategies were used to direct the transposase to the target sequence. Direct fusions of the E2C zinc finger to loop domains of the transposase led to higher on-target insertions. Direct fusion of the E2C zinc finger to the N-terminus also increased on-target insertion. By fusing the E2C zinc finger to a bridging domain that non-covalently connects to a second domain found on PB, the E2C zinc finger was non-covalently connected to both the N-terminus and loop domains which resulted in successful on-target insertions to the reporter plasmid near the E2C target sequence.

### Example 9-DNA Binding Domain can be Inserted into the N-Terminal Flexible Region and Result in Targeting

Sequence targeting activities of piggyBac transposase with DNA binding domain insertions with internal loop fusion within the flexible N-terminal domain was tested.

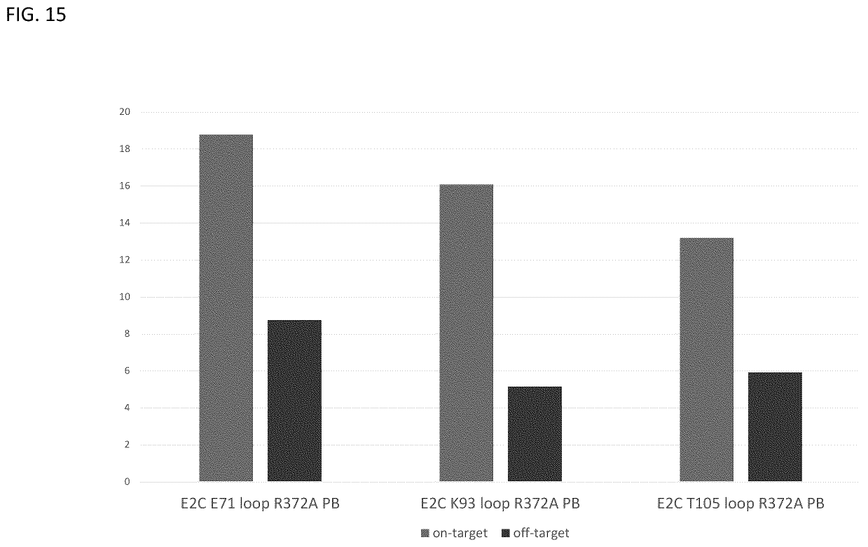

FIG. 15 depicts evidence of directing piggyBac insertions to a specific target sequence using DNA binding domains which were fused into the N terminal flexible loop region of pB that comprises of amino acids: 24-128. The plasmid to plasmid targeting assay is described in FIG. 14. Description of labels: E2C E71 loop R372A PB, E2C E71 loop R372A PB, and E2C E71 loop R372A PB, each contain the E2C ZF fused to the loop domain of PB after indicated amino acid R315 (example of direct fusion of a DNA binding domain to a loop). Bars indicate percent GFP glowing cells from either the on-target reporter or the off-target reporter.

### Example 10-Fusion of DNA Binding Domain to the N-Terminal Truncation Mutations Result in Targeting

Sequence targeting activities of PB transposase with DNA binding domain insertions with and without linking domains with N-terminal truncations were tested. Both direct fusions to the N-terminal truncation and using a linking domain result in targeting. Noteworthy, when the DNA binding domain is omitted, no integration occurs. This result suggests that the addition of the DNA binding domain is rescuing targeted integration.

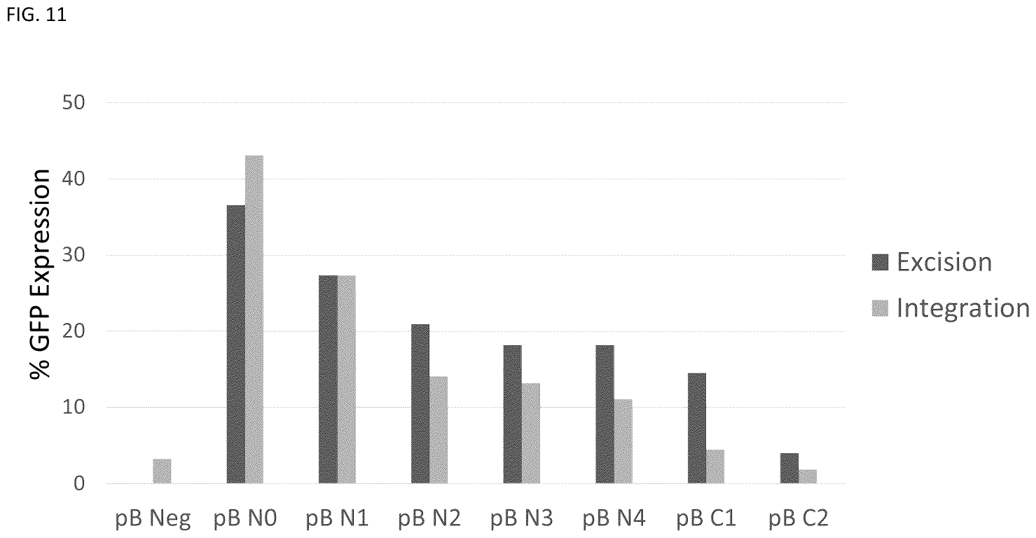

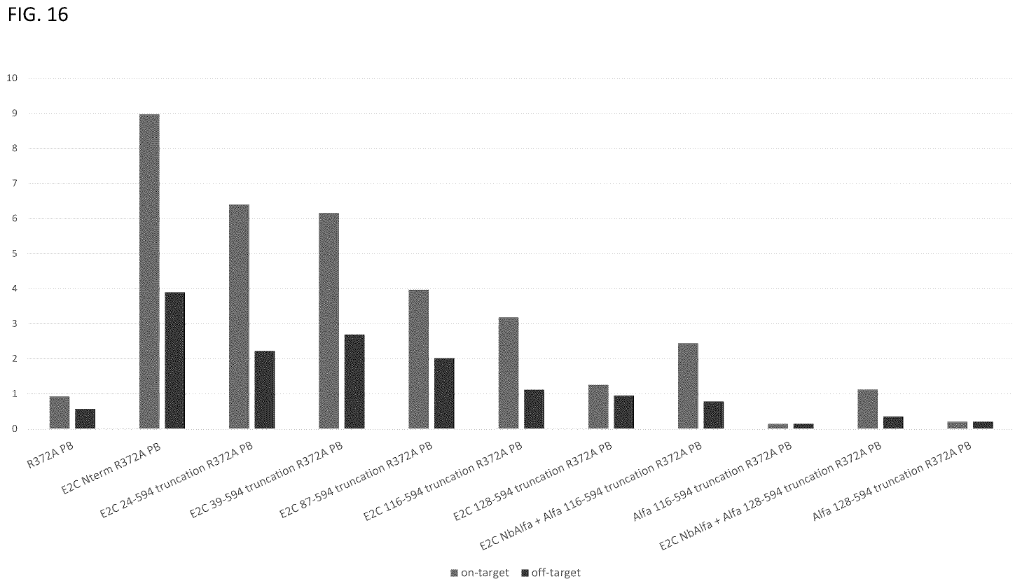

FIG. 16 depicts evidence of directing piggyBac insertions to a specific target sequence using DNA binding domains fused onto N terminal truncation mutants described in FIG. 11. Both direct fusions as well as fusions using the bridging strategy described above using one linking domain fused to the DBD and a second fused to N terminal truncated PB, resulted in targeted insertion. The plasmid to plasmid targeting assay is described in FIG. 14. Description of labels: R372A PB, transposase without an E2C fusion does not show an increase in targeting; E2C Nterm R372A PB, the E2C ZF fused to the N-terminus of PB; E2C truncation R372A PB, the E2C ZF was fused to the N-terminus of PB at the indicated truncation (24-594, 39-594, 87-594, 116-594). E2C NbAlfa+Alfa truncation R372A PB, co-transfection of two helper plasmids containing the Alfa tag and NbAlfa (nanobody) linking domains used to bridge the proteins. The Alfa tag was fused to the N-terminus of PB at the indicated truncation (87-594, 116-594); Alfa truncation R372A PB, control transfection omitting the DBD helper plasmid from the bridging approach. The Alfa tag was fused to the N-terminus of PB at the indicated truncation (87-594, 116-594) does not show an increase in targeting; Targeting rescue was demonstrated due to the addition of the second bridging helper containing the E2C DBD, compare E2C NbAlfa+Alfa 116-594 truncation R372A PB (targeting) to Alfa 116-594 truncation R372A PB (no targeting). Bars indicate percent GFP glowing cells from either the on-target reporter or the off-target reporter.

### Example 11-In the Absence of DNA Binding Domain, the Integration Negative Mutant Shows No Integration

This example tests for the rescue of sequence targeting activity by fusion of a DNA binding domain to DNA binding mutant piggyBac. The results suggest that without the DNA binding domain, the integration negative mutant, which is R315A/R372A in the instant example, has no integration on the targeting based on targeting assay. When the DNA binding domain is fused to the N-terminal or if it is connected by a linking domain, targeted integration is rescued.

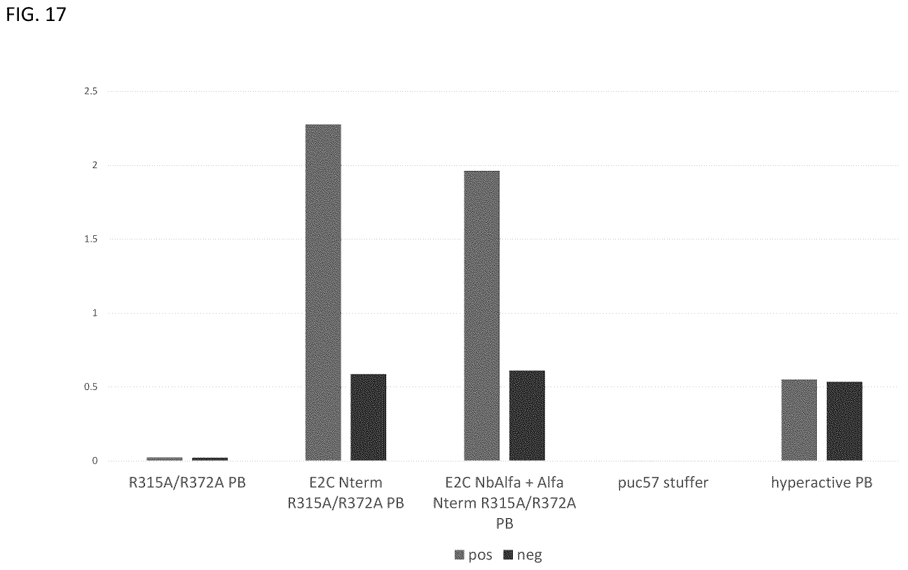

FIG. 17 depicts evidence that an excision+/integration− mutant R315A/R372A, highlighted in FIG. 8, fails to insert into a target plasmid. Addition of the E2C DNA binding domain rescues the targeting ability of the protein. Both covalent and non-covalent strategies rescue integration into the target plasmid. The plasmid to plasmid targeting assay is described in FIG. 14. Insertion rescue and sequence targeting was achieved by using a two-part bridging mechanism described above in which the E2C ZF was linked to PB via two linking domains that bind with each other to link the ZF to PB. One domain was fused to the ZF and the other to the N-terminus. Insertion rescue and sequence targeting was achieved by direct fusion of the E2C ZF to the N-terminus. Description of labels: R315A/R372A PB, Hyperactive PB containing the R315A/R372A excision+/integration− mutations without a targeting fusion that fails to target; E2C Nterm R315A/R372A PB, the E2C ZF fused to the N-terminus of PB, PB contains the R315A/R372A excision+/integration-mutations (example of direct fusion of a DNA binding domain to the N terminus); E2C NbAlfa+Alfa Nterm R315A/R372A PB, the E2C ZF fused to the Alfa nanobody cotransfected with the Alfa tag fused to the N-terminus of PB, PB contains the R315A/R372A excision+/integration− mutations (example of a bridging fusion approach by fusion the N terminus); Puc57 stuffer, negative control DNA that does not express PB; Hyperactive PB, PB without a targeting fusion. Bars indicate percent GFP glowing cells from either the on-target reporter or the off-target reporter.

### Example 12—the Loop Inserts are Functional, and the Constructs Show Specificity when Utilizing piggyBac that is Excision-Positive and Integration-Negative

The present example tests for sequence targeting activity by fusion of a DNA binding domain to N-terminal and internal loop domain of piggyBac.

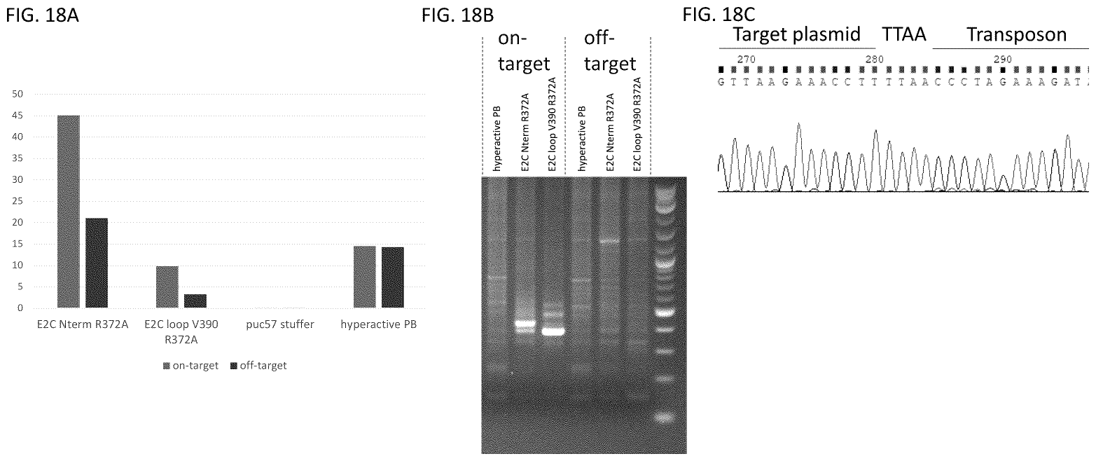

FIGS. 18A-C depicts evidence of directing piggyBac insertions to a specific target sequence using DNA binding domains fused into the loops of PB. The E2C ZF fused at the V390 loop was compared to a helper with an E2C fused at the N terminus and compared against a helper without an E2C fusion. The E2C loop insert simultaneously reduced off-target insertion, FIG. 18A (lowered off-target compared to hyperactive pB) and FIG. 18B (decreased PCR products) and increased the specificity of insertion at the target sequence FIG. 18B (single targeted PCR product near 400 bp). The plasmid to plasmid targeting assay is described in FIG. 14. The PCR was performed using cell lysates as template containing the two combined plasmids from the plasmid to plasmid targeting assay. The forward primer bound the reporter plasmid and the reverse primer bound the transposon. Products arose from a transfer of the transposon to the reporter plasmid. A product of approximately 400 bp indicates an insertion near the E2C target sequence. Both E2C Nterm R372A and E2C V390 loop R372A resulted in targeting the E2C sequence. The hyperactive PB without an E2C DBD did not result in a visible product at 400 bp. Reporters without an E2C target sequence did not result in targeting to the same location on the reporter plasmid (FIG. 18B). FIG. 18C shows chromatogram sequence verification that the PCR product from E2C V390 loop R372A results from a targeted insertion 9 bp from the E2C sequence on the reporter plasmid. Description of labels: E2C Nterm R372A PB, the E2C ZF fused to the N-terminus of PB (example of direct fusion of a DNA binding domain to the N terminus); E2C V390 loop R372A PB, the E2C ZF fused to the loop domain of PB after amino acid V390 (example of direct fusion of a DNA binding domain to a loop); Puc57 stuffer, negative control DNA that does not express PB; Hyperactive PB, PB without a targeting fusion. Bars indicate percent GFP glowing cells from either the on-target reporter or the off-target reporter.

### Site Specific Targeting 3F E2C Fused to piggyBac

The example utilizes a hyperactive pB (hypPB) with 10 mutants in pB amino acid sequence (594 aa) including 130V, S103P,G165S, M282V, S509G, N538K, and N571S; and 182N, V109A, and Q591R with Exc+/Int−mutants R372A, D450N. Three finger E2C site was fused to the N-terminus using a 4× linker to hypPB Exc+, Int−(R372A/D450N). Three finger E2C site was fused using 2G linkers on either side of the V390 site in hypPB Exc+, Int−(R372A/D450N). The construct includes an N-terminus NLS.

Plasmid to plasmid reporter assay in HEK293 uses landing pad E2C recognition sites in the “out” orientation separate by 52 base pairs and three TTAA sites (12 bp, 24 bp, and 34 bp) 3′ to the left E2C recognition site and 14 bp, 24 bp, 36 bp 5′- to the right E2C recognition site).

### Summary of the Results:

Amplicon PCR shows site specific targeting with little off-targets using hypPB fusion proteins when Znf are directly fused to the N-terminus fusion or inserted in a region at DNA binding loop (V390).

Targeting is increased with E2C-hypPB-Exc+, Int−(R372A/D450N).

Results show that integration is more selective with E2C (3 fingers) than with E2C (6 fingers).

### Example 13-Predicted AlphaFold Structure of the E2C DBD Fused to the V390 Loop Domain of pB and its Association with the Target DNA

An AlphaFold image was modeled to predict the structure of the E2C DBD fused to the V390 flexible loop domain of piggyBac. The predicted structure suggests that the ZF (purple) contacts the target DNA near the TTAA.

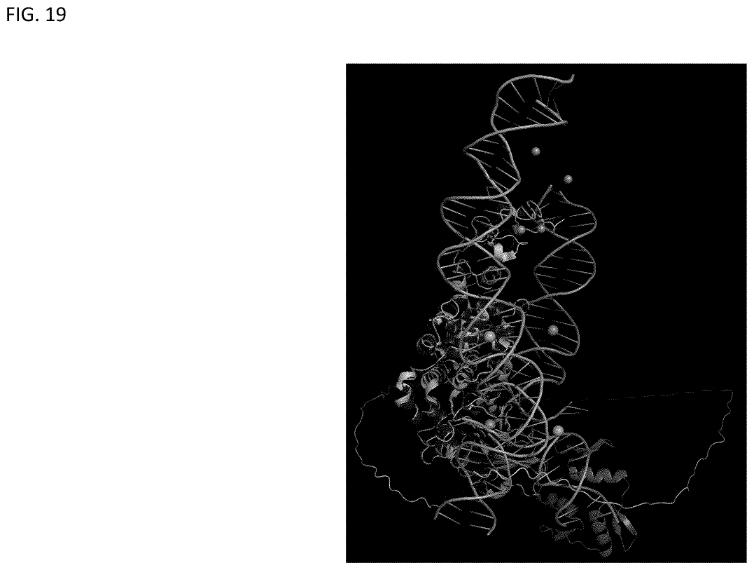

FIG. 19 shows AlphaFold image that was used to predict the structure of the E2C DBD fused to the V390 flexible loop domain of PB.

### Example 14-Sequence Targeting Activity by Fusion of a DNA Binding Domain to N-Terminal or Linking Domains of PB and Quantification of Targeting at a Single TTAA Target Sequence

In order to verify that the GFP positive cells from the targeting assay do represent true insertions, PCR was used to amplify the junction between the transposon and the target site. The products were deep sequenced using amplicon sequencing. The expected target TTAA junction was recovered. The results obtained by counting the frequency of the outputs suggest that high percentage of insertions at a single TTAA target sequence on the target plasmid.

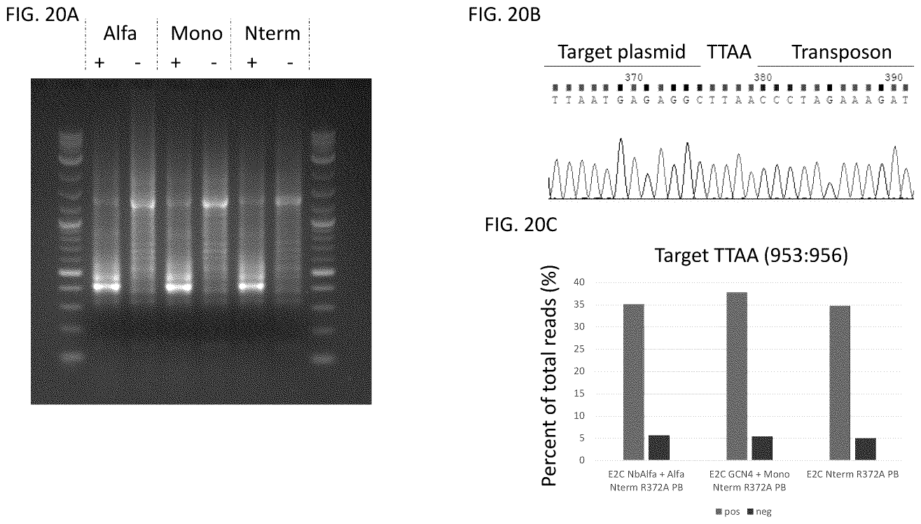

FIGS. 20A-C depict evidence of directing piggyBac insertions to a specific target sequence using direct fusions and linking domain fusions of DNA binding domains at the N terminus of PB. The plasmid to plasmid targeting assay was used as template for PCR as described in FIGS. 18A-C. In FIG. 20A, a direct fusion of E2C (Nterm) or fusions using the bridging strategy with linking domains Alfa tag and Monobody resulted in insertions near the E2C target sequence (expected size ˜400 bp) for reporter plasmids containing the E2C site (+) but not for plasmids without the E2C site (−). FIG. 20B shows sequence chromatogram verification that the PCR products from FIG. 20A resulted from targeted insertion 24 bp from the E2C sequence. FIG. 20C shows amplicon sequencing of all PCR products from FIG. 20A that was used to measure the frequency of insertions at the target TTAA located at bp 953-956 on the reporter plasmid that is 24 bp from the E2C site. Both bridging strategies using Alfa tag and monobody linking domains as well as the direct fusion to the N terminus resulted in high levels of targeted insertions on the reporter plasmid containing the E2C site (pos) but not for the reporter plasmid without the E2C site (neg).

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising an enzyme and a targeting element which directs the enzyme to a target site, optionally a genomic safe harbor site (GSHS), wherein the enzyme is a piggyBac transposase which comprises one or more mutations which cause decreased or ablated integration activity relative to an enzyme comprising an amino acid sequence of SEQ ID NO: 11 or functional equivalent thereof.

2. The composition of claim 1, wherein the piggyBac transposase comprises at least one substitution at positions corresponding to: 315, 372, 312, 324, 347, 374, and/or 375 of SEQ ID NO: 11, and/or wherein the enzyme comprises at least one substitution selected from R315A, R372A, Y312A, L324A, N347A, N374A, and K375A, wherein the positions are corresponding to positions of SEQ ID NO: 11.

3. The composition of claim 2, wherein the piggyBac transposase comprises one of R315A/R372A, R372A/K375A, N347A/R315A, L324A/Y312A, N374A, L324A/R315A, R315A/R372A/K375A, and L324A/N347A substitutions, wherein the positions are corresponding to positions of SEQ ID NO: 11.

4. The composition of claim 3, wherein the piggyBac transposase comprises R315A and R372A substitutions, wherein the positions are corresponding to positions of SEQ ID NO: 11.

5. The composition of claim 4, wherein the piggyBac transposase has an amino acid sequence of at least 90% identity to SEQ ID NO: 11 and R315A and R372A substitutions, wherein the positions are corresponding to positions of SEQ ID NO: 11.

6. The composition of claim 4, wherein the piggyBac transposase has an amino acid sequence of at least 95% identity to SEQ ID NO: 11 and R315A and R372A substitutions, wherein the positions are corresponding to positions of SEQ ID NO: 11.

7. The composition of claim 4, wherein the piggyBac transposase has an amino acid sequence of at least 98% identity to SEQ ID NO: 11 and R315A and R372A substitutions, wherein the positions are corresponding to positions of SEQ ID NO: 11.

8. The composition of any one of claims 1-7, wherein the GSHS is selected from adeno-associated virus site 1 (AAVS1), chemokine (C-C motif) receptor 5 (CCR5) gene, HIV-1 coreceptor, and human Rosa26 locus.

9. The composition of any one of claims 1-8, wherein the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

10. The composition of any one of claims 1-9, wherein the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11.

11. The composition of any one of claims 1-9, wherein the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N terminal deletion.

12. The composition of any one of claims 1-9, wherein the N terminal deletion yields reduced or ablated off-target effects of the enzyme compared to the enzyme without the N-terminal deletion.

13. The composition of any one of claims 1-12, wherein the enzyme comprises one or more N-terminal deletions of TABLE 11.

14. The composition of any one of claims 1-13, wherein the enzyme and the targeting element are fused to one another or linked via a linker or linker domain to one another.

15. The composition of claim 14, wherein the targeting element and/or the linker or linker domain are fused to the N- or C-terminus of the enzyme or inserted into the enzyme at one or more internal loops of the enzyme.

16. The composition of claim 15, wherein the enzyme comprises an insertion in a loop domain of selected from one or more the domains of TABLE 12, with reference to SEQ ID NO: 11.

17. The composition of claim 15, wherein the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11.

18. A composition comprising an enzyme and a targeting element which directs the enzyme to a target site, optionally a genomic safe harbor site (GSHS) and optionally a linker or linking domain which connects the enzyme and targeting element, wherein the enzyme is a piggyBac transposase and the targeting element and/or linker or linking domain are fused to the N- or C-terminus of the piggyBac transposase or inserted into the piggyBac transposase at one or more internal loops of the enzyme.

19. The composition of claim 18, wherein the enzyme comprises an insertion in a loop domain of selected from one or more the domains of TABLE 12, with reference to SEQ ID NO: 11.

20. The composition of claim 18 or 19, wherein the enzyme comprises an insertion at positions V371-1378, Y312-V322, K407-M413, S385-T392, A424-K432, and/or R275-K290 or positions V390, R315, G321, R376, S387, K409, and/or E428 or positions corresponding thereto, with reference to SEQ ID NO: 11.

21. The composition of any one of claims 18-20, wherein the GSHS is selected from adeno-associated virus site 1 (AAVS1), chemokine (C-C motif) receptor 5 (CCR5) gene, HIV-1 coreceptor, and human Rosa26 locus.

22. The composition of any one of claims 18-21, wherein the targeting element comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

23. The composition of any one of claims 18-22, wherein the piggyBac comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from the N-terminus and/or C-terminus of the polypeptide having an amino acid sequence of SEQ ID NO: 11.

24. The composition of any one of claims 18-23, wherein the piggyBac comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 11, wherein the deletion comprises an N or C terminal deletion.

25. The composition of any one of claims 18-24, wherein the N or C terminal deletion yields reduced or ablated off-target effects of the enzyme compared to the enzyme without the N- or C-terminal deletion.

26. The composition of any one of claims 18-25, wherein the enzyme comprises one or more N- or C-terminal deletions of TABLE 11.

27. The composition of any one of claims 1-26, wherein the composition is a nucleic acid, optionally an RNA.

28. The composition of any one of claims 1-27, wherein the composition further comprises a donor nucleic acid and/or is suitable for inserting a donor nucleic acid into a genome.

29. The composition of claim 28, wherein the donor nucleic acid is or comprises DNA.

30. The composition of any one of claims 1-29, wherein the composition is in the form of a lipid nanoparticle (LNP).

31. The composition of any one of claims 1-30, wherein the nucleic acid encoding the enzyme and the donor nucleic acid are in the same LNP.

32. A host cell comprising the LNP of claim 30 or 31.

33. A method for inserting a gene into the genome of a cell, comprising contacting a cell with the composition of any one of claims 1-32.

34. A method for treating a disease or disorder ex vivo, comprising contacting a cell with the composition of any one of claims 1-32 and administering the cell to a subject in need thereof.

35. A method for treating a disease or disorder in vivo, comprising administering the composition of any one of claims 1-32 to a subject in need thereof.


SEQUENCE LISTING (815 sequences)

SEQ ID NO: 1 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKPTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCRSCF

SEQ ID NO: 2 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKPTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCRSCF

SEQ ID NO: 3 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKSTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCRSCF

SEQ ID NO: 4 - protein, 1379 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 5 - DNA, 310 bp, synthetic construct
  source 1..310: mol_type = other DNA
atctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 6 - DNA, 205 bp, synthetic construct
  source 1..205: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaa

SEQ ID NO: 7 - DNA, 1785 bp, synthetic construct
  source 1..1785: mol_type = other DNA
atggggtcttcactggacgatgagcatattctgagcgccctgctgcagagcgatgacgagctggtgggagaggattccgattccgaggtcagtgaccacgtgtcagaggacgatgtgcagagcgacactgaggaagccttcatcgatgaggtccatgaagtgcagccaacaagctccggaagcgagatcctggatgaacagaacgtgattgaacagcctggctctagtctggcttccaataggaacctgacactgccacagcgaactattcggggcaagaacaagcactgctggagcacctccaagcctacacggagaagccgcgcgtccgccctgaacatcgtgagatcccagagggggccaacccgcatgtgccgaaatatctacgaccccctgctgtgctttaagctgttctttacagatgagatcattagtgaaatcgtgaagtggactaacgcagagatttcactgaaaaggcgcgaatctatgactagtgccaccttcagagacacaaatgaggatgaaatctacgctttctttggcattctggtcatgaccgcagtgaggaaggacaaccatatgtctacagacgatctgtttgatcgctctctgagtatggtgtatgtctcagtgatgagcagagacaggttcgattttttgatccggtgcctgagaatggacgataagagcattcgacctacactgcgggagaatgacgtgttcaccccagtgaggaaaatctgggatctgtttatccaccagtgtattcagaactacacacccggagcccatctgactatcgacgaacagctgctgggcttccgcgggcgatgcccttttcgcgtatacattccaaataagcccagcaaatatggcatcaagattctgatgatgtgcgattccgggaccaaatacatgatcaacggaatgccatatctgggacggggcacccagacaaatggagtccccctgggcgagtactatgtgaaggaactgtccaaacctgtccacgggtcttgcagaaacatcacctgtgacaattggttcacatctattcccctggccaagaacctgctgcaggagccttataaactgactatcgtcggaaccgtgagaagcaacaagagggagattcccgaagtgctgaagaacagccggagcagacctgtcggcacttctatgttctgctttgacgggccactgaccctggtgagttacaagcccaaacctgctaaaatggtgtatctgctgtcaagctgtgacgaggatgcaagcatcaatgaatccaccggcaagccccagatggtcatgtactataaccagactaaaggcggggtggataccctgaatcagatgtgctctgtcatgacctgtagtagaaagacaaacaggtggcctatggccctgctgtacgggatgatcaacattgcttgcattaattcattcatcatctacagccacaacgtgtcctctaagggggagaaagtccagtcccgcaagaaattcatgcgaaatctgtacatgggactgaccagtagcttcatgaggaagcgcctggaggcacccacactgaaaaggtatctgcgcgacaacatcagcaatattctgcctaaggaagtgccaggcacttccgacgattctaccgaggaaccagtgatgaagaaacggacatactgcacttattgtcccagcaagatccgacggaaagcctccgcttcttgcaagaagtgtaagaaagtgatctgtagagagcataacattgatatgtgccggtcctgtttttga

SEQ ID NO: 8 - DNA, 1785 bp, synthetic construct
  source 1..1785: mol_type = other DNA
atgggcagcagcctggacgacgagcacatcctgagcgccctgctgcagagcgacgacgagctggtgggcgaggacagcgacagcgaggtgagcgaccacgtgagcgaggacgacgtgcagagcgacaccgaggaggccttcatcgacgaggtgcacgaggtgcagcccaccagcagcggcagcgagatcctggacgagcagaacgtgatcgagcagcccggcagcagcctggccagcaaccgcaacctgaccctgccccagcgcaccatccgcggcaagaacaagcactgctggagcaccagcaagcccacccgccgcagccgcgccagcgccctgaacatcgtgcgcagccagcgcggccccacccgcatgtgccgcaacatctacgaccccctgctgtgcttcaagctgttcttcaccgacgagatcatcagcgagatcgtgaagtggaccaacgccgagatcagcctgaagcgccgcgagagcatgaccagcgccaccttccgcgacaccaacgaggacgagatctacgccttcttcggcatcctggtgatgaccgccgtgcgcaaggacaaccacatgagcaccgacgacctgttcgaccgcagcctgagcatggtgtacgtgagcgtgatgagccgcgaccgcttcgacttcctgatccgctgcctgcgcatggacgacaagagcatccgccccaccctgcgcgagaacgacgtgttcacccccgtgcgcaagatctgggacctgttcatccaccagtgcatccagaactacacccccggcgcccacctgaccatcgacgagcagctgctgggcttccgcggccgctgccccttccgcgtgtacatccccaacaagcccagcaaatacggcatcaagatcctgatgatgtgcgacagcggcaccaagtacatgatcaacggcatgccctacctgggccgcggcacccagaccaacggcgtgcccctgggcgagtactacgtgaaggagctgagcaagcccgtgcacggcagctgccgcaacatcacctgcgacaactggttcaccagcatccccctggccaagaacctgctgcaggagccctacaagctgaccatcgtgggcaccgtgcgcagcaacaagcgcgagatccccgaggtgctgaagaacagccgcagccgccccgtgggcaccagcatgttctgcttcgacggccccctgaccctggtgagctacaagcccaagcccgccaagatggtgtacctgctgagcagctgcgacgaggacgccagcatcaacgagagcaccggcaagccccagatggtgatgtactacaaccagaccaagggcggcgtggacaccctggaccagatgtgcagcgtgatgacctgcagccgcaagaccaaccgctggcccatggccctgctgtacggcatgatcaacatcgcctgcatcaacagcttcatcatctacagccacaacgtgagcagcaagggcgagaaggtgcagagccgcaagaagttcatgcgcaacctgtacatgggcctgaccagcagcttcatgcgcaagcgcctggaggcccccaccctgaagcgctacctgcgcgacaacatcagcaacatcctgcccaaggaggtgcccggcaccagcgacgacagcaccgaggagcccgtgatgaagaagcgcacctactgcacctactgccccagcaagatccgccgcaaggccagcgccagctgcaagaagtgcaagaaggtgatctgccgcgagcacaacatcgacatgtgccggagctgcttctaa

SEQ ID NO: 9 - DNA, 1785 bp, synthetic construct
  source 1..1785: mol_type = other DNA
atgggctctagcctggacgacgagcacattctgtctgccctgctgcagtccgacgatgaactcgtgggcgaagattccgactccgagatctctgaccacgtgtccgaggacgacgtgcagtctgataccgaggaagccttcatcgacgaggtgcacgaagtgcagcctacctcttccggctctgagatcctggacgagcagaacgtgatcgagcagcctggatcctctctggcctccaacagaatcctgacactgccccagagaaccatccggggcaagaacaagcactgctggtccacctccaagtctacccggcggtctagagtgtccgctctgaatattgtgcggtcccagaggggccccaccagaatgtgccggaacatctacgaccctctgctgtgtttcaagctgttcttcaccgacgagatcatcagcgagatcgtgaagtggaccaacgccgagatcagcctgaagcggcgggaatctatgaccggcgccaccttcagagacaccaacgaggatgagatctacgccttcttcggcatcctggtcatgacagccgtgcggaaggacaaccacatgtccaccgacgacctgttcgacagatccctgtccatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgatgaacagctgctgggcttcagaggcagatgccccttcagaatgtacatccccaacaagccctctaagtacggcatcaagatcctgatgatgtgcgactccggcaccaagtacatgatcaacggcatgccctacctcggcagaggcacccaaacaaatggcgtgccactgggcgagtactatgtgaaagaactgtccaagcctgtgcacggctcctgcagaaacatcacctgtgacaactggttcaccagcattcctctggccaagaacctgctgcaagagccctacaagctgacaatcgtgggcaccgtgcggtccaacaagcgggaaattcctgaggtgctgaagaactctcggtccagacctgtgggcacctccatgttctgtttcgacggccctctgacactggtgtcctacaagcctaagcctgccaagatggtgtacctgctgtcctcctgtgacgaggacgccagcatcaatgagtccaccggcaagccccagatggtcatgtactacaaccagaccaaaggcggcgtggacaccctggaccagatgtgctctgtgatgacctgctccagaaagaccaacagatggcccatggctctgctgtacggcatgatcaatatcgcctgcatcaacagcttcatcatctactcccacaacgtgtcctccaagggcgagaaggtgcagtcccggaagaaattcatgcggaacctgtatatgtccctgacctccagcttcatgagaaagcggctggaagcccctactctgaagagatacctgcgggacaacatctccaacatcctgcctaacgaggtgcccggcaccagcgacgattctacagaggaacctgtgatgaagaagcggacctactgcacctactgtccctccaagatccggcggaaggccaacgcctcttgcaaaaagtgcaagaaagtgatctgccgcgagcacaacatcgacatgtgccagtcttgtttctga

SEQ ID NO: 10 - DNA, 4137 bp, synthetic construct
  source 1..4137: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctggaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtg

SEQ ID NO: 11 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 12 - DNA, 2472 bp, synthetic construct
  source 1..2472: mol_type = other DNA
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 13-22 - skipped (10 entries)

SEQ ID NO: 23 - DNA, 19 bp, synthetic construct
  source 1..19: mol_type = other DNA
tggccggcctgaccactgg

SEQ ID NO: 24 - DNA, 19 bp, synthetic construct
  source 1..19: mol_type = other DNA
tgaaggcctggccggcctg

SEQ ID NO: 25 - DNA, 19 bp, synthetic construct
  source 1..19: mol_type = other DNA
tgagcactgaaggcctggc

SEQ ID NO: 26 - DNA, 19 bp, synthetic construct
  source 1..19: mol_type = other DNA
tccactgagcactgaaggc

SEQ ID NO: 27 - DNA, 19 bp, synthetic construct
  source 1..19: mol_type = other DNA
tggtttccactgagcactg

SEQ ID NO: 28 - DNA, 19 bp, synthetic construct
  source 1..19: mol_type = other DNA
tggggaaaatgacccaaca

SEQ ID NO: 29 - DNA, 19 bp, synthetic construct
  source 1..19: mol_type = other DNA
taggacagtggggaaaatg

SEQ ID NO: 30 - DNA, 19 bp, synthetic construct
  source 1..19: mol_type = other DNA
tccagggacacggtgctag

SEQ ID NO: 31 - DNA, 19 bp, synthetic construct
  source 1..19: mol_type = other DNA
tcagagccaggagtcctgg

SEQ ID NO: 32 - DNA, 19 bp, synthetic construct
  source 1..19: mol_type = other DNA
tccttcagagccaggagtc

SEQ ID NO: 33 - DNA, 19 bp, synthetic construct
  source 1..19: mol_type = other DNA
tcctccttcagagccagga

SEQ ID NO: 34 - DNA, 19 bp, synthetic construct
  source 1..19: mol_type = other DNA
tccagcccctcctccttca

SEQ ID NO: 35 - DNA, 19 bp, synthetic construct
  source 1..19: mol_type = other DNA
tccgagcttgacccttgga

SEQ ID NO: 36 - DNA, 19 bp, synthetic construct
  source 1..19: mol_type = other DNA
tggtttccgagcttgaccc

SEQ ID NO: 37 - DNA, 19 bp, synthetic construct
  source 1..19: mol_type = other DNA
tggggtggtttccgagctt

SEQ ID NO: 38 - DNA, 19 bp, synthetic construct
  source 1..19: mol_type = other DNA
tctgctggggtggtttccg

SEQ ID NO: 39 - DNA, 19 bp, synthetic construct
  source 1..19: mol_type = other DNA
tgcagagtatctgctgggg

SEQ ID NO: 40 - DNA, 14 bp, synthetic construct
  source 1..14: mol_type = other DNA
ccaatcccctcagt

SEQ ID NO: 41 - DNA, 15 bp, synthetic construct
  source 1..15: mol_type = other DNA
cagtgctcagtggaa

SEQ ID NO: 42 - DNA, 18 bp, synthetic construct
  source 1..18: mol_type = other DNA
gaaacatccggcgactca

SEQ ID NO: 43 - DNA, 19 bp, synthetic construct
  source 1..19: mol_type = other DNA
tcgcccctcaaatcttaca

SEQ ID NO: 44 - DNA, 19 bp, synthetic construct
  source 1..19: mol_type = other DNA
tcaaatcttacagctgctc

SEQ ID NO: 45 - DNA, 19 bp, synthetic construct
  source 1..19: mol_type = other DNA
tcttacagctgctcactcc

SEQ ID NO: 46 - DNA, 19 bp, synthetic construct
  source 1..19: mol_type = other DNA
tacagctgctcactcccct

SEQ ID NO: 47 - DNA, 19 bp, synthetic construct
  source 1..19: mol_type = other DNA
tgctcactcccctgcaggg

SEQ ID NO: 48 - DNA, 19 bp, synthetic construct
  source 1..19: mol_type = other DNA
tcccctgcagggcaacgcc

SEQ ID NO: 49 - DNA, 19 bp, synthetic construct
  source 1..19: mol_type = other DNA
tgcagggcaacgcccaggg

SEQ ID NO: 50 - DNA, 19 bp, synthetic construct
  source 1..19: mol_type = other DNA
tctcgattatgggcgggat

SEQ ID NO: 51 - DNA, 19 bp, synthetic construct
  source 1..19: mol_type = other DNA
tcgcttctcgattatgggc

SEQ ID NO: 52 - DNA, 19 bp, synthetic construct
  source 1..19: mol_type = other DNA
tgtcgagtcgcttctcgat

SEQ ID NO: 53 - DNA, 19 bp, synthetic construct
  source 1..19: mol_type = other DNA
tccatgtcgagtcgcttct

SEQ ID NO: 54 - DNA, 19 bp, synthetic construct
  source 1..19: mol_type = other DNA
tcgcctccatgtcgagtcg

SEQ ID NO: 55 - DNA, 19 bp, synthetic construct
  source 1..19: mol_type = other DNA
tcgtcatcgcctccatgtc

SEQ ID NO: 56 - DNA, 19 bp, synthetic construct
  source 1..19: mol_type = other DNA
tgatctcgtcatcgcctcc

SEQ ID NO: 57 - DNA, 14 bp, synthetic construct
  source 1..14: mol_type = other DNA
gcttcagcttccta

SEQ ID NO: 58 - DNA, 14 bp, synthetic construct
  source 1..14: mol_type = other DNA
ctgtgatcatgcca

SEQ ID NO: 59 - DNA, 16 bp, synthetic construct
  source 1..16: mol_type = other DNA
acagtggtacacacct

SEQ ID NO: 60 - DNA, 15 bp, synthetic construct
  source 1..15: mol_type = other DNA
ccaccccccactaag

SEQ ID NO: 61 - DNA, 14 bp, synthetic construct
  source 1..14: mol_type = other DNA
cattggccgggcac

SEQ ID NO: 62 - DNA, 16 bp, synthetic construct
  source 1..16: mol_type = other DNA
gcttgaacccaggaga

SEQ ID NO: 63 - DNA, 17 bp, synthetic construct
  source 1..17: mol_type = other DNA
acacccgatccactggg

SEQ ID NO: 64 - DNA, 14 bp, synthetic construct
  source 1..14: mol_type = other DNA
gctgcatcaacccc

SEQ ID NO: 65 - DNA, 16 bp, synthetic construct
  source 1..16: mol_type = other DNA
gccacaaacagaaata

SEQ ID NO: 66 - DNA, 15 bp, synthetic construct
  source 1..15: mol_type = other DNA
ggtggctcatgcctg

SEQ ID NO: 67 - DNA, 16 bp, synthetic construct
  source 1..16: mol_type = other DNA
gatttgcacagctcat

SEQ ID NO: 68 - DNA, 15 bp, synthetic construct
  source 1..15: mol_type = other DNA
aagctctgaggagca

SEQ ID NO: 69 - DNA, 13 bp, synthetic construct
  source 1..13: mol_type = other DNA
ccctagctgtccc

SEQ ID NO: 70 - DNA, 14 bp, synthetic construct
  source 1..14: mol_type = other DNA
gcctagcatgctag

SEQ ID NO: 71 - DNA, 15 bp, synthetic construct
  source 1..15: mol_type = other DNA
atgggcttcacggat

SEQ ID NO: 72 - DNA, 14 bp, synthetic construct
  source 1..14: mol_type = other DNA
gaaactatgcctgc

SEQ ID NO: 73 - DNA, 14 bp, synthetic construct
  source 1..14: mol_type = other DNA
gcaccattgctccc

SEQ ID NO: 74 - DNA, 14 bp, synthetic construct
  source 1..14: mol_type = other DNA
gacatgcaactcag

SEQ ID NO: 75 - DNA, 14 bp, synthetic construct
  source 1..14: mol_type = other DNA
acaccactaggggt

SEQ ID NO: 76 - DNA, 14 bp, synthetic construct
  source 1..14: mol_type = other DNA
gtctgctagacagg

SEQ ID NO: 77 - DNA, 15 bp, synthetic construct
  source 1..15: mol_type = other DNA
ggcctagacaggctg

SEQ ID NO: 78 - DNA, 15 bp, synthetic construct
  source 1..15: mol_type = other DNA
gaggcattcttatcg

SEQ ID NO: 79 - DNA, 15 bp, synthetic construct
  source 1..15: mol_type = other DNA
gcctggaaacgttcc

SEQ ID NO: 80 - DNA, 14 bp, synthetic construct
  source 1..14: mol_type = other DNA
gtgctctgacaata

SEQ ID NO: 81 - DNA, 14 bp, synthetic construct
  source 1..14: mol_type = other DNA
gttttgcagcctcc

SEQ ID NO: 82 - DNA, 15 bp, synthetic construct
  source 1..15: mol_type = other DNA
acagctgtggaacgt

SEQ ID NO: 83 - DNA, 15 bp, synthetic construct
  source 1..15: mol_type = other DNA
ggctctcttcctcct

SEQ ID NO: 84 - DNA, 15 bp, synthetic construct
  source 1..15: mol_type = other DNA
ctatcccaaaactct

SEQ ID NO: 85 - DNA, 14 bp, synthetic construct
  source 1..14: mol_type = other DNA
gaaaaactatgtat

SEQ ID NO: 86 - DNA, 15 bp, synthetic construct
  source 1..15: mol_type = other DNA
aggcaggctggttga

SEQ ID NO: 87 - DNA, 14 bp, synthetic construct
  source 1..14: mol_type = other DNA
caatacaaccacgc

SEQ ID NO: 88 - DNA, 15 bp, synthetic construct
  source 1..15: mol_type = other DNA
atgacggactcaact

SEQ ID NO: 89 - DNA, 14 bp, synthetic construct
  source 1..14: mol_type = other DNA
cacaacatttgtaa

SEQ ID NO: 90 - DNA, 14 bp, synthetic construct
  source 1..14: mol_type = other DNA
atttccagtgcaca

SEQ ID NO: 91 - DNA, 20 bp, synthetic construct
  source 1..20: mol_type = other DNA
gtttagctcacccgtgagcc

SEQ ID NO: 92 - DNA, 20 bp, synthetic construct
  source 1..20: mol_type = other DNA
cccaatattattgttctctg

SEQ ID NO: 93 - DNA, 20 bp, synthetic construct
  source 1..20: mol_type = other DNA
ggggtgggataggggatacg

SEQ ID NO: 94 - DNA, 20 bp, synthetic construct
  source 1..20: mol_type = other DNA
ggatccccctctacatttaa

SEQ ID NO: 95 - DNA, 20 bp, synthetic construct
  source 1..20: mol_type = other DNA
gtgatcttgtacaaatcatt

SEQ ID NO: 96 - DNA, 20 bp, synthetic construct
  source 1..20: mol_type = other DNA
ctacacagaatctgttagaa

SEQ ID NO: 97 - DNA, 20 bp, synthetic construct
  source 1..20: mol_type = other DNA
taagctagagaatagatctc

SEQ ID NO: 98 - DNA, 20 bp, synthetic construct
  source 1..20: mol_type = other DNA
tcaatacacttaatgattta

SEQ ID NO: 99 - DNA, 25 bp, synthetic construct
  source 1..25: mol_type = other DNA
caccgggagccacgaaaacagatcc

SEQ ID NO: 100 - DNA, 25 bp, synthetic construct
  source 1..25: mol_type = other DNA
caccgcgaaaacagatccagggaca

SEQ ID NO: 101 - DNA, 25 bp, synthetic construct
  source 1..25: mol_type = other DNA
caccgagatccagggacacggtgct

SEQ ID NO: 102 - DNA, 25 bp, synthetic construct
  source 1..25: mol_type = other DNA
caccggacacggtgctaggacagtg

SEQ ID NO: 103 - DNA, 25 bp, synthetic construct
  source 1..25: mol_type = other DNA
caccggaaaatgacccaacagcctc

SEQ ID NO: 104 - DNA, 25 bp, synthetic construct
  source 1..25: mol_type = other DNA
caccggcctggccggcctgaccact

SEQ ID NO: 105 - DNA, 25 bp, synthetic construct
  source 1..25: mol_type = other DNA
caccgctgagcactgaaggcctggc

SEQ ID NO: 106 - DNA, 25 bp, synthetic construct
  source 1..25: mol_type = other DNA
caccgtggtttccactgagcactga

SEQ ID NO: 107 - DNA, 25 bp, synthetic construct
  source 1..25: mol_type = other DNA
caccggatagccaggagtcctttcg

SEQ ID NO: 108 - DNA, 25 bp, synthetic construct
  source 1..25: mol_type = other DNA
caccggcgcttccagtgctcagact

SEQ ID NO: 109 - DNA, 25 bp, synthetic construct
  source 1..25: mol_type = other DNA
caccgcagtgctcagactagggaag

SEQ ID NO: 110 - DNA, 25 bp, synthetic construct
  source 1..25: mol_type = other DNA
caccggcccctcctccttcagagcc

SEQ ID NO: 111 - DNA, 25 bp, synthetic construct
  source 1..25: mol_type = other DNA
caccgtccttcagagccaggagtcc

SEQ ID NO: 112 - DNA, 25 bp, synthetic construct
  source 1..25: mol_type = other DNA
caccgtggtttccgagcttgaccct

SEQ ID NO: 113 - DNA, 25 bp, synthetic construct
  source 1..25: mol_type = other DNA
caccgctgcagagtatctgctgggg

SEQ ID NO: 114 - DNA, 25 bp, synthetic construct
  source 1..25: mol_type = other DNA
caccgcgttcctgcagagtatctgc

SEQ ID NO: 115 - DNA, 25 bp, synthetic construct
  source 1..25: mol_type = other DNA
aaacggatctgttttcgtggctccc

SEQ ID NO: 116 - DNA, 25 bp, synthetic construct
  source 1..25: mol_type = other DNA
aaactgtccctggatctgttttcgc

SEQ ID NO: 117 - DNA, 25 bp, synthetic construct
  source 1..25: mol_type = other DNA
aaacagcaccgtgtccctggatctc

SEQ ID NO: 118 - DNA, 25 bp, synthetic construct
  source 1..25: mol_type = other DNA
aaaccactgtcctagcaccgtgtcc

SEQ ID NO: 119 - DNA, 25 bp, synthetic construct
  source 1..25: mol_type = other DNA
aaacgaggctgttgggtcattttcc

SEQ ID NO: 120 - DNA, 25 bp, synthetic construct
  source 1..25: mol_type = other DNA
aaacagtggtcaggccggccaggcc

SEQ ID NO: 121 - DNA, 25 bp, synthetic construct
  source 1..25: mol_type = other DNA
aaacgccaggccttcagtgctcagc

SEQ ID NO: 122 - DNA, 25 bp, synthetic construct
  source 1..25: mol_type = other DNA
aaactcagtgctcagtggaaaccac

SEQ ID NO: 123 - DNA, 25 bp, synthetic construct
  source 1..25: mol_type = other DNA
aaaccgaaaggactcctggctatcc

SEQ ID NO: 124 - DNA, 25 bp, synthetic construct
  source 1..25: mol_type = other DNA
aaacagtctgagcactggaagcgcc

SEQ ID NO: 125 - DNA, 25 bp, synthetic construct
  source 1..25: mol_type = other DNA
aaaccttccctagtctgagcactgc

SEQ ID NO: 126 - DNA, 25 bp, synthetic construct
  source 1..25: mol_type = other DNA
aaacggctctgaaggaggaggggcc

SEQ ID NO: 127 - DNA, 25 bp, synthetic construct
  source 1..25: mol_type = other DNA
aaacggactcctggctctgaaggac

SEQ ID NO: 128 - DNA, 25 bp, synthetic construct
  source 1..25: mol_type = other DNA
aaacagggtcaagctcggaaaccac

SEQ ID NO: 129 - DNA, 25 bp, synthetic construct
  source 1..25: mol_type = other DNA
aaacccccagcagatactctgcagc

SEQ ID NO: 130 - DNA, 25 bp, synthetic construct
  source 1..25: mol_type = other DNA
aaacgcagatactctgcaggaacgc

SEQ ID NO: 131 - DNA, 20 bp, synthetic construct
  source 1..20: mol_type = other DNA
tcccctcccagaaagacctg

SEQ ID NO: 132 - DNA, 20 bp, synthetic construct
  source 1..20: mol_type = other DNA
tgggctccaagcaatcctgg

SEQ ID NO: 133 - DNA, 20 bp, synthetic construct
  source 1..20: mol_type = other DNA
gtggctcaggaggtacctgg

SEQ ID NO: 134 - DNA, 20 bp, synthetic construct
  source 1..20: mol_type = other DNA
gagccacgaaaacagatcca

SEQ ID NO: 135 - DNA, 20 bp, synthetic construct
  source 1..20: mol_type = other DNA
aagtgaacggggaagggagg

SEQ ID NO: 136 - DNA, 20 bp, synthetic construct
  source 1..20: mol_type = other DNA
gacaaaagccgaagtccagg

SEQ ID NO: 137 - DNA, 20 bp, synthetic construct
  source 1..20: mol_type = other DNA
gtggttgataaacccacgtg

SEQ ID NO: 138 - DNA, 20 bp, synthetic construct
  source 1..20: mol_type = other DNA
tgggaacagccacagcaggg

SEQ ID NO: 139 - DNA, 20 bp, synthetic construct
  source 1..20: mol_type = other DNA
gcaggggaacggggatgcag

SEQ ID NO: 140 - DNA, 20 bp, synthetic construct
  source 1..20: mol_type = other DNA
gagatggtggacgaggaagg

SEQ ID NO: 141 - DNA, 20 bp, synthetic construct
  source 1..20: mol_type = other DNA
gagatggctccaggaaatgg

SEQ ID NO: 142 - DNA, 20 bp, synthetic construct
  source 1..20: mol_type = other DNA
taaggaatctgcctaacagg

SEQ ID NO: 143 - DNA, 20 bp, synthetic construct
  source 1..20: mol_type = other DNA
tcaggagactaggaaggagg

SEQ ID NO: 144 - DNA, 20 bp, synthetic construct
  source 1..20: mol_type = other DNA
tataaggtggtcccagctcg

SEQ ID NO: 145 - DNA, 20 bp, synthetic construct
  source 1..20: mol_type = other DNA
ctggaagatgccatgacagg

SEQ ID NO: 146 - DNA, 20 bp, synthetic construct
  source 1..20: mol_type = other DNA
gcacagactagagaggtaag

SEQ ID NO: 147 - DNA, 20 bp, synthetic construct
  source 1..20: mol_type = other DNA
acagactagagaggtaaggg

SEQ ID NO: 148 - DNA, 20 bp, synthetic construct
  source 1..20: mol_type = other DNA
gagaggtgacccgaatccac

SEQ ID NO: 149 - DNA, 20 bp, synthetic construct
  source 1..20: mol_type = other DNA
gcacaggccccagaaggaga

SEQ ID NO: 150 - DNA, 20 bp, synthetic construct
  source 1..20: mol_type = other DNA
ccggagaggacccagacacg

SEQ ID NO: 151 - DNA, 20 bp, synthetic construct
  source 1..20: mol_type = other DNA
gagaggacccagacacgggg

SEQ ID NO: 152 - DNA, 20 bp, synthetic construct
  source 1..20: mol_type = other DNA
gcaacacagcagagagcaag

SEQ ID NO: 153 - DNA, 20 bp, synthetic construct
  source 1..20: mol_type = other DNA
gaagagggagtggaggaaga

SEQ ID NO: 154 - DNA, 20 bp, synthetic construct
  source 1..20: mol_type = other DNA
aagacggaacctgaaggagg

SEQ ID NO: 155 - DNA, 20 bp, synthetic construct
  source 1..20: mol_type = other DNA
agaaagcggcacaggcccag

SEQ ID NO: 156 - DNA, 20 bp, synthetic construct
  source 1..20: mol_type = other DNA
gggaaacagtgggccagagg

SEQ ID NO: 157 - DNA, 20 bp, synthetic construct
  source 1..20: mol_type = other DNA
gtccggactcaggagagaga

SEQ ID NO: 158 - DNA, 20 bp, synthetic construct
  source 1..20: mol_type = other DNA
ggcacagcaagggcactcgg

SEQ ID NO: 159 - DNA, 20 bp, synthetic construct
  source 1..20: mol_type = other DNA
gaagaggggaagtcgaggga

SEQ ID NO: 160 - DNA, 20 bp, synthetic construct
  source 1..20: mol_type = other DNA
gggaatggtaaggaggcctg

SEQ ID NO: 161 - DNA, 20 bp, synthetic construct
  source 1..20: mol_type = other DNA
gcagagtggtcagcacagag

SEQ ID NO: 162 - DNA, 20 bp, synthetic construct
  source 1..20: mol_type = other DNA
gcacagagtggctaagccca

SEQ ID NO: 163 - DNA, 20 bp, synthetic construct
  source 1..20: mol_type = other DNA
gacggggtgtcagcataggg

SEQ ID NO: 164 - DNA, 20 bp, synthetic construct
  source 1..20: mol_type = other DNA
gcccagggccaggaacgacg

SEQ ID NO: 165 - DNA, 20 bp, synthetic construct
  source 1..20: mol_type = other DNA
ggtggagtccagcacggcgc

SEQ ID NO: 166 - DNA, 20 bp, synthetic construct
  source 1..20: mol_type = other DNA
acaggccgccaggaactcgg

SEQ ID NO: 167 - DNA, 20 bp, synthetic construct
  source 1..20: mol_type = other DNA
actaggaagtgtgtagcacc

SEQ ID NO: 168 - DNA, 20 bp, synthetic construct
  source 1..20: mol_type = other DNA
atgaatagcagactgccccg

SEQ ID NO: 169 - DNA, 20 bp, synthetic construct
  source 1..20: mol_type = other DNA
acacccctaaaagcacagtg

SEQ ID NO: 170 - DNA, 20 bp, synthetic construct
  source 1..20: mol_type = other DNA
caaggagttccagcaggtgg

SEQ ID NO: 171 - DNA, 20 bp, synthetic construct
  source 1..20: mol_type = other DNA
aaggagttccagcaggtggg

SEQ ID NO: 172 - DNA, 20 bp, synthetic construct
  source 1..20: mol_type = other DNA
tggaaagaggagggaagagg

SEQ ID NO: 173 - DNA, 20 bp, synthetic construct
  source 1..20: mol_type = other DNA
tcgaattcctaactgccccg

SEQ ID NO: 174 - DNA, 20 bp, synthetic construct
  source 1..20: mol_type = other DNA
gacctgcccagcacaccctg

SEQ ID NO: 175 - DNA, 20 bp, synthetic construct
  source 1..20: mol_type = other DNA
ggagcagctgcggcagtggg

SEQ ID NO: 176 - DNA, 20 bp, synthetic construct
  source 1..20: mol_type = other DNA
gggagggagagcttggcagg

SEQ ID NO: 177 - DNA, 20 bp, synthetic construct
  source 1..20: mol_type = other DNA
gttacgtggccaagaagcag

SEQ ID NO: 178 - DNA, 20 bp, synthetic construct
  source 1..20: mol_type = other DNA
gctgaacagagaagagctgg

SEQ ID NO: 179 - DNA, 20 bp, synthetic construct
  source 1..20: mol_type = other DNA
tctgagggtggagggactgg

SEQ ID NO: 180 - DNA, 20 bp, synthetic construct
  source 1..20: mol_type = other DNA
ggagaggtgagggacttggg

SEQ ID NO: 181 - DNA, 20 bp, synthetic construct
  source 1..20: mol_type = other DNA
gtgaaccaggcagacaacga

SEQ ID NO: 182 - DNA, 20 bp, synthetic construct
  source 1..20: mol_type = other DNA
caggtacctcctgagccacg

SEQ ID NO: 183 - DNA, 20 bp, synthetic construct
  source 1..20: mol_type = other DNA
gggggagtaggggcatgcag

SEQ ID NO: 184 - DNA, 20 bp, synthetic construct
  source 1..20: mol_type = other DNA
gcaaatggccagcaagggtg

SEQ ID NO: 185 - DNA, 25 bp, synthetic construct
  source 1..25: mol_type = other DNA
caccgaatcgagaagcgactcgaca

SEQ ID NO: 186 - DNA, 25 bp, synthetic construct
  source 1..25: mol_type = other DNA
caccggtccctgggcgttgccctgc

SEQ ID NO: 187 - DNA, 25 bp, synthetic construct
  source 1..25: mol_type = other DNA
caccgccctgggcgttgccctgcag

SEQ ID NO: 188 - DNA, 25 bp, synthetic construct
  source 1..25: mol_type = other DNA
caccgccgtgggaagataaactaat

SEQ ID NO: 189 - DNA, 25 bp, synthetic construct
  source 1..25: mol_type = other DNA
caccgtcccctgcagggcaacgccc

SEQ ID NO: 190 - DNA, 25 bp, synthetic construct
  source 1..25: mol_type = other DNA
caccggtcgagtcgcttctcgatta

SEQ ID NO: 191 - DNA, 25 bp, synthetic construct
  source 1..25: mol_type = other DNA
caccgctgctgcctcccgtcttgta

SEQ ID NO: 192 - DNA, 25 bp, synthetic construct
  source 1..25: mol_type = other DNA
caccggagtgccgcaatacctttat

SEQ ID NO: 193 - DNA, 25 bp, synthetic construct
  source 1..25: mol_type = other DNA
caccgacactttggtggtgcagcaa

SEQ ID NO: 194 - DNA, 25 bp, synthetic construct
  source 1..25: mol_type = other DNA
caccgtctcaaatggtataaaactc

SEQ ID NO: 195 - DNA, 25 bp, synthetic construct
  source 1..25: mol_type = other DNA
caccgaatcccgcccataatcgaga

SEQ ID NO: 196 - DNA, 25 bp, synthetic construct
  source 1..25: mol_type = other DNA
caccgtcccgcccataatcgagaag

SEQ ID NO: 197 - DNA, 25 bp, synthetic construct
  source 1..25: mol_type = other DNA
caccgcccataatcgagaagcgact

SEQ ID NO: 198 - DNA, 25 bp, synthetic construct
  source 1..25: mol_type = other DNA
caccggagaagcgactcgacatgga

SEQ ID NO: 199 - DNA, 25 bp, synthetic construct
  source 1..25: mol_type = other DNA
caccggaagcgactcgacatggagg

SEQ ID NO: 200 - DNA, 25 bp, synthetic construct
  source 1..25: mol_type = other DNA
caccggcgactcgacatggaggcga

SEQ ID NO: 201 - DNA, 25 bp, synthetic construct
  source 1..25: mol_type = other DNA
aaactgtcgagtcgcttctcgattc

SEQ ID NO: 202 - DNA, 25 bp, synthetic construct
  source 1..25: mol_type = other DNA
aaacgcagggcaacgcccagggacc

SEQ ID NO: 203 - DNA, 25 bp, synthetic construct
  source 1..25: mol_type = other DNA
aaacctgcagggcaacgcccagggc

SEQ ID NO: 204 - DNA, 25 bp, synthetic construct
  source 1..25: mol_type = other DNA
aaacattagtttatcttcccacggc

SEQ ID NO: 205 - DNA, 25 bp, synthetic construct
  source 1..25: mol_type = other DNA
aaacgggcgttgccctgcaggggac

SEQ ID NO: 206 - DNA, 25 bp, synthetic construct
  source 1..25: mol_type = other DNA
aaactaatcgagaagcgactcgacc

SEQ ID NO: 207 - DNA, 25 bp, synthetic construct
  source 1..25: mol_type = other DNA
aaactacaagacgggaggcagcagc

SEQ ID NO: 208 - DNA, 25 bp, synthetic construct
  source 1..25: mol_type = other DNA
aaacataaaggtattgcggcactcc

SEQ ID NO: 209 - DNA, 25 bp, synthetic construct
  source 1..25: mol_type = other DNA
aaacttgctgcaccaccaaagtgtc

SEQ ID NO: 210 - DNA, 25 bp, synthetic construct
  source 1..25: mol_type = other DNA
aaacgagttttataccatttgagac

SEQ ID NO: 211 - DNA, 25 bp, synthetic construct
  source 1..25: mol_type = other DNA
aaactctcgattatgggcgggattc

SEQ ID NO: 212 - DNA, 25 bp, synthetic construct
  source 1..25: mol_type = other DNA
aaaccttctcgattatgggcgggac

SEQ ID NO: 213 - DNA, 25 bp, synthetic construct
  source 1..25: mol_type = other DNA
aaacagtcgcttctcgattatgggc

SEQ ID NO: 214 - DNA, 25 bp, synthetic construct
  source 1..25: mol_type = other DNA
aaactccatgtcgagtcgcttctcc

SEQ ID NO: 215 - DNA, 25 bp, synthetic construct
  source 1..25: mol_type = other DNA
aaaccctccatgtcgagtcgcttcc

SEQ ID NO: 216 - DNA, 25 bp, synthetic construct
  source 1..25: mol_type = other DNA
aaactcgcctccatgtcgagtcgcc

SEQ ID NO: 217 - DNA, 25 bp, synthetic construct
  source 1..25: mol_type = other DNA
caccgacagggttaatgtgaagtcc

SEQ ID NO: 218 - DNA, 25 bp, synthetic construct
  source 1..25: mol_type = other DNA
caccgtccccctctacatttaaagt

SEQ ID NO: 219 - DNA, 25 bp, synthetic construct
  source 1..25: mol_type = other DNA
caccgcatttaaagttggtttaagt

SEQ ID NO: 220 - DNA, 25 bp, synthetic construct
  source 1..25: mol_type = other DNA
caccgttagaaaatataaagaataa

SEQ ID NO: 221 - DNA, 25 bp, synthetic construct
  source 1..25: mol_type = other DNA
caccgtaaatgcttactggtttgaa

SEQ ID NO: 222 - DNA, 25 bp, synthetic construct
  source 1..25: mol_type = other DNA
caccgtcctgggtccagaaaaagat

SEQ ID NO: 223 - DNA, 25 bp, synthetic construct
  source 1..25: mol_type = other DNA
caccgttgggtggtgagcatctgtg

SEQ ID NO: 224 - DNA, 25 bp, synthetic construct
  source 1..25: mol_type = other DNA
caccgcggggagagtggagaaaaag

SEQ ID NO: 225 - DNA, 25 bp, synthetic construct
  source 1..25: mol_type = other DNA
caccggttaaaactctttagacaac

SEQ ID NO: 226 - DNA, 25 bp, synthetic construct
  source 1..25: mol_type = other DNA
caccggaaaatccccactaagatcc

SEQ ID NO: 227 - DNA, 25 bp, synthetic construct
  source 1..25: mol_type = other DNA
aaacggacttcacattaaccctgtc

SEQ ID NO: 228 - DNA, 25 bp, synthetic construct
  source 1..25: mol_type = other DNA
aaacactttaaatgtagagggggac

SEQ ID NO: 229 - DNA, 25 bp, synthetic construct
  source 1..25: mol_type = other DNA
aaacacttaaaccaactttaaatgc

SEQ ID NO: 230 - DNA, 25 bp, synthetic construct
  source 1..25: mol_type = other DNA
aaacttattctttatattttctaac

SEQ ID NO: 231 - DNA, 25 bp, synthetic construct
  source 1..25: mol_type = other DNA
aaacttcaaaccagtaagcatttac

SEQ ID NO: 232 - DNA, 25 bp, synthetic construct
  source 1..25: mol_type = other DNA
aaacatctttttctggacccaggac

SEQ ID NO: 233 - DNA, 25 bp, synthetic construct
  source 1..25: mol_type = other DNA
aaaccacagatgctcaccacccaac

SEQ ID NO: 234 - DNA, 25 bp, synthetic construct
  source 1..25: mol_type = other DNA
aaacctttttctccactctccccgc

SEQ ID NO: 235 - DNA, 25 bp, synthetic construct
  source 1..25: mol_type = other DNA
aaacgttgtctaaagagttttaacc

SEQ ID NO: 236 - DNA, 25 bp, synthetic construct
  source 1..25: mol_type = other DNA
aaacggatcttagtggggattttcc

SEQ ID NO: 237 - DNA, 20 bp, synthetic construct
  source 1..20: mol_type = other DNA
agtagcagtaatgaagctgg

SEQ ID NO: 238 - DNA, 20 bp, synthetic construct
  source 1..20: mol_type = other DNA
atacccagacgagaaagctg

SEQ ID NO: 239 - DNA, 20 bp, synthetic construct
  source 1..20: mol_type = other DNA
tacccagacgagaaagctga

SEQ ID NO: 240 - DNA, 20 bp, synthetic construct
  source 1..20: mol_type = other DNA
ggtggtgagcatctgtgtgg

SEQ ID NO: 241 - DNA, 20 bp, synthetic construct
  source 1..20: mol_type = other DNA
aaatgagaagaagaggcaca

SEQ ID NO: 242 - DNA, 20 bp, synthetic construct
  source 1..20: mol_type = other DNA
cttgtggcctgggagagctg

SEQ ID NO: 243 - DNA, 19 bp, synthetic construct
  source 1..19: mol_type = other DNA
gctgtagaaggagacagag

SEQ ID NO: 244 - DNA, 20 bp, synthetic construct
  source 1..20: mol_type = other DNA
gagctggttgggaagacatg

SEQ ID NO: 245 - DNA, 20 bp, synthetic construct
  source 1..20: mol_type = other DNA
ctggttgggaagacatgggg

SEQ ID NO: 246 - DNA, 20 bp, synthetic construct
  source 1..20: mol_type = other DNA
cgtgaggatgggaaggaggg

SEQ ID NO: 247 - DNA, 20 bp, synthetic construct
  source 1..20: mol_type = other DNA
atgcagagtcagcagaactg

SEQ ID NO: 248 - DNA, 20 bp, synthetic construct
  source 1..20: mol_type = other DNA
aagacatcaagcacagaagg

SEQ ID NO: 249 - DNA, 20 bp, synthetic construct
  source 1..20: mol_type = other DNA
tcaagcacagaaggaggagg

SEQ ID NO: 250 - DNA, 20 bp, synthetic construct
  source 1..20: mol_type = other DNA
aaccgtcaataggcaaaggg

SEQ ID NO: 251 - DNA, 20 bp, synthetic construct
  source 1..20: mol_type = other DNA
ccgtatttcagactgaatgg

SEQ ID NO: 252 - DNA, 20 bp, synthetic construct
  source 1..20: mol_type = other DNA
gagaggacaggtgctacagg

SEQ ID NO: 253 - DNA, 20 bp, synthetic construct
  source 1..20: mol_type = other DNA
aaccaaggaagggcaggagg

SEQ ID NO: 254 - DNA, 20 bp, synthetic construct
  source 1..20: mol_type = other DNA
gacctctgggtggagacaga

SEQ ID NO: 255 - DNA, 20 bp, synthetic construct
  source 1..20: mol_type = other DNA
cagatgaccatgacaagcag

SEQ ID NO: 256 - DNA, 20 bp, synthetic construct
  source 1..20: mol_type = other DNA
aacaccagtgagtagagcgg

SEQ ID NO: 257 - DNA, 20 bp, synthetic construct
  source 1..20: mol_type = other DNA
aggaccttgaagcacagaga

SEQ ID NO: 258 - DNA, 20 bp, synthetic construct
  source 1..20: mol_type = other DNA
tacagaggcagactaaccca

SEQ ID NO: 259 - DNA, 20 bp, synthetic construct
  source 1..20: mol_type = other DNA
acagaggcagactaacccag

SEQ ID NO: 260 - DNA, 20 bp, synthetic construct
  source 1..20: mol_type = other DNA
taaatgacgtgctagacctg

SEQ ID NO: 261 - DNA, 20 bp, synthetic construct
  source 1..20: mol_type = other DNA
agtaaccactcaggacaggg

SEQ ID NO: 262 - DNA, 20 bp, synthetic construct
  source 1..20: mol_type = other DNA
accacaaaacagaaacacca

SEQ ID NO: 263 - DNA, 20 bp, synthetic construct
  source 1..20: mol_type = other DNA
gtttgaagacaagcctgagg

SEQ ID NO: 264 - DNA, 20 bp, synthetic construct
  source 1..20: mol_type = other DNA
gctgaaccccaaaagacagg

SEQ ID NO: 265 - DNA, 20 bp, synthetic construct
  source 1..20: mol_type = other DNA
gcagctgagacacacaccag

SEQ ID NO: 266 - DNA, 20 bp, synthetic construct
  source 1..20: mol_type = other DNA
aggacaccccaaagaagctg

SEQ ID NO: 267 - DNA, 20 bp, synthetic construct
  source 1..20: mol_type = other DNA
ggacaccccaaagaagctga

SEQ ID NO: 268 - DNA, 20 bp, synthetic construct
  source 1..20: mol_type = other DNA
ccagtgcaatggacagaaga

SEQ ID NO: 269 - DNA, 20 bp, synthetic construct
  source 1..20: mol_type = other DNA
agaagagggagcctgcaagt

SEQ ID NO: 270 - DNA, 20 bp, synthetic construct
  source 1..20: mol_type = other DNA
gtgtttgggccctagagcga

SEQ ID NO: 271 - DNA, 20 bp, synthetic construct
  source 1..20: mol_type = other DNA
catgtgcctggtgcaatgca

SEQ ID NO: 272 - DNA, 20 bp, synthetic construct
  source 1..20: mol_type = other DNA
tacaaagaggaagataagtg

SEQ ID NO: 273 - DNA, 20 bp, synthetic construct
  source 1..20: mol_type = other DNA
gtcacagaatacaccactag

SEQ ID NO: 274 - DNA, 20 bp, synthetic construct
  source 1..20: mol_type = other DNA
gggttaccctggacatggaa

SEQ ID NO: 275 - DNA, 20 bp, synthetic construct
  source 1..20: mol_type = other DNA
catggaagggtattcactcg

SEQ ID NO: 276 - DNA, 20 bp, synthetic construct
  source 1..20: mol_type = other DNA
agagtggcctagacaggctg

SEQ ID NO: 277 - DNA, 20 bp, synthetic construct
  source 1..20: mol_type = other DNA
catgctggacagctcggcag

SEQ ID NO: 278 - DNA, 20 bp, synthetic construct
  source 1..20: mol_type = other DNA
agtgaaagaagagaaaattc

SEQ ID NO: 279 - DNA, 20 bp, synthetic construct
  source 1..20: mol_type = other DNA
tggtaagtctaagaaaccta

SEQ ID NO: 280 - DNA, 20 bp, synthetic construct
  source 1..20: mol_type = other DNA
cccacagcctaaccacccta

SEQ ID NO: 281 - DNA, 20 bp, synthetic construct
  source 1..20: mol_type = other DNA
aatatttcaaagccctaggg

SEQ ID NO: 282 - DNA, 20 bp, synthetic construct
  source 1..20: mol_type = other DNA
gcactcggaacagggtctgg

SEQ ID NO: 283 - DNA, 20 bp, synthetic construct
  source 1..20: mol_type = other DNA
agataggagctccaacagtg

SEQ ID NO: 284 - DNA, 20 bp, synthetic construct
  source 1..20: mol_type = other DNA
aagttagagcagccaggaaa

SEQ ID NO: 285 - DNA, 20 bp, synthetic construct
  source 1..20: mol_type = other DNA
tagagcagccaggaaaggga

SEQ ID NO: 286 - DNA, 20 bp, synthetic construct
  source 1..20: mol_type = other DNA
tgaatacccttccatgtcca

SEQ ID NO: 287 - DNA, 20 bp, synthetic construct
  source 1..20: mol_type = other DNA
cctgcattgcaccaggcaca

SEQ ID NO: 288 - DNA, 20 bp, synthetic construct
  source 1..20: mol_type = other DNA
tctagggcccaaacacacct

SEQ ID NO: 289 - DNA, 20 bp, synthetic construct
  source 1..20: mol_type = other DNA
tccctccatctatcaaaagg

SEQ ID NO: 290 - DNA, 20 bp, synthetic construct
  source 1..20: mol_type = other DNA
agccctgagacagaagcagg

SEQ ID NO: 291 - DNA, 20 bp, synthetic construct
  source 1..20: mol_type = other DNA
gccctgagacagaagcaggt

SEQ ID NO: 292 - DNA, 20 bp, synthetic construct
  source 1..20: mol_type = other DNA
aggagatgcagtgatacgca

SEQ ID NO: 293 - DNA, 20 bp, synthetic construct
  source 1..20: mol_type = other DNA
acaataccaagggtatccgg

SEQ ID NO: 294 - DNA, 20 bp, synthetic construct
  source 1..20: mol_type = other DNA
tgataaagaaaacaaagtga

SEQ ID NO: 295 - DNA, 20 bp, synthetic construct
  source 1..20: mol_type = other DNA
aaagaaaacaaagtgaggga

SEQ ID NO: 296 - DNA, 20 bp, synthetic construct
  source 1..20: mol_type = other DNA
gtggcaagtggagaaattga

SEQ ID NO: 297 - DNA, 20 bp, synthetic construct
  source 1..20: mol_type = other DNA
caagtggagaaattgaggga

SEQ ID NO: 298 - DNA, 20 bp, synthetic construct
  source 1..20: mol_type = other DNA
gtggtgatgattgcagctgg

SEQ ID NO: 299 - DNA, 20 bp, synthetic construct
  source 1..20: mol_type = other DNA
ctatgtgcctgacacacagg

SEQ ID NO: 300 - DNA, 20 bp, synthetic construct
  source 1..20: mol_type = other DNA
gggttggaccaggaaagagg

SEQ ID NO: 301 - DNA, 20 bp, synthetic construct
  source 1..20: mol_type = other DNA
gatgcctggaaaaggaaaga

SEQ ID NO: 302 - DNA, 20 bp, synthetic construct
  source 1..20: mol_type = other DNA
tagtatgcacctgcaagagg

SEQ ID NO: 303 - DNA, 20 bp, synthetic construct
  source 1..20: mol_type = other DNA
tatgcacctgcaagaggcgg

SEQ ID NO: 304 - DNA, 20 bp, synthetic construct
  source 1..20: mol_type = other DNA
aggggaagaagagaagcaga

SEQ ID NO: 305 - DNA, 20 bp, synthetic construct
  source 1..20: mol_type = other DNA
gctgaatcaagagacaagcg

SEQ ID NO: 306 - DNA, 20 bp, synthetic construct
  source 1..20: mol_type = other DNA
aagcaaataaatctcctggg

SEQ ID NO: 307 - DNA, 20 bp, synthetic construct
  source 1..20: mol_type = other DNA
agatgagtgctagagactgg

SEQ ID NO: 308 - DNA, 20 bp, synthetic construct
  source 1..20: mol_type = other DNA
ctgatggttgagcacagcag

SEQ ID NO: 309 - DNA, 20 bp, synthetic construct
  source 1..20: mol_type = other DNA
caaatggccagcaagggtgg

SEQ ID NO: 310 - DNA, 20 bp, synthetic construct
  source 1..20: mol_type = other DNA
gcagaacctgaggatatgga

SEQ ID NO: 311 - DNA, 20 bp, synthetic construct
  source 1..20: mol_type = other DNA
aatacacagaatgaaaatag

SEQ ID NO: 312 - DNA, 20 bp, synthetic construct
  source 1..20: mol_type = other DNA
ctggtgactagaataggcag

SEQ ID NO: 313 - DNA, 20 bp, synthetic construct
  source 1..20: mol_type = other DNA
tggtgactagaataggcagt

SEQ ID NO: 314 - DNA, 20 bp, synthetic construct
  source 1..20: mol_type = other DNA
taaaagaatgtgaaaagatg

SEQ ID NO: 315 - DNA, 20 bp, synthetic construct
  source 1..20: mol_type = other DNA
tcaggagttcaagaccaccc

SEQ ID NO: 316 - DNA, 20 bp, synthetic construct
  source 1..20: mol_type = other DNA
tgtagtcccagttatgcagg

SEQ ID NO: 317 - DNA, 20 bp, synthetic construct
  source 1..20: mol_type = other DNA
gggttcacaccacaaatgca

SEQ ID NO: 318 - DNA, 20 bp, synthetic construct
  source 1..20: mol_type = other DNA
ggcaaatggccagcaagggt

SEQ ID NO: 319 - DNA, 20 bp, synthetic construct
  source 1..20: mol_type = other DNA
agaaaccaatcccaaagcaa

SEQ ID NO: 320 - DNA, 20 bp, synthetic construct
  source 1..20: mol_type = other DNA
gccaaggacaccaaaaccca

SEQ ID NO: 321 - DNA, 20 bp, synthetic construct
  source 1..20: mol_type = other DNA
agtggtgataaggcaacagt

SEQ ID NO: 322 - DNA, 20 bp, synthetic construct
  source 1..20: mol_type = other DNA
cctgagacagaagtattaag

SEQ ID NO: 323 - DNA, 20 bp, synthetic construct
  source 1..20: mol_type = other DNA
aaggtcacacaatgaatagg

SEQ ID NO: 324 - DNA, 20 bp, synthetic construct
  source 1..20: mol_type = other DNA
caccatactagggaagaaga

SEQ ID NO: 325 - DNA, 20 bp, synthetic construct
  source 1..20: mol_type = other DNA
aataccctgcccttagtggg

SEQ ID NO: 326 - DNA, 20 bp, synthetic construct
  source 1..20: mol_type = other DNA
ttagtggggggtggagtggg

SEQ ID NO: 327 - DNA, 20 bp, synthetic construct
  source 1..20: mol_type = other DNA
caataccctgcccttagtgg

SEQ ID NO: 328 - DNA, 20 bp, synthetic construct
  source 1..20: mol_type = other DNA
gtggggggtggagtgggggg

SEQ ID NO: 329 - DNA, 20 bp, synthetic construct
  source 1..20: mol_type = other DNA
ggggggtggagtggggggtg

SEQ ID NO: 330 - DNA, 20 bp, synthetic construct
  source 1..20: mol_type = other DNA
ggggtggagtggggggtggg

SEQ ID NO: 331 - DNA, 20 bp, synthetic construct
  source 1..20: mol_type = other DNA
gggtggagtggggggtgggg

SEQ ID NO: 332 - DNA, 20 bp, synthetic construct
  source 1..20: mol_type = other DNA
gggggtggggaaagacatcg

SEQ ID NO: 333 - DNA, 20 bp, synthetic construct
  source 1..20: mol_type = other DNA
gcagctgtgaattctgatag

SEQ ID NO: 334 - DNA, 20 bp, synthetic construct
  source 1..20: mol_type = other DNA
gagatcagagaaaccagatg

SEQ ID NO: 335 - DNA, 20 bp, synthetic construct
  source 1..20: mol_type = other DNA
tctatactgattgcagccag

SEQ ID NO: 336-348 - skipped (13 entries)

SEQ ID NO: 349 - protein, 19 aa, synthetic construct
  SITE 1: note = K may be replaced with R
  SITE 2: note = K may be replaced with R
  SITE 13: note = The amino acid at this position may be omitted.
  SITE 14: note = The amino acid at this position may be omitted.
  REGION 15..19: note = At least three of the five amino acids is either K or R.
KKXXXXXXXXXXXXKKKKK

SEQ ID NO: 350 - protein, 9 aa, synthetic construct
PAAKRVKLD

SEQ ID NO: 351 - protein, 16 aa, synthetic construct
KRPAATKKAGQAKKKK

SEQ ID NO: 352 - protein, 7 aa, synthetic construct
PKKKRKV

SEQ ID NO: 353 - protein, 24 aa, synthetic construct
DPKKKRKVDPKKKRKVDPKKKRKV

SEQ ID NO: 354-422 - skipped (69 entries)

SEQ ID NO: 423 - DNA, 333 bp, synthetic construct
  source 1..333: mol_type = other DNA
ggcggatctggcggtagtgctgagtattgtctgagttacgaaacggaaatactcacggttgagtatgggcttcttccaattggcaaaatcgttgaaaagcgcatagagtgtacggtgtattccgtcgataacaacggtaatatctacacccagccggtagctcagtggcacgaccgaggcgaacaggaagtgttcgagtattgcttggaagatggctcccttatccgcgccactaaagaccataagtttatgacggttgacgggcagatgctgcctatagacgaaatatttgagagagagctggacttgatgagagtcgataatctgccaaat

SEQ ID NO: 424 - DNA, 180 bp, synthetic construct
  source 1..180: mol_type = other DNA
ggcggatctggcggtagtgggggttccggatccataaagatagctactaggaaatatcttggcaaacaaaacgtctatgacataggagttgagcgagatcacaattttgctttgaagaatgggttcatcgcgtctaattgcttcaacgctagcggcgggtcaggaggctctggtggaagc

SEQ ID NO: 425 - DNA, 20 bp, synthetic construct
  source 1..20: mol_type = other DNA
aatcgagaagcgactcgaca

SEQ ID NO: 426 - DNA, 20 bp, synthetic construct
  source 1..20: mol_type = other DNA
tgccctgcaggggagtgagc

SEQ ID NO: 427 - DNA, 20 bp, synthetic construct
  source 1..20: mol_type = other DNA
gaagcgactcgacatggagg

SEQ ID NO: 428 - DNA, 20 bp, synthetic construct
  source 1..20: mol_type = other DNA
cctgcaggggagtgagcagc

SEQ ID NO: 429-441 - skipped (13 entries)

SEQ ID NO: 442 - DNA, 20 bp, synthetic construct
  source 1..20: mol_type = other DNA
gtccctgggcgttgccctgc

SEQ ID NO: 443 - DNA, 20 bp, synthetic construct
  source 1..20: mol_type = other DNA
ccctgggcgttgccctgcag

SEQ ID NO: 444 - DNA, 20 bp, synthetic construct
  source 1..20: mol_type = other DNA
gagtgagcagctgtaagatt

SEQ ID NO: 445 - DNA, 20 bp, synthetic construct
  source 1..20: mol_type = other DNA
caggggagtgagcagctgta

SEQ ID NO: 446 - DNA, 20 bp, synthetic construct
  source 1..20: mol_type = other DNA
cgttgccctgcaggggagtg

SEQ ID NO: 447 - DNA, 20 bp, synthetic construct
  source 1..20: mol_type = other DNA
tgggcgttgccctgcagggg

SEQ ID NO: 448 - DNA, 20 bp, synthetic construct
  source 1..20: mol_type = other DNA
ttggtccctgggcgttgccc

SEQ ID NO: 449 - DNA, 20 bp, synthetic construct
  source 1..20: mol_type = other DNA
aagaatcccgcccataatcg

SEQ ID NO: 450 - DNA, 20 bp, synthetic construct
  source 1..20: mol_type = other DNA
aatcccgcccataatcgaga

SEQ ID NO: 451 - DNA, 20 bp, synthetic construct
  source 1..20: mol_type = other DNA
tcccgcccataatcgagaag

SEQ ID NO: 452 - DNA, 20 bp, synthetic construct
  source 1..20: mol_type = other DNA
cccataatcgagaagcgact

SEQ ID NO: 453 - DNA, 20 bp, synthetic construct
  source 1..20: mol_type = other DNA
gagaagcgactcgacatgga

SEQ ID NO: 454 - DNA, 20 bp, synthetic construct
  source 1..20: mol_type = other DNA
gcgactcgacatggaggcga

SEQ ID NO: 455 - DNA, 20 bp, synthetic construct
  source 1..20: mol_type = other DNA
ccgtgggaagataaactaat

SEQ ID NO: 456 - DNA, 20 bp, synthetic construct
  source 1..20: mol_type = other DNA
tcccctgcagggcaacgccc

SEQ ID NO: 457 - DNA, 20 bp, synthetic construct
  source 1..20: mol_type = other DNA
gtcgagtcgcttctcgatta

SEQ ID NO: 458 - DNA, 20 bp, synthetic construct
  source 1..20: mol_type = other DNA
cgacaccaactctagtccgt

SEQ ID NO: 459 - DNA, 20 bp, synthetic construct
  source 1..20: mol_type = other DNA
cagctgctcactcccctgca

SEQ ID NO: 460 - DNA, 20 bp, synthetic construct
  source 1..20: mol_type = other DNA
agtcgcttctcgattatggg

SEQ ID NO: 461 - DNA, 23 bp, synthetic construct
  source 1..23: mol_type = other DNA
acccttggaaggacctggctggg

SEQ ID NO: 462 - DNA, 20 bp, synthetic construct
  source 1..20: mol_type = other DNA
tccgagcttgacccttggaa

SEQ ID NO: 463 - DNA, 23 bp, synthetic construct
  source 1..23: mol_type = other DNA
ggagccacgaaaacagatccagg

SEQ ID NO: 464 - DNA, 23 bp, synthetic construct
  source 1..23: mol_type = other DNA
agatccagggacacggtgctagg

SEQ ID NO: 465 - DNA, 23 bp, synthetic construct
  source 1..23: mol_type = other DNA
gacacggtgctaggacagtgggg

SEQ ID NO: 466 - DNA, 23 bp, synthetic construct
  source 1..23: mol_type = other DNA
gaaaatgacccaacagcctctgg

SEQ ID NO: 467 - DNA, 23 bp, synthetic construct
  source 1..23: mol_type = other DNA
gcctggccggcctgaccactggg

SEQ ID NO: 468 - DNA, 23 bp, synthetic construct
  source 1..23: mol_type = other DNA
ctgagcactgaaggcctggccgg

SEQ ID NO: 469 - DNA, 23 bp, synthetic construct
  source 1..23: mol_type = other DNA
tggtttccactgagcactgaagg

SEQ ID NO: 470 - DNA, 23 bp, synthetic construct
  source 1..23: mol_type = other DNA
ggtgctttcctgaggaccgatag

SEQ ID NO: 471 - DNA, 23 bp, synthetic construct
  source 1..23: mol_type = other DNA
gcgcttccagtgctcagactagg

SEQ ID NO: 472 - DNA, 23 bp, synthetic construct
  source 1..23: mol_type = other DNA
cagtgctcagactagggaagagg

SEQ ID NO: 473 - DNA, 23 bp, synthetic construct
  source 1..23: mol_type = other DNA
gcccctcctccttcagagccagg

SEQ ID NO: 474 - DNA, 23 bp, synthetic construct
  source 1..23: mol_type = other DNA
tccttcagagccaggagtcctgg

SEQ ID NO: 475 - DNA, 23 bp, synthetic construct
  source 1..23: mol_type = other DNA
ccaagggtcaagctcggaaacca

SEQ ID NO: 476 - DNA, 23 bp, synthetic construct
  source 1..23: mol_type = other DNA
ctgcagagtatctgctggggtgg

SEQ ID NO: 477 - DNA, 23 bp, synthetic construct
  source 1..23: mol_type = other DNA
cgttcctgcagagtatctgctgg

SEQ ID NO: 478 - DNA, 20 bp, synthetic construct
  source 1..20: mol_type = other DNA
gtggggaaaatgacccaaca

SEQ ID NO: 479 - DNA, 20 bp, synthetic construct
  source 1..20: mol_type = other DNA
gaaggcctggccggcctgac

SEQ ID NO: 480 - DNA, 20 bp, synthetic construct
  source 1..20: mol_type = other DNA
actcctggctctgaaggagg

SEQ ID NO: 481 - DNA, 20 bp, synthetic construct
  misc_feature 1..20: note = Synthetic Sequence
  source 1..20: mol_type = other DNA
gggctgggggccaggactcc

SEQ ID NO: 482 - DNA, 20 bp, synthetic construct
  misc_feature 1..20: note = Synthetic Sequence
  source 1..20: mol_type = other DNA
gtccttccaagggtcaagct

SEQ ID NO: 483 - DNA, 20 bp, synthetic construct
  misc_feature 1..20: note = Synthetic Sequence
  source 1..20: mol_type = other DNA
tcaagctcggaaaccacccc

SEQ ID NO: 484-500 - skipped (17 entries)

SEQ ID NO: 501 - DNA, 1782 bp, synthetic construct
  source 1..1782: mol_type = other DNA
atgggctcttccttggacgacgagcacatcctgagcgccctgctgcagtccgatgacgagctggtgggtgaggactccgacagtgaggtgagcgatcatgtgtccgaggacgatgtgcagagcgacaccgaggaggccttcatcgatgaggtgcatgaagtgcagcccacatcctccggatccgagatcctggatgagcagaacgtgatcgagcagcccgggagctctctggcttccaacagaatcctgaccctgcctcagaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaaggcctccgcatcctgtaagaagtgcaagaaggtcatctgcagggaacacaatatcgacatgtgccagtcatgcttc

SEQ ID NO: 502 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 503 - DNA, 1713 bp, synthetic construct
  source 1..1713: mol_type = other DNA
atggactccgacagtgaggtgagcgatcatgtgtccgaggacgatgtgcagagcgacaccgaggaggccttcatcgatgaggtgcatgaagtgcagcccacatcctccggatccgagatcctggatgagcagaacgtgatcgagcagcccgggagctctctggcttccaacagaatcctgaccctgcctcagaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaaggcctccgcatcctgtaagaagtgcaagaaggtcatctgcagggaacacaatatcgacatgtgccagtcatgcttc

SEQ ID NO: 504 - protein, 571 aa, synthetic construct
MDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 505 - DNA, 1668 bp, synthetic construct
  source 1..1668: mol_type = other DNA
atgcagagcgacaccgaggaggccttcatcgatgaggtgcatgaagtgcagcccacatcctccggatccgagatcctggatgagcagaacgtgatcgagcagcccgggagctctctggcttccaacagaatcctgaccctgcctcagaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaaggcctccgcatcctgtaagaagtgcaagaaggtcatctgcagggaacacaatatcgacatgtgccagtcatgcttc

SEQ ID NO: 506 - protein, 556 aa, synthetic construct
MQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 507 - DNA, 1524 bp, synthetic construct
  source 1..1524: mol_type = other DNA
atgaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaaggcctccgcatcctgtaagaagtgcaagaaggtcatctgcagggaacacaatatcgacatgtgccagtcatgcttc

SEQ ID NO: 508 - protein, 508 aa, synthetic construct
MRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 509 - DNA, 1437 bp, synthetic construct
  source 1..1437: mol_type = other DNA
atgagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaaggcctccgcatcctgtaagaagtgcaagaaggtcatctgcagggaacacaatatcgacatgtgccagtcatgcttc

SEQ ID NO: 510 - protein, 479 aa, synthetic construct
MSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 511 - DNA, 1707 bp, synthetic construct
  source 1..1707: mol_type = other DNA
atgggctcttccttggacgacgagcacatcctgagcgccctgctgcagtccgatgacgagctggtgggtgaggactccgacagtgaggtgagcgatcatgtgtccgaggacgatgtgcagagcgacaccgaggaggccttcatcgatgaggtgcatgaagtgcagcccacatcctccggatccgagatcctggatgagcagaacgtgatcgagcagcccgggagctctctggcttccaacagaatcctgaccctgcctcagaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatgaaaaagaggacctactgcacctactgcccctcaaagatccgcaggaag

SEQ ID NO: 512 - protein, 569 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRK

SEQ ID NO: 513 - DNA, 1659 bp, synthetic construct
  source 1..1659: mol_type = other DNA
atgggctcttccttggacgacgagcacatcctgagcgccctgctgcagtccgatgacgagctggtgggtgaggactccgacagtgaggtgagcgatcatgtgtccgaggacgatgtgcagagcgacaccgaggaggccttcatcgatgaggtgcatgaagtgcagcccacatcctccggatccgagatcctggatgagcagaacgtgatcgagcagcccgggagctctctggcttccaacagaatcctgaccctgcctcagaggactatcagagggaaaaataagcactgctggtccacttcaaagccaaccaggcggtctagggtgtccgccctgaacatcgtgagaagccagagaggtccaacccggatgtgcagaaacatctacgatcccctgctgtgtttcaagctgttttttactgacgagattatcagcgagatcgtgaagtggaccaacgccgagatctctctgaagcggagagaaagcatgacctccgccacattccgcgataccaacgaagacgagatctacgcctttttcggcatcttggtgatgactgccgtgaggaaggacaatcacatgagcaccgatgacctgttcgatcgcagcctgagcatggtgtacgtgtccgtgatgtccagggacaggttcgacttcctgatcaggtgcctgagaatggatgacaagagcattagacctaccctgcgcgagaatgacgtgttcacccccgtcaggaagatctgggacctgtttattcaccagtgcattcagaactatacccctggcgcccacctgaccattgatgagcagctcctgggattcaggggccggtgtcccttcagagtgtatatacctaacaagccctctaagtacggcatcaaaatcctgatgatgtgcgactccggaaccaagtacatgattaacggaatgccctatctgggacgcgggacccagaccaatggagtgcctctgggcgagtattacgtgaaagagctgtccaaacccgtgcacgggtcctgtcggaatatcacctgcgacaactggttcacttcaatccccctggccaaaaacctgctgcaggagccttacaaattgaccattgtgggcactgtgcgctccaataagagagaaattcctgaggtgctgaagaactccaggtccagacccgtgggcactagcatgttctgcttcgacgggcctctgaccctggtgagctataagcccaagccagccaagatggtgtacctgctgagcagctgcgacgaggatgcctcaatcaacgagagcaccggcaagccccagatggtgatgtactacaatcagaccaaaggcggcgtggataccctggaccagatgtgttccgtgatgacctgtagcaggaagaccaaccggtggccaatggccctgctgtatggaatgatcaacattgcctgcatcaacagcttcattatttactcccacaatgtgtcctctaagggggagaaggtgcagtctagaaaaaagtttatgaggaatctgtatatgggcctgacctcttccttcatgcggaagcgcctggaggcacccacactgaagaggtaccttcgcgacaatatctccaatattctgccgaaagaggtgcccggcacctccgacgacagcacagaagagcccgtgatg

SEQ ID NO: 514 - protein, 553 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVM

SEQ ID NO: 515 - DNA, 720 bp, synthetic construct
  source 1..720: mol_type = other DNA
atggcgcctaagaagaagagaaaggtcggcggcctcgaaccaggcgagaagccttatgcctgtcctgagtgtggcaaatccttctcaagaaaagactctctggttagacaccagagaacacatacaggggagaaaccctataaatgccccgaatgcggaaagtccttttcccagagcggcgatctccggaggcatcagagaactcatacaggcgagaaaccatataagtgccccgagtgtgggaaatccttttccgattgtagagacctggccagacatcaaaggacacatacaggcaagaagaccgctagcggtggatccggagggtccggaggtagtggcggcagcctcgaggaagtccagctccaagaaagcggcgggggcctcgtgcagccaggcgggtccctgaggctgagctgtaccgcatctggagtcaccatctctgcccttaacgcaatggctatggggtggtatcggcaggcgcccggcgagaggagagtcatggtagcagccgttagcgaaagggggaatgcgatgtaccgagagagtgttcagggacgatttactgtcactcgggattttacgaacaaaatggtttcattgcaaatggacaatttgaaaccagaggacaccgctgtttactactgtcacgtcctggaagatcgagtagatagcttccatgactattggggccaaggaacacaagtgactgtcagctcc

SEQ ID NO: 516 - protein, 240 aa, synthetic construct
MAPKKKRKVGGLEPGEKPYACPECGKSFSRKDSLVRHQRTHTGEKPYKCPECGKSFSQSGDLRRHQRTHTGEKPYKCPECGKSFSDCRDLARHQRTHTGKKTASGGSGGSGGSGGSLEEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 517 - DNA, 1899 bp, synthetic construct
  source 1..1899: mol_type = other DNA
atggcgcctaagaagaagagaaaggtcggcggctccagactggaagaggaactgagaagaaggctcacagaagctagcggtggatccggagggtccggaggtagtggcggcagcctcgaggggtcttcactggacgatgagcatattctgagcgccctgctgcagagcgatgacgagctggtgggagaggattccgattccgaggtcagtgaccacgtgtcagaggacgatgtgcagagcgacactgaggaagccttcatcgatgaggtccatgaagtgcagccaacaagctccggaagcgagatcctggatgaacagaacgtgattgaacagcctggctctagtctggcttccaataggaacctgacactgccacagcgaactattcggggcaagaacaagcactgctggagcacctccaagcctacacggagaagccgcgcgtccgccctgaacatcgtgagatcccagagggggccaacccgcatgtgccgaaatatctacgaccccctgctgtgctttaagctgttctttacagatgagatcattagtgaaatcgtgaagtggactaacgcagagatttcactgaaaaggcgcgaatctatgactagtgccaccttcagagacacaaatgaggatgaaatctacgctttctttggcattctggtcatgaccgcagtgaggaaggacaaccatatgtctacagacgatctgtttgatcgctctctgagtatggtgtatgtctcagtgatgagcagagacaggttcgattttttgatccggtgcctgagaatggacgataagagcattcgacctacactgcgggagaatgacgtgttcaccccagtgaggaaaatctgggatctgtttatccaccagtgtattcagaactacacacccggagcccatctgactatcgacgaacagctgctgggcttccgcgggcgatgcccttttcgcgtatacattccaaataagcccagcaaatatggcatcaagattctgatgatgtgcgattccgggaccaaatacatgatcaacggaatgccatatctgggagccggcacccagacaaatggagtccccctgggcgagtactatgtgaaggaactgtccaaacctgtccacgggtcttgcagaaacatcacctgtgacaattggttcacatctattcccctggccaagaacctgctgcaggagccttataaactgactatcgtcggaaccgtggccagcaacaagagggagattcccgaagtgctgaagaacagccggagcagacctgtcggcacttctatgttctgctttgacgggccactgaccctggtgagttacaagcccaaacctgctaaaatggtgtatctgctgtcaagctgtgacgaggatgcaagcatcaatgaatccaccggcaagccccagatggtcatgtactataaccagactaaaggcggggtggataccctgaatcagatgtgctctgtcatgacctgtagtagaaagacaaacaggtggcctatggccctgctgtacgggatgatcaacattgcttgcattaattcattcatcatctacagccacaacgtgtcctctaagggggagaaagtccagtcccgcaagaaattcatgcgaaatctgtacatgggactgaccagtagcttcatgaggaagcgcctggaggcacccacactgaaaaggtatctgcgcgacaacatcagcaatattctgcctaaggaagtgccaggcacttccgacgattctaccgaggaaccagtgatgaagaaacggacatactgcacttattgtcccagcaagatccgacggaaagcctccgcttcttgcaagaagtgtaagaaagtgatctgtagagagcataacattgatatgtgccggtcctgtttt

SEQ ID NO: 518 - protein, 633 aa, synthetic construct
MAPKKKRKVGGSRLEEELRRRLTEASGGSGGSGGSGGSLEGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKPTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGAGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCRSCF

SEQ ID NO: 519 - DNA, 1875 bp, synthetic construct
  source 1..1875: mol_type = other DNA
atgccgaaaaaaaaacgaaaggtgtacccctacgatgtaccggactatgcaggaagcgggtcttcactggacgatgagcatattctgagcgccctgctgcagagcgatgacgagctggtgggagaggattccgattccgaggtcagtgaccacgtgtcagaggacgatgtgcagagcgacactgaggaagccttcatcgatgaggtccatgaagtgcagccaacaagctccggaagcgagatcctggatgaacagaacgtgattgaacagcctggctctagtctggcttccaataggaacctgacactgccacagcgaactattcggggcaagaacaagcactgctggagcacctccaagcctacacggagaagccgcgcgtccgccctgaacatcgtgagatcccagagggggccaacccgcatgtgccgaaatatctacgaccccctgctgtgctttaagctgttctttacagatgagatcattagtgaaatcgtgaagtggactaacgcagagatttcactgaaaaggcgcgaatctatgactagtgccaccttcagagacacaaatgaggatgaaatctacgctttctttggcattctggtcatgaccgcagtgaggaaggacaaccatatgtctacagacgatctgtttgatcgctctctgagtatggtgtatgtctcagtgatgagcagagacaggttcgattttttgatccggtgcctgagaatggacgataagagcattcgacctacactgcgggagaatgacgtgttcaccccagtgaggaaaatctgggatctgtttatccaccagtgtattcagaactacacacccggagcccatctgactatcgacgaacagctgctgggcttccgcgggcgatgcccttttcgcgtatacattccaaataagcccagcaaatatggcatcaagattctgatgatgtgcgattccgggaccaaatacatgatcaacggaatgccatatctgggagccggcacccagacaaatggagtccccctgggcgagtactatgtgaaggaactgtccaaacctgtccacgggtcttgcagaaacatcacctgtgacaattggttcacatctattcccctggccaagaacctgctgcaggagccttataaactgactatcgtcggaaccgtggccagcaacaagagggagattcccgaagtgctgaagaacagccggagcagacctgtcggcacttctatgttctgctttgacgggccactgaccctggtgagttacaagcccaaatccagactggaagaggaactgagaagaaggctcacagaacctgctaaaatggtgtatctgctgtcaagctgtgacgaggatgcaagcatcaatgaatccaccggcaagccccagatggtcatgtactataaccagactaaaggcggggtggataccctgaatcagatgtgctctgtcatgacctgtagtagaaagacaaacaggtggcctatggccctgctgtacgggatgatcaacattgcttgcattaattcattcatcatctacagccacaacgtgtcctctaagggggagaaagtccagtcccgcaagaaattcatgcgaaatctgtacatgggactgaccagtagcttcatgaggaagcgcctggaggcacccacactgaaaaggtatctgcgcgacaacatcagcaatattctgcctaaggaagtgccaggcacttccgacgattctaccgaggaaccagtgatgaagaaacggacatactgcacttattgtcccagcaagatccgacggaaagcctccgcttcttgcaagaagtgtaagaaagtgatctgtagagagcataacattgatatgtgccggtcctgtttt

SEQ ID NO: 520 - protein, 625 aa, synthetic construct
MPKKKRKVYPYDVPDYAGSGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKPTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGAGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKSRLEEELRRRLTEPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCRSCF

SEQ ID NO: 521 - DNA, 1875 bp, synthetic construct; identical to SEQ ID NO: 519
  source 1..1875: mol_type = other DNA

SEQ ID NO: 522 - protein, 137 aa, synthetic construct
MAPKKKRKVGGLEPGEKPYACPECGKSFSRKDSLVRHQRTHTGEKPYKCPECGKSFSQSGDLRRHQRTHTGEKPYKCPECGKSFSDCRDLARHQRTHTGKKTASGGSGGSGGSGGSLEEELLSKNYHLENEVARLKK

SEQ ID NO: 523 - DNA, 411 bp, synthetic construct
  source 1..411: mol_type = other DNA
atggcgcctaagaagaagagaaaggtcggcggcctcgaaccaggcgagaagccttatgcctgtcctgagtgtggcaaatccttctcaagaaaagactctctggttagacaccagagaacacatacaggggagaaaccctataaatgccccgaatgcggaaagtccttttcccagagcggcgatctccggaggcatcagagaactcatacaggcgagaaaccatataagtgccccgagtgtgggaaatccttttccgattgtagagacctggccagacatcaaaggacacatacaggcaagaagaccgctagcggtggatccggagggtccggaggtagtggcggcagcctcgaggaagaacttttgagcaagaattatcatcttgagaacgaagtggctcgtcttaagaaa

SEQ ID NO: 524 - protein, 137 aa, synthetic construct; identical to SEQ ID NO: 522

SEQ ID NO: 525 - DNA, 2598 bp, synthetic construct
  source 1..2598: mol_type = other DNA
atggcgcctaagaagaagagaaaggtcggcggcggacctgatattgtcatgacacaaagccctagctccctgagcgctagcgtgggcgatagagtcacaataacatgcagatccagcacaggcgctgtgactacgtccaattacgcttcttgggtccaggagaaacctggaaagctgttcaaagggctcataggaggcactaataatcgggcccctggcgtcccgtctagattttccggcagcctcattggggacaaggctaccctgaccattagctccctccaaccagaggacttcgctacgtatttttgtgctctgtggtattccaaccattgggtgtttggccaaggtactaaggtcgaactcaaacggggtggtggcgggtccggaggaggagggtctggaggaggggggtcctctggagggggcagcgaggttaagctcctggagtctggaggtgggcttgtccaacccggcggatcactgaaactgagctgcgctgtctccggtttttccctcaccgactacggggtgaactgggttcgccaggcgccaggccggggattggagtggattggtgtaatatggggtgatgggatcacagactacaacagcgctcttaaagatcggtttatcatctctaaagataatggcaagaatacggtctatctgcaaatgtctaaagtgagatccgacgacaccgccctctattactgtgtcaccggactcttcgactactggggccaaggcacactggtcacagtcagcagcgctagcggtggatccggagggtccggaggtagtggcggcagcctcgaggggtcttcactggacgatgagcatattctgagcgccctgctgcagagcgatgacgagctggtgggagaggattccgattccgaggtcagtgaccacgtgtcagaggacgatgtgcagagcgacactgaggaagccttcatcgatgaggtccatgaagtgcagccaacaagctccggaagcgagatcctggatgaacagaacgtgattgaacagcctggctctagtctggcttccaataggaacctgacactgccacagcgaactattcggggcaagaacaagcactgctggagcacctccaagcctacacggagaagccgcgcgtccgccctgaacatcgtgagatcccagagggggccaacccgcatgtgccgaaatatctacgaccccctgctgtgctttaagctgttctttacagatgagatcattagtgaaatcgtgaagtggactaacgcagagatttcactgaaaaggcgcgaatctatgactagtgccaccttcagagacacaaatgaggatgaaatctacgctttctttggcattctggtcatgaccgcagtgaggaaggacaaccatatgtctacagacgatctgtttgatcgctctctgagtatggtgtatgtctcagtgatgagcagagacaggttcgattttttgatccggtgcctgagaatggacgataagagcattcgacctacactgcgggagaatgacgtgttcaccccagtgaggaaaatctgggatctgtttatccaccagtgtattcagaactacacacccggagcccatctgactatcgacgaacagctgctgggcttccgcgggcgatgcccttttcgcgtatacattccaaataagcccagcaaatatggcatcaagattctgatgatgtgcgattccgggaccaaatacatgatcaacggaatgccatatctgggagccggcacccagacaaatggagtccccctgggcgagtactatgtgaaggaactgtccaaacctgtccacgggtcttgcagaaacatcacctgtgacaattggttcacatctattcccctggccaagaacctgctgcaggagccttataaactgactatcgtcggaaccgtggccagcaacaagagggagattcccgaagtgctgaagaacagccggagcagacctgtcggcacttctatgttctgctttgacgggccactgaccctggtgagttacaagcccaaacctgctaaaatggtgtatctgctgtcaagctgtgacgaggatgcaagcatcaatgaatccaccggcaagccccagatggtcatgtactataaccagactaaaggcggggtggataccctgaatcagatgtgctctgtcatgacctgtagtagaaagacaaacaggtggcctatggccctgctgtacgggatgatcaacattgcttgcattaattcattcatcatctacagccacaacgtgtcctctaagggggagaaagtccagtcccgcaagaaattcatgcgaaatctgtacatgggactgaccagtagcttcatgaggaagcgcctggaggcacccacactgaaaaggtatctgcgcgacaacatcagcaatattctgcctaaggaagtgccaggcacttccgacgattctaccgaggaaccagtgatgaagaaacggacatactgcacttattgtcccagcaagatccgacggaaagcctccgcttcttgcaagaagtgtaagaaagtgatctgtagagagcataacattgatatgtgccggtcctgtttt

SEQ ID NO: 526 - protein, 866 aa, synthetic construct
MAPKKKRKVGGGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSASGGSGGSGGSGGSLEGSSLDDEHILSALLQSDDELVGEDSDSEVSDHVSEDDVQSDTEEAFIDEVHEVQPTSSGSEILDEQNVIEQPGSSLASNRNLTLPQRTIRGKNKHCWSTSKPTRRSRASALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGAGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCRSCF

SEQ ID NO: 527 - DNA, 2598 bp, synthetic construct; identical to SEQ ID NO: 525
  source 1..2598: mol_type = other DNA

SEQ ID NO: 528 - protein, 866 aa, synthetic construct; identical to SEQ ID NO: 526

SEQ ID NO: 529 - DNA, 23 bp, synthetic construct
  source 1..23: mol_type = other DNA
gtgggttagggtggttaactggg

SEQ ID NO: 530 - DNA, 23 bp, synthetic construct
  source 1..23: mol_type = other DNA
aggtgcaaaaaggttgctgtggg

SEQ ID NO: 531 - DNA, 23 bp, synthetic construct
  source 1..23: mol_type = other DNA
cgtgacaaggcaaagtggcgtgg

SEQ ID NO: 532 - DNA, 23 bp, synthetic construct
  source 1..23: mol_type = other DNA
gaaggactgcccctgacgtcagg

SEQ ID NO: 533 - DNA, 23 bp, synthetic construct
  source 1..23: mol_type = other DNA
ctgcccctgacgtcaggagttgg

SEQ ID NO: 534 - DNA, 23 bp, synthetic construct
  source 1..23: mol_type = other DNA
tgtgggttagggtggttaactgg

SEQ ID NO: 535 - DNA, 23 bp, synthetic construct
  source 1..23: mol_type = other DNA
acccttttagagttttctgctgg

SEQ ID NO: 536 - DNA, 23 bp, synthetic construct
  source 1..23: mol_type = other DNA
aacttcctgccatggatgaaagg

SEQ ID NO: 537 - DNA, 23 bp, synthetic construct
  source 1..23: mol_type = other DNA
gcaaaaaggttgctgtgggttgg

SEQ ID NO: 538 - DNA, 23 bp, synthetic construct
  source 1..23: mol_type = other DNA
aatttgggggtagataggcatgg

SEQ ID NO: 539 - DNA, 23 bp, synthetic construct
  source 1..23: mol_type = other DNA
agaaaactctaaaagggtatagg

SEQ ID NO: 540 - DNA, 23 bp, synthetic construct
  source 1..23: mol_type = other DNA
attagcattcctttcatccatgg

SEQ ID NO: 541 - DNA, 23 bp, synthetic construct
  source 1..23: mol_type = other DNA
cccagcagaaaactctaaaaggg

SEQ ID NO: 542 - DNA, 23 bp, synthetic construct
  source 1..23: mol_type = other DNA
caggtgcaaaaaggttgctgtgg

SEQ ID NO: 543 - DNA, 23 bp, synthetic construct
  source 1..23: mol_type = other DNA
gcaagagatgaaattccatatgg

SEQ ID NO: 544 - DNA, 23 bp, synthetic construct
  source 1..23: mol_type = other DNA
gggctgttctaacgaagtctggg

SEQ ID NO: 545 - DNA, 23 bp, synthetic construct
  source 1..23: mol_type = other DNA
tgtccattcagcgaccctagagg

SEQ ID NO: 546 - DNA, 23 bp, synthetic construct
  source 1..23: mol_type = other DNA
ggctgttctaacgaagtctgggg

SEQ ID NO: 547 - DNA, 23 bp, synthetic construct
  source 1..23: mol_type = other DNA
gtccattcagcgaccctagaggg

SEQ ID NO: 548 - DNA, 23 bp, synthetic construct
  source 1..23: mol_type = other DNA
ggggctgttctaacgaagtctgg

SEQ ID NO: 549 - DNA, 23 bp, synthetic construct
  source 1..23: mol_type = other DNA
ggctgaatcagcatgcgaaaggg

SEQ ID NO: 550 - DNA, 23 bp, synthetic construct
  source 1..23: mol_type = other DNA
ttccaatggggggcatagcctgg

SEQ ID NO: 551 - DNA, 23 bp, synthetic construct
  source 1..23: mol_type = other DNA
taccctctagggtcgctgaatgg

SEQ ID NO: 552 - DNA, 23 bp, synthetic construct
  source 1..23: mol_type = other DNA
atcctcttgggccttataagagg

SEQ ID NO: 553 - DNA, 23 bp, synthetic construct
  source 1..23: mol_type = other DNA
ggccaggctatgccccccattgg

SEQ ID NO: 554 - DNA, 23 bp, synthetic construct
  source 1..23: mol_type = other DNA
ctagaggaccagaacaactctgg

SEQ ID NO: 555 - DNA, 23 bp, synthetic construct
  source 1..23: mol_type = other DNA
ctagaggaccagaacaactctgg

SEQ ID NO: 556 - DNA, 23 bp, synthetic construct
  source 1..23: mol_type = other DNA
aggctgaatcagcatgcgaaagg

SEQ ID NO: 557 - DNA, 23 bp, synthetic construct
  source 1..23: mol_type = other DNA
ggaccagaacaactctggcctgg

SEQ ID NO: 558 - DNA, 23 bp, synthetic construct
  source 1..23: mol_type = other DNA
gggcttttatttggcccagcagg

SEQ ID NO: 559 - DNA, 23 bp, synthetic construct
  source 1..23: mol_type = other DNA
gtcgctgaatggacagactctgg

SEQ ID NO: 560 - DNA, 23 bp, synthetic construct
  source 1..23: mol_type = other DNA
ctcatgagttttaccctctaggg

SEQ ID NO: 561 - DNA, 23 bp, synthetic construct
  source 1..23: mol_type = other DNA
tcctcttgggccttataagaggg

SEQ ID NO: 562 - DNA, 23 bp, synthetic construct
  source 1..23: mol_type = other DNA
tcttgggccttataagagggagg

SEQ ID NO: 563 - DNA, 23 bp, synthetic construct
  source 1..23: mol_type = other DNA
tagaacagccccccacacagtgg

SEQ ID NO: 564-583 - skipped (20 entries)

SEQ ID NO: 584 - DNA, 20 bp, synthetic construct
  source 1..20: mol_type = other DNA
tcgcccctcaaatcttacag

SEQ ID NO: 585 - DNA, 20 bp, synthetic construct
  source 1..20: mol_type = other DNA
tcaaatcttacagctgctca

SEQ ID NO: 586 - DNA, 20 bp, synthetic construct
  source 1..20: mol_type = other DNA
tcttacagctgctcactccc

SEQ ID NO: 587 - DNA, 20 bp, synthetic construct
  source 1..20: mol_type = other DNA
tacagctgctcactcccctg

SEQ ID NO: 588 - DNA, 20 bp, synthetic construct
  source 1..20: mol_type = other DNA
tgctcactcccctgcagggc

SEQ ID NO: 589 - DNA, 20 bp, synthetic construct
  source 1..20: mol_type = other DNA
tgcagggcaacgcccaggga

SEQ ID NO: 590 - DNA, 20 bp, synthetic construct
  source 1..20: mol_type = other DNA
tctcgattatgggcgggatt

SEQ ID NO: 591 - DNA, 20 bp, synthetic construct
  source 1..20: mol_type = other DNA
tcgcttctcgattatgggcg

SEQ ID NO: 592 - DNA, 20 bp, synthetic construct
  source 1..20: mol_type = other DNA
tgtcgagtcgcttctcgatt

SEQ ID NO: 593 - DNA, 20 bp, synthetic construct
  source 1..20: mol_type = other DNA
tccatgtcgagtcgcttctc

SEQ ID NO: 594 - DNA, 20 bp, synthetic construct
  source 1..20: mol_type = other DNA
tcgcctccatgtcgagtcgc

SEQ ID NO: 595 - DNA, 20 bp, synthetic construct
  source 1..20: mol_type = other DNA
tcgtcatcgcctccatgtcg

SEQ ID NO: 596 - DNA, 20 bp, synthetic construct
  source 1..20: mol_type = other DNA
tgatctcgtcatcgcctcca

SEQ ID NO: 597 - DNA, 20 bp, synthetic construct
  source 1..20: mol_type = other DNA
tggccggcctgaccactggg

SEQ ID NO: 598 - DNA, 20 bp, synthetic construct
  source 1..20: mol_type = other DNA
tgaaggcctggccggcctga

SEQ ID NO: 599 - DNA, 20 bp, synthetic construct
  source 1..20: mol_type = other DNA
tgagcactgaaggcctggcc

SEQ ID NO: 600 - DNA, 20 bp, synthetic construct
  source 1..20: mol_type = other DNA
tccactgagcactgaaggcc

SEQ ID NO: 601 - DNA, 20 bp, synthetic construct
  source 1..20: mol_type = other DNA
tggtttccactgagcactga

SEQ ID NO: 602 - DNA, 20 bp, synthetic construct
  source 1..20: mol_type = other DNA
tggggaaaatgacccaacag

SEQ ID NO: 603 - DNA, 20 bp, synthetic construct
  source 1..20: mol_type = other DNA
taggacagtggggaaaatga

SEQ ID NO: 604 - DNA, 20 bp, synthetic construct
  source 1..20: mol_type = other DNA
tccagggacacggtgctagg

SEQ ID NO: 605 - DNA, 20 bp, synthetic construct
  source 1..20: mol_type = other DNA
tcagagccaggagtcctggc

SEQ ID NO: 606 - DNA, 20 bp, synthetic construct
  source 1..20: mol_type = other DNA
tccttcagagccaggagtcc

SEQ ID NO: 607 - DNA, 20 bp, synthetic construct
  source 1..20: mol_type = other DNA
tcctccttcagagccaggag

SEQ ID NO: 608 - DNA, 20 bp, synthetic construct
  source 1..20: mol_type = other DNA
tccagcccctcctccttcag

SEQ ID NO: 609 - DNA, 20 bp, synthetic construct
  source 1..20: mol_type = other DNA
tggggtggtttccgagcttg

SEQ ID NO: 610 - DNA, 20 bp, synthetic construct
  source 1..20: mol_type = other DNA
tctgctggggtggtttccga

SEQ ID NO: 611 - DNA, 20 bp, synthetic construct
  source 1..20: mol_type = other DNA
tgcagagtatctgctggggt

SEQ ID NO: 612-631 - skipped (20 entries)

SEQ ID NO: 632 - DNA, 26 bp, synthetic construct
  source 1..26: mol_type = other DNA
tcttcctagtctcttctctacccagt

SEQ ID NO: 633 - DNA, 25 bp, synthetic construct
  source 1..25: mol_type = other DNA
tacactccagcctgggaaacagagt

SEQ ID NO: 634 - DNA, 19 bp, synthetic construct
  source 1..19: mol_type = other DNA
tcttttccttaggacggct

SEQ ID NO: 635 - DNA, 17 bp, synthetic construct
  source 1..17: mol_type = other DNA
tcgctcaggcctgtcat

SEQ ID NO: 636 - DNA, 16 bp, synthetic construct
  source 1..16: mol_type = other DNA
tccatatggaagactt

SEQ ID NO: 637 - DNA, 18 bp, synthetic construct
  source 1..18: mol_type = other DNA
tacccagttaaccaccct

SEQ ID NO: 638 - DNA, 27 bp, synthetic construct
  source 1..27: mol_type = other DNA
tggcgcatgcctgtaatcccagctact

SEQ ID NO: 639 - DNA, 26 bp, synthetic construct
  source 1..26: mol_type = other DNA
tatacgaggagaaaattagcattcct

SEQ ID NO: 640 - DNA, 23 bp, synthetic construct
  source 1..23: mol_type = other DNA
tctgcctcccaggttcacgcaat

SEQ ID NO: 641 - DNA, 22 bp, synthetic construct
  source 1..22: mol_type = other DNA
tgccttgtcacgttttcacagt

SEQ ID NO: 642 - DNA, 25 bp, synthetic construct
  misc_feature 1..25: note = Synthetic Sequence
  source 1..25: mol_type = other DNA
tgtcaccttctgtatgtgcaaccat

SEQ ID NO: 643 - DNA, 17 bp, synthetic construct
  misc_feature 1..17: note = Synthetic Sequence
  source 1..17: mol_type = other DNA
tctgtatgtgcaaccat

SEQ ID NO: 644 - DNA, 17 bp, synthetic construct
  misc_feature 1..17: note = Synthetic Sequence
  source 1..17: mol_type = other DNA
tagtcaagcaacaggat

SEQ ID NO: 645 - DNA, 18 bp, synthetic construct
  misc_feature 1..18: note = Synthetic Sequence
  source 1..18: mol_type = other DNA
tccaagataattccccat

SEQ ID NO: 646 - DNA, 16 bp, synthetic construct
  misc_feature 1..16: note = Synthetic Sequence
  source 1..16: mol_type = other DNA
tctgcaagatcctttt

SEQ ID NO: 647 - DNA, 29 bp, synthetic construct
  misc_feature 1..29: note = Synthetic Sequence
  source 1..29: mol_type = other DNA
tgctatgtaaggtagcaaaaaggtaacct

SEQ ID NO: 648 - DNA, 16 bp, synthetic construct
  misc_feature 1..16: note = Synthetic Sequence
  source 1..16: mol_type = other DNA
tctctctcctcctgct

SEQ ID NO: 649 - DNA, 19 bp, synthetic construct
  source 1..19: mol_type = other DNA
tccaaatgctattctctct

SEQ ID NO: 650 - DNA, 17 bp, synthetic construct
  source 1..17: mol_type = other DNA
tgctgattcagcctcct

SEQ ID NO: 651 - DNA, 21 bp, synthetic construct
  source 1..21: mol_type = other DNA
tagaacagccccccacacagt

SEQ ID NO: 652-799 - skipped (148 entries)

SEQ ID NO: 800 - DNA, 20 bp, synthetic construct
  source 1..20: mol_type = other DNA
ggagccacgaaaacagatcc

SEQ ID NO: 801 - DNA, 20 bp, synthetic construct
  source 1..20: mol_type = other DNA
ggagccacgaaaacagatcc

SEQ ID NO: 802 - DNA, 20 bp, synthetic construct
  source 1..20: mol_type = other DNA
agatccagggacacggtgct

SEQ ID NO: 803 - DNA, 20 bp, synthetic construct
  source 1..20: mol_type = other DNA
gacacggtgctaggacagtg

SEQ ID NO: 804 - DNA, 20 bp, synthetic construct
  source 1..20: mol_type = other DNA
gaaaatgacccaacagcctc

SEQ ID NO: 805 - DNA, 20 bp, synthetic construct
  source 1..20: mol_type = other DNA
gcctggccggcctgaccact

SEQ ID NO: 806 - DNA, 20 bp, synthetic construct
  source 1..20: mol_type = other DNA
ctgagcactgaaggcctggc

SEQ ID NO: 807 - DNA, 20 bp, synthetic construct
  source 1..20: mol_type = other DNA
tggtttccactgagcactga

SEQ ID NO: 808 - DNA, 20 bp, synthetic construct
  source 1..20: mol_type = other DNA
gatagccaggagtcctttcg

SEQ ID NO: 809 - DNA, 20 bp, synthetic construct
  source 1..20: mol_type = other DNA
gcgcttccagtgctcagact

SEQ ID NO: 810 - DNA, 20 bp, synthetic construct
  source 1..20: mol_type = other DNA
cagtgctcagactagggaag

SEQ ID NO: 811 - DNA, 20 bp, synthetic construct
  source 1..20: mol_type = other DNA
gcccctcctccttcagagcc

SEQ ID NO: 812 - DNA, 20 bp, synthetic construct
  source 1..20: mol_type = other DNA
tccttcagagccaggagtcc

SEQ ID NO: 813 - DNA, 20 bp, synthetic construct
  source 1..20: mol_type = other DNA
tggtttccgagcttgaccct

SEQ ID NO: 814 - DNA, 20 bp, synthetic construct
  source 1..20: mol_type = other DNA
ctgcagagtatctgctgggg

SEQ ID NO: 815 - DNA, 20 bp, synthetic construct
  source 1..20: mol_type = other DNA
cgttcctgcagagtatctgc